(19) United States
(12) Patent Application Publication
(10) Pub. No.: US 2017/0037465 A1
(43) Pub. Date: Feb. 9, 2017

(54) Methods for Nucleic Acid Base Determination

(71) Applicant: Dimitra Tsavachidou, Houston, TX (US)

(72) Inventor: Dimitra Tsavachidou, Houston, TX (US)

(21) Appl. No.: 15/298,092

(22) Filed: Oct. 19, 2016

Related U.S. Application Data
(63) Continuation of application No. PCT/US15/27686, filed on Apr. 26, 2015.

(51) Int. Cl. C12Q 1/68

(52) U.S. Cl. CPC C12Q 1/6869; C12Q 1/6806

(57) ABSTRACT

Methods for constructing tails, associating tails with nucleic acid molecules and attaching tail tags to nucleic acid molecules are disclosed. Methods for using tails and tail tags to perform sequencing of nucleic acid molecules are also disclosed. Tails and tail tags are constructs associated with nucleic acid molecules based on their nucleotide base composition. In many embodiments, a removable tail is associated with a nucleotide comprising a specific base type and incorporated into a nucleic acid molecule. The removable tail facilitates attachment of a tail tag to the nucleic acid molecule, said tail tag representing the base type of said nucleotide. Removal of the removable tail and repetition of the process generates a series of attached tail tags that represent the sequence of the nucleic acid molecule. The series of attached tail tags can be readily detected by nanopore devices, thus revealing the sequence of the nucleic acid molecule.

### RELATED APPLICATIONS

This application is a continuation of PCT/US15/27686, filed on Apr. 26, 2015, which claims the benefit of U.S. Provisional Application Ser. No. 61/985,097, filed on Apr. 28, 2014, and Ser. No. 62/099,962, filed on Jan. 5, 2015, which are incorporated by reference herein.

### SEQUENCE LISTING

The sequence listing, containing the file named 2016TSAV1019_ST25.txt which comprises the sequences described herein is 8 KB in size, was created on Oct. 19, 2016, and is hereby incorporated by reference in its entirety.

### FIELD

The methods provided herein relate to the field of nucleic acid sequencing.

### BACKGROUND

Nucleic acid sequence information is important for scientific research and medical purposes. The sequence information enables medical studies of genetic predisposition to diseases, studies that focus on altered genomes such as the genomes of cancerous tissues, and the rational design of drugs that target diseases. Sequence information is also important for genomic, evolutionary and population studies, genetic engineering applications, and microbial studies of epidemiologic importance. Reliable sequence information is also critical for paternity tests and forensics.

There is a constant need for new technologies that will lower the cost and increase the quality and amount of sequenced output. A promising technology that has the potential to revolutionize sequencing by simplifying the process and lowering the cost is nanopore-based detection. Nanopores are tiny holes that allow DNA translocation through them, which causes detectable disruptions in ionic current according to the sequence of the traversing DNA. Nanopore devices are able to differentiate between short DNA segments with distinct sequences, but they have difficulty performing sequencing at single-nucleotide resolution. Sequencing at single-nucleotide resolution is not feasible with solid-state nanopores, and is performed with reported error rates around 25-50% when using biological nanopores (Goodwin et al., 2015).

Problems arising from nanopore sequencing at single-nucleotide resolution can be circumvented by using expanded versions of nucleic acid molecules that can be readily detected by nanopore devices. Such expanded constructs preserve the sequence information of the nucleic acid molecules that they represent. Methods to generate expanded versions of nucleic acid molecules have been proposed previously, but they are difficult to use, because they are based on inefficient circularization steps (Lexow, 2008) (Buzby et al., 2012) (Meller and Weng, 2012), or on complex and inefficient hybridization steps, and expandable nucleotides with complex structures (Kokoris and McRuer, 2013).

### SUMMARY

The methods disclosed herein relate to nucleic acid sequencing. Methods for constructing tails, associating tails with nucleic acid molecules and attaching tail tags to nucleic acid molecules are disclosed. Methods for using tails and tail tags to perform sequencing of nucleic acid molecules are also disclosed. Tails and tail tags are constructs associated with nucleic acid molecules based on their nucleotide base composition.

Certain embodiments disclosed herein pertain to a method of associating a removable tail with a nucleotide comprising a predetermined base type, said removable tail not being associated with said nucleotide prior to its incorporation into a nucleic acid molecule, said method applied to one or more nucleic acid molecules, and said method comprising the steps of: (i) exposing a nucleic acid molecule comprising an extendable 3′ end to a solution and conditions to cause incorporation of a nucleotide comprising said predetermined base type into said nucleic acid molecule; (ii) subjecting said nucleic acid molecule to a process to cause association of a blocking tail with said nucleic acid molecule, said association occurring in the event that no incorporation occurs in step (i); and (iii) subjecting said nucleic acid molecule to a process to cause association of a removable tail with a nucleotide incorporated in step (i), said association occurring in the event that incorporation occurs in step (i).

In related embodiments, step (ii) precedes step (i); step (iii) is replaced by a step following step (ii) and preceding step (i), said step comprising subjecting the nucleic acid molecule to a process to cause association of a removable tail with the nucleic acid molecule, said association occurring in the event that no blocking tail is associated with the nucleic acid molecule in step (ii); and step (i) is conducted last and comprises subjecting the nucleic acid molecule to a process to cause removal of the removable tail that may be associated with the nucleic acid molecule, restoring the extendable 3′ end of the nucleic acid molecule, and exposing the nucleic acid molecule to a solution and conditions to cause incorporation of a nucleotide comprising a predetermined base type at said extendable 3′ end.

In other related embodiments, a removable nucleotide tail extending from the 3′ end of a nucleotide comprising a predetermined base type is constructed; and construction of a removable nucleotide tail in step (iii) is preceded by or concurrently conducted with unblocking in the event that the solution in step (i) comprises blocked nucleotides.

In other related embodiments, steps (i) and (ii) are conducted simultaneously; and the blocking nucleotide tail is constructed to comprise a single nucleotide that is blocked and cleavable.

In other related embodiments, the removable nucleotide tail is a ligatable removable nucleotide tail, and said embodiments further comprise step (iv) comprising a process to cause attachment of a tail tag to the nucleic acid molecule, said attachment occurring in the event that a ligatable removable nucleotide tail is constructed in step (iii), and said tail tag comprising one or more specific sequences, or one or more labels, or one or more other detectable features, or a combination thereof, designated to represent the predetermined base type in step (i).

Other related embodiments further comprise the steps of: (iv) detecting the presence of the removable nucleotide tail constructed in step (iii), and removing the blocking nucleotide tail that may be constructed in step (ii) and the removable nucleotide tail that may be constructed in step (iii); and (v) repeating steps (i) through (iv) at least one time, thereby allowing sequencing of the nucleic acid molecule.

In other related embodiments, the removable nucleotide tail is a ligatable removable nucleotide tail. Such embodiments further comprise step (iv) comprising a process to cause attachment of a tail tag to the nucleic acid molecule, said attachment occurring in the event that a ligatable removable nucleotide tail is constructed in step (iii), said step (iv) optionally conducted concurrently with step (iii), and said tail tag comprising one or more specific sequences, or one or more labels, or one or more other detectable features, or a combination thereof, designated to represent the predetermined base type in step (i).

In other related embodiments, step (ii) is omitted; and step (i) comprises exposing the nucleic acid molecule to conditions to cause nucleotide incorporation into said nucleic acid molecule, and to a polymerization reaction solution comprising a population of blocked nucleotides to complement the nucleic acid molecule, said population comprising: (a) nucleotides comprising one base type, that are reversibly blocked with a terminator type that is different from the types of terminators comprised in the nucleotides comprising other base types, and (b) one base type being a predetermined base type of step (i).

In other related embodiments, steps (i) and (ii) are conducted simultaneously; any constructed blocking nucleotide tail comprises a single nucleotide that is blocked and cleavable; and the combined steps (i) and (ii) comprise exposing the nucleic acid molecule to conditions to cause nucleotide incorporation into said nucleic acid molecule, and to a polymerization reaction solution comprising reversibly blocked nucleotides comprising a predetermined base type, and blocked cleavable nucleotides not comprising the predetermined base type.

In other related embodiments, the nucleic acid molecule comprises more than one extendable 3′ ends.

In other related embodiments, step (iv) is followed by steps (v) and (vi), said step (v) comprising subjecting the nucleic acid molecule to a process to cause removal of any nucleotide tails that may be constructed in previous steps, and said step (vi) comprising repeating steps (i) through (v) at least once.

In some related embodiments, tail tags comprise labels causing changes in conductivity or specific sequences causing changes in conductivity or both, and at least part of the nucleic acid molecule comprising tail tags passes through a nanopore of a nanopore device, thereby allowing detection of labels or specific sequences or both.

In some other related embodiments, tail tags comprise labels causing changes in conductivity or specific sequences causing changes in conductivity; the predetermined base type in step (i) is represented by at least two different label types or at least two different tail tag sequences; and at least part of the nucleic acid molecule comprising tail tags passes through a nanopore of a nanopore device, thereby allowing detection of labels or specific sequences.

In other related embodiments, step (ii) precedes step (i); step (ii) is preceded by a step comprising forming a single-base gap beginning at the extendable 3′ end of the nucleic acid molecule; and step (i) comprises exposing the nucleic acid molecule to conditions to cause nucleotide incorporation into said single-base gap.

In some other related embodiments, step (ii) precedes step (i); and step (ii) is followed by a step comprising subjecting the nucleic acid molecule to a process to cause formation of a single-base gap beginning at the extendable 3′ end of the nucleic acid molecule, said formation occurring in the event that there is no blocking nucleotide tail constructed in step (ii).

Other embodiments disclosed herein concern a method of of incorporating a nucleotide into a nucleic acid molecule comprising an extendable 3′ end, said nucleotide comprising a predetermined base type and a 3′ end suitable for constructing a removable nucleotide tail, said method applied to one or more nucleic acid molecules, and said method comprising the steps of: (i) exposing the nucleic acid molecule to conditions to cause nucleotide incorporation, and to a polymerization reaction solution comprising blocked nucleotides comprising a predetermined base type; (ii) subjecting the nucleic acid molecule to a process to cause construction of a blocking nucleotide tail extending from the extendable 3′ end of the nucleic acid molecule, said construction occurring in the event that no nucleotide incorporation occurs in step (i); and (iii) subjecting the nucleic acid molecule to a process to cause replacement of a blocked nucleotide by an unblocked nucleotide comprising the predetermined type of step (i), said replacement occurring in the event that nucleotide incorporation occurs in step (i), and said unblocked nucleotide maintaining an extendable 3′-end.

Still further, certain embodiments disclosed herein pertain to a method of constructing a removable nucleotide tail extending from the 3′ end of a nucleotide incorporated into a nucleic acid molecule, said nucleotide comprising a predetermined base type, said nucleic acid molecule comprising an extendable 3′ end, said method applied to one or more nucleic acid molecules, and said method comprising the steps of: (i) exposing the nucleic acid molecule to conditions to cause nucleotide incorporation, and to a polymerization reaction solution comprising cleavable nucleotides comprising a predetermined base type; (ii) subjecting the nucleic acid molecule to a process to cause a single cleavable nucleotide with extendable 3′ end to remain incorporated into the nucleic acid molecule, said nucleotide being incorporated during step (i); (iii) subjecting the nucleic acid molecule to a process to cause construction of a terminal blocking nucleotide tail, said construction occurring in the event that no nucleotide incorporation occurs in step (i); (iv) subjecting the nucleic acid molecule to a process to cause construction of a removable nucleotide tail extending from the 3′ end of the cleavable nucleotide in step (ii), said construction occurring in the event that nucleotide incorporation occurs in step (i); and (v) subjecting the nucleic acid molecule to a process to cause replacement of the cleavable nucleotide in step (ii) with a non-cleavable nucleotide, said replacement occurring in the event that nucleotide incorporation occurs in step (i).

In related embodiments, the removable nucleotide tail is ligatable, step (iv) is followed by a step comprising a process to cause tail tag ligation, said ligation occurring in the event that a ligatable removable nucleotide tail is constructed in step (iv), and the process of replacement in step (v) comprises gap formation and subsequent filling, and said tail tag comprising one or more specific sequences, or one or more labels, or one or more other detectable features, or a combination thereof, designated to represent the predetermined base type in step (i).

### BRIEF DESCRIPTION OF THE DRAWINGS

In the detailed description of various embodiments usable within the scope of the present disclosure, presented below, reference is made to the accompanying drawings, in which:

FIGS. 1A through 1C are schematic diagrams of methods for constructing removable nucleotide tails using single-nucleotide blocking nucleotide tails;

FIG. 2 is a schematic diagram of a method for the construction of a removable nucleotide tail;

FIG. 3 is a schematic diagram of a method for the construction of a removable nucleotide tail by template-independent polymerization;

FIG. 4 is a schematic diagram of a method for the construction of a removable nucleotide tail by template-dependent and template-independent polymerization;

FIGS. 5A through 5C are schematic diagrams of a method for replacing a removable nucleotide tail;

FIG. 6 is a schematic diagram of a method for replacing a removable nucleotide tail;

FIG. 7 is a schematic diagram of a method for constructing four different removable nucleotide tails;

FIGS. 8A and 8B are schematic diagrams of a method for constructing a removable nucleotide tail;

FIGS. 9A through 9C are schematic diagrams of a method for constructing a removable nucleotide tail;

FIG. 10 is a schematic diagram of a method for the attachment of a tail tag;

FIG. 11 is a schematic diagram of four tail tags;

FIGS. 12A through 12C are schematic diagrams of a method for attaching a protective tail tag and a tail tag to a nucleic acid molecule;

FIGS. 13A through 13C are schematic diagrams of a method for attaching a tail tag to a nucleic acid molecule with a previously attached tail tag;

FIG. 14 is a schematic diagram of a method for constructing a non-ligatable blocking nucleotide tail by using ligation;

FIGS. 15A and 15B are schematic diagrams of a method for attaching a tail tag to a nucleic acid molecule with a previously attached tail tag;

FIGS. 16A through 16C are schematic diagrams of a method for attaching tail tags to a nucleic acid molecule;

FIG. 17 is a schematic diagram of a hairpin tail tag attached to a nucleic acid molecule;

FIG. 18 is a schematic diagram of four tail tags;

FIG. 19 is a schematic diagram of two nucleic acid molecules with attached labeled tail tags;

FIG. 20 is a schematic diagram of a method for detecting tail tags using a nanopore device;

FIG. 21 is a schematic diagram of two nucleic acid molecules with attached tail tags;

FIGS. 22A through 22E are schematic diagrams of a method for attaching tail tags to a nucleic acid molecule;

FIG. 23 is a schematic diagram of a hairpin tail tag comprising a restriction endonuclease site;

FIG. 24 is a schematic diagram of a method for testing ribonucleotide incorporation by polymerases;

FIG. 25 shows the photographs of samples resolved using agarose gel electrophoresis; and

FIG. 26 shows the photographs of samples resolved using agarose gel electrophoresis.

### DETAILED DESCRIPTION

Methods disclosed herein can generate surrogates of nucleic acid molecules that comprise tail tags reliably detectable by nanopores. Each tail tag represents a specific nucleotide base. Tail tags can be short nucleic acid segments with distinct sequences, and are arranged in a surrogate in the order that their corresponding nucleotide bases appear in the nucleic acid molecule represented by the surrogate. Nanopore-based detection of tail tags in surrogates results in sequencing of the surrogates and consequently their corresponding nucleic acid molecules.

The sequential arrangement of tail tags is based on constructing removable tails. Removable tails can be associated with nucleic acid molecules in the event that incorporation of nucleotides comprising predetermined base types takes place. In several embodiments described herein, removable tails can be detected using nanopore devices or other detection methods, thus revealing the identities of the bases comprised in the incorporated nucleotides that said removable tails represent, and providing another way of sequencing in addition to detecting tail tags.

We show the particulars herein by way of example and for purposes of illustrative discussion of the embodiments. We present these particulars to provide what we believe to be the most useful and readily understood description of the principles and conceptual aspects of various embodiments of the disclosure. In this regard, we make no attempt to show structural details in more detail than is necessary for the fundamental understanding of the disclosed methods. We intend that the description should be taken with the drawings. This should make apparent to those skilled in the art how the several forms of the disclosed methods are embodied in practice.

### TERMS AND DEFINITIONS

We mean and intend that the following definitions and explanations are controlling in any future construction unless clearly and unambiguously modified in the following examples or when application of the meaning renders any construction meaningless or essentially meaningless. In cases where the construction of the term would render it meaningless or essentially meaningless, we intend that the definition should be taken from Webster's Dictionary 3rd Edition.

“Nucleotide” as used herein refers to a phosphate ester of a nucleoside, e.g., a mono-, or a triphosphate ester. A nucleoside is a compound consisting of a purine, deazapurine, or pyrimidine nucleoside base, e.g., adenine, guanine, cytosine, uracil, thymine, 7-deazaadenine, that can be linked to the anomeric carbon of a pentose sugar, such a ribose, 2′-deoxyribose, or 2′, 3′-di-deoxyribose. The most common site of esterification is the hydroxyl group connected to the C-5 position of the pentose (also referred to herein as 5′ position or 5′ end). The C-3 position of the pentose is also referred to herein as 3′ position or 3′ end. The term “deoxyribonucleotide” refers to nucleotides with the pentose sugar 2′-deoxyribose. The term “ribonucleotide” refers to nucleotides with the pentose sugar ribose. The term “dideoxyribonucleotide” refers to nucleotides with the pentose sugar 2′, 3′-di-deoxyribose.

A nucleotide may be incorporated and/or blocked and/or cleavable and/or otherwise modified, in the event that it is stated as such, or implied or allowed by context.

“Incorporated nucleotide”: A nucleotide that is stated to be incorporated into a nucleic acid molecule or nucleic acid construct (e.g., a nucleic acid extending strand, primer, blocking nucleotide tail, removable nucleotide tail, etc.), is a nucleotide having its 5′ end participating in a backbone bond in a nucleic acid molecule or nucleic acid construct. In the event that the incorporated nucleotide has a free 3′ end (e.g., said nucleotide is located at the 3′ end of a nucleic acid molecule, or at a nick or gap), said nucleotide is considered to have a hydroxyl group at the 3′ position that is capable of participating in backbone or other bonds, unless stated or implied otherwise.

Unless stated or implied otherwise, an “incorporated nucleotide” refers to a nucleotide that becomes part of a nucleic acid molecule via template-dependent polymerization.

Unless stated or implied otherwise, the term “incorporation” refers to the process of a nucleotide becoming part of a nucleic acid molecule via template-dependent polymerization.

The term “backbone bond” refers to the bond between the 3′ end of one nucleotide and the 5′ end of another nucleotide. The backbone bond is a phosphodiester bond in the event that a hydroxyl group and a phosphate group react to form the bond, or it can be another type of bond involving modified groups (e.g., a phosphorothioate bond).

The term “cleavable nucleotide” refers to a nucleotide that is capable of participating in backbone bonds that can be cleaved upon exposure to specific conditions and/or reagents including, but not limited to, enzymatic digestion, chemical treatment, etc. Cleavage may be specific to either the 5′ end of the cleavable nucleotide, or the 3′ end of the cleavable nucleotide, or both ends of the cleavable nucleotide.

Unless otherwise stated or implied, cleavable nucleotides can form backbone bonds, and be incorporated into nucleic acid molecules or constructs during polymerization reactions (template-dependent and -independent).

The type of a cleavable nucleotide depends on the context (i.e., the type of nucleic acid molecule the cleavable nucleotide interacts with). For example, ribonucleotides are suitable cleavable nucleotides when incorporated into DNA, and can be specifically cleaved from DNA by using ribonucleases, whereas using ribonucleases is not desirable in the event that ribonucleotides are incorporated into RNA.

“Blocking modification”, “block” or “terminator” refers to a molecule bound to, or a chemical modification applied to a nucleotide or nucleic acid molecule or nucleic acid construct, preventing the 3′ end of said nucleotide or nucleic acid molecule or construct from participating in the formation of a backbone bond during polymerization reactions. Such modification may be reversible or irreversible.

“Reversibly terminated” or “reversibly blocked” nucleotide is a nucleotide comprising a terminator (either at the 3′ end or elsewhere) that can be removed (e.g., cleaved, damaged, excised), restoring the ability of the 3′ end of said nucleotide to form a backbone bond in polymerization reactions. Unless stated or implied otherwise, a reversibly blocked (or reversibly terminated) nucleotide can be incorporated into a nucleic acid molecule or nucleic acid construct during a polymerization reaction. A reversibly blocked or terminated nucleotide that has its terminator or block removed is said to be “unblocked”. The process of removing a terminator may be referred to as “unblocking”. A removable terminator or removable blocking modification or block stated to be of different type from another terminator or blocking modification or block, is removed under different conditions (e.g., temperature, buffers, reagents, incubation time, UV exposure, enzymes) from the other terminator or blocking modification or block.

“Irreversibly terminated” or “irreversibly blocked” nucleotide is a permanently modified nucleotide that, when incorporated, does not allow further nucleotide incorporation in polymerization reactions. Unless stated or implied otherwise, an irreversibly blocked (or irreversibly terminated) nucleotide can be incorporated into a nucleic acid molecule or nucleic acid construct during a polymerization reaction. Non-limiting examples include dideoxyribonucleotides lacking 3′-OH, and acyclonucleotides.

A nucleic acid molecule or nucleic acid construct (tail, tail tag, etc.) or 3′ end of a nucleic acid molecule or nucleic acid construct is said to be “terminated”, when it cannot be extended by polymerization, said polymerization referring to either template-dependent polymerization or template-independent polymerization, or both. A non-limiting example includes the existence of a reversibly or irreversibly terminated nucleotide occupying the 3′ end of the nucleic acid molecule or construct. Other non-limiting examples include protruding or blunt 3′ ends, or 3′ ends that are not complementary to the template strand. These 3′ ends are “terminated” in the context of template-dependent polymerization, because they do not allow template-dependent polymerization to proceed, even though they may allow template-independent polymerization to proceed.

“Moiety” is one of two or more parts into which something may be divided, such as, for example, the various parts of a nucleotide, or a label in a labeled molecule.

The term “nucleotide type” refers to a category or population of nucleotide molecules having a certain common feature (e.g., base type, sugar type, modification) or combination of common features specific for that type.

A “nucleotide base” or “nucleoside base” is a heterocyclic base such as adenine, guanine, cytosine, thymine, uracil, inosine, xanthine, hypoxanthine, or a heterocyclic derivative, analog, or tautomer thereof, and can be naturally occurring or synthetic. The term “base type” refers to the kind of base comprised in a nucleotide (e.g., adenine, cytosine, guanine, uracil, thymine), whereas the term “base moiety” refers to the base itself, said base being part of a nucleotide molecule, and said nucleotide being unblocked or blocked, cleavable or non-cleavable, etc. Non-limiting examples of base types are adenine, guanine, thymine, cytosine, uracil, xanthine, hypoxanthine, 8-azapurine, purines substituted at the 8 position with methyl or bromine, 9-oxo-N6-methyl adenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring bases described in U.S. Pat. No. 5,432,272 (Benner and CH, 1995) and U.S. Pat. No. 6,150,510 (Seela and Thomas, 2000) and PCT applications WO 92/002258 (Cook and Sanghvi, 1992), WO 93/10820 (Froehler et al., 1993), WO 94/22892 (Cook and Delecki, 1994), and WO 94/24144 (Brian and Mark, 1995), and Fasman (“Practical Handbook of Biochemistry and Molecular Biology”, pp. 385-394, 1989, CRC Press, Boca Raton, La.) (Fasman, 1989), all herein incorporated by reference in their entireties.

The term “nucleotide comprising a predetermined base type” refers to a nucleotide comprising a base moiety of a specific base type which is selected and known in advance.

“Sequencing” refers to the determination of the type and relative position of at least two bases in a nucleic acid molecule.

“Complementary” generally refers to specific nucleotide duplexing to form canonical Watson-Crick base pairs, as is understood by those skilled in the art. For example, two nucleic acid strands or parts of two nucleic acid strands are said to be complementary or to have complementary sequences in the event that they can form a perfect base-paired double helix with each other.

“To complement a nucleic acid molecule” means to construct a segment complementary to the template strand of said nucleic acid molecule, said segment comprising one or more nucleotides.

The terms “hybridization” and “annealing” are used interchangeably and refer to non-covalent bonding through base pairing.

“Nucleic acid molecule” is a polymer of nucleotides consisting of at least two nucleotides covalently linked together. A nucleic acid molecule can be a polynucleotide or an oligonucleotide. A nucleic acid molecule can be deoxyribonucleic acid (DNA), ribonucleic acid (RNA), or a combination of both. A nucleic acid molecule may be single stranded or double stranded, as specified. A double stranded nucleic acid molecule may comprise non-complementary segments.

Nucleic acid molecules generally comprise phosphodiester bonds, although in some cases, they may have alternate backbones, comprising, for example, phosphoramide ((Beaucage and Iyer, 1993) and references therein; (Letsinger and Mungall, 1970); (Sprinzl et al., 1977); (Letsinger et al., 1986); (Sawai, 1984); and (Letsinger et al., 1988)), phosphorothioate ((Mag et al., 1991); and U.S. Pat. No. 5,644,048 (Yau, 1997)), phosphorodithioate (Brill et al., 1989), O-methylphosphoroamidite linkages (Eckstein, 1992), and peptide nucleic acid backbones and linkages ((Egholm et al., 1992); (Meier and Engels, 1992); (Egholm et al., 1993); and (Carlsson et al., 1996)). Other analog nucleic acids include those with bicyclic structures including locked nucleic acids, (Koshkin et al., 1998); positive backbones (Dempcy et al., 1995); non-ionic backbones (U.S. Pat. No. 5,386,023 (Cook and Sanghvi, 1992), U.S. Pat. No. 5,637,684 (Cook et al., 1997), U.S. Pat. No. 5,602,240 (Mesmaeker et al., 1997), U.S. Pat. No. 5,216,141 (Benner, 1993) and U.S. Pat. No. 4,469,863 (Ts'o and Miller, 1984); (von Kiedrowski et al., 1991); (Letsinger et al., 1988); (Jung et al., 1994); (Sanghvi and Cook, 1994); (De Mesmaeker et al., 1994); (Gao and Jeffs, 1994); (Horn et al., 1996)) and non-ribose backbones, including those described in U.S. Pat. No. 5,235,033 (Summerton et al., 1993) and U.S. Pat. No. 5,034,506 (Summerton and Weller, 1991), and (Sanghvi and Cook, 1994). Nucleic acids containing one or more carbocyclic sugars are also included within the definition of nucleic acids (Jenkins and Turner, 1995). Several nucleic acid analogs are described in Rawls, C & E News Jun. 2, 1997 page 35 (RAWLS, 1997).

All methods described herein to be performed on “a nucleic acid molecule”, can be applied to a single nucleic acid molecule, or more than one nucleic acid molecules. For example, said methods can apply to many identical nucleic acid molecules, such as PCR copies derived from a single nucleic acid molecule. In another example, said methods can also apply to many nucleic acid molecules of diverse sequences, such as extracted and sheared fragments of genomic DNA molecules. In another example, said methods can also apply to a plurality of groups of nucleic acid molecules, each group comprising copies of a specific nucleic acid molecule, such as the combination of products derived from multiple PCR assays. Examples mentioned above are non-limiting.

A nucleic acid molecule may be linked to a surface (e.g., functionalized solid support, adaptor-coated beads, primer-coated surfaces, etc.).

A “nucleic acid construct” refers in general to constructed oligonucleotides or polynucleotides, single-stranded or double-stranded, such as adaptors, tail tags, removable nucleotide tails, blocking nucleotide tails, etc.

Unless stated otherwise, a “nucleic acid molecule” that participates in reactions, or is said to be exposed to conditions or subjected to processes (or other equivalent phrase) to cause a reaction or event to occur, comprises the nucleic acid molecule and everything associated with it (sometimes referred to as “parts” or “surroundings”). Incorporated nucleotides, attached adaptors, hybridized primers or strands, attached tail tags, connected or constructed removable or blocking tails, etc., that are associated (e.g., bound, hybridized, attached, incorporated, ligated, etc.) with the nucleic acid molecule prior to or during a method described herein, are or become part of the nucleic acid molecule, and are comprised in the term “nucleic acid molecule”. For example, a nucleotide that is incorporated into the nucleic acid molecule in a step becomes part of the nucleic acid molecule in the next steps. For example, an adaptor that is already attached to the nucleic acid molecule prior to being subjected to methods described herein, is part of the nucleic acid molecule.

“Construction” of a tail refers to the gradual building of said tail starting from a nucleotide position or a position in a nucleic acid molecule and gradually adding said tail's components.

“Association” of a nucleotide or nucleic acid molecule with a tail refers to: (i) either constructing a tail starting from a nucleotide position or a position in a nucleic acid molecule and gradually adding said tail's components, (ii) or connecting a premade tail to a nucleotide or nucleic acid molecule. A non-limiting case of (i) is the construction of a removable nucleotide tail extending from the 3′ end of an incorporated nucleotide, said construction comprising the gradual incorporation of nucleotides that constitute said tail. A non-limiting case of (ii) is the ligation of an oligonucleotide to the 3′ end of a nucleic acid molecule, said oligonucleotide being complementary to the nucleic acid molecule, and constituting a blocking tail.

“Linker” is a molecule or moiety that joins two molecules or moieties or combinations thereof, and provides spacing between the two molecules or moieties such that they are able to function in their intended manner. Coupling of linkers to nucleotides and substrate constructs of interest can be accomplished through the use of coupling reagents that are known in the art (see, e.g., (Efimov et al., 1999)). Methods of derivatizing and coupling organic molecules are well known in the arts of organic and bioorganic chemistry. A linker may also be cleavable or reversible.

The term “adaptor” refers to an oligonucleotide or polynucleotide, single-stranded or double-stranded, of known sequence. Adaptors may include no sites, or one or more sites for restriction endonuclease recognition, or recognition and cutting.

The term “primer” refers to a single-stranded oligonucleotide or polynucleotide that comprises a free 3′-OH group and thus, when hybridized to a template strand, is capable of acting as a site of initiation of polymerization.

The term “polymerization” refers to the process of covalently connecting nucleotides to form a nucleic acid molecule (or a nucleic acid construct), or covalently connecting nucleotides via backbone bonds, one nucleotide at a time, to an existing nucleic acid molecule or a nucleic acid construct. The latter case is also termed “extension by polymerization”. Polymerization (extension by polymerization) can be template-dependent or template-independent. In template-dependent polymerization, the produced strand is complementary to another strand which serves as a template during the polymerization reaction, whereas in template-independent polymerization, addition of nucleotides to a strand does not depend on complementarity.

“Template strand”: As known by those skilled in the art, the term “template strand” refers to the strand of a nucleic acid molecule that serves as a guide for nucleotide incorporation into the nucleic acid molecule comprising an extendable 3′ end, in the event that the nucleic acid molecule is subjected to a template-dependent polymerization reaction. The template strand guides nucleotide incorporation via base-pair complementarity, so that the newly formed strand is complementary to the template strand.

“Extendable 3′ end” refers to a free 3′ end of a nucleic acid molecule or nucleic acid construct, said 3′ end being capable of forming a backbone bond with a nucleotide during template-dependent polymerization. “Extendable strand” is a strand of a nucleic acid molecule that comprises an extendable 3′ end.

A nucleic acid construct (such as a removable nucleotide tail) is said to “extend from a 3′ end”, in the case that said nucleic acid construct is constructed by polymerization starting at said 3′ end.

“Segment”: When referring to nucleic acid molecules, or nucleic acid constructs, “segment” is a part of a nucleic acid molecule (e.g., template strand) or a nucleic acid construct (e.g., removable nucleotide tail, tail tag, etc.) comprising at least one nucleotide.

Unless stated or implied otherwise, the term “filling” refers to the filling of a gap in a strand of a nucleic acid molecule or nucleic acid construct. Filling is accomplished by using polymerase molecules that do not displace or destroy the part of the strand following the gap. After completion, filling leaves a nick which can be sealed by ligation.

“Ligation” refers to the formation of backbone bonds between nucleotides in the same nucleic acid molecule (or nucleic acid construct) or different nucleic acid molecules or nucleic acid constructs or combinations thereof (e.g., a nucleic acid molecule and a tail tag) catalyzed by ligase, as known by those skilled in the art. “TA ligation” refers to the ligation of two double-strand ends, one comprising a single-nucleotide overhang containing adenine, and the other comprising a single-nucleotide overhang containing thymine.

Unless stated or implied otherwise, the terms “attached” and “ligated” have the same meaning and are used interchangeably.

The term “off-site extension by polymerization” or “off-site polymerization” refers to polymerization that initiates or continues from an undesirable position.

“First nucleotide” refers to a nucleotide whose 5′ end is the 5′ end of the strand or segment of a nucleic acid molecule or construct (e.g., template strand, removable nucleotide tail, etc.) said nucleotide belongs to.

“Last nucleotide” refers to a nucleotide whose 3′ end is the 3′ end of the strand or segment of a nucleic acid molecule or construct (e.g., template strand, removable nucleotide tail, etc.) said nucleotide belongs to.

“Excision” of a nucleotide refers to the cleavage of the backbone bond at the 3′ end of a nucleotide whose 5′ end is free, or the cleavage of the backbone bond at the 5′ end of a nucleotide whose 3′ end is free, or the cleavage of the backbone bonds at both ends of a nucleotide whose both ends participate in backbone bonds.

The term “removable tail” refers to a modification or construct that is: (a) associated with a nucleotide incorporated into a nucleic acid molecule, said nucleotide comprising a predetermined base type, or (b) associated with a nucleic acid molecule after said nucleic acid molecule fails to associate with a blocking tail. Examples include, but are not limited to, oligonucleotides capable of hybridizing to a nucleic acid molecule and being ligated to the 3′ end of an incorporated nucleotide comprising a predetermined base type. A removable tail may be unlabeled or comprise one or more labels.

The term “removable nucleotide tail” refers to a type of removable tail that is an oligo- or poly-nucleotide construct that extends from: (a) the 3′ end of a nucleotide comprising a predetermined base type that is incorporated into a nucleic acid molecule; or (b) the 3′ end of a nucleic acid molecule after a preceding process to construct a blocking nucleotide tail extending from said 3′ end does not produce a blocking nucleotide tail and leaves said 3′ end unaltered. At the time of incorporation, a nucleotide comprising a predetermined base type may be cleavable or not cleavable, modified or not modified, blocked or unblocked or not terminated. Said nucleotide is referred to as “the incorporated nucleotide”, and said nucleic acid molecule is referred to as “the nucleic acid molecule” in the following sentences describing removable nucleotide tails.

Processes to cause construction of a removable nucleotide tail comprise at least one step using extension by polymerization. A removable nucleotide tail comprises: a) one cleavable nucleotide bound to the extendable 3′ end of the incorporated nucleotide or the extendable 3′ end of the nucleic acid molecule, said cleavable nucleotide referred to as “first nucleotide”; b) no additional cleavable nucleotides, or one or more additional cleavable nucleotides of one or more types; c) no non-cleavable nucleotides, or one or more non-cleavable nucleotides located at any position after the first nucleotide; and d) an optionally terminated 3′ end.

“Non-cleavable” refers to nucleotides that are not cleaved when exposed to conditions and reagents that cleave the cleavable nucleotides in the removable nucleotide tail.

The term “ligatable removable nucleotide tail” refers to a removable nucleotide tail that renders a nucleic acid molecule capable of ligating to a tail tag (said nucleic acid molecule being without tail tags, or comprising previously attached tail tag or tail tags or protective tail tag or protective tail tags or combinations thereof). Said nucleic acid molecule is referred to as “the nucleic acid molecule” in the following sentences describing ligatable removable nucleotide tails.

Processes to cause construction of a ligatable removable nucleotide tail comprise using extension by polymerization to generate a removable nucleotide tail, and creating a ligatable end.

A process to cause construction of a ligatable removable nucleotide tail comprises at least one template-dependent polymerization reaction step. Additional steps may be included in said process, to generate a ligatable end, said end comprising the 5′ end of the template strand of the nucleic acid molecule, and the 3′ end of the ligatable removable nucleotide tail. For example, ligatable removable nucleotide tails participating in a TA ligation are subjected to incubation with Taq polymerase to add an adenine-comprising nucleotide as an overhang. In another example, incubation with T4 polynucleotide kinase is added to the process of constructing a ligatable removable nucleotide tail, to phosphorylate the 5′ end of the template strand of the nucleic acid molecule (in the event that it does not have a phosphate) so that it can successfully participate in a ligation reaction. Examples of methods constructing ligatable removable nucleotide tails include but are not limited to: (a) using template-dependent polymerization to construct a segment of cleavable nucleotides forming a blunt end suitable for blunt-end ligation; (b) using template-dependent polymerization to construct a segment of cleavable nucleotides reaching the end of the template strand of the nucleic acid molecule, and using Taq polymerase to create an overhang suitable for TA ligation; (c) using strand-displacing polymerases to displace parts of a previously constructed tail and the removable part of a previously attached tail tag, and constructing a segment of cleavable nucleotides reaching the end of the template strand of the nucleic acid molecule, and using Taq polymerase to create an overhang suitable for TA ligation; (d) using template-dependent polymerization to fully complement the template strand of the nucleic acid molecule, and using a restriction enzyme recognizing a site generated during construction of the ligatable removable nucleotide tail, to cleave a previously attached tail tag or protective tail tag in a manner that renders said tail tags' end ligatable. This can be accomplished for example, when the previously attached tail tag comprises a free 5′ end overhang comprising at least part of a restriction site. Since the at least part of said restriction site is not complementary to another strand, it cannot be recognized by its corresponding restriction endonuclease. During construction of the ligatable removable nucleotide tail, the at least part of said restriction site is fully complemented, thus rendered double-stranded and recognizable by the corresponding restriction endonuclease. Cutting by said restriction endonuclease generates an end that can be ligated to another tail tag comprising an appropriate end. Restriction sites can be, for example, asymmetric (e.g., site recognized by BbvCI).

The structure of a ligatable removable nucleotide tail is chosen based on the type of ligation and the structure of the tail tag to be ligated. For example, a removable nucleotide tail comprising a single-nucleotide overhang containing adenine is suitable for TA ligation of a tail tag containing a matching thymine-containing single-nucleotide overhang.

A “ligatable protective tail” is a special case of ligatable removable nucleotide tail, and it has the same features with a ligatable removable nucleotide tail, except that: (a) it is constructed in the event that a nucleotide comprising a predetermined base type is not incorporated into a nucleic acid molecule and a blocking nucleotide tail is not constructed, and: (b) it renders a nucleic acid molecule capable of ligating to a protective tail tag.

The term “blocking tail” refers to a modification or construct that is associated with a nucleic acid molecule comprising an extendable 3′ end, said tail being associated with said nucleic acid molecule in the event that no nucleotide comprising a predetermined base type can be incorporated at said extendable 3′ end in a template-dependent polymerization reaction, because of lack of complementarity. Said template-dependent polymerization reaction may precede or follow the process to cause association of said blocking tail with said nucleic acid molecule. A blocking tail may be unlabeled or comprise one or more labels.

The term “blocking nucleotide tail” refers to a type of blocking tail that is an oligo- or poly-nucleotide construct that extends from an extendable 3′ end of a nucleic acid molecule in the event that no nucleotide comprising a predetermined base type can be incorporated at said extendable 3′ end in a template-dependent polymerization reaction, because of lack of complementarity. A nucleotide comprising a predetermined base type may be non-cleavable or cleavable. Said nucleotide may be modified or not modified. Said nucleotide may be blocked or unblocked or not terminated. Said template-dependent polymerization reaction may precede or follow the process to cause construction of said blocking nucleotide tail. Said nucleic acid molecule is referred to as “the nucleic acid molecule” in the following sentences describing blocking nucleotide tails.

Processes to cause construction of a blocking nucleotide tail may comprise at least one step using extension by polymerization. A blocking nucleotide tail comprises: a) a terminated 3′ end; b) one cleavable nucleotide bound to the extendable 3′ end of the nucleic acid molecule, said nucleotide referred to as “first nucleotide”; c) no additional cleavable nucleotides, or one or more additional cleavable nucleotides of one or more types; and d) no non-cleavable nucleotides, or one or more non-cleavable nucleotides located at any position after the first nucleotide. A blocking nucleotide tail may also be constructed without extension by polymerization, but by sealing the extendable 3′ end of the nucleic acid molecule using ligation, thereby restoring a previously formed blocking nucleotide tail. This process may be referred to as “formation of blocking nucleotide tail by ligation”.

“Terminal blocking nucleotide tail” is a special case of a blocking nucleotide tail, which does not comprise cleavable nucleotides. A terminal blocking nucleotide tail prevents future formation (regeneration) of an extendable 3′ end in a nucleic acid molecule comprising said tail, thereby excluding said nucleic acid molecule from participating in future processes (e.g., construction of removable nucleotide tail, etc.). A terminal blocking nucleotide tail may participate in ligation to a tail tag, but it prevents participation in further ligations of other tail tags.

“Non-cleavable” refers to nucleotides that are not cleaved when exposed to conditions and reagents that cleave the cleavable nucleotides in the blocking nucleotide tail.

The term “non-ligatable blocking nucleotide tail” refers to a type of blocking nucleotide tail that prevents ligation of a tail tag to a nucleic acid molecule (said nucleic acid molecule being without tail tags, or comprising previously attached tail tag or tail tags or protective tail tag or protective tail tags or combinations thereof). Said nucleic acid molecule is referred to as “the nucleic acid molecule” in the following sentences describing non-ligatable blocking nucleotide tails.

A process to cause construction of a non-ligatable blocking nucleotide tail may comprise at least one polymerization reaction step. The process of constructing a non-ligatable blocking nucleotide tail results in the generation of a non-ligatable end, said end comprising the 5′ end of the template strand of the nucleic acid molecule, and the 3′ end of the non-ligatable blocking nucleotide tail. An end can become non-ligatable by either having a conformation that prevents ligation with a tail tag (for example, a non-ligatable blocking nucleotide tail with a recessive end cannot successfully participate in blunt ligation with a blunt-ended tail tag), or having a modified 3′ end (such as a dideoxyribonucleotide) or both.

A non-ligatable blocking nucleotide tail may also be constructed with no polymerization step, but by sealing the extendable 3′ end of the nucleic acid molecule using ligation, thereby restoring a previously formed non-ligatable blocking nucleotide tail. This process may be referred to as “formation of non-ligatable blocking nucleotide tail by ligation”.

Methods of constructing a non-ligatable blocking nucleotide tail include but are not limited to methods of using extension by polymerization to generate a blocking nucleotide tail with a non-ligatable 3′ end. Examples of these types of methods include: a) using template-dependent polymerization to construct a segment of cleavable nucleotides terminated by incorporating a dideoxyribonucleotide; b) using template-independent polymerization to construct a segment of cleavable nucleotides that is non-complementary to the template strand of the nucleic acid molecule; c) using strand-displacing polymerases to displace part of a partially removed, previously constructed tail and constructing a segment of cleavable nucleotides terminated by incorporating a dideoxyribonucleotide; and d) using template-dependent polymerization to fully complement the template strand of the nucleic acid molecule, and using a restriction enzyme recognizing a site generated during construction of the non-ligatable blocking nucleotide tail, to cleave a previously attached tail tag or protective tail tag in a manner that renders said tail tags' end non-ligatable.

Methods of constructing a non-ligatable blocking nucleotide tail also include methods of filling at least partially an excised part from a previously constructed tail ending at a non-ligatable end or associated with or attached to another construct ending at a non-ligatable end (e.g., a ligatable removable nucleotide tail attached to a tail tag, said tail tag comprising a free end that is non-ligatable; or a non-ligatable blocking nucleotide tail). Examples of these types of methods include: a) using polymerase molecules without strand-displacing and without 5′-to-3′ exonuclease activity to completely fill the gap previously generated by cleaving a segment comprising the first nucleotide of a previously constructed tail, and ligase molecules to seal the remaining nick; and b) using polymerase molecules without strand-displacing and without 5′-to-3′ exonuclease activity to fill a gap previously generated by cleaving a segment comprising the first nucleotide of a previously constructed tail, and then using polymerase molecules with strand-displacing or 5′-to-3′ exonuclease activity or both to incorporate an irreversibly terminated nucleotide.

Still further, methods of constructing a non-ligatable blocking nucleotide tail include methods of partially replacing part of a previously constructed tail, said part comprising at least the first nucleotide of the previously constructed tail, and said tail ending at a non-ligatable end or associated with or attached to another construct (such as a tail tag) ending at a non-ligatable end. An example is to incorporate a cleavable reversibly blocked nucleotide.

The term “removal” that pertains to a blocking or removable tail associated with a nucleic acid molecule or incorporated nucleotide, refers to at least the disassociation of said tails from said nucleic acid molecule or incorporated nucleotide (said nucleic acid molecule and said incorporated nucleotide may be referred to as “the nucleic acid molecule” and “the incorporated nucleotide” in the following sentences describing removal). For example, when the term “removal” pertains to a blocking nucleotide tail extending from the 3′ end of a nucleic acid molecule, said term refers to at least the cleavage of the backbone bond between the first nucleotide of the blocking nucleotide tail and the 3′ end of the nucleic acid molecule. When the term “removal” pertains to a removable nucleotide tail extending from the 3′ end of a nucleotide incorporated into a nucleic acid molecule, said term refers to at least the cleavage of the backbone bond between the first nucleotide of the removable nucleotide tail and the 3′ end of said incorporated nucleotide. When the term “removal” pertains to a removable nucleotide tail extending from the 3′ end of a nucleic acid molecule, said term refers to at least the cleavage of the backbone bond between the first nucleotide of the removable nucleotide tail and the 3′ end of said nucleic acid molecule.

“Removal” of a removable nucleotide tail or a blocking nucleotide tail may comprise one of the following: a) Cleavage of the backbone bond between the first nucleotide of the tail and the 3′ end of the nucleic acid molecule or incorporated nucleotide, said cleavage rendering said 3′ end extendable; b) same as (a), further comprising damaging or removing labels within the tail; c) same as (a), further comprising cleavage of at least one backbone bond within the tail; d) same as (b), further comprising cleavage of at least one backbone bond within the tail; e) cleavage of the backbone bond between the first nucleotide of the tail and the 3′ end of the nucleic acid molecule or incorporated nucleotide, said cleavage leaving said 3′ end non-extendable and said cleavage followed by a step to render the 3′ end extendable (for example, dephosphorylation of the 3′ end using CIP); f) same as (e), further comprising damaging or removing labels within the tail; g) same as (e), further comprising cleavage of at least one backbone bond within the tail; and h) same as (f), further comprising cleavage of at least one backbone bond within the tail.

In the event that at least part of the blocking or removable nucleotide tail remains hybridized (i.e., non-covalently bound through base pairing) to the nucleic acid molecule, said part can be replaced by a new tail. For example, as a new tail is constructed by extending from the 3′ end of the nucleic acid molecule, it displaces the previous. Such displacement can be achieved by using strand-displacing polymerases to construct the new tail. Another example includes digesting the hybridized part of the previous tail as the new tail is constructed. Such digestion can be achieved by using polymerases possessing 5′-to-3′ exonuclease activity to construct the new tail.

The term “ligatable 5′ end” or “ligatable 3′ end” refers to the 5′ or 3′ end of a nucleic acid molecule or a nucleic acid construct, said end being able to form a backbone bond in a ligation reaction, in the presence of a suitable ligation substrate and ligation conditions and reagents.

The term “ligatable end” refers to an end of a double-stranded nucleic acid molecule or nucleic acid construct, said end comprising the 5′ end of one strand and the 3′ end of its complementary strand, and said end being able to interact with another end, and participate successfully in a ligation reaction with said another end. An end is considered successfully ligated when only its 5′ end formed a new backbone bond, or when only its 3′ end formed a new backbone bond, or when both its 5′ and 3′ ends formed new backbone bonds.

In the context of a specific ligation reaction, the term “non-ligatable 3′ end” or “non-ligatable 5′ end” or “non-ligatable end” refers to a 3′ end or 5′ end or end that is modified (e.g., phosphorylated 3′ end), or does not have the appropriate conformation to interact with another ligation substrate (e.g., a protruding 3′ end whereas the other ligation substrate is blunt), or both, and is therefore unable to participate successfully in the ligation reaction.

Blunt end is an end of a double-stranded nucleic acid molecule or nucleic acid construct wherein neither the 5′ end nor the 3′ end is protruding.

Protruding 5′ or 3′ end, also referred to as overhang, is a non-complementary stretch in the end of a double-stranded nucleic acid molecule or nucleic acid construct comprising at least one unpaired nucleotide.

“Tail tags” are constructs that can ligate to a nucleic acid molecule (said nucleic acid molecule being without tail tags, or comprising previously attached tail tag or tail tags or protective tail tag or protective tail tags or combinations thereof), said nucleic acid molecule comprising a ligatable removable nucleotide tail or a terminal blocking nucleotide tail. A tail tag can ligate to the 5′ end of the template strand of said nucleic acid molecule, or to both the 5′ end of the template strand and the 3′ end of the ligatable removable nucleotide tail (or the terminal blocking nucleotide tail). A tail tag can be an oligonucleotide or polynucleotide, single-stranded or double-stranded, DNA or RNA or a combination thereof, that can ligate to a nucleic acid molecule as described. A tail tag comprises at least two nucleotides or base pairs, preferably at least eight nucleotides or base pairs. A tail tag may comprise modified nucleotides, such as labeled nucleotides, cleavable nucleotides, blocked nucleotides, etc. A tail tag may comprise modifications such as spacers. A tail tag may comprise recognition sites for restriction endonucleases.

A double-stranded tail tag comprises a strand that can ligate to the 5′ end of the template strand of a nucleic acid molecule, said strand termed the “remaining part”, and another strand that can optionally ligate to the 3′ end of the ligatable removable nucleotide tail comprised in the nucleic acid molecule, said strand termed the “removable part”. A single-stranded tail tag can ligate to the 5′ end of the template strand of a nucleic acid molecule, and is also termed the “remaining part”. A single-stranded tail tag may be a hairpin (a single strand with at least partial self-complementarity). A hairpin tail tag may ligate to the 5′ end of the template strand of a nucleic acid molecule and to the 3′ end of the ligatable removable nucleotide tail (or terminal blocking nucleotide tail) comprised in the nucleic acid molecule. Whole or part of a hairpin tail tag may become a “remaining part” during, for example, construction of a new ligatable removable nucleotide tail using a strand-displacing or a 5′-to-3′ exonuclease-comprising polymerase respectively.

A double-stranded tail tag may comprise non-complementary parts of strands, internally or at an end or both. A double-stranded tail tag may have blunt ends, or a blunt end and a 5′ end overhang comprising at least one nucleotide, or a blunt end and a 3′ end overhang comprising at least one nucleotide, or one 5′ end overhang comprising at least one nucleotide and a 3′ end overhang comprising at least one nucleotide, or two 5′ end overhangs comprising at least one nucleotide, or two 3′ end overhangs comprising at least one nucleotide.

Tail tags may comprise specific sequences, or labels, or other detectable features, or combinations thereof that are designated to represent specific nucleotide base types. A tail tag that represents a specific base type may be attached to a nucleic acid molecule in the event that a nucleotide comprising the specific base type is incorporated into the nucleic acid molecule. At the time of incorporation, said nucleotide may be cleavable or not cleavable, modified or not modified, blocked or unblocked or not terminated. Successive nucleotide incorporation events, each of which is followed by attachment of a tail tag that represents the base type of the incorporated nucleotide, leads to a series of tail tags attached in order reflecting the sequence of the nucleic acid molecule.

A tail tag that represents a specific base type may be attached to a nucleic acid molecule in the event that a ligatable removable nucleotide tail directly extends from an extendable 3′ end of the nucleic acid molecule. A tail tag that represents a specific base type may also be attached to a nucleic acid molecule before said nucleic acid molecule is subjected to processes to cause incorporation of a nucleotide comprising the specific base type represented by the tail tag. In this case, the attached tail tag (the remaining part) may participate in a future ligation to another tail tag only in the event that the nucleic acid molecule is eventually subjected to processes that cause incorporation of a nucleotide comprising the specific base type represented by the tail tag.

A “protective tail tag” is a special type of tail tag that, unlike tail tags, is attached to a nucleic acid molecule in the event that there is no incorporation of a nucleotide comprising a predetermined base type, said nucleic acid molecule comprising a ligatable protective tail. A protective tail tag may not represent a specific predetermined nucleotide base type.

For simplification, “tail tag” may refer to the remaining part of the tail tag attached to a nucleic acid molecule, depending on context.

The term “label” refers to a signaling element, molecular complex, compound, molecule, atom, chemical group, moiety or combinations thereof that, when linked (covalently, non-covalently, etc.) to nucleotides or polynucleotides or other molecules or constructs, render them directly or indirectly detectable using known detection methods, e.g., spectroscopic, photochemical, radioactive, biochemical, immunochemical, enzymatic, chemical or electrical methods. Exemplary labels include but are not limited to fluorophores, chromophores, radioisotopes, spin labels, enzyme labels, infrared labels, chemiluminescent labels and labels that alter conductivity. Methods of detecting such labels are well known to those of skill in the art.

A label or labels stated to be of different type from another label or labels, has different detection features from the other label or labels, so that said label or labels can be differentiated from the other label or labels upon detection.

The term “probes” refers to molecules or constructs that can bind to nucleic acid molecules or nucleic acid constructs (e.g., tail tags) in a specific way, enabling detection. For example, a probe is a labeled oligonucleotide that is complementary to the sequence of a tail tag.

### Nucleic Acid Molecules

Nucleic acid molecules can be obtained from several sources using methods known in the art.

In some embodiments, nucleic acid molecules of interest are genomic DNA molecules. Nucleic acid molecules can be naturally occurring or genetically altered or synthetically prepared.

In some embodiments, the nucleic acid molecules are mRNAs or cDNAs.

### Processing and Anchoring of Nucleic Acid Molecules

In some embodiments, the nucleic acid molecules are anchored to the surface of a substrate. Examples of relevant methods are described in U.S. Pat. No. 7,981,604 (Quake, 2011), U.S. Pat. No. 7,767,400 (Harris, 2010), U.S. Pat. No. 7,754,429 (Rigatti and Ost, 2010), U.S. Pat. No. 7,741,463 (Gormley et al., 2010) and WO 2010048386 A1 (Pierceall et al., 2010), included by reference herein in their entirety.

In some embodiments, the nucleic acid molecules are anchored to a surface prior to hybridization to primers or ligation to adaptors. In certain embodiments, the nucleic acid molecules are hybridized to primers first or ligated to adaptors first and then anchored to the surface. In still some embodiments, primers (or adaptors) are anchored to a surface, and nucleic acid molecules hybridize to the primers or attach to the adaptors. In some embodiments, the primer is hybridized to the nucleic acid molecule prior to providing nucleotides for the polymerization reaction. In some, the primer is hybridized to the nucleic acid molecule while the nucleotides are being provided. In still some embodiments, the polymerizing agent is immobilized to the surface.

Various methods known in the art can be used to anchor or immobilize the nucleic acid molecules or the primers or the adaptors to the surface of the substrate, such as, the surface of the synthesis channels or reaction chambers.

In some embodiments, the nucleic acid molecules are ligated to adaptors. Relevant methods are described in U.S. Pat. No. 7,741,463 (Gormley et al., 2010) and U.S. Pat. No. 7,754,429 (Rigatti and Ost, 2010), whose contents are incorporated herein by reference in their entirety. Adaptors can be ligated to nucleic acid molecules prior to anchoring to the solid support, or they may be anchored to the solid support prior to ligation to the nucleic acid molecule. The adaptors are typically oligonucleotides or polynucleotides (double stranded or single stranded) that may be synthesized by conventional methods. In some embodiments, adaptors have a length of about 10 to about 250 nucleotides. In certain embodiments, adaptors have a length of about 50 nucleotides. The adaptors may be connected to the 5′ and 3′ ends of nucleic acid molecules by a variety of methods (e.g. subcloning, ligation, etc). In order to initiate sequencing, an extendable 3′ end is formed in the nucleic acid molecule. One way is to denature the nucleic acid molecule linked to the adaptor and hybridize a primer that is complementary to a specific sequence within the adaptor. Another way is to create a nick in the nucleic acid molecule by using a restriction endonuclease that recognizes a specific sequence within the adaptor and cleaves only one of the strands. This can be accomplished, for example, by using a nicking endonuclease that has a non-palindromic recognition site. Suitable nicking endonucleases are known in the art. Nicking endonucleases are available, for example from New England BioLabs. Suitable nicking endonucleases are also described in (Walker et al., 1992); (Wang and Hays, 2000); (Higgins et al., 2001); (Morgan et al., 2000); (Xu et al., 2001); (Heiter et al., 2005); (Samuelson et al., 2004); and (Zhu et al., 2004), which are incorporated herein by reference in their entirety for all purposes. Additional methods and details can be found in U.S. Pat. No. 8,518,640 (Drmanac and Callow, 2013) and US 2013/0327644 (Turner and Korlach, 2013) which are included herein by reference in their entirety.

In another embodiment, the nucleic acid molecule is subject to a 3′-end tailing reaction. Example of this method is described in WO 2010/048386 A1 (Pierceall et al., 2010), which is referenced herein in its entirety. A poly-A tail is generated on the free 3′-OH of the nucleic acid molecule. The tail may be enzymatically generated using terminal deoxynucleotidyl transferase (TdT) and dATP. Typically, a poly-A tail containing 50 to 70 adenine-containing nucleotides is constructed. The poly-A tail facilitates hybridization of the nucleic acid molecule to poly-dT primer molecules anchored to a surface. In principle, nucleic acid molecule tailing can be carried out with a variety of dNTPs (or heterogeneous combinations), e.g., dATP. dATP can be used because TdT adds dATP with predictable kinetics useful to synthesize a 50-70 nucleotide tail. Similarly, RNA may be labeled with poly-A polymerase enzyme and ATP.

In some embodiments, the nucleic acid molecules are sequenced individually, as single molecules. In one embodiment, a single nucleic acid molecule is anchored to a solid surface and sequenced. In another embodiment, various nucleic acid molecules are anchored on a solid surface in conditions that allow individual single molecule sequencing. Examples of nucleic acid molecule concentrations and conditions allowing single molecule sequencing of multiple nucleic acid molecules are given in U.S. Pat. No. 7,767,400 (Harris, 2010). In another embodiment, one nucleic acid molecule is first amplified and then some of its copies are sequenced. In another embodiment, some nucleic acid molecules that are copies of the same nucleic acid molecule are amplified and sequenced. In another embodiment, various single nucleic acid molecules are first amplified forming distinct colonies or clusters and then sequenced simultaneously. Examples are described in U.S. Pat. No. 8,476,044 (Mayer et al., 2013) and US 2012/0270740 (Edwards, 2012), which are included herein as references in their entirety.

In some embodiments, nucleic acid molecules are anchored to surfaces that can be exposed to various sequencing reagents and washed in an automated manner. In other embodiments, nucleic acid molecules are anchored to surfaces that are housed in a flow chamber of a microfluidic device having an inlet and outlet to allow for renewal of reactants which flow past the immobilized moieties. Examples are described in U.S. Pat. No. 7,981,604 (Quake, 2011), U.S. Pat. No. 6,746,851 (Tseung et al., 2004), US 2013/0260372 (Buermann et al., 2013), and US 2013/0184162 (Bridgham et al., 2013), which are included herein as references in their entirety.

The methods described herein can apply to a single nucleic acid molecule or to more than one nucleic acid molecules. Methods to capture and handle individual nucleic acid molecules are known in the art. For examples, dilution methods are known that allow the presence of a single nucleic acid molecule inside a well, a microwell, a tube, a microtube, a nanowell, etc. Several methods are known that allow binding of a single nucleic acid molecule on a bead, on a well surface, etc. Methods are also known that allow single nucleic acid molecules to be linked onto a surface at a distance from other single nucleic acid molecules. Such single nucleic acid molecules can be, for example, detected by sensitive methods such as TIRF microscopy for the presence of labels, or they can be subjected to amplification leading to the formation of isolated clusters. Representative references describing methods using single nucleic acid molecules are the following: (Shuga et al., 2013); (Thompson and Steinmann, 2010); (Efcavitch and Thompson, 2010); (Hart et al., 2010); (Chiu et al., 2009); (Ben Yehezkel et al., 2008); (Metzker, 2010).

### Reversibly Blocked Nucleotides

In several embodiments, reversibly blocked deoxyribonucleotides are incorporated into nucleic acid molecules. Suitable reversibly blocked nucleotides include nucleotides carrying modifications at the 3′-OH group. Such nucleotides can still be recognized by polymerases and incorporated into the extending strand of the nucleic acid molecule, but their modifications act as terminators, blocking further elongation of the extending strand. The terminators are reversible and can be removed by chemical cleavage or photocleavage or other methods, leaving an intact 3′-OH. Examples include, but are not limited to, 3′-O-allyl-dNTPs and dNTPs with methoxymethyl (MOM) group at their 3′ end. These are described in (Metzker et al., 1994). Both terminators are chemically cleaved with high yield (Kamal et al., 1999); (Ireland and Varney, 1986). For example, the cleavage of the allyl group takes 3 minutes with more than 93% yield (Kamal et al., 1999), while the MOM group is reported to be cleaved with close to 100% yield (Ireland and Varney, 1986). Cleavage of the MOM group includes acid, whereas the cleavage of the terminator allyl group from the 3′-O-allyl-dNTPs includes Pd-catalyzed deallylation in aqueous buffer solution (Ju et al., 2006).

Another example of reversibly terminated nucleotides is the 3′-O-azidomethyl-deoxyribonucleotides (Guo et al., 2008). These nucleotides become unblocked by performing cleavage with phosphines (TCEP).

Another example of reversibly terminated nucleotides is the deoxyribonucleotides blocked with 3′-ONH2. Cleavage of this group and unblocking of the nucleotides is achieved by using mild nitrite and NaOAc buffers (Hutter et al., 2010).

Another example includes the 3′-O-(2-nitrobenzyl)-dNTPs. The photocleavable 2-nitrobenzyl moiety has been used to link biotin to DNA and protein for efficient removal by UV light (350 nm) ((Olejnik et al., 1995); (Olejnik et al., 1999); (Metzker et al., 1994)). A photolysis setup (described in U.S. Pat. No. 7,635,578 (Ju et al., 2009b)) can be used which allows a high throughput of monochromatic light from a 1000 watt high pressure xenon lamp (LX1000UV, ILC) in conjunction with a monochromator (Kratos, Schoeffel Instruments).

Other types of reversibly blocked nucleotides comprise terminators that are not connected to the 3′-OH but to other active groups in the molecule (Gardner et al., 2012).

Additional details for reversibly blocked nucleotides are provided in U.S. Pat. No. 7,635,578 (Ju et al., 2009b), US 2009/0263791 (Ju et al., 2009a); (Metzker, 2010); (Wu et al., 2007); (Metzker, 2005); (Ju et al., 2006); (Guo et al., 2008).

In certain embodiments, reversibly blocked cleavable nucleotides are useful to construct blocking nucleotide tails comprising a single nucleotide. In another embodiment, a reversibly blocked cleavable nucleotide comprising a predetermined base type is incorporated into a nucleic acid molecule, is unblocked and extended by a labeled removable nucleotide tail, thereby allowing sequencing. The nucleotide is then cleaved in order to allow re-sequencing of the same position that the nucleotide occupies in the nucleic acid molecule. Examples of such nucleotides include, but not limited to 2′-modified ribonucleotides (Gelfand and Gupta, 2012), 2′-nitrobenzyl-modified ribonucleotides (described in U.S. Pat. No. 8,299,226 (Piepenburg et al., 2012)), azidomethyl derivatives of ribonucleotides (Zavgorodny et al., 2000), or reversibly terminated phosphorothioate modified nucleotides (US 2013/0053252 (Xie et al., 2013)).

### Irreversibly Blocked Nucleotides

In certain embodiments, it is desired that blocking nucleotide tails or removable nucleotide tails or other constructs are blocked reversibly or irreversibly. Irreversible blocking is an option. In these cases, readily available nucleotides such as acyclonucleotides or dideoxyribonucleotides can be used (Barnes, 1987); (Gardner and Jack, 2002). In some embodiments, it is desirable that blocking nucleotide tails comprise a single terminated cleavable nucleotide. A non-limiting example is phosphorothioate-modified dideoxyribonucleotides, which are readily available by commercial manufacturers (p. ex.: TriLink Biotechnologies; 2′,3′-Dideoxyadenosine-5′-O-(1-Thiotriphosphate); 2′,3′-Dideoxycytidine-5′-O-(1-Thiotriphosphate); 2′,3′-Dideoxythymidine-5′-O-(1-Thiotriphosphate); 2′,3′-Dideoxyuridine-5′-O-(1-Thiotriphosphate); 2′,3′-Dideoxyguanosine-5′-O-(1-Thiotriphosphate)).

### Polymerases

Several polymerizing agents can be used in the polymerization reactions described herein. For example, depending on the nucleic acid molecule, a DNA polymerase, an RNA polymerase, or a reverse transcriptase can be used in template-dependent polymerization reactions. For template-independent polymerization reactions, terminal transferase (TdT) can be used. DNA polymerases and their properties are described in detail in (Kornberg and Baker, 2005). For DNA templates, many DNA polymerases are available. Examples include, but are not limited to, E. coli DNA polymerase I (Lecomte and Doubleday, 1983), Sequence 2.0®, T4 DNA polymerase or the Klenow fragment of DNA polymerase 1, T3, or Vent polymerase.

In some embodiments, thermostable polymerases are used, such as Therminator® (New England Biolabs), ThermoSequenase™ (Amersham) or Taquenase™ (ScienTech, St Louis, Mo.), Pyrococcus kodakaraensis KOD DNA polymerase (Takagi et al., 1997), JDF-3 DNA polymerase (from thermococcus sp. JDF-3; WO 01/32887 (Hansen et al., 2001)), Pyrococcus GB-D (PGB-D) DNA polymerase (also referred as Deep Vent® DNA polymerase; (Juncosa-Ginesta et al., 1994); New England Biolabs), Stoffel fragment, Vent®, and mutants, variants and derivatives thereof. Further examples include Pyrococcus furiosus (Pfu) DNA polymerase ((Lundberg et al., 1991); Stratagene), Pyrococcus woesei (Pwo) DNA polymerase ((Hinnisdaels et al., 1996); Boehringer Mannheim), Thermus thermophilus (Tth) DNA polymerase (Myers and Gelfand, 1991), Thermococcus litoralis (Tli) DNA polymerase (also referred to as Vent® DNA polymerase; (Cariello et al., 1991); New England Biolabs), 9° Nm® DNA polymerase (New England Biolabs), Thermotoga maritima (Tma) DNA polymerase (Diaz and Sabino, 1998), Thermus aquaticus (Taq) DNA polymerase (Chien et al., 1976), Tgo DNA polymerase (from thermococcus gorgonarius; Roche Molecular Biochemicals).

In some embodiments, polymerases which lack 3′-to-5′ exonuclease activity can be used (e.g., modified T7 DNA polymerase). The use of DNA polymerases lacking 3′-to-5′ exonuclease activity limits exonucleolytic degradation of the extending strand during sequencing in the absence of complementary dNTPs.

DNA polymerases lacking 3′-to-S′ exonuclease activity that have the ability to perform incorporation of ribonucleotides, dideoxyribonucleotides, modified nucleotides such as phosphorothioate-modified nucleotides or reversibly blocked nucleotides or nucleotides carrying labels, are used, for example, for the construction of removable nucleotide tails described herein. For example, some embodiments employ polymerizing agents that have increased ability to perform incorporation of modified, fluorophore-labeled nucleotides into a growing complementary strand. Examples of such polymerases have been described in U.S. Pat. No. 5,945,312 (Goodman and Reha-Krantz, 1999) and in US 2008/632,742 which is incorporated by reference herein. Procedures for selecting suitable nucleotide and polymerase combinations can be adapted from Ruth et al. (1981) Molecular Pharmacology 20:415-422 (Ruth and Cheng, 1981); (Chidgeavadze et al., 1984); (Chidgeavadze et al., 1985).

The ability of polymerases to perform incorporation of modified nucleotides such as ddNTPs and acyclic NTPs is described in (Gardner and Jack, 2002).

Mutants of native polymerases have been produced that are able to perform incorporation of ribonucleotides to DNA templates. These polymerases can perform incorporation of a limited number of ribonucleotides. For example, treatment with Vent polymerase variant A488L may result in incorporating 20 ribonucleotides, with incorporation beyond that point dropping dramatically (Gardner and Jack, 1999). Also, an experiment described in Example 9 herein showed that Therminator DNA polymerase performs ribonucleotide incorporation producing shorter extension products than the products produced during deoxyribonucleotide incorporation.

Therminator DNA polymerase is capable of performing modified nucleotide incorporation (such as acyclic nucleotides; data for acyclic nucleotide incorporation are available by the supplier, New England BioLabs, Inc., Ipswich, Mass.; https://www.neb.com/products/n0460-acyclonucleotide-set) and ribonucleotide incorporation.

Therminator III, 9° N DNA polymerase(exo-) A485L/Y409V and other mutants can perform incorporation of azidomethyl-dNTPs (Guo et al., 2008) (Bentley et al., 2008)(Gardner et al., 2012).

a-S-ddNTPs can be incorporated by Thermosequenase at 100 uM in an extension reaction. (Sauer et al., 2000).

Useful polymerases can be processive or non-processive. By processive is meant that a DNA polymerase is able to continuously perform incorporation of nucleotides using the same primer, for a substantial length without dissociating from either the extended primer or the template strand or both the extended primer and the template strand. In some embodiments, processive polymerases used herein remain bound to the template during the extension of up to at least 50 nucleotides to about 1.5 kilobases, up to at least about 1 to about 2 kilobases, and in some embodiments at least 5 kb-10 kb, during the polymerization reaction. This is desirable for certain embodiments, for example, where efficient construction of long removable nucleotide tails is performed.

In some embodiments, DNA polymerases are capable of displacing, either alone or in combination with a compatible strand displacement factor, a hybridized strand encountered during extension. The property of strand displacement is desirable for some embodiments, where segments from previous constructs (removable nucleotide tails, etc.) are removed and replaced.

In some embodiments, DNA polymerases possess 5′-to-3′ exonuclease activity, in order to remove parts of previous constructs, such as parts of removable nucleotide tails or blocking nucleotide tails.

In some embodiments, DNA polymerases that perform gap filling can be used. Such polymerases do not possess 5′-to-3′ exonuclease activity and do not cause strand displacement. Polymerases with these properties may exhibit 3′-to-5′ exonuclease activity (such as T4 and T7 DNA polymerases) or no exonuclease activity (such as Sulfolobus DNA polymerase IV)(Choi et al., 2011). Gap-filling polymerases such as T4 and T7 DNA polymerases can also perform incorporation of certain modified nucleotides, as a-S-dNTP (Yang et al., 2007)(Romaniuk and Eckstein, 1982)(R S Brody, 1982).

In certain embodiments that perform sequencing of RNA templates, reverse transcriptases can be used which include, but are not limited to, reverse transcriptases from HIV, HTLV-1, HTLV-II, FeLV, FIV, SIV, AMV, MMTV, MoMuLV and other retroviruses (Levin, 1997); (Verma, 1977); (Wu and Gallo, 1975).

Detailed descriptions of polymerases are found in US 2007/0048748 (Williams et al., 2007), U.S. Pat. No. 6,329,178 (Patel and Loeb, 2001), U.S. Pat. No. 6,602,695 (Patel and Loeb, 2003), U.S. Pat. No. 6,395,524 (Loeb et al., 2002), U.S. Pat. No. 7,981,604 (Quake, 2011), U.S. Pat. No. 7,767,400 (Harris, 2010), U.S. Pat. No. 7,037,687 (Williams et al., 2006), and U.S. Pat. No. 8,486,627 (Ma, 2013) which are incorporated by reference herein.

In certain embodiments using tail tags, the construction of new ligatable removable nucleotide tails can be performed with DNA polymerases that can use template strands comprising modified or labeled nucleotides (such as the remaining parts of tail tags that comprise labels). There are numerous DNA polymerases with this feature, such as Taq and Vent exo-polymerases, and polymerases used in commercially available PCR labeling kits.

In certain embodiments, parts of removable nucleotide tails comprising ribonucleotides are further extended using polymerases that can initiate polymerization from an RNA primer. There are numerous such polymerases, including, but not limited to, Bst and Bsu polymerases, E. coli DNA polymerase I, phi29 DNA polymerase, Therminator.

### Cleavable Nucleotides and Cleavage Reagents

Several constructs described herein, such as removable nucleotide tails, ligatable removable nucleotide tails, etc., comprise cleavable nucleotides that can be selectively removed enzymatically, or chemically, or by using photocleavage, or other methods. Examples of such nucleotides include, but are not limited to, ribonucleotides, phosphorothioate-modified nucleotides and phosphoroamidate-modified nucleotides. Representative examples and detailed descriptions are provided in U.S. Pat. No. 8,349,565 (Kokoris and McRuer, 2013), U.S. Pat. No. 5,380,833 (Urdea, 1995) and EP 1117838 B1 (Kawate et al., 2009).

In some embodiments, phosphorothioate-modified nucleotides can be used. Phosphorothioate-modified nucleotides can form phosphorothioate backbone bonds when participating in polymerization reactions. Such backbone bonds can be selectively cleaved by any number of techniques known to one skilled in the art, including, but not limited to, cleavage with metal cations (Mag et al., 1991); (Vyle et al., 1992); incubation with iodine in ethanol (Blanusa et al., 2010) or with iodoethanol (Gish and Eckstein, 1988).

In other embodiments wherein phosphoroamidate-modified nucleotides are used, removal of the phosphoroamidate-modified nucleotides can be achieved by cleaving the phosphoroamidate bond. Such selective cleavage can be accomplished, for example, by acid catalyzed cleavage (Mag and Engels, 1989); (Obika et al., 2007).

In embodiments wherein certain phosphorothioate-modified nucleotides or phosphoroamidate-modified nucleotides or combinations thereof or certain other modified nucleotides are used, the removal of the modified nucleotides may leave a phosphorylated 3′-end. The phosphorylated 3′-end can be dephosphorylated by incubating, for example, with alkaline phosphatase (such as calf intestinal (CIP) alkaline phosphatase or shrimp alkaline phosphatase (SAP), New England Biolabs), which removes the phosphate, rendering the 3′ end extendable.

In certain embodiments, ribonucleotides are used, that can be incorporated into DNA molecules and cleaved when needed, using ribonucleases or other methods such as alkaline hydrolysis or other chemical cleavage. Suitable chemical cleavage agents capable of selectively cleaving the phosphodiester bond between ribonucleotides or between a ribonucleotide and a deoxyribonucleotide include, but are not limited to, metal ions, for example rare-earth metal ions ((Chen et al., 2002); (Komiyama et al., 1999); U.S. Pat. No. 7,754,429 (Rigatti and Ost, 2010)), Fe(3) or Cu(3).

Unlike alkaline hydrolysis, lanthanides can be used for ribonucleotide cleavage at normal pH not causing denaturation of templates (Kamitani et al., 1998)(Matsumura and Komiyama, 1997).

Ribonucleases (RNases) are enzymes that catalyze the hydrolysis of RNA into smaller components. The RNases H are a family of ribonucleases which are present in all organisms examined to date. There are two primary classes of RNase H: RNase H1 and RNase H2. Retroviral RNase H enzymes are similar to the prokaryotic RNase H1. All of these enzymes share the characteristic that they are able to cleave the RNA component of an RNA/DNA hybrid double-stranded molecule (Cerritelli and Crouch, 1998). A third family of prokaryotic RNases has been proposed, rnhc (RNase H3)(Ohtani et al., 1999).

E. coli RNase H1 has been extensively characterized and prefers multiple RNA bases in the substrate for full activity. Full activity is observed with a stretch of at least four consecutive RNA bases within a double-stranded molecule (Hogrefe et al., 1990). An RNase H1 from Thermus thermophilus which has only 56% amino acid identity with the E. coli enzyme but which has similar catalytic properties (Itaya and Kondo, 1991).

The human RNase H1 gene (Type I RNase H) was cloned in 1998 (Cerritelli and Crouch, 1998); (Wu et al., 1998). This enzyme prefers a 5 base RNA stretch in DNA/RNA hybrids for cleavage to occur. Maximal activity is observed in 1 mM Mg++ buffer at neutral pH and Mn++ ions are inhibitory (Wu et al., 1999). Cleavage is not observed when 2′-modified nucleosides (such as 2′-OMe, 2′-F, etc.) are substituted for RNA.

The human Type II RNase H was first purified and characterized by Eder and Walder in 1991 (Eder and Walder, 1991). Unlike the Type I enzymes which are active in Mg++ but inhibited by Mn++ ions, the Type II enzymes are active with a wide variety of divalent cations. Optimal activity of human Type II RNase H is observed with 10 mM Mg++, 5 mM Co++, or 0.5 mM Mn++.

The E. coli RNase H2 gene has been cloned (Itaya, 1990) and characterized (Ohtani et al., 2000). Like the human enzyme, the E. coli enzyme functions with Mn++ions and is actually more active with manganese than magnesium.

RNase H2 genes have been cloned and the enzymes characterized from a variety of eukaryotic and prokaryotic sources. The RNase H2 from Pyrococcus kodakaraensis (KOD1) has been cloned and studied in detail (Haruki et al., 1998); (Mukaiyama et al., 2004). The RNase H2 from the related organism Pyrococcus furious has also been cloned but has not been as thoroughly characterized (Sato et al., 2003).

RNase HII creates a nick at the 5′ side of a single ribonucleotide embedded in a DNA strand, leaving 5′ phosphate and 3′ hydroxyl ends (Rydberg and Game, 2002); (Eder et al., 1993).

RNase HII can also digest the bonds in between multiple ribonucleotides that form an RNA segment in a DNA/RNA double-stranded hybrid molecule. In a previous study (Haruki et al., 2002), the authors have analyzed the cleavage specificities of various prokaryotic ribonucleases, including RNases HIT from Bacillus subtilis and Thermococcus kodakaraensis, on hybrid DNA/RNA substrates. Such RNases can cleave at the 5′ end of the first ribonucleotide of an RNA segment embedded in a double-stranded DNA/RNA hybrid molecule.

In a certain embodiment, ribonucleotides are used as cleavable nucleotides to construct blocking and removable nucleotide tails in DNA molecules. RNase HII is a suitable ribonuclease to use for cleavage, because of its ability to cleave the backbone bond connecting the 3′ end of a deoxyribonucleotide to the 5′ end of a ribonucleotide, leaving an extendable DNA 3′-end.

In some embodiments, it is desirable to completely remove a single ribonucleotide incorporated into a DNA strand. This is accomplished by the flap endonuclease FEN1, which acts in concert with RNase HII. In particular, this is a two-step process, with the bond at the 5′ side of the ribonucleotide being cleaved by RNase H2, and said ribonucleotide being excised by the flap endonuclease FEN1 (Sparks et al., 2012); (Rydberg and Game, 2002).

Since RNase HII usually does not remove the last ribonucleotide of an RNA segment within a DNA strand of a double-stranded hybrid molecule, this may need to be removed in certain embodiments by the action of a 5′-to-3′ exonuclease or by strand displacement during the construction of a new construct (e.g., removable nucleotide tail) during a following sequencing cycle.

5′-to-3′ exonucleases that can remove ribonucleotides include, but are not limited to, the Terminator 5′-phosphate-dependent RNA exonuclease (Epicentre, an Illumina company), RTH-1 nuclease (Turchi et al., 1994); (Huang et al., 1996), and RNases described previously (Ohtani et al., 2008); (Ohtani et al., 2004).

Ribonucleotide or ribonucleotides remaining at the 5′-end of the DNA segment of a construct such as a removable nucleotide tail can also be removed by DNA exonucleases such as the 5′-to-3′ DNA exonuclease T7 from T7 gene 6 (Shinozaki and Okazaki, 1978).

In some embodiments, removable nucleotide tails comprise a DNA segment following a segment comprising cleavable nucleotides. In some embodiments, 5′-to-3′ exonucleases such as T7 exonuclease can be used to remove the DNA segment. Such exonucleases require the existence of a free 5′-end (blunt or recessive). Such a free 5′-end is generated after removing the preceding segment comprising cleavable nucleotides as described above. In order to use 5′-to-3′ exonuclease, the 5′ ends of the primer strand and the nucleic acid template strand need to be protected in advance, by methods including, but not limited to, modifying the 5′-ends or ligating adaptors or hybridizing to primers, which include protruding 5′ ends, or phosphorothioate-modified deoxyribonucleotides (Nikiforov et al., 1994).

In certain embodiments, 3′-to-S′ exonucleases such as exonuclease III (Roychoudhury and Wu, 1977) can be used to remove a DNA segment of a removable nucleotide tail or other construct. In this case, it is useful to use phosphorothioate or other modified nucleotides to construct a first segment of the removable nucleotide tail. In such a setting, the removal of the removable nucleotide tail comprises incubating first with a 3′-to-S′ exonuclease, which removes the DNA segment of the removable nucleotide tail, but it is unable to digest the phosphorothioate-modified nucleotide segment of the removable nucleotide tail, thus protecting the extending strand of the nucleic acid molecule from destruction. Then, the phosphorothioate-modified nucleotide segment can be removed accordingly, with methods described herein. The Sp diastereomer of the phosphorothioate bond can inhibit digestion. Sp diastereomers of phosphorothioate nucleotides can be isolated using HPLC as described in U.S. Pat. No. 5,620,963 (Cook and Hoke, 1997).

### Tail Tag Construction

In several embodiments, tail tags are used that represent specific nucleotide base types and are attached to a nucleic acid molecule in order according to its sequence. In some embodiments, tail tags are double-stranded DNA molecules around 25 to 40 base pairs long. In some other embodiments, they are at least 8 base pairs long. In other embodiments, tail tags can be more than 40 base pairs long, and less than 500. Tail tags can have blunt ends, or 3′-end overhangs, or 5′-end overhangs, or combinations thereof.

Tail tags can be constructed using techniques known to those skilled in the art. For example, double-stranded tail tags comprising oligonucleotides can be constructed by first chemically synthesizing oligonucleotides of two sequences with at least partial complementarity, and annealing the oligonucleotides to produce double-stranded constructs. Chemical synthesis of oligonucleotides is well known and practiced (Brown, 1993), and is broadly available as a routine service provided by biochemical and chemical manufacturers (Sigma Aldrich, IDT, etc.). Annealing protocols are known to those skilled in the art. Software programs for designing oligonucleotides (calculation of annealing temperature, probability for self-annealing, etc.) are known and available (e.g., (Kibbe, 2007)). One skilled in the art can design complementary oligonucleotides that can form a dimer. Such double-stranded constructs can have a variety of features. For example, they can have specific sequences that can be recognized by labeled probes. In another example, tail tags have embedded amino-dT nucleotides that can easily link to labels such as fluorescent dyes, or they can comprise other modified nucleotides that either carry labels or can be linked to labels using known methods (Telser et al., 1989); (Agrawal); (Vaghefi, 2005). In another example, a tail tag has an adenine-containing overhang that can successfully participate in TA ligation.

Since there is a limit to the length of nucleic acid constructs that can be synthesized chemically (approximately 100 to 200 nucleotides long, depending on the method), other known methods can be used to construct tail tags of longer sizes. For example, oligonucleotides constructed individually by using automated solid-phase synthesizers, can be connected by annealing and standard ligation or polymerase reactions, in order to form longer nucleic acid constructs. Several such methods are used, such as the ligation of phosphorylated overlapping oligonucleotides (Khorana et al., 1972), the Fok I method (Mandecki and Bolling, 1988) and a modified form of ligase chain reaction for gene synthesis (Edge et al., 1981). Additionally, several PCR assembly approaches have been described, which usually use oligonucleotides of 40-50 nucleotides long that overlap each other. These oligonucleotides are designed to cover most of the sequence of both strands, and the full-length molecule is generated progressively by overlap extension (OE) PCR (Fuhrmann et al., 1999), thermodynamically balanced inside-out (TBIO) PCR (Gao et al., 2003) or combined approaches (Stemmer et al., 1995). Sizes can be from 200 to 1,200 base pairs, although longer constructs can also be made.

### Ligases

Tail tags can be attached to nucleic acid molecules by using ligation. Several types of ligases are suitable and used in embodiments. Ligases include, but are not limited to, NAD+-dependent ligases including tRNA ligase, Taq DNA ligase, Thermus filiformis DNA ligase, Escherichia coli DNA ligase, Tth DNA ligase, Thermus scotoductus DNA ligase, thermostable ligase, Ampligase thermostable DNA ligase, VanC-type ligase, 9° N DNA Ligase, Tsp DNA ligase, and novel ligases discovered by bioprospecting. Ligases also include, but are not limited to, ATP-dependent ligases including T4 RNA ligase, T4 DNA ligase, T7 DNA ligase, Pfu DNA ligase, DNA ligase 1, DNA ligase III, DNA ligase IV, and novel ligases including wild-type, mutant isoforms, and genetically engineered variants. There are enzymes with ligase activity such as topoisomerases (Schmidt et al., 1994).

### Labels

In several embodiments, nucleic acid constructs such as removable nucleotide tails and tail tags are labeled. Labels can be introduced to these constructs by, for example, including modified nucleotides comprising the labels. In the case of double-stranded oligonucleotide tail tags for example, including labeled nucleotides can be accomplished during chemical synthesis of the oligonucleotides forming the tail tags. In the case of removable nucleotide tails, labeled nucleotides can be incorporated during polymerization using appropriate polymerase molecules such as Taq polymerase and Vent exo-(Anderson et al., 2005). An appropriate mixture of labeled and unlabeled nucleotides is used in such polymerization reactions, with composition depending on the type of label. For example, a fluorescein-12-dUTP/unlabeled dTTP ratio of 1:3 is used in some embodiments, for polymerization-based labeling using fluorescein as the label.

Labels can also be linked to nucleic acid constructs either directly through modification of the nucleotides already contained in the construct, or indirectly. Such indirect labeling can include for example a labeled aptamer specifically recognizing and bound to a tail tag.

A “label” is a signaling element, molecular complex, compound, molecule or atom that has detection characteristics. Patents teaching the use of labels include but are not limited to U.S. Pat. No. 3,817,837 (Rubenstein and Ullman, 1974); U.S. Pat. No. 3,850,752 (Schuurs and Van, 1974); U.S. Pat. No. 3,939,350 (Kronick and Little, 1976); U.S. Pat. No. 3,996,345 (Ullman and Schwarzberg, 1976); U.S. Pat. No. 4,277,437 (Maggio, 1981); U.S. Pat. No. 4,275,149 (Litman et al., 1981); and U.S. Pat. No. 4,366,241 (Tom and Rowley, 1982).

In some embodiments, the tail tags comprise labeled nucleotide analogs. Such nucleotide analogs comprise labels connected to the base moiety of the nucleotide either directly or by using a linker (tether).

The tether is generally resistant to entanglement or is folded so as to be compact. Polyethylene glycol (PEG), polyethylene oxide (PEO), methoxypolyethylene glycol (mPEG), and a wide variety of similarly constructed PEG derivatives (PEGs) are broadly available polymers that can be utilized in several embodiments.

Labels and linkers included herewith are by no means limited to these groups of compounds.

In one embodiment, nucleic acid molecules are sequentially extended with tail tags and sequenced by passing through nanopore devices detecting changes in conductivity.

The tail tags can comprise nucleotides that are modified with the addition of PEG to their base moieties. PEG can be connected alone or in combination with another moiety such as biotin. Nucleotides that comprise biotin-PEG in various lengths of PEG are commercially available (e.g., Enzo Life Sciences) and they can be produced according to procedures found in US 2012/0252691 (Etienne et al., 2012). Experiments in US 2013/0264207 (Ju et al., 2013) and (Kumar et al., 2012) have shown that PEGs of various lengths connected to nucleotides yield distinct patterns of current blockade when passing through a nanopore. The current blockade that each PEG moiety yields is specific for the length and overall mass of that specific PEG moiety.

In another embodiment, the nucleic acid molecule is sequenced by using sequential excision and detection of the labels contained in the tail tags as they pass through the nanopore. Detecting cleaved labels using nanopores is described in US2013/0264207 (Ju et al., 2013) and (Kumar et al., 2012). Labels can be removed by excising the labeled nucleotides from the tail tags by using exonuclease (or other nuclease) digestion. The nuclease is anchored to the proximity of the opening of the nanopore, so that it sequentially removes nucleotides from the nucleic acid molecule and its tail tags and releases them inside the nanopore, where they can be detected by changes in conductivity.

The labels and linkers listed here are examples, and one skilled in the art can come up with a suitable linker-label combination which can be linked to the nucleotide and detected by nanopore devices.

### Label Removal

In some embodiments, labels comprised in some nucleic acid constructs such as removable nucleotide tails, are removed after detection. To facilitate removal of a label, a label may be linked to the nucleotide via a chemically or photochemically cleavable linker using methods such as those described by (Metzker et al., 1994) and (Burgess et al., 1997).

In a certain embodiment, labels in removable nucleotide tails are fluorescent and are photobleached after detection. Photobleaching can be performed according to methods, e.g., as described (Jacobson et al., 1983); (Okabe and Hirokawa, 1993); (Wedekind et al., 1994); and (Close and Anderson, 1973).

Another way of removing labels in nucleic acid constructs is to destroy the constructs themselves. Enzymatic digestion of removable nucleotide tails and other constructs is described elsewhere herein.

### Detection of Labeled Nucleic Acid Constructs

Any detection method may be used that is compatible with the type of label employed. Thus, examples include radioactive detection, optical absorbance detection, e.g., UV-visible absorbance detection, optical emission detection, e.g., fluorescence or chemiluminescence.

Single molecule detection can be achieved using flow cytometry where flowing samples are passed through a focused laser with a spatial filter used to define a small volume. U.S. Pat. No. 4,979,824 (Mathies et al., 1990) describes a device for this purpose. U.S. Pat. No. 4,793,705 (Shera, 1988) describes and claims in detail a detection system for identifying individual molecules in a flow train of the particles in a flow cell.

Detailed descriptions of example detection methods can be found in U.S. Pat. No. 7,767,400 (Harris, 2010), U.S. Pat. No. 8,530,154 (Williams, 2013), U.S. Pat. No. 7,981,604 (Quake, 2011), U.S. Pat. No. 8,436,999 (Pratt and Bryant, 2013), and U.S. Pat. No. 8,652,810 (Adessi et al., 2014), which are herein incorporated by reference in their entirety.

### Nanopore Devices

Nanopore devices are known in the art and nanopores and methods employing them are disclosed in U.S. Pat. No. 7,005,264 B2 (Su and Berlin, 2006); U.S. Pat. No. 7,846,738 (Golovchenko et al., 2010); U.S. Pat. No. 6,617,113 (Deamer, 2003); U.S. Pat. No. 6,746,594 (Akeson et al., 2004); U.S. Pat. No. 6,673,615 (Denison et al., 2004); U.S. Pat. No. 6,627,067 (Branton et al., 2003a); U.S. Pat. No. 6,464,842 (Golovchenko et al., 2002); U.S. Pat. No. 6,362,002 (Denison et al., 2002); U.S. Pat. No. 6,267,872 (Akeson et al., 2001); U.S. Pat. No. 6,015,714 (Baldarelli et al., 2000); U.S. Pat. No. 5,795,782 (Church et al., 1998); and U.S. Publication Nos. 2004/0121525 (Chopra et al., 2004), and 2003/0104428 (Branton et al., 2003b), each of which are hereby incorporated by reference in their entirety.

A “nanopore device” includes, for example, a structure comprising (a) a first and a second compartment (reservoir) separated by a physical barrier, which barrier has at least one pore with a diameter, for example, of from about 1 to 10 nm, and (b) an apparatus for applying an electric field across the barrier so that a charged molecule such as DNA, can pass from the first compartment through the pore to the second compartment. The nanopore device further comprises electrodes and a detection circuit for measuring changes in conductivity as molecules pass through the pore. The nanopore barrier may be synthetic or naturally occurring in part. Barriers can include, for example, lipid bilayers having therein a-hemolysin, oligomeric protein channels such as porins, synthetic peptides, etc. Barriers can also include inorganic sheets having one or more holes of a suitable size.

The application of a constant DC voltage between the two reservoirs of the nanopore device results in a baseline ionic current that is measured. In the event that an analyte is introduced into a reservoir, it may pass through the pore and change the observed current, due to a difference in conductivity between the electrolyte solution and analyte. The magnitude of the change in current depends on the volume of electrolyte displaced by the analyte while it is in the pore. The duration of the current change is related to the amount of time that the analyte takes to pass through the nanopore.

In the case of DNA translocation through a nanopore, the physical translocation is driven by the electrophoretic force generated by an applied DC voltage between the two reservoirs. See, e.g., (Riehn et al., 2005), which is incorporated herein by reference in its entirety. As DNA passes through the nanopore, the conductivity between the sensing electrodes is typically reduced as DNA is less conductive than the buffer solution (See (de Pablo et al., 2000), which is incorporated by reference in its entirety). When the passing DNA carries bulky modifications such as PEG, the conductivity changes further.

In some embodiments, nanopores in nanopore devices are biological nanopores (Hague et al., 2013b). Biological nanopores are protein channels embedded in planar lipid membranes, liposomes or polymer membranes that can be housed inside an electrochemical chamber. Large scale production and purification of various channel proteins are possible by using standard molecular biology techniques. Examples of protein channels include, but are not limited to, α-Hemolysin, MspA channel, and Phi29 connector channel.

In some cases, the nanopore can be a solid state nanopore. Solid state nanopores can be produced as described in U.S. Pat. No. 7,258,838 (Li et al., 2007). In some cases the nanopore comprises a hybrid protein/solid state nanopore in which a nanopore protein is incorporated into a solid state nanopore. Suitable nanopores are described, for example in (Mager and Melosh, 2008); (White et al., 2006); (Venkatesan et al., 2011). Suitable solid state nanopores are described in: (Storm et al., 2003); (Venkatesan et al., 2009); (Kim et al., 2006); (Nam et al., 2009) and (Healy et al., 2007) which are incorporated herein by reference in their entirety for all purposes.

In some cases, graphene can be used, as described in: (Geim, 2009); (Fischbein and Drndié, 2008).

Other nanopore structures include hybrid nanopores as described, for example, in US2010/0331194 (Turner et al., 2010); (Iqbal et al., 2007); (Wanunu and Meller, 2007); (Siwy and Howorka, 2010); (Kowalczyk et al., 2011); (Yusko et al., 2011); and (Hall et al., 2010) which are incorporated herein by reference in their entirety for all purposes.

Nanopores can also be linked to types of detectors other than electronic. For example, it has been shown that an optical detection system using CCD camera can detect fluorescent dyes bound to DNA as it passes through a nanopore (Atas et al., 2012).

In one embodiment, tail tags attached to a nucleic acid molecule are labeled with fluorescent labels. Specifically, the remaining part of each tail tag carries a combination of fluorescent labels that uniquely corresponds to a single base type. For example, the remaining part of one tail tag type carries the combination Atto647 (A647) and Atto680 (A680), another tail tag type carries the combination A680 and A647, another tail tag type carries two A680 labels, another tail tag type carries two A647 labels. The nucleic acid molecule passes through a less than 2 nm-wide solid-state nanopore and splits into two strands of which only one passes through the nanopore. The procedure of DNA unzipping by passing through a nanopore is described in (McNally et al., 2008). In the event that the labeled strand passes through the nanopore, the fluorescent labels can be detected using methods described in (Atas et al., 2012).

In another embodiment, the nanopore system that is used to detect tail tags is a silicon nitride (SiNx) solid-state nanopore described in (Venta et al., 2013). This type of nanopore can detect changes in conductivity caused by single-stranded DNA homopolymer sequences of 30 bases long. The remaining parts of the tail tags used in this embodiment are designed to be at least 30 bases long, preferably 50 bases long. Said parts comprise a middle section comprising a homopolymer sequence 30 bases long having either adenine, or cytosine, or thymine, or guanine. Said middle section is flanked by 10-base-long sequences that comprise the appropriate ends for ligation of the tail tag to a nucleic acid molecule. Nucleic acid molecules that have such tail tags attached are denatured using methods known to those skilled in the art, to produce two single strands for each nucleic acid molecule that can pass through the nanopore.

In another embodiment, the nanopore system used to detect tail tags attached to nucleic acid molecules is a phi29 nanochannel that is 3.6 nm-wide and allows double-stranded DNA to pass through (Hague et al., 2013a). Tail tags used in this system can comprise stretches of homopolymer sequences. These can be detected, as double-stranded DNA attached to such tail tags passes through the nanochannel. In a similar embodiment, tail tags further comprise labels that are bulky enough to cause changes in conductivity as they pass through the pore. Non-limiting examples of such labels include biotin, PEG, etc., as described in (Kumar et al., 2012).

In a certain embodiment, the nanopore device combines the highly sensitive mutated form of the protein pore Mycobacterium smegmatis porin A (MspA) with phi29 DNA polymerase (DNAP), which controls the rate of DNA translocation through the pore (described in detail in (Manrao et al., 2012)). As phi29 DNAP synthesizes DNA, it functions like a motor to pull a single-stranded template through MspA. As the DNA molecule passes through, changes in conductivity are recorded. This nanopore device has difficulty detecting individual bases within DNA molecules, but can differentiate between very short motifs (for example 3 or 4 bases long). Short-sized tail tags that are long enough to be differentiated from one another are particularly useful in this embodiment.

### Data Analysis

Analysis of the data generated by the methods described herein is generally performed using software and/or statistical algorithms that perform various data conversions, e.g., conversion of signal emissions into basecalls. Such software, statistical algorithms, and use thereof are described in detail, e.g., in U.S. Patent Publication No. 2009/0024331 (Tomaney et al., 2009) and U.S. Pat. No. 8,370,079 (Sorenson et al., 2013).

### Sequencing of Nucleic Acid Molecules and De Iection of Tail Tags Using Probes

In other embodiments, one or more nucleic acid molecules comprise multiple extendable 3′ ends. For example, single-stranded DNA molecules of 1 kb or more are subjected to poly-A tailing with terminal transferase, and hybridized to oligo-dT primers anchored to a solid support. The DNA molecules are subjected to a polymerization reaction that extends the primers using a mixture of deoxyribonucleotides and dUTP (for example, dUTP:dTTP ratio of 1:25) or ribonucleotides or other cleavable nucleotides, and long-range polymerase molecules, such as long-range Taq from New England BioLabs, that maximizes the length of the produced strands. Incubation with UDG/EndoIV or ribonucleases or appropriate cleavage agents generates dispersed nicks or gaps throughout the strand. The benefit of this method is that all the nicks or gaps are generated in one strand only, so double strand breaks during extension are avoided.

DNA molecules comprising multiple extendable 3′ ends can be subjected to a process of constructing labeled removable nucleotide tails extending from nucleotides incorporated into each 3′ end according to the specific base types of the incorporated nucleotides. Detection of the labeled removable nucleotide tails can be achieved by methods that stretch the labeled DNA molecules on a surface and detect the type of labels and the order they are arranged in the DNA molecules, thereby allowing sequencing of the locations near the 3′ ends.

In other embodiments, tail tags are attached to nucleic acid molecules, said tags comprising specific sequences that can be recognized and bound by labeled probes. Suitable probe construction (such as labeled oligonucleotides complementary to tail tag sequences) and hybridization techniques are well known to those skilled in the art. Stretching the nucleic acid molecules comprising tail tags enables detection of the labeled probes in the order their matched tail tags are arranged, thereby allowing sequencing.

Methods of immobilizing nucleic acid molecules, stretching them and orienting them onto a surface, and detecting labeled segments arranged in a particular order are known in the art (see U.S. Pat. No. 8,415,102 (Geiss et al., 2013)).

In certain embodiments, nucleic acid molecules can be stretched, or oriented, or both, in an electric or magnetic field. The field is strong enough to stretch or orient the nucleic acid molecules according to the judgment of one of skill in the art. Exemplary techniques are described in (Matsuura et al., 2002); (Ferree and Blanch, 2003); (Stigter and Bustamante, 1998); (Matsuura et al., 2001); (Ferree and Blanch, 2004); the contents of which are hereby incorporated by reference in their entirety.

In certain embodiments, hydrodynamic force is applied to nucleic acid molecules to stretch, or orient them, or both. The hydrodynamic force is strong enough to stretch or orient the nucleic acid molecules according to the judgment of one of skill in the art. Exemplary techniques are described in (Bensimon et al., 1994); (Henegariu et al., 2001); (Kraus et al., 1997); (Michalet et al., 1997); (Yokota et al., 1997); (Otobe and Ohtani, 2001); (Zimmermann and Cox, 1994), and U.S. Pat. No. 6,548,255 (Bensimon et al., 2003); U.S. Pat. No. 6,344,319 (Bensimon et al., 2002); U.S. Pat. No. 6,303,296 (Bensimon et al., 2001a); U.S. Pat. No. 6,265,153 (Bensimon et al., 2001b); U.S. Pat. No. 6,225,055 (Bensimon and Bensimon, 2001); U.S. Pat. No. 6,054,327 (Bensimon et al., 2000); and U.S. Pat. No. 5,840,862 (Bensimon et al., 1998), the contents of which are hereby incorporated by reference in their entirety.

In certain embodiments, the force of gravity can be combined with, for example, hydrodynamic force to stretch or orient or both stretch and orient nucleic acid molecules. In certain embodiments, the force is strong enough to stretch or orient the nucleic acid molecule according to the judgment of one of skill in the art. Exemplary techniques for extending a nucleic acid molecule with gravity are described in (Michalet et al., 1997); (Yokota et al., 1997); (Kraus et al., 1997), the contents of which are hereby incorporated by reference in their entirety.

In particular embodiments, the force is applied through a moving meniscus. Those of skill in the art recognize that a moving meniscus can apply various forces to nucleic acid molecules including hydrodynamic force, surface tension and any other force recognized by those of skill in the art. The meniscus can be moved by any technique apparent to those of skill in the art including evaporation and gravity. Exemplary techniques are described in, for example, U.S. Pat. No. 6,548,255 (Bensimon et al., 2003); U.S. Pat. No. 6,344,319 (Bensimon et al., 2002); U.S. Pat. No. 6,303,296 (Bensimon et al., 2001a); U.S. Pat. No. 6,265,153 (Bensimon et al., 2001b); U.S. Pat. No. 6,225,055 (Bensimon and Bensimon, 2001); U.S. Pat. No. 6,054,327 (Bensimon et al., 2000); and U.S. Pat. No. 5,840,862 (Bensimon et al., 1998), the contents of which are hereby incorporated by reference in their entireties.

In particular embodiments, nucleic acid molecules can be stretched or oriented or both stretched and oriented by an optical trap or optical tweezers. For instance, a nucleic acid molecule can comprise or can be linked, covalently or noncovalently, to a particle capable of being trapped or moved by an appropriate source of optical force. Useful techniques for moving particles with optical traps or optical tweezers are described in (Ashkin et al., 1986); (Ashkin and Dziedzic, 1987); (Ashkin et al., 1987); (Perkins et al., 1994); (Simmons et al., 1996); (Block et al., 1990); and (Grier, 2003); the contents of which are hereby incorporated by reference in their entireties.

In certain embodiments, the nucleic acid molecule can be stretched or oriented or both by combinations of the above forces that are apparent to those of skill in the art.

In some embodiments, only the one end or a part close to the one end of a nucleic acid molecule is anchored to a surface. In other embodiments, one end or part close to the one end of a nucleic acid molecule is anchored to a surface, then the nucleic acid molecule is stretched and then the other end or part close to the other end of the nucleic acid molecule is anchored to the surface. Anchoring can be achieved using methods described herein. In brief, examples include reactive moieties present in the ends of nucleic acid molecules, said moieties being capable of being bound to the substrate by photoactivation. The surface could comprise the photoreactive moiety, or the end of the nucleic acid molecule could comprise the photoreactive moiety. Some examples of photoreactive moieties include aryl azides, such as N4-((2-pyridyldithio) ethyl)-4-azidosalicylamide; fluorinated aryl azides, such as 4-azido-2,3,5,6-tetrafluorobenzoic acid; benzophenone-based reagents, such as the succinimidyl ester of 4-benzoylbenzoic acid; and 5-Bromo-deoxyuridine.

In certain embodiments, the end or part close to the end of a nucleic acid molecule can comprise a member of a binding pair that is capable of binding with a member of a binding pair on the surface to form one or more non-covalent bonds. Exemplary useful surfaces include those that comprise a binding moiety selected from the group consisting of ligands, antigens, carbohydrates, nucleic acids, receptors, lectins, and antibodies. Other useful surfaces comprise epoxy, aldehyde, gold, hydrazide, sulfhydryl, NHS-ester, amine, thiol, carboxylate, maleimide, hydroxymethyl phosphine, imidoester, isocyanate, hydroxyl, pentafluorophenyl-ester, psoralen, pyridyl disulfide or vinyl sulfone, or mixtures thereof. Such surfaces can be obtained from commercial sources or prepared according to standard techniques.

In certain embodiments, the one or both ends of a nucleic acid molecule can be immobilized to the surface of a substrate via an avidin-biotin binding pair. In certain embodiments, the nucleic acid molecule can comprise a biotin moiety in its one or both ends. Useful surfaces comprising avidin are commercially available including TB0200 (Accelr8), SAD6, SAD20, SAD100, SAD500, SAD2000 (Xantec), SuperAvidin (Array-It), streptavidin slide (catalog #IVIPC 000, Xenopore) and STREPTAVIDINnslide (catalog #439003, Greiner Bio-one).

In further embodiments, the one end of a nucleic acid molecule can comprise avidin, and the surface can comprise biotin. Useful substrates comprising biotin are commercially available including Optiarray-biotin (Accelr8), BD6, BD20, BD100, BD500 and BD2000 (Xantec).

### EXAMPLES

Methods described herein may employ conventional techniques and descriptions of fields such as organic chemistry, polymer technology, molecular biology, cell biology, and biochemistry, which are within the skill of the art. Such conventional techniques include, but are not limited to, polymerization, hybridization, ligation, label detection, and detection of hybridization using a label. Such conventional techniques and descriptions can be found in standard laboratory manuals such as “Genome Analysis: A Laboratory Manual Series (Vols. I-IV)” (Green, 1997), “PCR Primer: A Laboratory Manual” (Dieffenbach and Dveksler, 2003), “Molecular Cloning: A Laboratory Manual” (Green and Sambrook, 2012), and others (Berg, 2006); (Gait, 1984); (Nelson and Cox, 2012), all of which are herein incorporated in their entirety by reference for all purposes.

All referenced publications (e.g., patents, patent applications, journal articles, books) are included herein in their entirety.

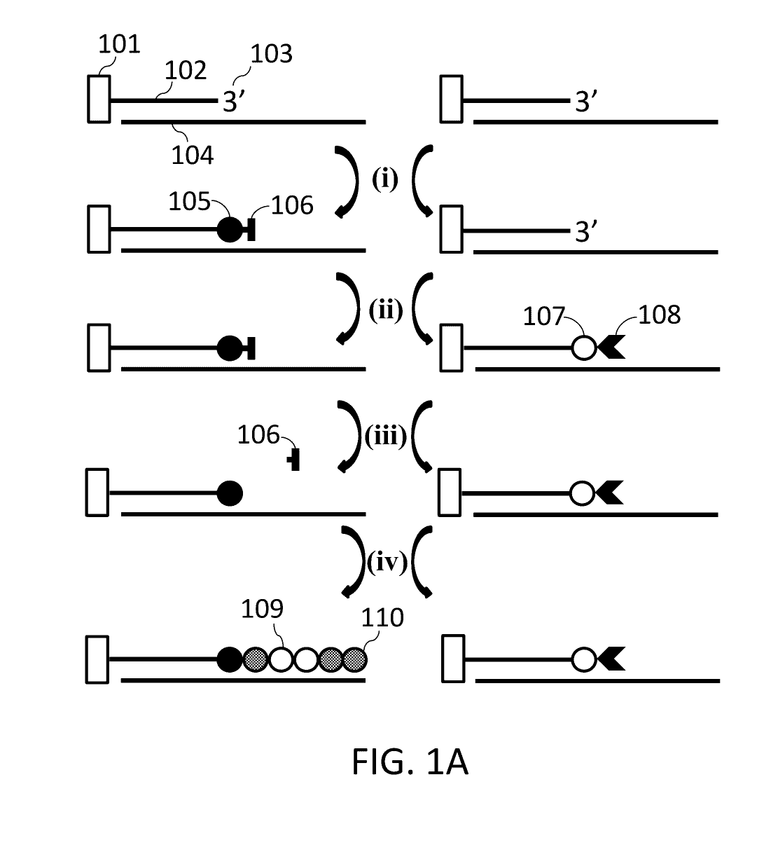

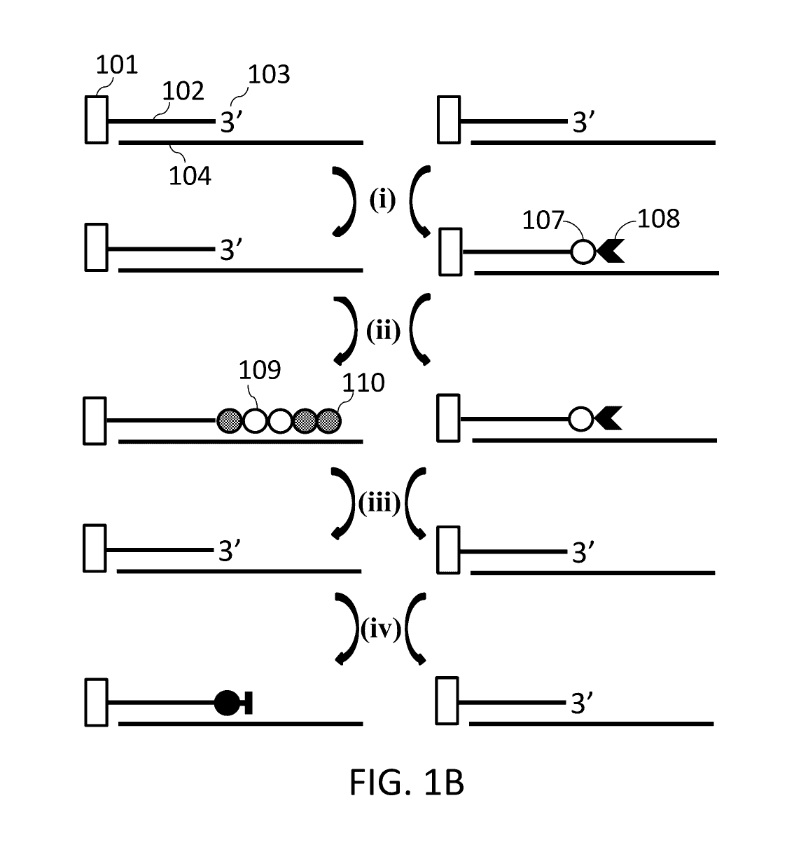

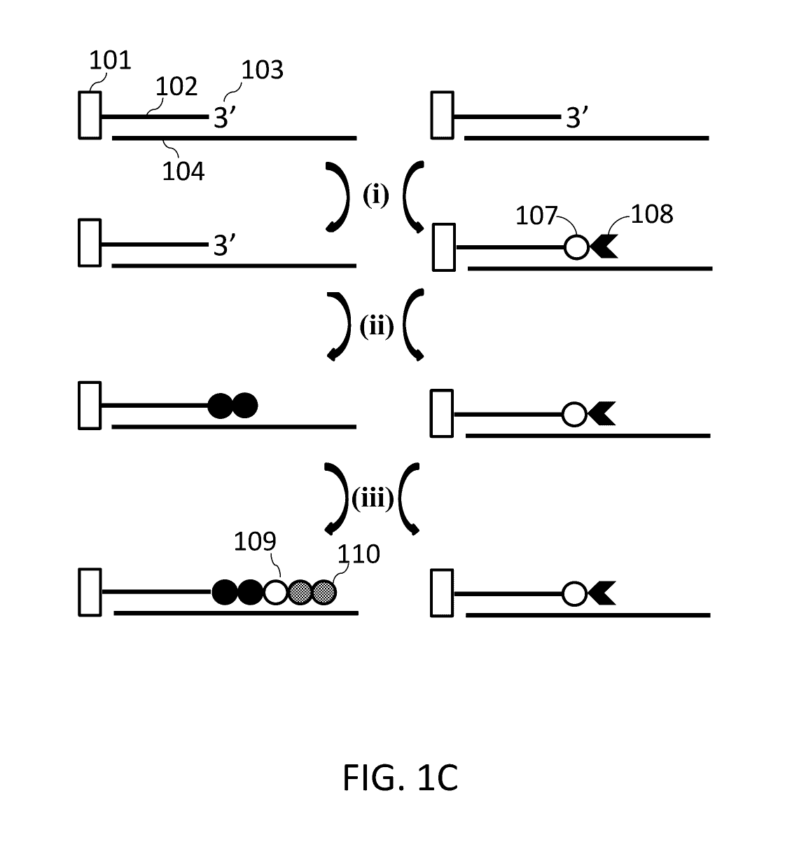

In one embodiment shown in FIG. 1A, a nucleic acid molecule 104 is a DNA strand hybridized to another DNA strand 102 that is anchored to a solid support 101. The anchored strand 102 has an extendable 3′ end 103, which can be extended by polymerization. In FIG. 1A, the left side shows the nucleic acid molecule 104 participating in steps (i) through (iv) in the event that the nucleic acid molecule 104 incorporates a nucleotide comprising a predetermined base type in step (i), whereas the right side of FIG. 1A shows the same nucleic acid molecule 104 participating in the same steps (i) through (iv) in the event that no incorporation takes place during step (i). The method can apply to a mixture of nucleic acid molecules, wherein there are nucleic acid molecules that behave like the nucleic acid molecule in the left side, and others that behave like the nucleic acid molecule in the right side.

During step (i) in FIG. 1A, 104 and its surroundings are exposed to conditions to cause nucleotide incorporation, and to a template-dependent polymerization reaction solution comprising reversibly terminated nucleotides comprising a predetermined (known in advance) base type.

In the embodiment of FIG. 1A, a nucleotide 105 comprising the predetermined base type is incorporated into the nucleic acid molecule shown at the left side of FIG. 1A. The nucleotide comprises a reversible terminator 106. The right side of FIG. 1A shows that no incorporation takes place. In this case, nucleotides comprising the predetermined base type are not complementary to the nucleic acid molecule at the specific position following the extendable 3′ end.

In this embodiment, the process continues with step (ii), during which a blocking nucleotide tail is constructed in the event that no nucleotide incorporation occurs during step (i). The purpose of the blocking nucleotide tail is to prevent removable nucleotide tail construction in a nucleic acid molecule that does not incorporate the predetermined nucleotide type of step (i). In this embodiment, the constructed blocking nucleotide tail comprises a single cleavable nucleotide 107 comprising a terminator 108. Step (ii) comprises exposing the nucleic acid molecule and its parts to polymerization conditions, and to a template-dependent polymerization reaction solution comprising terminated cleavable nucleotides to complement the nucleic acid molecule. Irreversibly terminated cleavable nucleotides may be used. In the event that reversibly terminated cleavable nucleotides are used, the reversible terminators of these nucleotides are different from the reversible terminators of the predetermined nucleotide type of step (i) (i.e. the reversible terminators of the nucleotides of step (ii) can be removed by conditions and reagents different from the conditions and reagents used to remove the reversible terminators of step (i)). In the event that no incorporation occurs in step (i), step (ii) yields the product shown in the right side of FIG. 1A, which is an incorporated cleavable nucleotide 107 comprising a terminator 108. In the event that there is incorporation of a nucleotide during step (i), step (ii) does not have any effect, as shown in the left side of FIG. 1A.

In another embodiment, steps (i) and (ii) are combined in a single step, comprising reversibly blocked nucleotides comprising the predetermined base type, and blocked cleavable nucleotides that do not comprise the predetermined base type.

In another embodiment, steps (i) and (ii) are combined in a single step, comprising reversibly terminated cleavable nucleotides comprising base types other than the predetermined base type, and also comprising reversibly terminated nucleotides comprising the predetermined base type. Additionally, said cleavable nucleotides do not comprise base types with the same complementarity properties with the predetermined base type (e.g., in the event that thymine is the predetermined base type, uracil is not included in the reaction). Also, the reversibly terminated cleavable nucleotides comprise reversible terminators of a different type from the reversible terminators comprised in the nucleotides comprising the predetermined base type. In another embodiment, each nucleotide type present in the polymerization reaction solution comprises a type of reversible terminator different from the types of reversible terminators comprised in the other nucleotide types.

During step (iii) in FIG. 1A, the reversible terminator 106 is removed by exposing the nucleic acid molecule and its surroundings to appropriate conditions and reagents, which are described elsewhere herein. In the event that there is a blocking nucleotide tail constructed during step (ii), step (iii) has no effect.

During step (iv), the construction of a removable nucleotide tail may occur. In this embodiment, step (iv) comprises exposing the nucleic acid molecule 104 and its parts to polymerization conditions, and to a template-dependent polymerization reaction solution that comprises a mixture of unlabeled and labeled cleavable nucleotides to complement the nucleic acid molecule 104. In the event that no nucleotide is incorporated into the nucleic acid molecule 104 during step (i), step (iv) has no effect and the nucleic acid molecule 104 remains carrying the blocking nucleotide tail, as shown in FIG. 1A, right side. In the event that a nucleotide is incorporated into the nucleic acid molecule 104 during step (i), step (iv) produces a removable nucleotide tail 109 comprising unlabeled and labeled cleavable nucleotides 110, as shown in FIG. 1A, left side. In some embodiments, nucleotide labels can be moieties causing changes in conductivity when passing through a nanopore. In such embodiments, the presence of the removable nucleotide tail is detected by using a nanopore device. Labels, labeling reactions, detection methods and other relevant materials, equipment, reagents and conditions are described elsewhere herein. Washing and other treatments may be applied in between described steps as recognized and known by those skilled in the art.

In another embodiment shown in FIG. 1B, the blocking nucleotide tail which comprises a single cleavable and blocked nucleotide 107 is constructed first, during step (i). Then, during step (ii), the labeled removable nucleotide tail 109 is constructed by extending the 3′ end of the nucleic acid molecule in the event that the nucleic acid molecule does not incorporate a blocked cleavable nucleotide in step (i). The next step, step (iii), cleaves blocking and removable nucleotide tails that may be formed in previous steps, and then in step (iv), the nucleic acid molecule is exposed to a reaction solution and conditions to cause incorporation of a reversibly blocked nucleotide comprising the predetermined base type. In subsequent cycles, the reversibly blocked nucleotide can be unblocked, and the process can restart. Sequential construction and detection of labeled removable nucleotide tails allows sequencing. Methods for removing cleavable nucleotides and other relevant reagents and methods are described elsewhere herein.

In another embodiment shown in FIG. 1C, the blocking nucleotide tail which comprises a single cleavable and blocked nucleotide 107 is constructed first, during step (i). Then, during step (ii), the nucleic acid molecule is exposed to polymerization conditions, and to a polymerization reaction solution comprising nucleotides comprising the predetermined base type that are not blocked. This allows the incorporation of more than one nucleotide into the nucleic acid molecule in the event that there is a homopolymer sequence. For example, in FIG. 1C, two nucleotides are incorporated. This approach may not be suitable for base-by-base sequencing, but it can enable base determination, by constructing a labeled removable nucleotide tail 109 in step (iii), which is formed in the event that at least one nucleotide comprising the predetermined base type is incorporated.

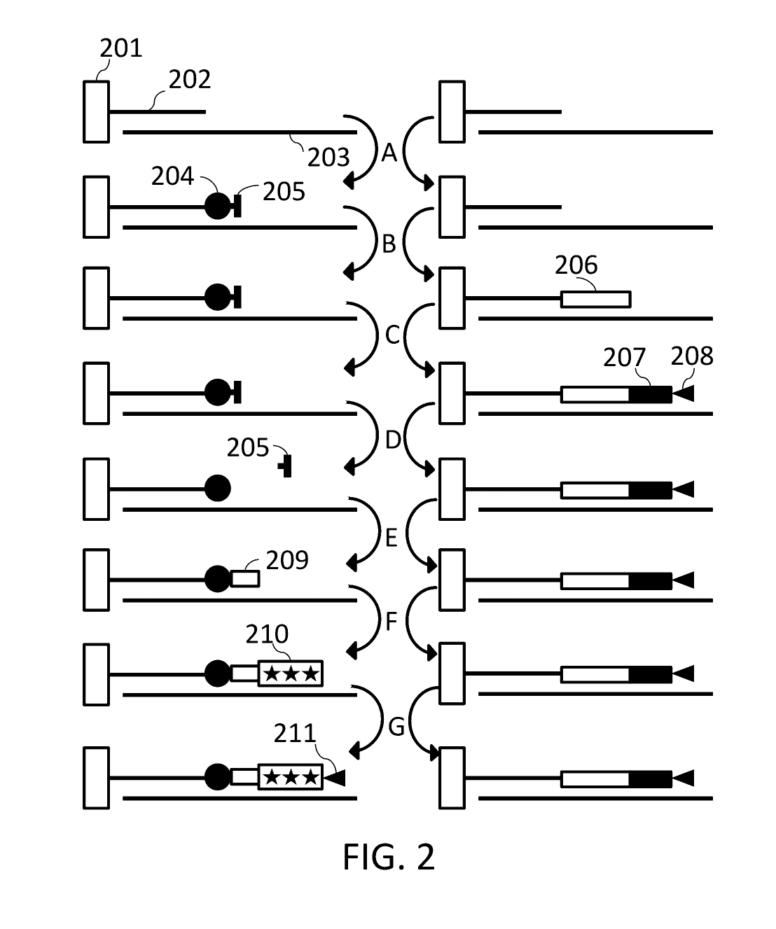

In a certain embodiment shown in FIG. 2, a nucleic acid molecule 203 is a single DNA strand hybridized to another DNA strand 202 that is anchored to a solid support 201. The anchored strand 202 has an extendable 3′ end, which can be extended by polymerization. In FIG. 2, the left side shows the nucleic acid molecule 203 participating in steps (A) through (G) in the event that the nucleic acid molecule 203 incorporates a nucleotide comprising a predetermined base type in step (A), whereas the right side of FIG. 2 shows the same nucleic acid molecule 203 participating in the same steps (A) through (G) in the event that no incorporation takes place during step (A). The method can apply to a mixture of nucleic acid molecules, wherein there are nucleic acid molecules that behave like the nucleic acid molecule in the left side, and others that behave like the nucleic acid molecule in the right side.

During step (A) in FIG. 2, 203 and its surroundings are exposed to conditions to cause polymerization, and to a template-dependent polymerization reaction solution comprising reversibly terminated nucleotides comprising a predetermined base type.

In the embodiment of FIG. 2, a nucleotide 204 comprising the predetermined base type is successfully incorporated into the nucleic acid molecule shown at the left side of FIG. 2. The nucleotide comprises a reversible terminator 205. The right side of FIG. 2 shows that no incorporation takes place. In this case, nucleotides comprising the predetermined base type are not complementary to the nucleic acid molecule at the specific position following the extendable 3′ end.

In this embodiment, the process continues with steps (B) and (C), during which a blocking nucleotide tail is constructed in the event that no nucleotide incorporation occurs during step (A). The purpose of the blocking nucleotide tail is to prevent construction of a removable nucleotide tail in a nucleic acid molecule that does not incorporate the predetermined nucleotide type of step (A). In this embodiment, the constructed blocking nucleotide tail comprises two segments, a first one comprising cleavable nucleotides and a second one comprising deoxyribonucleotides. The second segment ends with a terminated nucleotide, such as a dideoxyribonucleotide. Step (B) comprises exposing the nucleic acid molecule and its parts to conditions to cause polymerization, and to a template-dependent polymerization reaction solution comprising cleavable nucleotides to complement the nucleic acid molecule 203. In the event that no incorporation occurs in step (A), step (B) produces segment 206 which is complementary to the nucleic acid molecule 203. In the event that there is incorporation of a nucleotide during step (A), step (B) does not have any effect, as shown in the left side of FIG. 2.

The segment 206 can be further extended during step (C). In this embodiment, step (C) comprises exposing the nucleic acid molecule and its parts to conditions to cause polymerization, and to a template-dependent polymerization reaction solution comprising deoxyribonucleotides and dideoxyribonucleotides to complement the nucleic acid molecule 203. Step (C) produces segment 207 which comprises deoxyribonucleotides and is irreversibly terminated with the incorporation of dideoxyribonucleotide 208. The incorporation of 208 prevents construction of a removable nucleotide tail in the event that there is no nucleotide incorporation during step (A). In another embodiment, step (C) uses only dideoxyribonucleotides, in order to irreversibly terminate the blocking nucleotide tail segment 206. In another embodiment, the template-dependent polymerization reaction solution of step (C) comprises a mixture of labeled and unlabeled deoxyribonucleotides, and step (C) is followed by another step which comprises a template-dependent polymerization reaction to incorporate dideoxyribonucleotides. Including labeled deoxyribonucleotides in the blocking nucleotide tail enables detection of the tail. Said detection serves to differentiate the absence of a removable nucleotide tail due to non-incorporation of a nucleotide in step (A), from the absence of said tail due to a technical error. The labels used for the construction of the blocking nucleotide tail are different from the labels used for the construction of the removable nucleotide tail during subsequent steps, so that they produce distinct signal. In the event that there is incorporation of a nucleotide during step (A), step (C) does not have any effect, as shown in the left side of FIG. 2.

During step (D) in FIG. 2, the reversible terminator 205 is removed by exposing the nucleic acid molecule and its surroundings to appropriate conditions and reagents, which are described elsewhere herein. In the event that there is a blocking nucleotide tail constructed into the nucleic acid molecule 203 during step (B), step (D) has no effect.

During step (E), the construction of a first segment of a removable nucleotide tail may occur. In this embodiment, step (E) comprises exposing the nucleic acid molecule 203 and its surroundings to conditions to cause polymerization, and to a template-dependent polymerization reaction solution that comprises a mixture of labeled and unlabeled cleavable nucleotides to complement the nucleic acid molecule. As the nucleic acid molecule 203 is DNA, in one example the cleavable nucleotides can be ribonucleotides, and the reaction solution comprises fluorescein-labeled UTP. In the event that no nucleotide is incorporated into the nucleic acid molecule 203 during step (A), step (E) has no effect and the nucleic acid molecule 203 remains carrying the blocking nucleotide tail, as shown in FIG. 2, right side. In the event that a nucleotide is incorporated into the nucleic acid molecule 203 during step (A), step (E) produces segment 209 comprising cleavable nucleotides, as shown in FIG. 2, left side.

The presence of the cleavable segments 206 and 209 enable cleavage of the blocking and removable nucleotide tails, and subsequent sequencing, as it is described in more detail in later figures herein. 206 and 209 may be short, because cleavable nucleotides are usually modified nucleotides that are incorporated into nucleic acid molecules at significantly lower rates or lower numbers or both than unmodified nucleotides. For example, Pol ∈, which is a polymerase that can perform incorporation of ribonucleotides into DNA molecules, does so 10-fold less efficiently than incorporating deoxyribonucleotides (Goksenin et al., 2012). Detailed descriptions of polymerases and production of short ribonucleotide segments or other cleavable nucleotide segments of short length are given in the “Polymerases” section, and Example 9. Cleavable segments can be further extended. 209 can be further extended during step (F), which comprises exposing the nucleic acid molecule 203 and its parts to conditions to cause polymerization, and to a template-dependent polymerization reaction solution comprising a mixture of unlabeled and labeled deoxyribonucleotides to complement the nucleic acid molecule. During step (F), the labeled segment 210 of the removable nucleotide tail is constructed, in the event that a nucleotide is incorporated into the nucleic acid molecule during step (A), as shown in FIG. 2, left side. In the event that no incorporation occurs during step (A), step (F) has no effect, as shown in FIG. 2, right side.

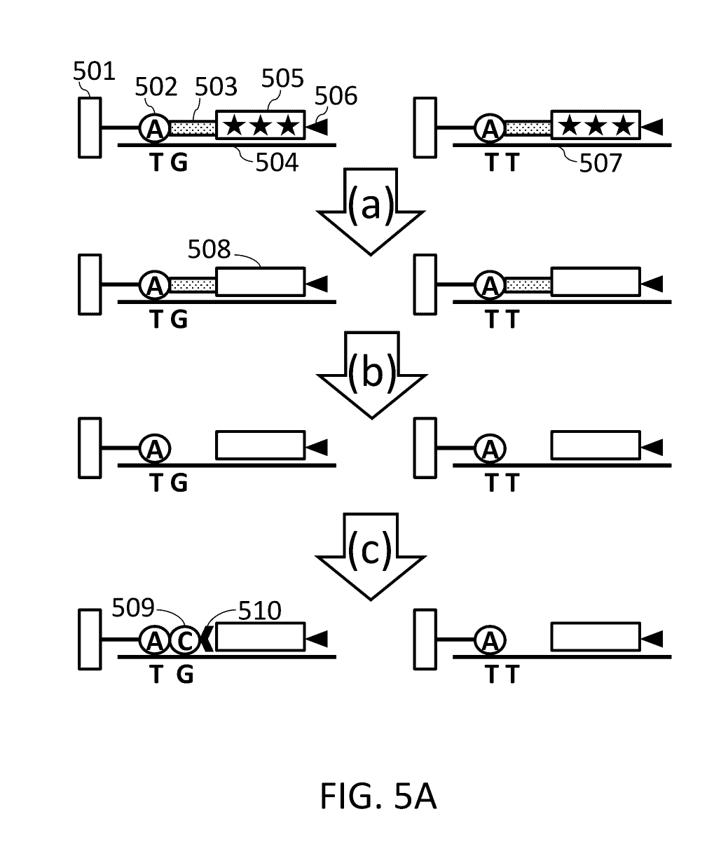

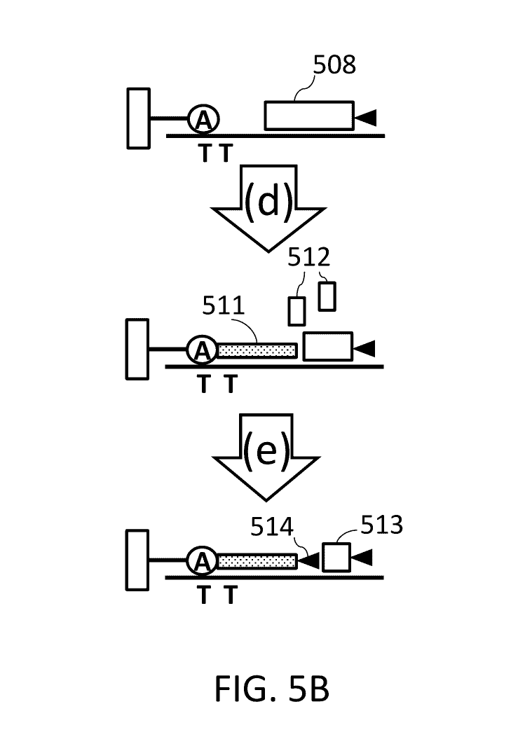

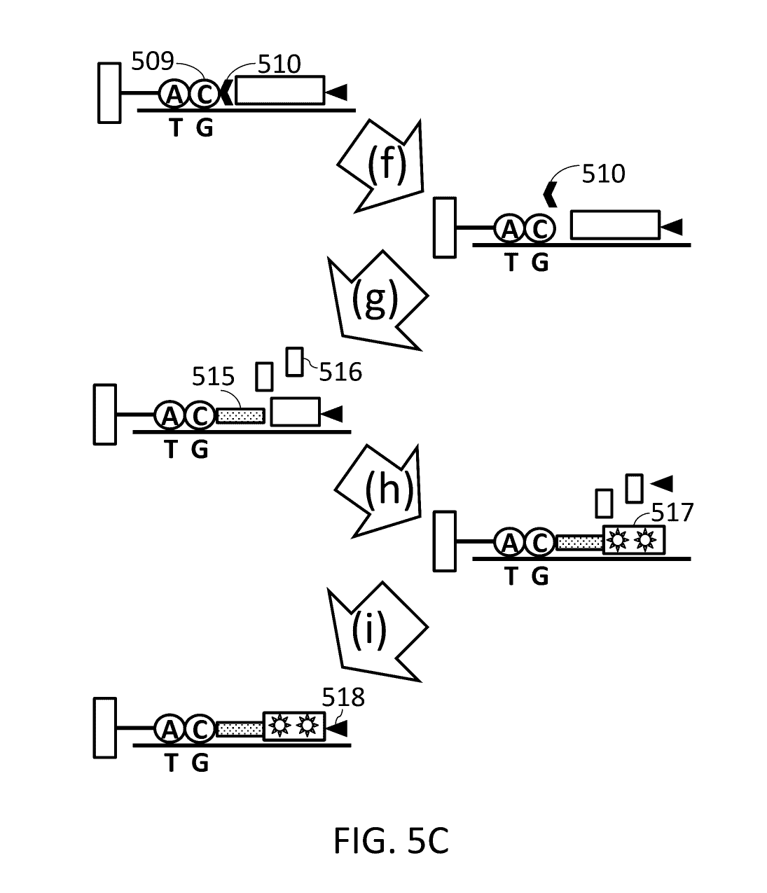

The last step, step (G) comprises exposing the nucleic acid molecule and its parts to conditions to cause polymerization, and to a template-dependent polymerization reaction solution comprising dideoxyribonucleotides to complement the nucleic acid molecule. Incorporation of a dideoxyribonucleotide 211 prevents off-site polymerization in the event that the nucleic acid molecule and its parts are subjected to future cycles of constructing new removable nucleotide tails, as it is shown in more detail in FIG. 5A. Step (G) causes termination of 210 in the event that 210 does not reach the end of 203 during step (F). In the event that no incorporation occurs during step (A), step (G) has no effect, as shown in FIG. 2, right side.

Washing and other treatments may be applied in between described steps as recognized and known by those skilled in the art. Labels, labeling reactions, cleavable nucleotides, and other reagents and conditions are discussed in more detail in elsewhere herein.

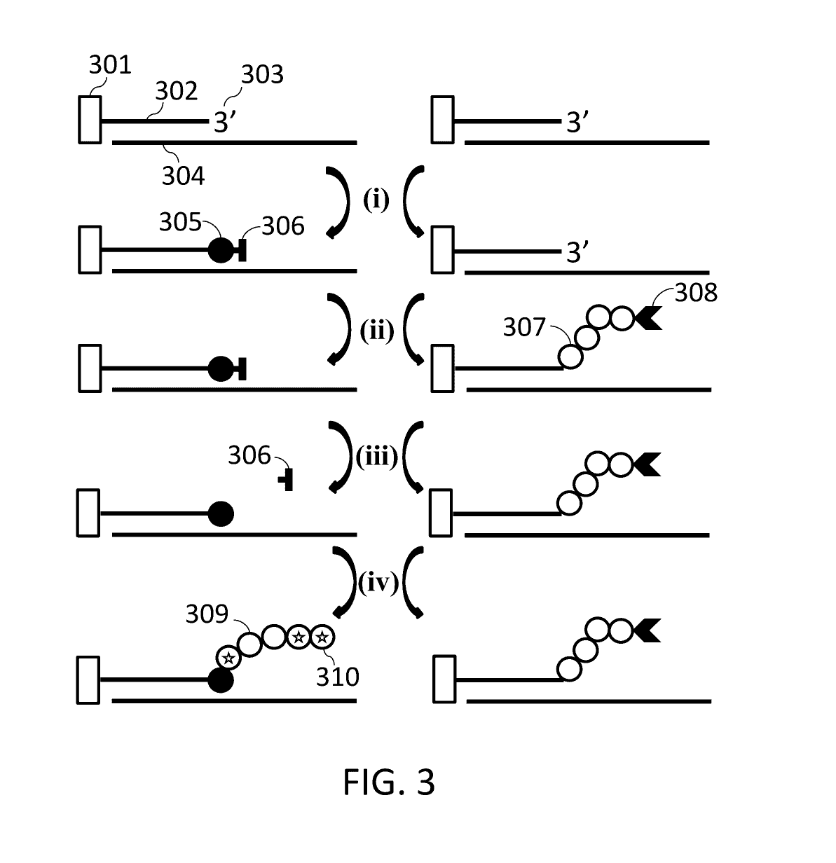

In one embodiment shown in FIG. 3, a nucleic acid molecule 304 is a single DNA strand hybridized to another DNA strand 302 that is anchored to a solid support 301. The anchored strand 302 has an extendable 3′ end 303, which can be extended by polymerization. In FIG. 3, the left side shows the nucleic acid molecule 304 participating in steps (i) through (iv) in the event that the nucleic acid molecule 304 incorporates a nucleotide comprising a predetermined base type in step (i), whereas the right side of FIG. 3 shows the same nucleic acid molecule 304 participating in the same steps (i) through (iv) in the event that no incorporation takes place during step (i). The method can apply to a mixture of nucleic acid molecules, wherein there are nucleic acid molecules that behave like the nucleic acid molecule in the left side, and others that behave like the nucleic acid molecule in the right side.

During step (i) in FIG. 3, 304 and its surroundings are exposed to conditions to cause polymerization, and to a template-dependent polymerization reaction solution comprising reversibly terminated nucleotides comprising a predetermined base type.

In the embodiment of FIG. 3, a nucleotide 305 comprising the predetermined base type is successfully incorporated into the nucleic acid molecule shown at the left side of FIG. 3. The nucleotide comprises a reversible terminator 306. The right side of FIG. 3 shows that no incorporation takes place. In this case, nucleotides comprising the predetermined base type are not complementary to nucleic acid molecule at the specific position following the extendable 3′ end.

In this embodiment, the process continues with step (ii), during which a blocking nucleotide tail is constructed in the event that no nucleotide incorporation occurs during step (i). The purpose of the blocking nucleotide tail is to prevent the construction of a removable nucleotide tail in the event that a nucleic acid molecule does not incorporate the predetermined nucleotide type of step (i). In this embodiment, the constructed blocking nucleotide tail is a segment that is not complementary to 304. Step (ii) comprises exposing the nucleic acid molecule and its parts to conditions to cause polymerization, and to a template-independent polymerization reaction solution comprising terminal deoxynucleotidyl transferase (TdT) molecules, cleavable nucleotides, and cleavable nucleotides comprising terminators. The population of said nucleotides can comprise one base type, or two base types, or more. The terminators of said nucleotides are either irreversible or reversible. In the event that said terminators are reversible, they are different from the reversible terminators of the predetermined nucleotide type of step (i) (i.e. the reversible terminators of the nucleotides of step (ii) can be removed by conditions and reagents different from the conditions and reagents used to remove or damage the reversible terminators of step (i)). In the event that no incorporation occurs in step (i), step (ii) yields the product shown in the right side of FIG. 3, which is a blocking nucleotide tail 307 that is non-complementary to 304 and is terminated by adding a cleavable nucleotide comprising terminator 308. In the event that there is incorporation of a nucleotide during step (i), step (ii) does not have any effect, as shown in the left side of FIG. 3.

During step (iii) in FIG. 3, the reversible terminator 306 is removed by exposing the nucleic acid molecule and its surroundings to the appropriate conditions and reagents, which are described elsewhere herein. In the event that there is a blocking nucleotide tail constructed during step (ii), step (iii) has no effect.

During step (iv), the construction of a removable nucleotide tail may occur. In this embodiment, step (iv) comprises exposing the nucleic acid molecule 304 and its parts to conditions to cause polymerization, and to a template-independent polymerization reaction solution that comprises TdT molecules and a mixture of unlabeled and labeled cleavable nucleotides. The population of said nucleotides can comprise one base type, or two base types, or more. In the event that no nucleotide is incorporated into the nucleic acid molecule 304 during step (i), step (iv) has no effect and the nucleic acid molecule 304 remains carrying the blocking nucleotide tail, as shown in FIG. 3, right side. In the event that a nucleotide is incorporated into the nucleic acid molecule 304 during step (i), step (iv) produces a removable nucleotide 309 comprising unlabeled and labeled cleavable nucleotides 310, as shown in FIG. 3, left side.

Washing and other treatments may be applied in between described steps as recognized and known by those skilled in the art. Labels, labeling, cleavable nucleotide and other reagents and conditions are described elsewhere herein.

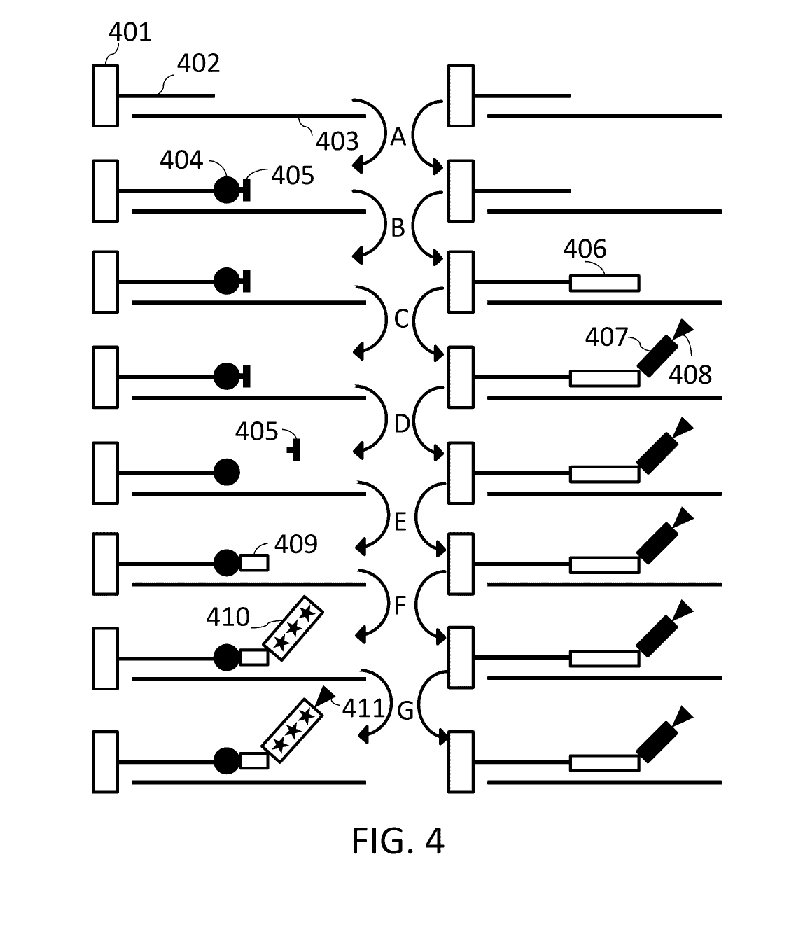

In one embodiment shown in FIG. 4, a nucleic acid molecule 403 is a single DNA strand hybridized to another DNA strand 402 that is anchored to a solid support 401. The anchored strand 402 has an extendable 3′ end, which can be extended by polymerization. In FIG. 4, the left side shows the nucleic acid molecule 403 participating in steps (A) through (G) in the event that the nucleic acid molecule 403 incorporates a nucleotide comprising a predetermined base type in step (A), whereas the right side of FIG. 4 shows the same nucleic acid molecule 403 participating in the same steps (A) through (G) in the event that no incorporation takes place during step (A). The method can apply to a mixture of nucleic acid molecules, wherein there are nucleic acid molecules that behave like the nucleic acid molecule in the left side, and others that behave like the nucleic acid molecule in the right side.

During step (A) in FIG. 4, 403 and its surroundings are exposed to conditions to cause polymerization, and to a template-dependent polymerization reaction solution comprising reversibly terminated nucleotides comprising a predetermined base type.

In the embodiment of FIG. 4, a nucleotide 404 comprising the predetermined base type is successfully incorporated into the nucleic acid molecule shown at the left side of FIG. 4. The nucleotide comprises a reversible terminator 405. The right side of FIG. 4 shows that no incorporation takes place. In this case, nucleotides comprising the predetermined base type are not complementary to the nucleic acid molecule at the specific position following the extendable 3′ end.

In this embodiment, the process continues with steps (B) and (C), during which a blocking nucleotide tail is constructed in the event that no nucleotide incorporation occurs during step (A). The purpose of the blocking nucleotide tail is to prevent construction of a removable nucleotide tail in a nucleic acid molecule that does not incorporate the predetermined nucleotide type of step (A). In this embodiment, the constructed blocking nucleotide tail comprises two segments, a first one comprising cleavable nucleotides and a second one comprising deoxyribonucleotides. The second segment ends with a terminated nucleotide, such as a dideoxyribonucleotide. Step (B) comprises exposing the nucleic acid molecule and its parts to conditions to cause polymerization, and to a template-dependent polymerization reaction solution comprising cleavable nucleotides to complement the nucleic acid molecule 403. In the event that no incorporation occurs in step (A), step (B) produces segment 406 which is complementary to the nucleic acid molecule 403. In the event that there is incorporation of a nucleotide during step (A), step (B) does not have any effect, as shown in the left side of FIG. 4.

The segment 406 can be further extended during step (C). In this embodiment, step (C) comprises exposing the nucleic acid molecule and its parts to conditions to cause polymerization, and to a template-independent polymerization reaction solution comprising TdT molecules, deoxyribonucleotides and dideoxyribonucleotides. The population of said deoxyribonucleotides and dideoxyribonucleotides can comprise one base type, or two base types, or more. Step (C) produces segment 407 which comprises deoxyribonucleotides and is irreversibly terminated with the addition of dideoxyribonucleotide 408. The addition of 408 prevents construction of a removable nucleotide tail in the event that there is no nucleotide incorporation during step (A). In another embodiment, the template-independent polymerization reaction solution of step (C) comprises a mixture of labeled and unlabeled deoxyribonucleotides, and step (C) is followed by another step which comprises a template-independent polymerization reaction to incorporate dideoxyribonucleotides. The populations of said deoxyribonucleotides and dideoxyribonucleotides can comprise one base type, or two base types, or more. Including labeled deoxyribonucleotides in the blocking nucleotide tail enables detection of the tail. Said detection serves to differentiate the absence of a removable nucleotide tail due to non-incorporation of a nucleotide in step (A), from the absence of said tail due to a technical error. The labels used for the construction of the blocking nucleotide tail are different from the labels used for the construction of the removable nucleotide tail during subsequent steps, so that they produce distinct signal. In the event that there is incorporation of a nucleotide during step (A), step (C) does not have any effect, as shown in the left side of FIG. 4.

During step (D) in FIG. 4, the reversible terminator 405 is removed by exposing the nucleic acid molecule and its surroundings to appropriate conditions and reagents, which are described elsewhere herein. In the event that there is a blocking nucleotide tail constructed into the nucleic acid molecule 403 during step (B), step (D) has no effect.

During step (E), the construction of a first segment of a removable nucleotide tail may occur. In this embodiment, step (E) comprises exposing the nucleic acid molecule 403 and its surroundings to conditions to cause polymerization, and to a template-dependent polymerization reaction solution that comprises a mixture of unlabeled and labeled cleavable nucleotides to complement the nucleic acid molecule 403. In the event that no nucleotide is incorporated into the nucleic acid molecule 403 during step (A), step (E) has no effect and the nucleic acid molecule 403 remains carrying the blocking nucleotide tail, as shown in FIG. 4, right side. In the event that a nucleotide is incorporated into the nucleic acid molecule 403 during step (A), step (E) produces segment 409 comprising cleavable nucleotides, as shown in FIG. 4, left side.

For reasons explained in FIG. 2, cleavable segments of removable nucleotide tails may be further extended. 409 can be further extended during step (F), which comprises exposing the nucleic acid molecule 403 and its parts to conditions to cause polymerization, and to a template-independent polymerization reaction solution comprising TdT molecules and a mixture of unlabeled and labeled deoxyribonucleotides. The population of said deoxyribonucleotides can comprise one base type, or two base types, or more. During step (F), labeled segment 410 of the removable nucleotide tail is constructed, in the event that a nucleotide is incorporated into the nucleic acid molecule during step (A), as shown in FIG. 4, left side. In the event that no incorporation occurs during step (A), step (F) has no effect, as shown in FIG. 4, right side.

The last step, step (G) comprises exposing the nucleic acid molecule and its parts to conditions to cause polymerization, and to a template-independent polymerization reaction solution comprising TdT molecules and dideoxyribonucleotides comprising one base type, or two base types, or more. Addition of a dideoxyribonucleotide 411 prevents off-site polymerization in the event that the nucleic acid molecule and its parts are subjected to future cycles of constructing new removable nucleotide tails, as it is shown in more detail in FIG. 5A. In the event that no incorporation occurs during step (A), step (G) has no effect, as shown in FIG. 4, right side.

Washing and other treatments may be applied in between described steps as recognized and known by those skilled in the art. Labels, labeling, cleavable nucleotides and other reagents and conditions are described in more detail elsewhere herein.

In a certain embodiment shown in FIG. 5A, FIG. 5B and FIG. 5C, a blocking nucleotide tail and a removable nucleotide tail are constructed in two nucleic acid molecules already having previously constructed removable nucleotide tails, in a manner that enables sequencing of the nucleic acid molecules. FIG. 5A shows two nucleic acid molecules, one is 504 and another 507. In FIG. 5, the same numbers apply to refer to drawn parts that have the same features in both 504 and 507. The nucleic acid molecule 504 is a DNA strand with its complementary extendable strand anchored to a solid support 501. 504 has a thymine (T) at a specific position, which is immediately followed by a guanine (G). The thymine is bound to its complementary adenine (A), which is comprised in deoxyribonucleotide 502 in the strand complementary to 504. 502 is extended by a removable nucleotide tail. Said tail comprises a first segment 503 and a second segment 505. Segment 503 comprises cleavable nucleotides, whereas segment 505 comprises unlabeled and labeled deoxyribonucleotides. Segment 505 has a dideoxyribonucleotide 506 at its 3′ end. The labels within 505 are specific for the presence of the base type adenine, meaning that detection of said labels indicates the presence of adenine in the deoxyribonucleotide (502) preceding (i.e., associated with the 5′ end of) the removable nucleotide tail. In this embodiment, the labels are fluorescent. For more details on labels, see elsewhere herein. The method can apply to a mixture of nucleic acid molecules, wherein there are nucleic acid molecules that behave like the nucleic acid molecule in the left side, and others that behave like the nucleic acid molecule in the right side.

Next to the nucleic acid molecule 504 in FIG. 5A there is another nucleic acid molecule shown, 507. Nucleic acid molecule 507 has the same features with 504, except thymine is followed by another thymine (T), and not guanine.

During step (a) in FIG. 5A, both nucleic acid molecules 504 and 507, and their surroundings, are exposed to photobleaching as described elsewhere herein, in order to damage the labels. 508 is the resulting photobleached removable nucleotide tail (the same applies to the tail in nucleic acid molecule 507). Photobleaching is a useful method, because photobleached removable nucleotide tails do not interfere with the labels of subsequently constructed labeled tails, in the event that said photobleached tails are not removed completely (this becomes more evident in FIGS. 5B and 5C).

During step (b), both nucleic acid molecules 504 and 507 are exposed to conditions and reagents that release the cleavable nucleotides of the first segments of the removable nucleotide tails (503). Said conditions and reagents are suitable for the type of cleavable nucleotides used in the tails, and are described in detail elsewhere herein. Upon completion of step (b), the 3′ end of the deoxyribonucleotide 502 (in both 504 and 507) becomes available for extension by polymerization (i.e. said end regains a —OH group).

During step (c), both nucleic acid molecules 504 and 507, and their surroundings, are exposed to conditions to cause polymerization, and to a template-dependent polymerization reaction solution comprising reversibly terminated deoxyribonucleotides comprising a predetermined base type, which in this case is cytosine (C).

As shown in FIG. 5A, a deoxyribonucleotide 509 comprising cytosine is successfully incorporated into the nucleic acid molecule 504. Said nucleotide comprises a reversible terminator 510. There is no incorporation occurring in 507, because 507 has a thymine instead of a guanine.

The following steps (d) and (e) shown in FIG. 5B may construct a blocking nucleotide tail. Both nucleic acid molecules 504 and 507, and their surroundings, are exposed to the same conditions and reagents during steps (d) and (e). The reversible terminator 510 prevents further extension, and for that reason it prevents construction of a blocking nucleotide tail during steps (d) and (e). The nucleic acid molecule 504 remains unchanged during steps (d) and (e), and for that reason it is not shown in FIG. 5B.

In this embodiment, the blocking nucleotide tail constructed in nucleic acid molecule 507 comprises cleavable nucleotides and is terminated by the addition of a dideoxyribonucleotide. Step (d) comprises exposing the nucleic acid molecule and its parts to conditions to cause polymerization, and to a template-dependent polymerization reaction solution comprising cleavable nucleotides to complement the nucleic acid molecule 507. In this embodiment, polymerases used in step (d) possess 5′-to-3′ exonuclease activity and are therefore capable of digesting part of the previous removable nucleotide tail. In other embodiments, strand-displacing polymerases can be used. As shown in FIG. 5B, step (d) leads to the construction of the segment 511 and simultaneous digestion of the previous removable nucleotide tail, releasing its parts 512.

During step (e), the nucleic acid molecule and its parts are exposed to conditions to cause polymerization, and to a template-dependent polymerization reaction solution comprising dideoxyribonucleotides to complement the nucleic acid molecule. Segment 511 is irreversibly terminated with the incorporation of dideoxyribonucleotide 514. The incorporation of 514 prevents construction of a removable nucleotide tail in the event that a nucleotide comprising cytosine is not incorporated during step (c) in FIG. 5A. For reasons related to reduced rates of incorporation of cleavable nucleotides, as explained in FIG. 2, the segment 511 may be short and not reaching the end of the nucleic acid molecule 507. In this case, the part 513 from the previous tail remains, as shown in FIG. 5B. 513 does not interfere with following steps, because it is terminated and photobleached.

The following steps (f) through (i) shown in FIG. 5C may construct a labeled removable nucleotide tail, that is specific for the presence of cytosine in the incorporated nucleotide of step (c). Both nucleic acid molecules 504 and 507, and their surroundings, are exposed to the same conditions and reagents during steps (f) through (i). 514 prevents construction of a removable nucleotide tail during steps (f) through (i), so that nucleic acid molecule 507 remains unchanged during steps (f) through (i). For that reason, 507 is not shown in FIG. 5C.

During step (f) in FIG. 5C, the reversible terminator 510 is removed by exposing the nucleic acid molecule and its surroundings to appropriate conditions and reagents, which are described elsewhere herein.

During step (g), the construction of a first segment of a removable nucleotide tail may occur. In this embodiment, step (g) comprises exposing the nucleic acid molecule and its surroundings to conditions to cause polymerization, and to a template-dependent polymerization reaction solution that comprises a mixture of labeled and unlabeled cleavable nucleotides to complement the nucleic acid molecule 504. Labels in this step are different from those used in the previous removable nucleotide tail, and are specific for the presence of cytosine. In this embodiment, polymerases used in step (g) possess 5′-to-3′ exonuclease activity and are therefore capable of digesting part of the previous removable nucleotide tail. In other embodiments, strand-displacing polymerases can be used. As shown in FIG. 5C, step (g) leads to the construction of the segment 515 and simultaneous digestion of the previous removable nucleotide tail, releasing its parts 516.

For reasons related to reduced rates of incorporation of cleavable nucleotides, as explained in FIG. 2, 515 may be further extended. 515 can be further extended during step (h), which comprises exposing the nucleic acid molecule and its parts to conditions to cause polymerization, and to a template-dependent polymerization reaction solution comprising a mixture of unlabeled and labeled deoxyribonucleotides to complement the nucleic acid molecule 504. During step (h), labeled segment 517 of the removable nucleotide tail is constructed, which comprises labels specific for the presence of cytosine in the incorporated nucleotide of step (c), and are thus different from the labels in 505 of FIG. 5A which are specific for the presence of adenine.

The last step, step (i) comprises exposing the nucleic acid molecule and its parts to conditions to cause polymerization, and to a template-dependent polymerization reaction solution comprising dideoxyribonucleotides to complement the nucleic acid molecule. Incorporation of a dideoxyribonucleotide 518 prevents off-site nucleotide incorporation, or off-site construction of a blocking nucleotide tail, or off-site construction of a removable nucleotide tail, in the event that the nucleic acid molecule 504 is subjected again to steps (a) through (i). Repeating the process described in FIGS. 5A, B and C at least one time enables determining at least a part of the sequence of the nucleic acid molecules 504 and 507. Nucleotides comprising a different predetermined base type in step (c) may be used each time. Washing and other treatments may be applied in between described steps as recognized and known by those skilled in the art. Labels, labeling, cleavable nucleotides and other reagents and conditions are described in more detail elsewhere herein.

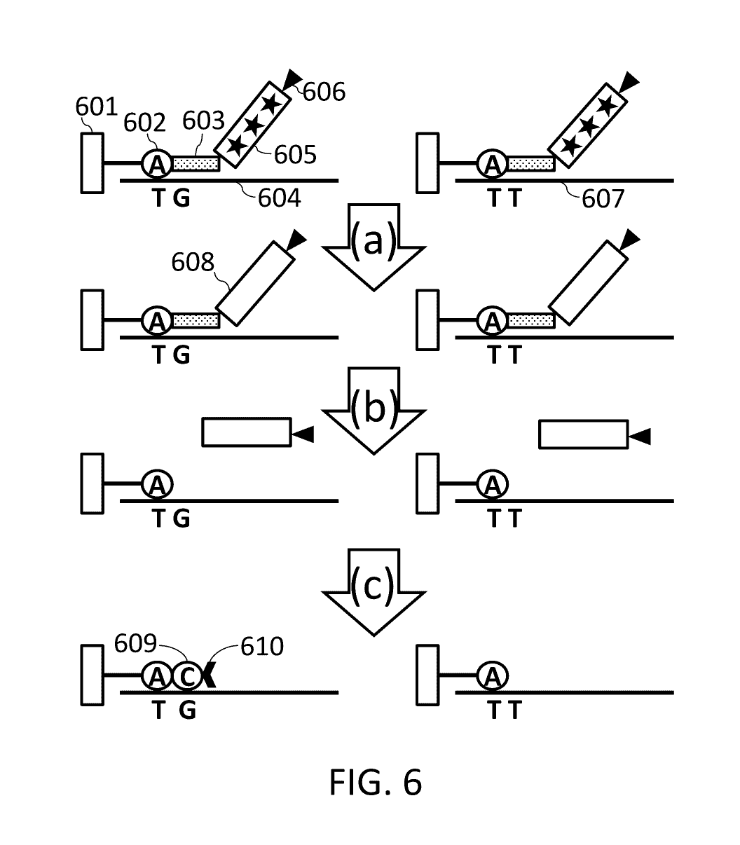

In one embodiment shown in FIG. 6, a blocking nucleotide tail and a removable nucleotide tail are constructed in two nucleic acid molecules already having previously constructed removable nucleotide tails, in a manner that enables sequencing of the nucleic acid molecules. FIG. 6 shows two nucleic acid molecules, one is 604 and another 607. In FIG. 6, the same numbers apply to refer to drawn parts that have the same features in both 604 and 607. The nucleic acid molecule 604 is a DNA strand with its complementary extendable strand anchored to a solid support 601. 604 has a thymine (T) at a specific position, which is immediately followed by a guanine (G). The thymine is bound to its complementary adenine (A), which is comprised in deoxyribonucleotide 602 in the strand complementary to 604. 602 is extended by a removable nucleotide tail. Said tail comprises a first segment 603 and a second segment 605. Segment 603 comprises cleavable nucleotides, whereas segment 605 comprises unlabeled and labeled deoxyribonucleotides. Segment 605 is previously constructed by template-independent polymerization and has a dideoxyribonucleotide 606 at its 3′ end. The labels within 605 are specific for the presence of the base type adenine, meaning that detection of said labels indicates the presence of adenine in the deoxyribonucleotide (602) preceding (i.e., associated with the 5′ end of) the removable nucleotide tail. In this embodiment, the labels are fluorescent.

Next to the nucleic acid molecule 604 in FIG. 6 there is another nucleic acid molecule shown, 607. Nucleic acid molecule 607 has the same features with 604, except thymine is followed by another thymine (T), and not guanine. The method can apply to a mixture of nucleic acid molecules, wherein there are nucleic acid molecules that behave like the nucleic acid molecule in the left side, and others that behave like the nucleic acid molecule in the right side.

During step (a) in FIG. 6, both nucleic acid molecules 604 and 607, and their surroundings, are exposed to photobleaching as described elsewhere herein, in order to damage the labels. 608 is the resulting photobleached removable nucleotide tail (the same applies to the tail in 607). Photobleaching is a useful method, because photobleached removable nucleotide tails do not interfere with the labels of subsequently constructed labeled tails, in the event that said photobleached tails are not removed completely.

During step (b), both nucleic acid molecules 604 and 607 are exposed to conditions and reagents that release the cleavable nucleotides of the first segments of the removable nucleotide tails (603). Said conditions and reagents are suitable for the type of cleavable nucleotides used in the tails, and are described in detail elsewhere herein. Upon completion of step (b), the 3′ end of the deoxyribonucleotide 602 (in both 604 and 607) becomes available for extension by polymerization (i.e. said end regains a OH group). Complete removal of said first segment (603) causes removal of the second segment (608), as shown in FIG. 6. In the event that the removal is partial and 608 remains associated with the nucleic acid molecule, 606 prevents off-site extension during subsequent steps.

During step (c), both nucleic acid molecules 604 and 607, and their surroundings, are exposed to conditions to cause polymerization, and to a template-dependent polymerization reaction comprising reversibly terminated deoxyribonucleotides comprising a predetermined base type, which in this case is cytosine (C). As shown in FIG. 6, a deoxyribonucleotide 609 comprising cytosine is successfully incorporated into the nucleic acid molecule 604. Said nucleotide comprises a reversible terminator 610. There is no incorporation occurring in 607, because 607 has a thymine instead of a guanine. The following steps can be conducted as shown in FIG. 5B and FIG. 5C.

Washing and other treatments may be applied in between described steps as recognized and known by those skilled in the art. Labels, labeling, cleavable nucleotides and other reagents and conditions are described in more detail elsewhere herein.

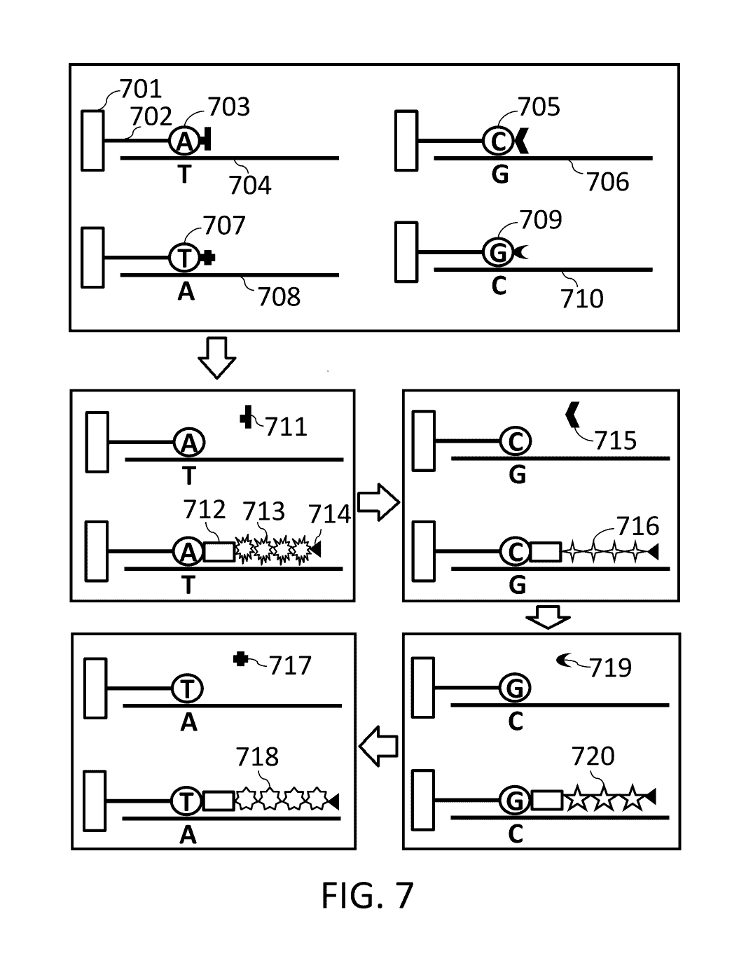

In one embodiment shown in FIG. 7, nucleic acid molecules 704, 706, 708 and 710 are DNA strands with their complementary extendable strand (702) anchored to a solid support (701). The nucleic acid molecules are exposed to conditions to cause polymerization, and to a template-dependent polymerization reaction solution comprising nucleotides to complement said nucleic acid molecules. Said nucleotides are deoxyribonucleotides comprising reversible terminators. Nucleotides comprising a specific base type comprise reversible terminators of a different type from the terminators of nucleotides comprising other base types. Each terminator type comprised in the population of said nucleotides can be removed by different conditions from other terminator types. Reversible terminators are described elsewhere herein. Upon completion of the polymerization reaction, each nucleic acid molecule incorporates a single reversibly terminated deoxyribonucleotide comprising a complementary base type. Nucleic acid molecule 704 incorporates deoxyribonucleotide 703 comprising adenine (A), nucleic acid molecule 706 incorporates deoxyribonucleotide 705 comprising cytosine (C), nucleic acid molecule 708 incorporates deoxyribonucleotide 707 comprising thymine (T), and nucleic acid molecule 710 comprises deoxyribonucleotide 709 comprising guanine (G).

In order to construct a removable nucleotide tail specific for the presence of adenine, the reversible terminator 711 comprised in the adenine-containing nucleotide is removed. The reversible terminators specific for the other base types remain intact. A removable nucleotide tail comprising segment 712 comprising cleavable nucleotides, segment 713 comprising unlabeled and labeled deoxyribonucleotides, and dideoxyribonucleotide 714, is constructed as shown for previously described embodiments. The labels within 713 are specific for the presence of adenine.

In another separate step, the reversible terminator 715 comprised in the cytosine-containing nucleotide is removed. The reversible terminators specific for the other base types remain intact. A removable nucleotide tail is constructed comprising a segment 716 that is labeled specifically for the presence of cytosine.

In another separate step, the reversible terminator 719 comprised in the guanine-containing nucleotide is removed. The reversible terminators specific for the other base type remain intact. A removable nucleotide tail is constructed comprising a segment 720 that is labeled specifically for the presence of guanine.

In another separate step, the reversible terminator 717 comprised in the thymine-containing nucleotide is removed. A removable nucleotide tail is constructed comprising a segment 718 that is labeled specifically for the presence of thymine.

Detection of the labels in 713, 716, 720 and 718 enables base determination of the nucleotides incorporated at specific positions of the nucleic acid molecules 704, 706, 710 and 708 respectively.

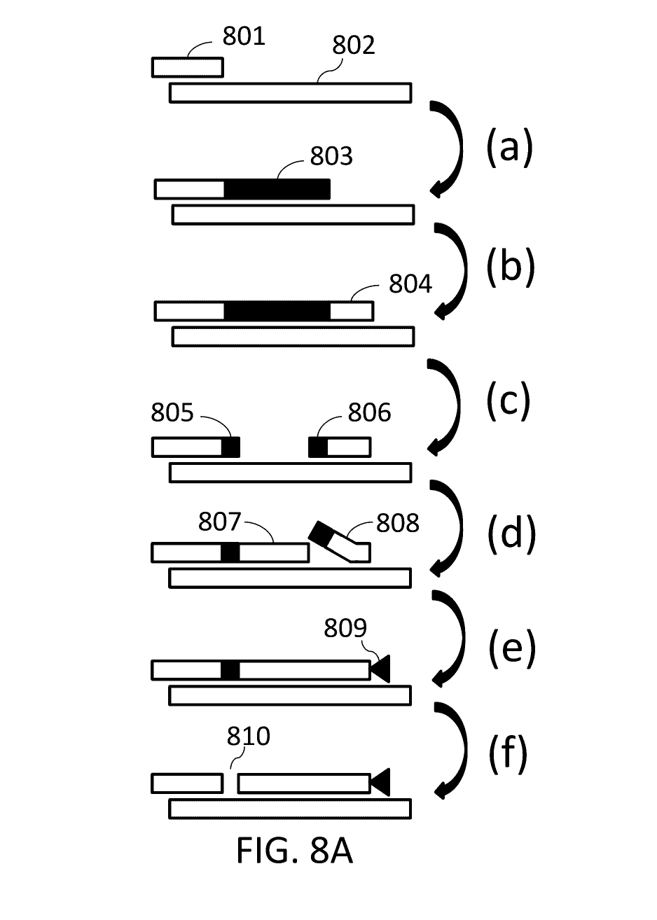

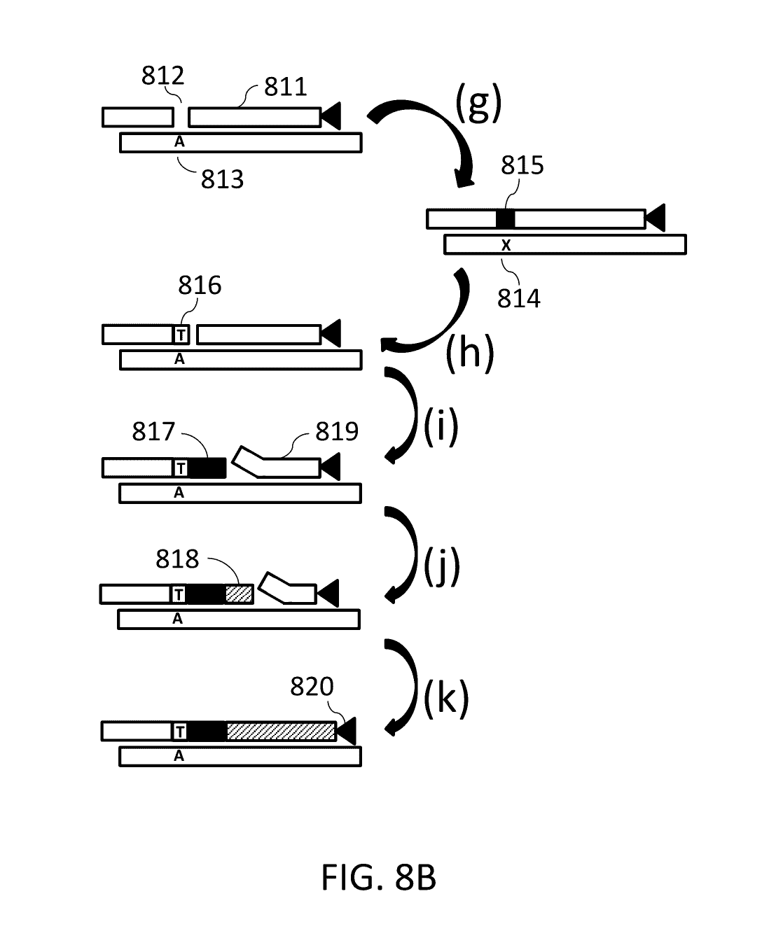

In one embodiment shown in FIG. 8A and FIG. 8B, a removable nucleotide tail is constructed in the event that a nucleotide comprising a predetermined base type is incorporated into a nucleic acid molecule. A difference of this embodiment with previously described embodiments is that there is no use of reversibly terminated nucleotides. In this embodiment, the nucleic acid molecule 802 is a DNA strand hybridized to primer 801 comprising an extendable 3′ end. 801 may be anchored to a solid surface (not shown). The method can apply to a mixture of nucleic acid molecules.

During step (a) in FIG. 8A, the nucleic acid molecule is exposed to polymerization conditions, and to a template-dependent polymerization reaction solution comprising ribonucleotides to complement 802, resulting in the production of the RNA segment 803.

During an optional step (b), the nucleic acid molecule is exposed to polymerization conditions, and to a template-dependent polymerization reaction solution comprising deoxyribonucleotides to complement 802, resulting in the production of the segment 804.

During step (c), the nucleic acid molecule is exposed to conditions and reagents that cleave phosphodiester bonds between adjacent ribonucleotides, but not the bond between the 3′ end of a deoxyribonucleotide and the 5′ end of the ribonucleotide. Examples of such conditions include treatment with RNase HI, lanthanides or alkaline hydrolysis. In the event that RNase HI is used, the phosphodiester bonds between adjacent ribonucleotides are cleaved, but not the junction bonds (i.e., the phosphodiester bond between a ribonucleotide and a deoxyribonucleotide). During step (c), the RNA segment 803 is digested, with the exception of the two ribonucleotides 805 and 806 next to the DNA segments 801 and 804.

During step (d), the nucleic acid molecule and its surroundings are exposed to polymerization conditions, and to a template-dependent polymerization reaction solution comprising deoxyribonucleotides to complement the nucleic acid molecule, resulting in the production of the segment 807 (FIG. 8A shows 807 production being in progress, so 807 is not shown in its final length). Polymerases used in the reaction possess strand-displacing activity and displace 808 as they produce 807. In another embodiment, the polymerases used possess 5′-to-3′ activity and digest part of 808 as they produce 807.

During step (e), the nucleic acid molecule and its surroundings are exposed to polymerization conditions, and to a template-dependent polymerization reaction solution comprising dideoxyribonucleotides to complement the nucleic acid molecule. Polymerases used in the reaction are strand-displacing or possess 5′-to-3′ exonuclease activity. During this step, 807 is irreversibly terminated with the incorporation of dideoxyribonucleotide 809.

During step (f), the nucleic acid molecule and its parts are exposed to conditions to create a single-base gap. Such conditions may include, for example, using active RNase HII and FEN1 molecules. RNase HII is a ribonuclease that is able to cleave the phosphodiester bond between the 3′ end of a deoxyribonucleotide and the 5′ end of a ribonucleotide within a double-stranded nucleic acid molecule. FEN1 is a flap endonuclease that participates with RNase HII in the excision of single ribonucleotides embedded in double-stranded DNA molecules. In other embodiments, treatment with RNase HII is performed first, followed by alkaline hydrolysis or hydrolysis with lanthanide salts. Treatments such as alkaline hydrolysis may denature double strands, and interfere with single-base gap formation. In embodiments that employ such treatments, it may be suitable to use modifications or constructs that hold strands together, such as crosslinking or hairpin adaptors (as shown and explained elsewhere herein). Step (f) generates the single-base gap 810.

FIG. 8B shows the construction of a labeled removable nucleotide tail in the event that adenine (A) is in the position 813 of the nucleic acid molecule, said position facing the gap 812 of strand 811. In the event that there is a base type other than adenine (marked with X in position 814) in said position, the gap is filled and sealed during step (g), forming a terminal blocking nucleotide tail. Step (g) comprises exposing the nucleic acid molecule and its parts to conditions to cause polymerization and ligation, and to a template-dependent polymerization and ligation reaction solution comprising deoxyribonucleotides that do not comprise a predetermined base type, which in this case is thymine. The polymerases used in this step do not possess strand-displacing activity, and do not possess 5′-to-3′exonuclease activity, and are suitable for filling the gap. The gap is filled with a deoxyribonucleotide (815) by polymerase and sealed by ligase. Since deoxyribonucleotides are not cleavable nucleotides in this context, step (g) leads to the formation of a terminal blocking nucleotide tail. In another embodiment, cleavable blocked nucleotides not comprising the predetermined base type (thymine) are used instead of deoxyribonucleotides, and no ligation is used, leading to the formation of a blocking nucleotide tail. An example of a cleavable blocked nucleotide is a-S-ddNTP that can be incorporated by using Thermosequenase.

The next steps show the processes of constructing a labeled removable nucleotide tail in the event that thymine is complementary to the base exposed by the gap. After step (g), and during step (h), the nucleic acid molecule and its parts are exposed to polymerization conditions, and to a template-dependent polymerization reaction solution comprising deoxyribonucleotides comprising thymine. The polymerases used in this step do not possess strand-displacing activity, and do not possess 5′-to-3′exonuclease activity. Upon completion of the reaction, deoxyribonucleotide 816 comprising thymine fills the gap. Sealing does not take place, because there is no ligase present in the reaction, thus leaving a free 3′ end that can be extended further in subsequent steps.

Indeed, during step (i), the nucleic acid molecule and its parts are exposed to polymerization conditions, and to a template-dependent polymerization reaction solution comprising a mixture of labeled and unlabeled cleavable nucleotides to complement the nucleic acid molecule. Polymerases used in said reaction have strand-displacement capability, and displace 819, as 817 is produced, as shown in FIG. 8B. In another embodiment, polymerases having 5′-to-3′ exonuclease activity are used instead.

During step (j), the nucleic acid molecule and its parts are exposed to polymerization conditions, and to a template-dependent polymerization reaction solution comprising a mixture of labeled and unlabeled deoxyribonucleotides to complement the nucleic acid molecule. The polymerases used in the reaction are strand-displacing, as in step (i). Segment 818 is constructed during this step (FIG. 8B shows 818 production being in progress, so 818 is not shown in its final length).

During step (k), the nucleic acid molecule and its parts are exposed to polymerization conditions, and to a template-dependent polymerization reaction solution comprising polymerase molecules and dideoxyribonucleotides to complement the nucleic acid molecule. During this step, 818 is irreversibly terminated with the incorporation of dideoxyribonucleotide 820.

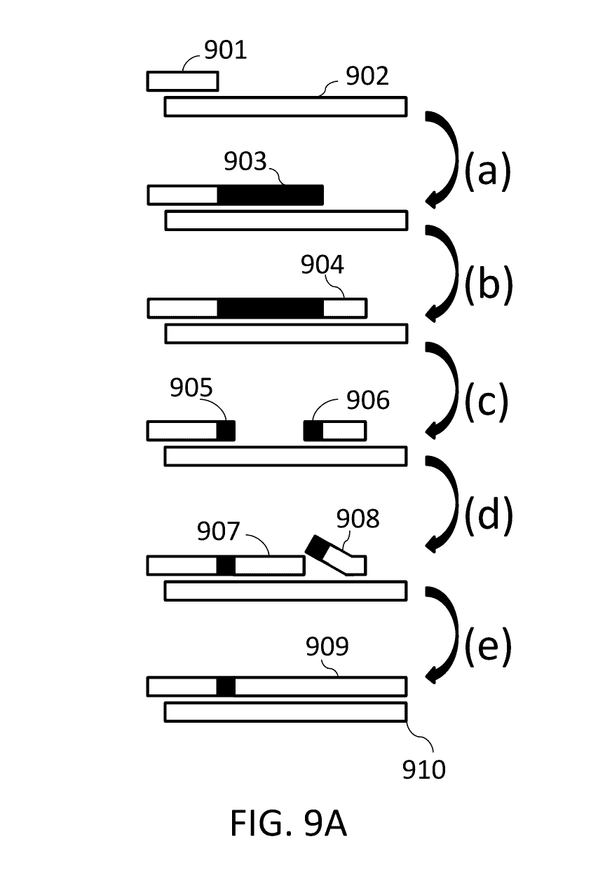

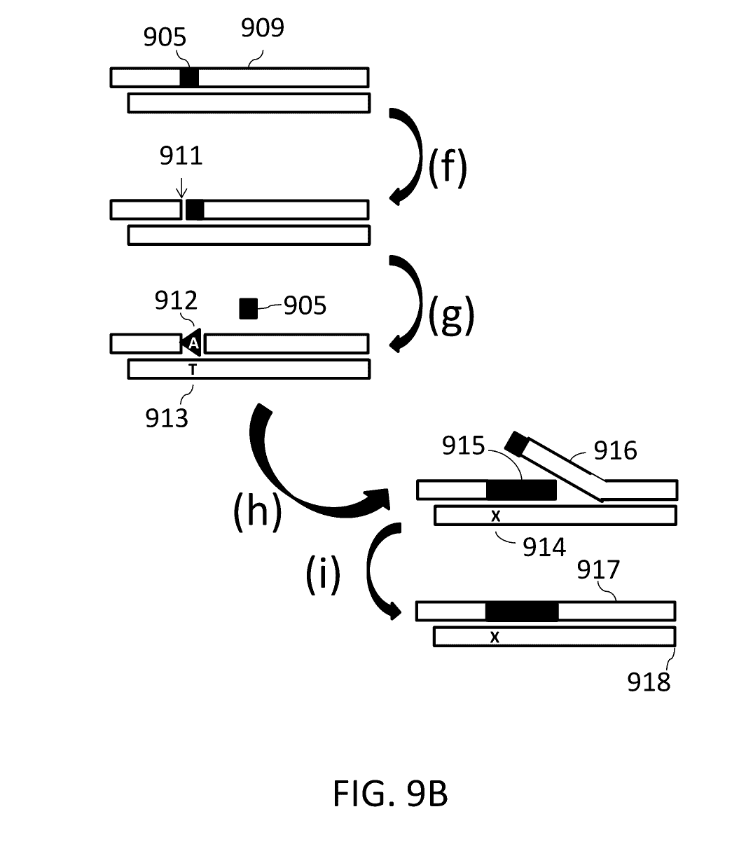

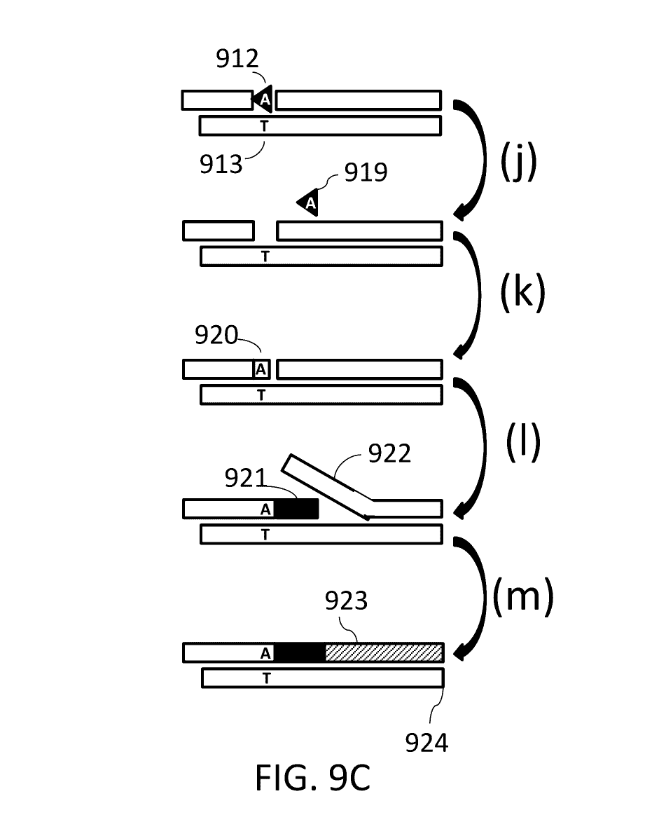

In one embodiment shown in FIG. 9A, FIG. 9B and FIG. 9C, a removable nucleotide tail is constructed in the event that a nucleotide comprising a predetermined base type is incorporated into a nucleic acid molecule. Similarly to the embodiment described in FIGS. 8A and B, there is no use of reversibly terminated nucleotides. In this embodiment, the nucleic acid molecule 902 is a DNA strand hybridized to primer 901 comprising an extendable 3′ end. 901 may be anchored to a solid surface (not shown). The method can apply to a mixture of nucleic acid molecules.

During step (a) in FIG. 9A, the nucleic acid molecule is exposed to polymerization conditions, and to a template-dependent polymerization reaction solution comprising ribonucleotides to complement 902, resulting in the production of the RNA segment 903.

During optional step (b), the nucleic acid molecule is exposed to polymerization conditions, and to a template-dependent polymerization reaction solution comprising deoxyribonucleotides to complement 902, resulting in the production of the segment 904.

During step (c), the nucleic acid molecule is exposed to conditions and reagents that cleave phosphodiester bonds between adjacent ribonucleotides, but not the bond between the 3′ end of a deoxyribonucleotide and the 5′ end of a ribonucleotide. Examples of such conditions and reagents include treatment with RNase HI, lanthanides or alkaline hydrolysis. In the event that RNase HI is used, the phosphodiester bonds between adjacent ribonucleotides are cleaved, but not the junction bonds (i.e., the phosphodiester bond between a ribonucleotide and a deoxyribonucleotide). During step (c), the RNA segment 903 is digested, with the exception of the two ribonucleotides 905 and 906 next to the DNA segments 901 and 904.

During steps (d) and (e), the nucleic acid molecule and its surroundings are exposed to polymerization conditions, and to a template-dependent polymerization reaction solution comprising deoxyribonucleotides to complement the nucleic acid molecule, resulting in the production of the segment 907. FIG. 9A, step (d), shows 907 production being in progress, so 907 is not shown in its final length, whereas FIG. 9A, step (e), shows 907 in its final state, 909, which reaches the 5′ end side (910) of the nucleic acid molecule 902. Polymerases used in the reaction possess strand-displacing activity and displace 908 as they produce 907. In another embodiment, polymerases used possess 5′-to-3′ activity and digest 908 as they produce 907.

During step (f) shown in FIG. 9B, the nucleic acid molecule and its parts are exposed to active RNase HII molecules. RNase HII cleaves the phosphodiester bond between the ribonucleotide 905 and the deoxyribonucleotide bound to the 5′ end of said ribonucleotide, thus creating nick 911.

During step (g), the nucleic acid molecule and its parts are exposed to polymerization conditions, and to a template-dependent polymerization reaction solution comprising dideoxyribonucleotides comprising a predetermined base type. The polymerases used in this step may possess 5′-to-3′ exonuclease, so that they excise ribonucleotide 905. In the event that the polymerases in this step do not possess 5′-to-3′ exonuclease activity, ribonucleotide 905 can be excised by other methods, such as treatment with lanthanide salts. The incorporated dideoxyribonucleotide 912 terminates the reaction by preventing any further extension. In the example shown in FIG. 9B, the predetermined base type is adenine, and a dideoxyribonucleotide 912 comprising adenine (A) is successfully incorporated, in the event that a thymine (T) is found in the specific position 913 of the nucleic acid molecule.

Subsequent steps (h) and (i) construct a blocking nucleotide tail in the event that no incorporation takes place in step (g) because a base type other than thymine occupies position 913 (base marked with X, 914). In the event that incorporation takes place in step (g), the nucleic acid molecule remains unaltered during steps (h) and (i). During step (h), the nucleic acid molecule and its parts are exposed to polymerization conditions, and to a template-dependent polymerization reaction solution comprising cleavable nucleotides to complement the nucleic acid molecule. Polymerases used in the reaction possess strand-displacing activity and displace 916 as they produce 915. In another embodiment, polymerases used possess 5′-to-3′ activity and digest part of 916 as they produce 915.

During step (i), the nucleic acid molecule and its parts are exposed to polymerization conditions, and to a template-dependent polymerization reaction solution comprising deoxyribonucleotides to complement the nucleic acid molecule, resulting in the production of the segment 917. 917 reaches the 5′ end side (918) of the nucleic acid molecule 902. Polymerases used in the reaction possess strand-displacing activity. In another embodiment, the polymerases used possess 5′-to-3′ activity.

FIG. 9C shows the construction of a labeled removable nucleotide tail in the event that thymine (T) is in the position 913 of the nucleic acid molecule, and dideoxyribonucleotide 912 is incorporated during step (g). During step (j), the nucleic acid molecule and its parts are exposed to conditions to cause pyrophosphorolysis, and to a pyrophosphorolysis reaction solution comprising suitable polymerase molecules and pyrophosphate (PPi) molecules, as described in (Liu and Sommer, 2004). The result of the reaction in this step is the removal of the dideoxyribonucleotide (919) that is incorporated during step (g).

During step (k), the nucleic acid molecule and its parts are exposed to polymerization conditions, and to a template-dependent polymerization reaction solution comprising deoxyribonucleotides comprising the predetermined base type (which is adenine in this example). The polymerases used in this step do not possess strand-displacing activity, and do not possess 5′-to-3′ exonuclease activity, and are thus suitable for filling the gap generated in the previous step (j). The gap is filled with a deoxyribonucleotide comprising adenine (A in 920). Said deoxyribonucleotide has a free 3′ end (end is not sealed, as shown in FIG. 9C).

During step (l), the nucleic acid molecule and its parts are exposed to polymerization conditions, and to a template-dependent polymerization reaction solution comprising a mixture of labeled and unlabeled cleavable nucleotides to complement the nucleic acid molecule. Polymerases used in said reaction have strand-displacement capability, and thus produce 921 and displace 922, as shown in FIG. 9C. In another embodiment, polymerases having 5′-to-3′ exonuclease activity are used instead.

During step (m), the nucleic acid molecule and its parts are exposed to polymerization conditions, and to a template-dependent polymerization reaction solution comprising a mixture of labeled and unlabeled deoxyribonucleotides to complement the nucleic acid molecule. Polymerases used in the reaction are strand-displacing, as in step (l). Segment 923 is constructed during this step, which reaches the 5′ end side (924) of the nucleic acid molecule.

In an embodiment similar to the previous embodiment of FIG. 9, the nucleotide incorporated in step (g) is not a dideoxyribonucleotide, but instead a cleavable terminated nucleotide such as phosphorothioate-modified dideoxyribonucleotide, and step (j) does not comprise pyrophosphorolysis, but instead a cleavage method that excises the nucleotide in step (g) (e.g., iodoethanol, in the event that phosphorothioate-modified nucleotide is incorporated in step (g)).

Washing and other treatments may be applied in between described steps as recognized and known by those skilled in the art.

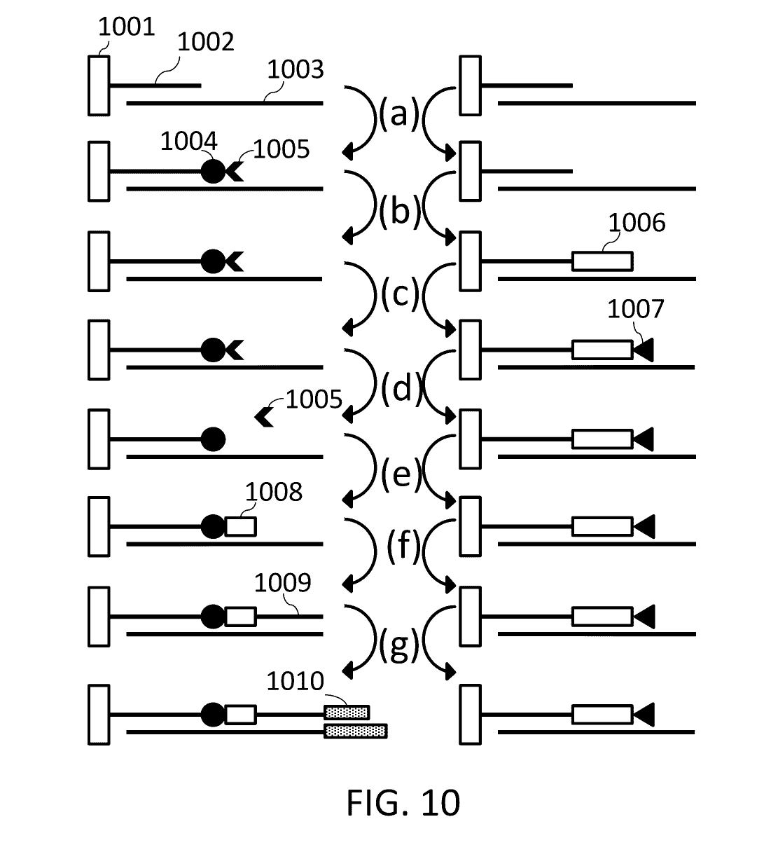

In one embodiment shown in FIG. 10, a tail tag is attached in the event that a nucleotide comprising a predetermined base type is incorporated into a nucleic acid molecule. A nucleic acid molecule 1003 is a single DNA strand hybridized to another DNA strand 1002 that is anchored to a solid support 1001. 1003 has a ligatable 5′ end. The anchored strand 1002 has an extendable 3′ end, which can be extended by polymerization. In FIG. 10, the left side shows the nucleic acid molecule 1003 participating in steps (a) through (g) in the event that the nucleic acid molecule 1003 incorporates a nucleotide comprising a predetermined base type in step (a), whereas the right side of FIG. 10 shows the same nucleic acid molecule 1003 participating in the same steps (a) through (g) in the event that no incorporation takes place during step (a). The method can be applied to a mixture of nucleic acid molecules, wherein there are nucleic acid molecules that behave like the nucleic acid molecule in the left side, and others that behave like the nucleic acid molecule in the right side.

During step (a) in FIG. 10, 1003 and its surroundings are exposed to polymerization conditions, and to a template-dependent polymerization reaction solution comprising reversibly terminated nucleotides comprising a predetermined base type.

In the embodiment of FIG. 10, a nucleotide 1004 comprising the predetermined base type is successfully incorporated into the nucleic acid molecule shown at the left side of FIG. 10. The nucleotide comprises a reversible terminator 1005. The right side of FIG. 10 shows that no incorporation takes place. In this case, nucleotides comprising the predetermined base type are not complementary to the nucleic acid molecule at the specific position following the extendable 3′ end.

In this embodiment, the process continues with steps (b) and (c), during which a non-ligatable blocking nucleotide tail is constructed in the event that no nucleotide incorporation occurs during step (a). The purpose of the non-ligatable blocking nucleotide tail is to prevent construction of a ligatable removable nucleotide tail and attachment of a tail tag in the event that the nucleic acid molecule does not incorporate the predetermined nucleotide type of step (a). In this embodiment, the constructed non-ligatable blocking nucleotide tail comprises a segment of cleavable nucleotides terminated with the addition of a dideoxyribonucleotide. Step (b) comprises exposing the nucleic acid molecule and its parts to polymerization conditions, and to a template-dependent polymerization reaction solution comprising cleavable nucleotides to complement the nucleic acid molecule 1003. In the event that no incorporation occurs in step (a), step (b) produces segment 1006 which is complementary to the nucleic acid molecule 1003. In the event that there is incorporation of a nucleotide during step (a), step (b) does not have any effect, as shown in the left side of FIG. 10.

The segment 1006 can be terminated during step (c). In this embodiment, step (c) comprises exposing the nucleic acid molecule and its parts to polymerization conditions, and to a template-dependent polymerization reaction solution comprising dideoxyribonucleotides to complement the nucleic acid molecule 1003. Step (c) leads to incorporation of 1007. The incorporation of 1007 prevents construction of a ligatable removable nucleotide tail in the event that there is no nucleotide incorporation during step (a). In the event that there is incorporation of a nucleotide during step (a), step (c) does not have any effect, as shown in the left side of FIG. 10.

During step (d) in FIG. 10, the reversible terminator 1005 is removed by exposing the nucleic acid molecule and its surroundings to appropriate conditions and reagents, which are described elsewhere herein. In the event that there is a non-ligatable blocking nucleotide tail constructed into the nucleic acid molecule 1003 during step (b), step (d) has no effect.

During step (e), the construction of a first segment of a ligatable removable nucleotide tail may occur. In this embodiment, step (e) comprises exposing the nucleic acid molecule 1003 and its surroundings to conditions to cause polymerization, and to a template-dependent polymerization reaction solution that comprises cleavable nucleotides to complement the nucleic acid molecule 1003. In the event that no nucleotide is incorporated into the nucleic acid molecule 1003 during step (a), step (e) has no effect and the nucleic acid molecule 1003 remains carrying the non-ligatable blocking nucleotide tail, as shown in FIG. 10, right side. In the event that a nucleotide is incorporated into the nucleic acid molecule 1003 during step (a), step (e) produces segment 1008 comprising cleavable nucleotides, as shown in FIG. 10, left side. For reasons explained in FIG. 2 that involve reduced rates of cleavable nucleotide incorporation, 1008 may not reach the end of 1003. Achieving full extension reaching the end of the 1003 strand is desirable in this embodiment, to allow ligation to a tail tag. 1008 can be further extended during step (f), which comprises exposing the nucleic acid molecule 1003 and its parts to conditions to cause polymerization, and to a template-dependent polymerization reaction solution comprising deoxyribonucleotides to complement the nucleic acid molecule 1003. During step (f), segment 1009 of the ligatable removable nucleotide tail is constructed, in the event that a nucleotide is incorporated into the nucleic acid molecule during step (a), as shown in FIG. 10, left side. In the event that no incorporation occurs during step (a), step (f) has no effect, as shown in FIG. 10, right side. As shown in FIG. 10, segment 1009 reaches the 5′ end of the nucleic acid molecule 1003, forming a ligatable blunt end.

The last step, step (g) comprises attaching a tail tag to the ligatable blunt end of the previous step. This is accomplished by exposing the nucleic acid molecule and its parts to conditions to cause ligation, and to a ligation reaction solution comprising tail tags representing the predetermined base type in step (a). FIG. 10, left side, shows the tail tag 1010 being attached to the nucleic acid molecule and the ligatable removable nucleotide tail. In the event that no incorporation occurs during step (a), step (g) has no effect, as shown in FIG. 10, right side. Washing and other treatments may be applied in between described steps as recognized and known by those skilled in the art. Ligases, cleavable nucleotides, terminated nucleotides and other reagents and conditions are described in more detail elsewhere herein.

Tail tags are constructs that can ligate to a nucleic acid molecule, said nucleic acid molecule comprising a ligatable removable nucleotide tail. A tail tag can ligate to only the 5′ end of the template strand of said nucleic acid molecule, or to both the 5′ end of the template strand and the 3′ end of the ligatable removable nucleotide tail. A tail tag can be an oligonucleotide or polynucleotide, single-stranded or double-stranded, that can ligate to a nucleic acid molecule as described. A tail tag comprises at least two nucleotides. Some tail tags may comprise eight or more nucleotides or base pairs. Other tail tags may comprise 20 or more nucleotides or base pairs. A tail tag may be double-stranded, comprising oligonucleotides or polynucleotides that are at least partially complementary to one another and can anneal to form a dimer. Methods of annealing and methods of designing appropriate oligonucleotide and polynucleotide sequences to achieve annealing are known to people skilled in the art. A double-stranded tail tag comprises a strand that can ligate to the 5′ end of the template strand of a nucleic acid molecule, said strand termed the “remaining part”, and another strand that can optionally ligate to the 3′ end of the ligatable removable nucleotide tail comprised in the nucleic acid molecule, said strand termed the “removable part”. A double-stranded tail tag comprises one end that ligates to a nucleic acid molecule and another end that may be non-ligatable, said end comprising the 3′ end of the removable part and the 5′ of the remaining part of the tail tag. The non-ligatable end cannot ligate to other tail tags and cannot ligate to the nucleic acid molecule.

In certain embodiments, tail tags comprise specific sequences, or labels, or other detectable features, or combinations thereof that are designated to represent specific nucleotide base types. Each type of tail tag may represent one base type. A tail tag that represents a specific base type can be attached to a nucleic acid molecule in the event that a nucleotide comprising the specific base type is incorporated into the nucleic acid molecule. Successive nucleotide incorporation events, each of which is followed by attachment of a tail tag that represents the base type of the incorporated nucleotide, leads to a series of tail tags attached in order reflecting the sequence of the nucleic acid molecule.

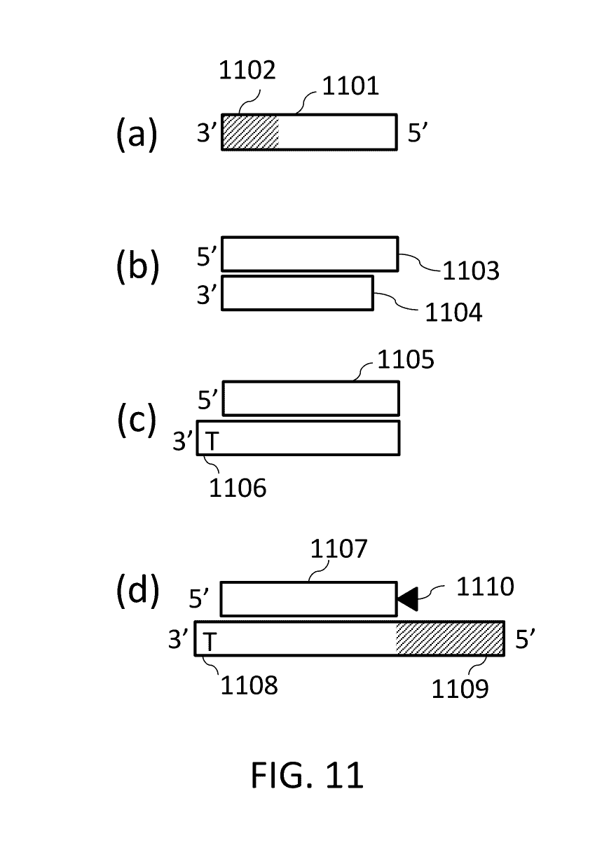

As shown in FIG. 11, different types of tail tags can be used. The tail tags shown in FIG. 11 are non-limiting examples. In specific embodiments, tail tags are DNA constructs. In another embodiment, a single-stranded DNA tail tag 1101 is used, with structure as shown in (a). 1101 comprises a section 1102 that is complementary to the end part of a ligatable removable nucleotide tail (not shown), that renders 1101 able to ligate to the 5′ end of the nucleic acid molecule comprising said ligatable removable nucleotide tail.

Another example of a tail tag is shown in (b). The tail tag in (b) is a double-stranded tail tag, comprising the removable part 1103 which can ligate to a ligatable removable nucleotide tail with its 5′ end, and the remaining part 1104 which can ligate (with its 3′ end) to a nucleic acid molecule comprising said ligatable removable nucleotide tail. The tail tag shown in (b) is suitable for blunt ligations.

Another example of a tail tag is shown in (c). The tail tag 1105 in (c) is a double-stranded tail tag that is suitable for TA ligation reactions because of its thymine (T)-containing single-nucleotide overhang 1106. The other end of the tail tag 1105 is blunt to prevent inappropriate ligation.

One example of a tail tag is shown in (d). The tail tag 1107 is a double-stranded DNA construct suitable for TA ligation reactions because of its thymine (T)-containing single-nucleotide overhang 1108. 1107 also comprises a protruding 5′ end 1109 (shown as shaded area) which protects the tail tag from T7 exonuclease digestion, as described in a later Figure herein. Both 5′ ends of the tail tag are phosphorylated. 1107 also comprises a terminated nucleotide such as dideoxyribonucleotide at 1110, which prevents off-site polymerization, inappropriate ligatable removable nucleotide tail formation, etc.

As mentioned above, a tail tag has at least one strand, which can be attached to a nucleic acid molecule, said strand termed the “remaining part”, because it is not removed after attachment. On the other hand, a strand termed “removable part” is the strand that may be attached to a ligatable removable nucleotide tail, and may be removed when a new ligatable removable nucleotide tail is constructed. This is demonstrated in later figures herein. In the event that a tail tag is labeled, the tail tag may be constructed in such a way that at least the remaining part is labeled.

Those skilled in the art can design tail tags with many different features.

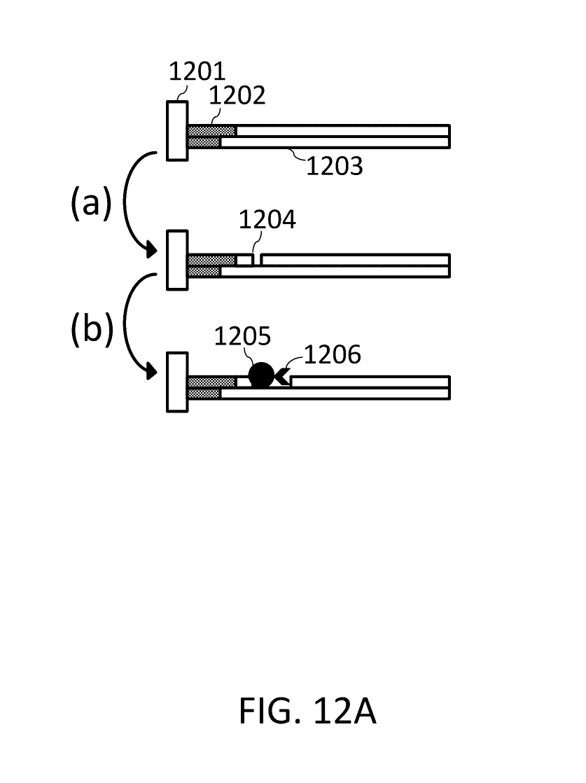

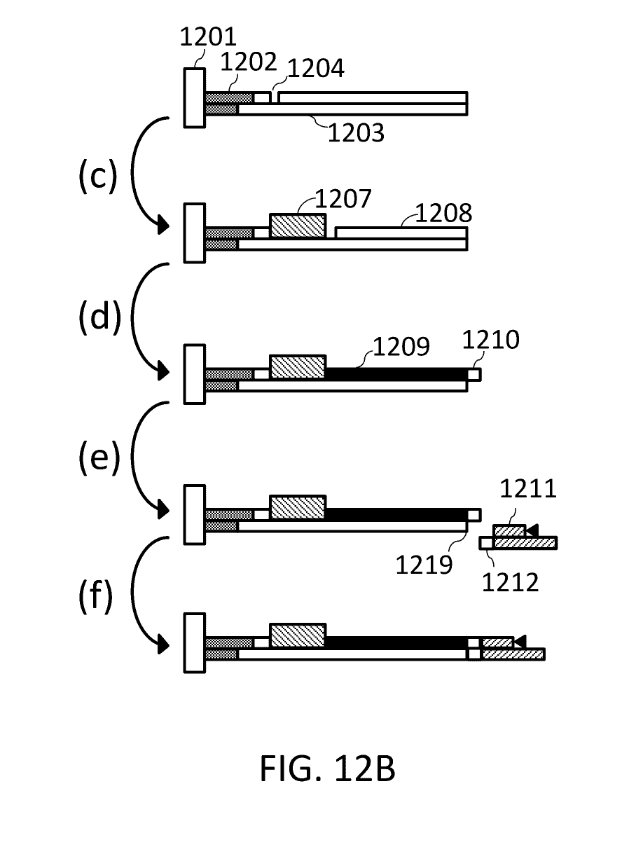

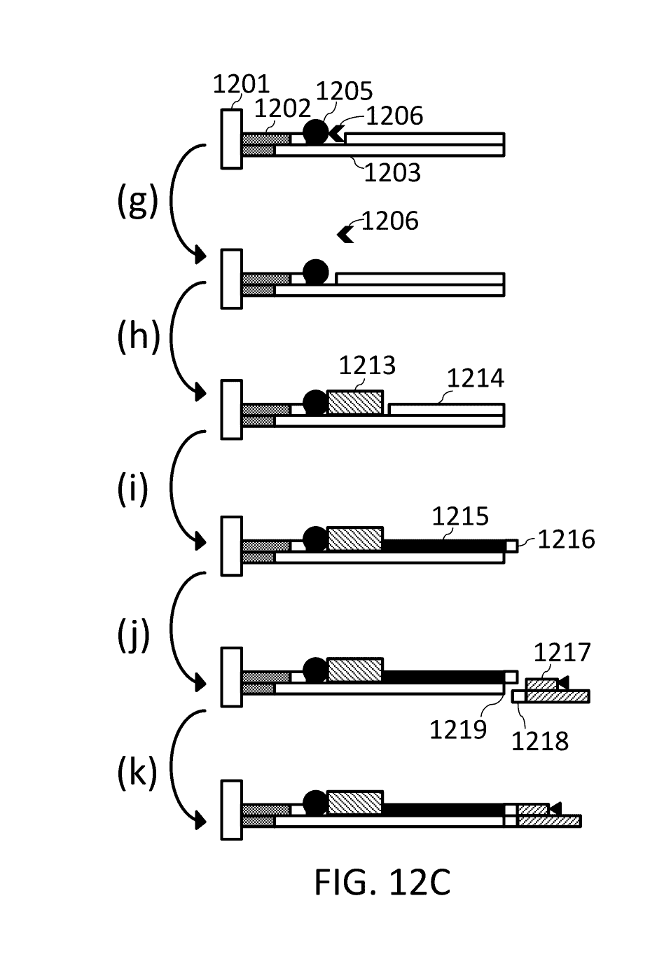

In FIGS. 12A, 12B and 12C, an embodiment is described for the attachment of a protective tail tag and an initial tail tag. FIG. 12A shows nucleic acid molecule 1203. Said nucleic acid molecule is double-stranded DNA attached to adaptor 1202, and its free 5′ end is ligatable. Said adaptor is anchored to a solid support 1201 and comprises a recognition site of a nicking endonuclease. Said endonuclease can create a nick within the nucleic acid molecule 1203, close to the 3′ end of the adaptor 1202, said end being attached to the nucleic acid molecule 1203.

During step (a) in FIG. 12A, the nucleic acid molecule 1203 and its parts are exposed to conditions to cause digestion, and to an endonuclease reaction solution comprising nicking endonuclease molecules that specifically bind to said recognition site within the adaptor. A nick within the nucleic acid molecule is created during the reaction. Said nick has an extendable 3′ end (1204).

During step (b), the nucleic acid molecule and its parts are exposed to conditions to cause polymerization, and to a template-dependent polymerization reaction solution comprising reversibly terminated deoxyribonucleotides comprising a predetermined base type. Polymerases used in the reaction possess 5′-to-3′ exonuclease activity. In another embodiment, said polymerases have strand-displacing activity. In the event that a nucleotide comprising the predetermined base type is complementary to the nucleic acid molecule at the specific position following the extendable 3′ end, incorporation takes place, as shown in FIG. 12A, where nucleotide 1205 is incorporated into the nucleic acid molecule, said nucleotide comprising a reversible terminator 1206.

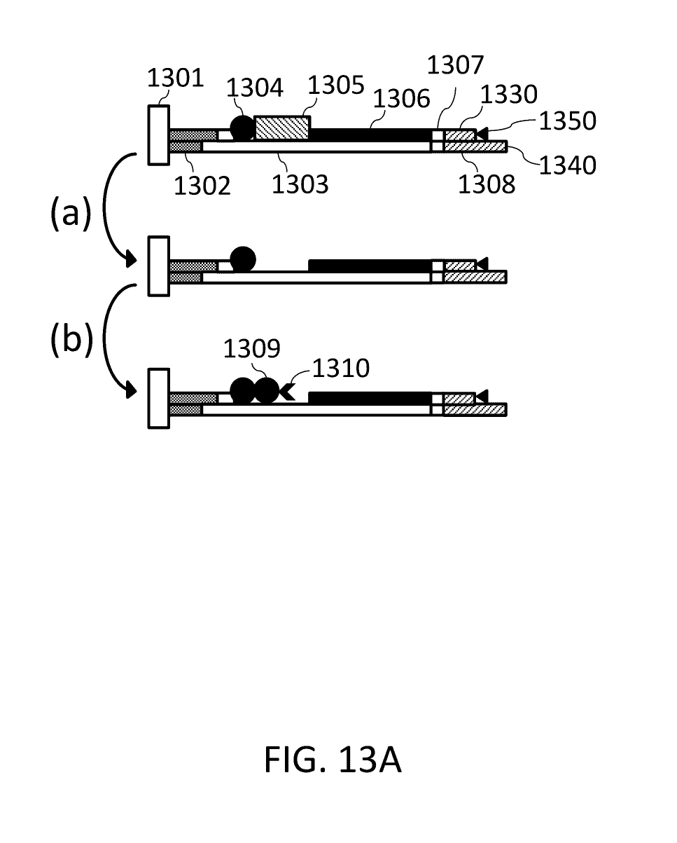

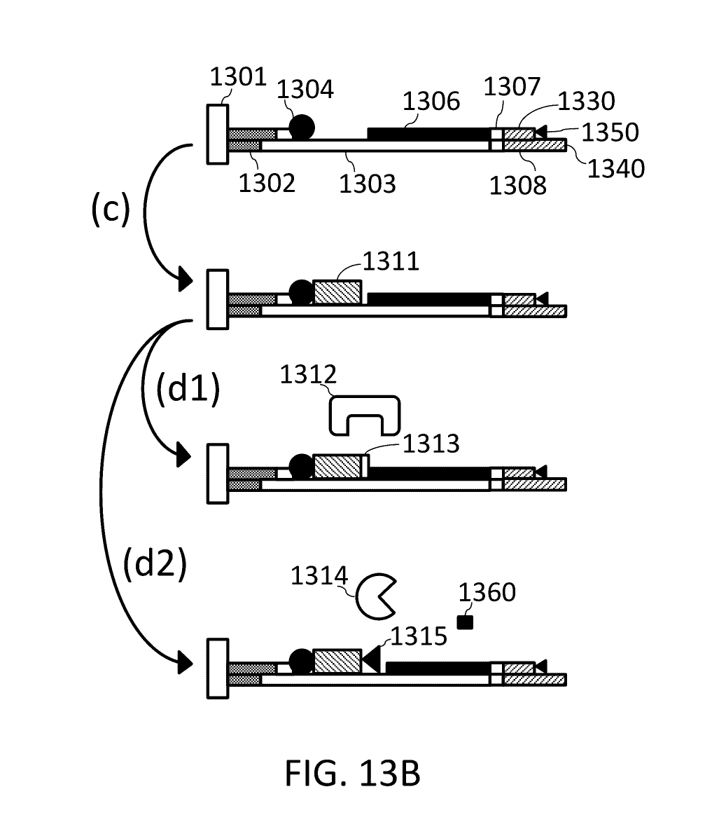

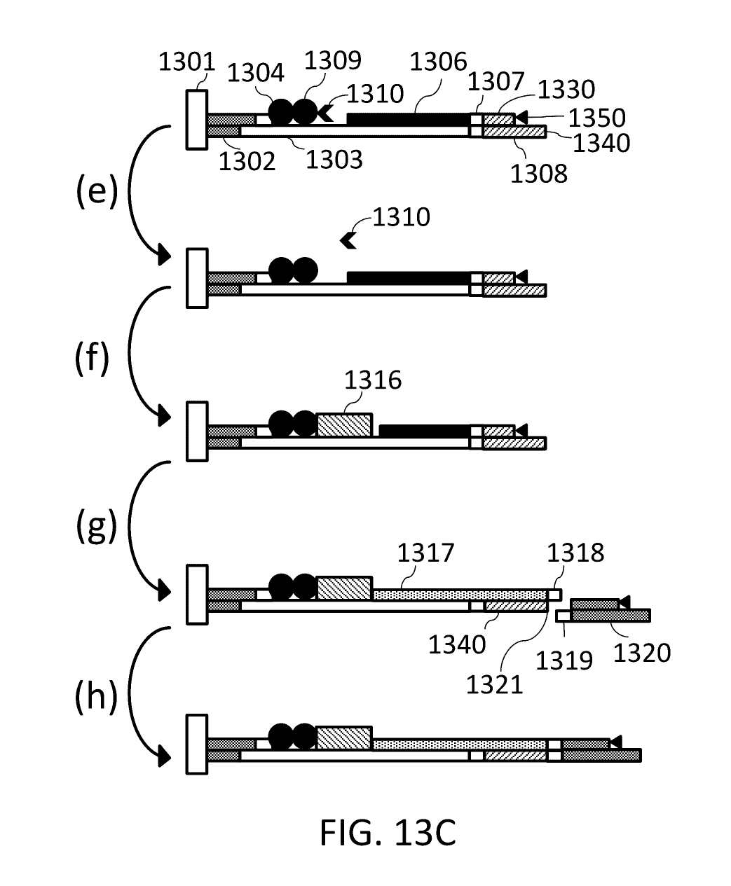

FIG. 12B shows the attachment of a protective tail tag. Said attachment takes place during steps (c) through (f) in the event that the nucleic acid molecule does not incorporate a nucleotide during step (b). In the event that the nucleic acid molecule incorporates a nucleotide during step (b), the nucleic acid molecule remains unaltered during steps (c) through (f) (and thus not shown in FIG. 12B). The role of the protective tail tag attachment is to protect the nucleic acid molecule from digestion during subsequent cycles of attaching tail tags, as explained in the description of FIG. 13. During step (c), the nucleic acid molecule 1203 and its parts are exposed to conditions to cause polymerization, and to a template-dependent polymerization reaction solution comprising cleavable nucleotides to complement the nucleic acid molecule 1203, resulting in the production of segment 1207. Polymerases used in the reaction possess 5′-to-3′ exonuclease activity, so that they digest part of 1208 as they produce 1207. In another embodiment, said polymerases have strand-displacing activity.

During step (d), the nucleic acid molecule 1203 and its parts are exposed to conditions to cause polymerization, and to a template-dependent polymerization reaction solution comprising deoxyribonucleotides to complement the nucleic acid molecule 1203. The reaction results in the production of segment 1209 that has a single-nucleotide overhang 1210. Taq polymerase molecules may be used in the reaction. Taq polymerase has 5′-to-3′ exonuclease activity to digest 1208, and creates overhang 1210 which comprises adenine. Said overhang is suitable for TA ligation. In order to generate a fully extended segment 1209 and an overhang, adequate extension time is given. Taq polymerase typically operates at 1 min extension time per 1 kb of template (New England BioLabs).

During step (e), the nucleic acid molecule 1203 and its parts are exposed to conditions to cause ligation, and to a ligation reaction solution comprising tail tags 1211. Said tail tags have a thymine at the single-nucleotide overhang 1212, and have the structure (d) described in FIG. 11. As mentioned previously, the free 5′ end 1219 of the nucleic acid molecule is ligatable. For the sake of clarity, the tail tag is shown before ligation is finalized. FIG. 12B (f) shows the final product of step (e), which is the nucleic acid molecule with an attached tail tag. Said tail tag is named “protective tail tag” because of its purpose, which is to protect the nucleic acid molecule from digestion, as explained in FIG. 13.

FIG. 12C shows the construction of a ligatable removable nucleotide tail and the attachment of an initial tail tag. Said construction takes place during steps (g) through (i), and said attachment takes place during steps (j) and (k) in the event that the nucleic acid molecule incorporates a nucleotide during step (b) in FIG. 12A. In the event that the nucleic acid molecule does not incorporate a nucleotide during step (b), the nucleic acid molecule acquires a protective tail tag during steps (c) through (f) in FIG. 12B, and remains unaltered during steps (g) through (k) (and thus not shown in FIG. 12C). The term “initial tail tag” is used to distinguish the tail tag being the first to attach to a nucleic acid molecule, from subsequently attached tail tags.

During step (g), the nucleic acid molecule 1203 and its parts are exposed to conditions and reagents suitable to remove the reversible terminator 1206 from the incorporated nucleotide 1205 comprising the predetermined base type.

During step (h), the nucleic acid molecule 1203 and its parts are exposed to conditions to cause polymerization, and to a template-dependent polymerization reaction solution comprising cleavable nucleotides to complement the nucleic acid molecule 1203, resulting in the production of segment 1213. Polymerases used in the reaction possess 5′-to-3′ exonuclease activity, so that they digest part of 1214 as they produce 1213. In another embodiment, said polymerases have strand-displacing activity.

During step (i), the nucleic acid molecule 1203 and its parts are exposed to conditions to cause polymerization, and to a template-dependent polymerization reaction solution comprising deoxyribonucleotides to complement the nucleic acid molecule 1203. The reaction results in the production of segment 1215 that has a single-nucleotide overhang 1216. Taq polymerase molecules can be used in the reaction. Taq polymerase has 5′-to-3′ exonuclease activity to digest 1214, and creates overhang 1216 which comprises adenine. Said overhang is suitable for TA ligation.

During step (j), the nucleic acid molecule 1203 and its parts are exposed to conditions to cause ligation, and to a ligation reaction solution comprising tail tags 1217. Said tail tags have a thymine at the single-nucleotide overhang 1218, and have the structure (d) described in FIG. 11. As mentioned previously, the free 5′ end 1219 of the nucleic acid molecule is ligatable. For the sake of clarity, the tail tag is shown before ligation is finalized. FIG. 12C (k) shows the final product of step (j), which is the nucleic acid molecule with an attached tail tag. Said tail tag is named “initial tail tag” for the reason described previously.

In FIGS. 13A, 13B and 13C, an embodiment is described for the attachment of a tail tag to a nucleic acid molecule that already has an initial tail tag attached to it. FIG. 13A shows nucleic acid molecule 1303. Said nucleic acid molecule is double-stranded DNA attached to adaptor 1302. Said adaptor is anchored to a solid support 1301. The nucleic acid molecule 1303 is already subjected to a round of: (i) incorporating a nucleotide 1304 comprising a specific base type, (ii) having a ligatable removable nucleotide tail constructed, and (iii) having an initial tail tag 1308 attached, as described in FIG. 12C. Said ligatable removable nucleotide tail comprises segment 1305 comprising cleavable nucleotides, segment 1306 comprising deoxyribonucleotides, and the adenine-comprising single-nucleotide overhang 1307, as described in FIG. 12C. Said initial tail tag is irreversibly terminated with the presence of dideoxyribonucleotide 1350, and comprises a removable part 1330 and a remaining part 1340, as described in (d) of FIG. 11.

During step (a), the nucleic acid molecule and its parts are exposed to conditions and reagents that excise the cleavable nucleotides of segment 1305. Said conditions and reagents are suitable for the type of cleavable nucleotides used to construct 1305, and are described in detail elsewhere herein. Upon completion of step (a), the 3′ end of the deoxyribonucleotide 1304 becomes available for extension by polymerization (i.e. said end regains a OH group).

During step (b), the nucleic acid molecule and its parts are exposed to conditions to cause polymerization, and to a template-dependent polymerization reaction solution comprising reversibly terminated deoxyribonucleotides comprising a predetermined base type. Polymerases used in the reaction possess 5′-to-3′ exonuclease activity. In another embodiment, said polymerases have strand-displacing activity. In the event that a nucleotide comprising the predetermined base type is complementary to the nucleic acid molecule at the specific position following the extendable 3′ end, incorporation takes place, as shown in FIG. 13A, where nucleotide 1309 is incorporated into the nucleic acid molecule, said nucleotide comprising a reversible terminator 1310.

FIG. 13B shows the construction of a non-ligatable blocking nucleotide tail during steps (c) and (d) with option (d1) and option (d2). Said construction takes place in the event that the nucleic acid molecule does not incorporate a nucleotide during step (b). In the event that the nucleic acid molecule incorporates a nucleotide during step (b), the nucleic acid molecule remains unaltered during steps (c) and (d) (and thus not shown in FIG. 13B).

During step (c), the nucleic acid molecule and its parts are exposed to conditions to cause polymerization, and to a template-dependent polymerization reaction solution comprising cleavable nucleotides to complement the nucleic acid molecule 1303. The polymerases used in this step do not possess strand-displacing activity, and do not possess 5′-to-3′ exonuclease activity, and fill the gap created during step (a) in FIG. 13A, without displacing or digesting 1306, 1307 and the removable part 1330 of the tail tag. During this step, segment 1311 is constructed, which has an extendable 3′ end.

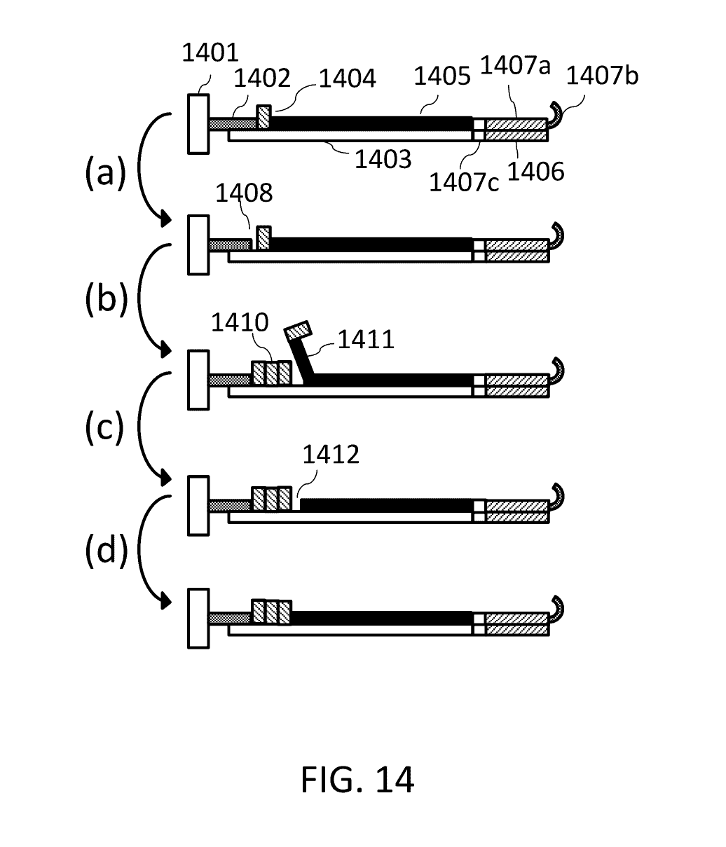

In another embodiment, the polymerases used in step (c) have strand displacing activity. Step (c) is complemented with treatment with DNA endonucleases that cleave any displaced strand segments. This approach is described in more detail in FIG. 14.

During step (d), said extendable 3′ end of 1311 is either sealed or terminated. One option is to seal using step (d1), whereas another option is to terminate using step (d2). During step (d1), the nucleic acid molecule and its parts are exposed to conditions to cause ligation, and to a ligation reaction solution comprising ligase molecules 1312. Ligation creates a backbone bond 1313 between the last nucleotide of 1311 and the first nucleotide of 1306. During step (d2), the nucleic acid molecule and its parts are exposed to conditions to cause polymerization, and to a template-dependent polymerization reaction solution comprising polymerase molecules 1314 and terminated nucleotides to complement the nucleic acid molecule 1303. The polymerases 1314 used in this step comprise 5′-to-3′ exonuclease activity and remove nucleotide 1360 from segment 1306 upon incorporation of the terminated nucleotide 1315. Polymerases with strand displacing activity may also be used.

In another embodiment, step (c) comprises using strand-displacing polymerases to construct segment 1311. For reasons explained in FIG. 2 and elsewhere herein, 1311 is expected to be short, thus not replacing the entire length of the previously generated strand (1306, 1307 and 1330). During step (d), 1311 can be terminated by an incorporated blocked nucleotide 1315.

FIG. 13C shows the construction of a ligatable removable nucleotide tail and the attachment of a tail tag. Said construction takes place during steps (e) through (g), and said attachment takes place during step (h) in the event that the nucleic acid molecule incorporates a nucleotide during step (b) in FIG. 13A. In the event that the nucleic acid molecule does not incorporate a nucleotide during step (b), the nucleic acid molecule acquires a non-ligatable blocking nucleotide tail during steps (c) and (d) in FIG. 13B, and remains unaltered during steps (e) through (h) (and thus not shown in FIG. 13C).

During step (e), the nucleic acid molecule 1303 and its parts are exposed to conditions and reagents suitable to remove the reversible terminator 1310 from the incorporated nucleotide 1309 comprising the predetermined base type.

During step (f), the nucleic acid molecule 1303 and its parts are exposed to conditions to cause polymerization, and to a template-dependent polymerization reaction solution comprising cleavable nucleotides to complement the nucleic acid molecule 1303, resulting in the production of segment 1316. Polymerases used in the reaction possess 5′-to-3′ exonuclease activity, so that they digest part of 1306 as they produce 1316. In another embodiment, said polymerases have strand-displacing activity.

During step (g), the nucleic acid molecule 1303 and its parts are exposed to conditions to cause polymerization, and to a template-dependent polymerization reaction solution comprising deoxyribonucleotides to complement the nucleic acid molecule 1303 (and its previously attached tail tag 1308, which is considered part of the nucleic acid molecule 1303). The reaction results in the production of segment 1317 that has a single-nucleotide overhang 1318. Taq polymerase molecules can be used in the reaction. Taq polymerase has 5′-to-3′ exonuclease activity to digest 1306, 1307 and the removable part 1330, and creates overhang 1318 which comprises adenine. Said overhang is suitable for TA ligation.

During step (h), the nucleic acid molecule 1303 and its parts are exposed to conditions to cause ligation, and to a ligation reaction solution comprising tail tags 1320. Said tail tags have a thymine at the single-nucleotide overhang 1319, and have the structure (d) described in FIG. 11. As mentioned in FIG. 11, the free 5′ end 1321 of the remaining part 1340 of the previously attached tail tag 1308 is ligatable. For the sake of clarity, the tail tag 1320 is shown before and after ligation is finalized.

In another embodiment, steps (g) and (h) are performed simultaneously, using commercially available kits that can perform combined extension/ligation (e.g., TruSeq custom amplicon assay, Illumina).

The final product of FIG. 13C is optionally further subjected to incubation with 5′-to-3′ exonuclease molecules, such as T7 exonuclease, which digest blunt and 5′ recessive ends, but not 5′ overhangs. Said incubation causes enzymatic digestion of nucleic acid molecules that fail to attach tail tags, removing them from further processing. Said incubation does not affect nucleic acid molecules that attach a tail tag as shown in FIG. 13C, nucleic acid molecules that remain with a previously attached tail tag as shown in FIG. 13B, and nucleic acid molecules that do not have a tail tag but have a protective tail tag as shown in FIG. 12B.

In FIG. 14, an example of constructing a non-ligatable blocking nucleotide tail is shown. Template DNA strand 1403 is anchored to a surface 1401 by annealing to an adaptor 1402. 1403 has already gone through processing that led to the formation of a non-ligatable blocking nucleotide tail comprising a cleavable nucleotide 1404, a DNA segment 1405 and the removable part 1407a of a protective tail tag 1406. The protective tail tag 1406 has a T overhang 1407c in its one end, suitable for TA ligation, and another blunt end carrying a 3′ end modification 1407b. Modification 1407b prevents self-ligation of protective tail tags, unwanted ligations, and overhang formations. Examples of modifications include, but are not limited to, spacers, phosphorylation, biotinylation, etc.

During step (a), 1403 and its surroundings are exposed to conditions and reagents to cause selective cleavage of the backbone bond between 1402 and 1404, forming a nick 1408. For example, in the event that 1404 is a ribonucleotide, the bond at its 5′ end can be cleaved by using RNase HII, as described herein. In a subsequent step that is not shown, 1403 and its surroundings are exposed to polymerization conditions, and to a template-dependent polymerization reaction solution comprising nucleotides comprising a predetermined base type. There is no incorporation of such nucleotides in the template strand. The procedure continues with the formation of a non-ligatable blocking nucleotide tail.

During step (b), 1403 and its surroundings are exposed to polymerization conditions, and to a template-dependent polymerization reaction solution that comprises nucleotides comprising 3 base types that are not the predetermined base type. In this embodiment, step (b) produces segment 1410 by displacing segment 1411.

During step (c), 1403 and its surroundings are exposed to conditions that activate enzymes that can perform cleavage of single-stranded and non-complementary segments, and to a solution comprising such enzymes. Non-limiting examples include mung bean nuclease or CELI (Surveyor©; Integrated DNA Technologies, Inc., Coralville, Iowa) or other nucleases, which can digest single strands, and non-complementary nucleotides. Such nucleases are described in (Till et al., 2004). During step (c), segment 1411 is cleaved, and nick 1412 is formed.

During step (d), 1403 and its surroundings are exposed to conditions to cause ligation, and to a ligation reaction solution. During this step, the nick 1412 is sealed, thus concluding the formation of a non-ligatable blocking nucleotide tail.

In some embodiments, terminal blocking nucleotide tails are produced instead of blocking nucleotide tails. Such tails do not allow regeneration of an extendable 3′ end, preventing participation of the template in future sequencing cycles.

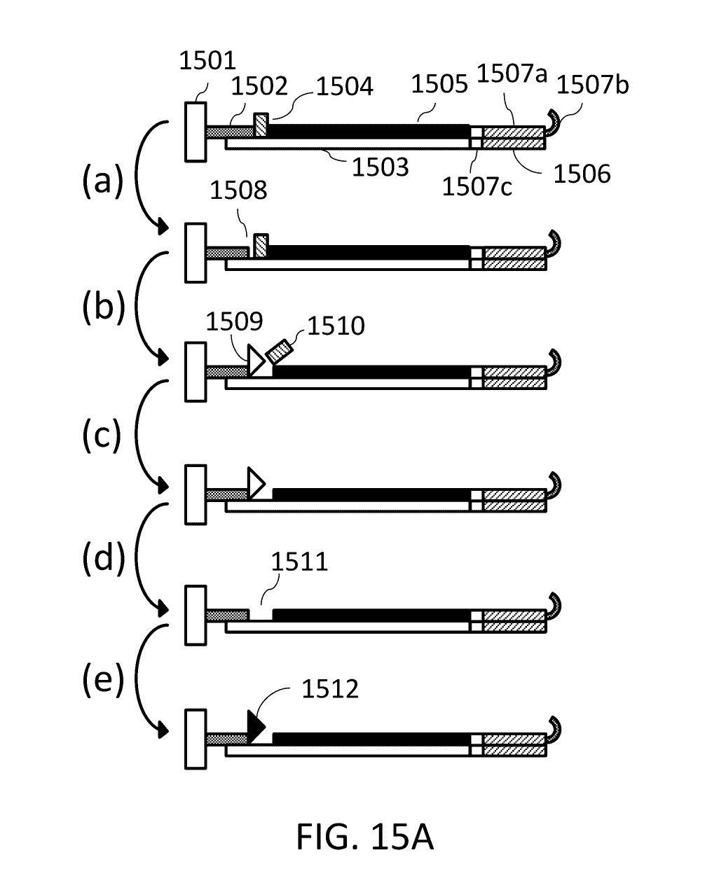

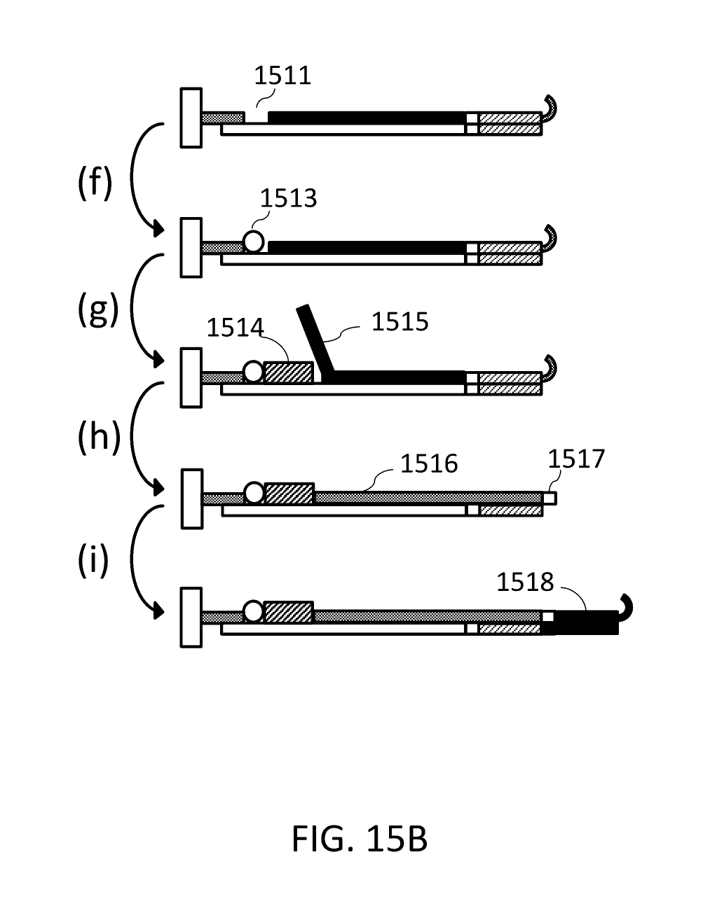

In one embodiment, a terminal blocking nucleotide tail is formed as shown in FIG. 15A. Template DNA strand 1503 is hybridized to an adaptor 1502, which is anchored to a surface 1501. 1503 has already gone through processing that led to the formation of a blocking nucleotide tail comprising a cleavable nucleotide 1504, a DNA segment 1505 and the removable part 1507a of a protective tail tag 1506. The protective tail tag 1506 has a T overhang 1507c in its one end, suitable for TA ligation, and another blunt end carrying a 3′ end modification 1507b. Modification 1507b prevents self-ligation of protective tail tags, unwanted ligations, and overhang formations. Examples of modifications include, but are not limited to, spacers, phosphorylation, biotinylation, etc.

During step (a), 1503 and its surroundings are exposed to conditions and reagents to cause selective cleavage of the backbone bond between 1502 and 1504, forming a nick 1508. For example, in the event that 1504 is a ribonucleotide, the bond at its 5′ end can be cleaved by using RNase HII, as described herein.

During step (b), 1503 and its surroundings are exposed to conditions to cause polymerization, and to a template-dependent polymerization reaction solution that comprises irreversibly blocked cleavable nucleotides. Said irreversibly blocked cleavable nucleotides in said solution comprise adenine, thymine, cytosine and guanosine. Examples include, but are not limited to, a-S-ddNTP. During this step, nucleotide 1509 is incorporated by displacing the cleavable nucleotide (1510).

During step (c), 1503 and its surroundings are exposed to conditions to cause activation of enzymes that can perform cleavage of single-stranded and non-complementary segments, and to a solution comprising such enzymes. Non-limiting examples include mung bean nuclease or CELI (Surveyor©; Integrated DNA Technologies, Inc., Coralville, Iowa) or other nucleases, which can digest single strands, and non-complementary nucleotides. Such specific nucleases are described in (Till et al., 2004). In another embodiment, the displaced cleavable nucleotide 1510 is a ribonucleotide and step (c) comprises exposing 1503 and its surroundings to a solution comprising lanthanide salts that can cleave at the 3′ end of 1510. Lanthanides are discussed elsewhere herein. During step (c), 1510 is cleaved, and a nick is formed.

During step (d), 1503 and its surroundings are exposed to conditions and reagents favoring cleavage of the cleavable irreversibly blocked nucleotide 1509, leaving a single-base gap 1511. In the event that the cleavable irreversibly blocked nucleotide is a-S-ddNTP, the solution used for cleavage may comprise iodoethanol. Cleavage reagents are discussed elsewhere herein. In the event that cleavage produces a non-extendable 3′ end, step (d) also comprises treatment with appropriate reagents that render the 3′ end extendable.

During step (e), 1503 and its surroundings are exposed to conditions to cause polymerization, and to a template-dependent polymerization reaction solution comprising blocked nucleotides comprising base types other than a predetermined base type. During this step, a terminal blocking nucleotide tail is formed, in the event that the base of 1503 exposed by the single-base gap 1511 is not complementary to the predetermined base type. The terminal blocking nucleotide tail formed during this step comprises non-cleavable blocked nucleotide 1512. In another embodiment, step (e) precedes step (b).

In a different embodiment, a blocking nucleotide tail is formed during step (e), wherein 1512 is cleavable. 1512 may be blocked or unblocked or not modified. In the event that 1512 is a cleavable unmodified nucleotide, gap-filling polymerases that lack 5′-to-3′ exonuclease and strand-displacing activities are used, followed by ligase treatment that seals the nick left after nucleotide incorporation.

During step (f) in FIG. 15B, 1503 and its surroundings are exposed to conditions to cause polymerization, and to a template-dependent polymerization reaction solution comprising nucleotides comprising the predetermined base type which is not comprised in the reaction solution of the previous step. In the event that there is no nucleotide incorporation during step (e), nucleotide 1513 is incorporated during this step.

During step (g), 1503 and its surroundings are exposed to conditions to cause polymerization, and to a template-dependent polymerization reaction solution comprising cleavable nucleotides. During this step, the formation of a ligatable removable nucleotide tail starts, which comprises segment 1514 comprising cleavable nucleotides. Production of 1514 may occur with simultaneous displacement of segment 1515 of the previous strand.

During step (h), the formation of the ligatable removable nucleotide tail is completed. During this step, 1503 and its surroundings are exposed to conditions to cause polymerization, and to a template-dependent polymerization reaction solution comprising deoxyribonucleotides. Strand segment 1516 is formed. 1503 and its surroundings can be further treated with a polymerase, such as Taq polymerase, which can perform incorporation of a single-base A overhang 1517, suitable for TA ligation.

During step (i), 1503 and its surroundings are exposed to conditions to cause ligation, and to a ligation reaction solution. During this step, tail tag 1518 carrying a T-overhang is ligated to 1503 and its ligatable removable nucleotide tail. Tail tag 1518 represents the base type of 1513.

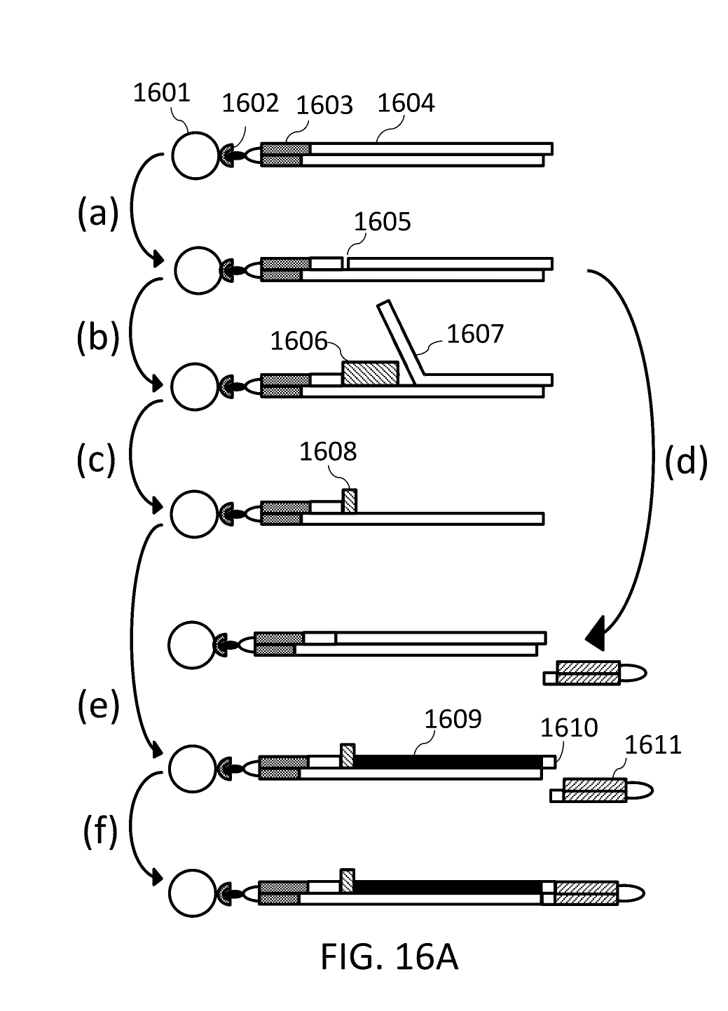

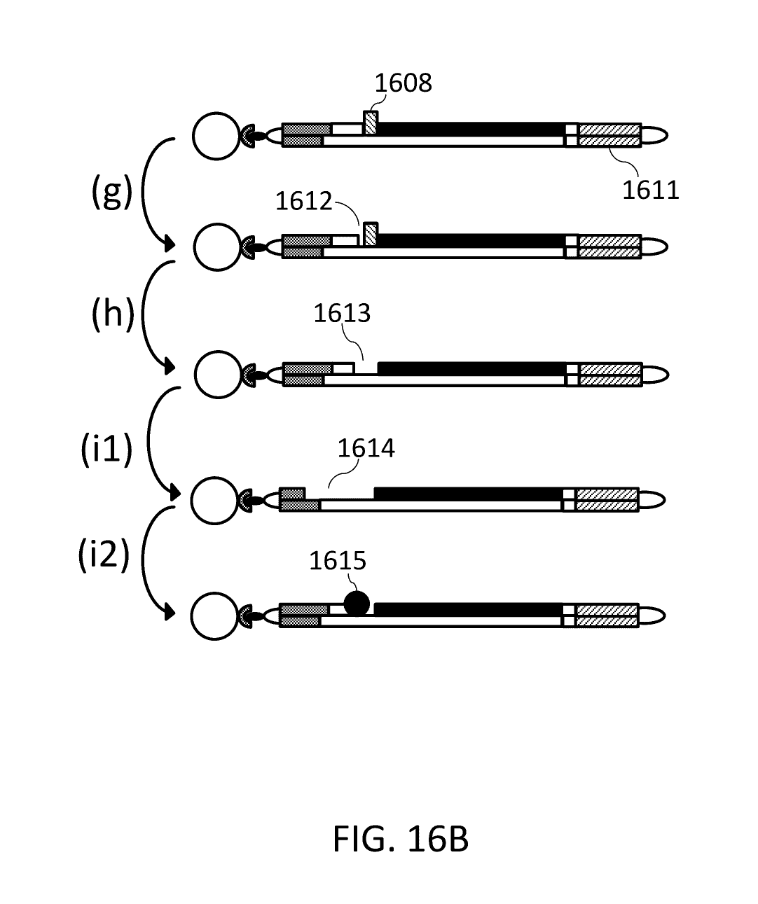

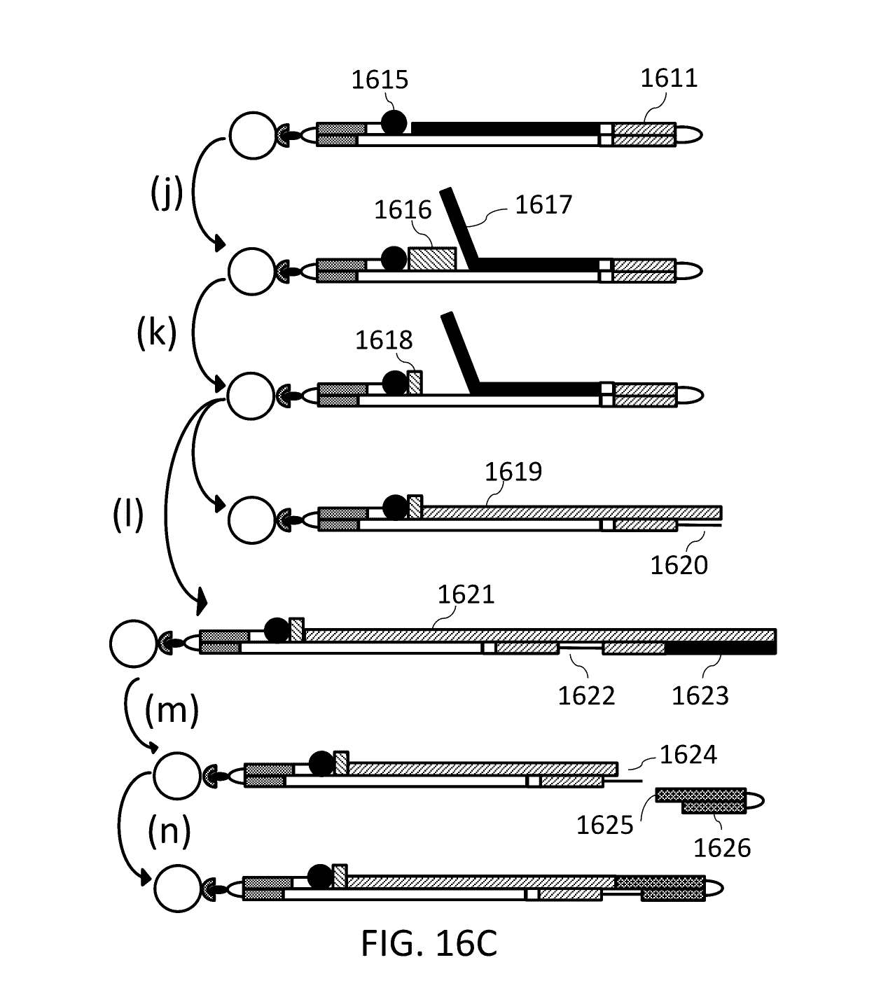

In one embodiment shown in FIG. 16, a nucleic acid molecule 1604 is a double-stranded DNA molecule with single-nucleotide 3′ end overhangs comprising adenine. 1604 is TA-ligated to a hairpin adaptor 1603. 1603 comprises at least one biotin-labeled nucleotide which binds streptavidin (1602), and a T overhang at the 3′ end, suitable for TA ligation. 1603 also comprises a restriction site that can be recognized by a nicking endonuclease that catalyzes a single strand break a few bases away from its recognition sequence, and into 1604. Examples include, but are not limited to, Nt.BstNBI which recognizes the sequence 5′-GAGTC-3′ and creates a nick at the 3′ end of the 4th base following the 3′ end of its recognition sequence; Nt.AlwI which recognizes the sequence 5′-GGATC-3′ and creates a nick at the 3′ end of the 4^{th} base following the 3′ end of its recognition sequence; Nt.BsmAI which recognizes the sequence 5′-GTCTC-3′ and creates a nick at the 3′ end of the first base following the 3′ end of its recognition sequence; Nt.BspQI which recognizes the sequence 5′-GCTCTTC-3′ and creates a nick at the 3′ end of the first base following the 3′ end of its recognition sequence.

During step (a) of FIG. 16A, 1604 and its surroundings are exposed to conditions to cause restriction enzyme activation, and to a reaction solution comprising nicking endonuclease molecules that recognize the restriction sites present in 1603. Nicking endonuclease molecules create nick 1605, thus introducing a 3′ end that can be extended by polymerization.

During step (b), 1604 and its surroundings are exposed to polymerization conditions, and to a template-dependent polymerization reaction solution comprising cleavable nucleotides comprising a predetermined base type, and polymerase molecules with strand-displacement ability. In the situation that two or more consecutive bases in the nucleic acid molecule are complementary to the predetermined base type (homopolymer segment), step (b) produces segment 1606 comprising cleavable nucleotides, which starts from the 3′ end at nick 1605. During 1606 production, segment 1607 which is part of 1604 is displaced.

During step (c), 1604 and its surroundings are exposed to conditions and reagents to release the cleavable nucleotides of 1606 leaving a single cleavable nucleotide 1608 bound with its 5′ end to 1604. Said conditions and reagents are suitable for the type of cleavable nucleotides used, and are described in detail in Examples 7 and 10, and elsewhere herein. For example, in the event that the cleavable nucleotides are ribonucleotides, treatment with NaOH or lanthanides can cause hydrolysis leaving a single ribonucleotide still bound to DNA with its 5′ end. In the event that cleavage renders the 3′ end of the remaining cleavable nucleotide 1608 non-extendable, step (c) also comprises treatment with appropriate reagents (phosphatases, such as rSAP, for example, in the event that 3′ ends are phosphorylated).

During step (d), 1604 and its surroundings are exposed to conditions to cause ligation, and to a ligation reaction solution comprising ligase molecules. In the event that there is no incorporation of cleavable nucleotides in step (b), step (d) seals nick 1605, forming a terminal blocking nucleotide tail. The absence of cleavable nucleotides and an extendable 3′ end in the nucleic acid molecule prevents the nucleic acid molecule from participating in future processes of constructing ligatable removable nucleotide tails, in the event that the nucleic acid molecule does not incorporate cleavable nucleotides comprising the predetermined base type in step (b).

During step (e), 1604 and its surroundings are exposed to polymerization conditions, and to a template-dependent polymerization reaction solution comprising deoxyribonucleotides, and polymerase molecules capable of initiating polymerization from the remaining cleavable nucleotide 1608. Such polymerases are described elsewhere herein. Step (e) produces DNA segment 1609, which can be further treated with Taq DNA polymerase or other suitable polymerase that adds an A overhang (single nucleotide comprising adenine) 1610 at the 3′ end of 1609.

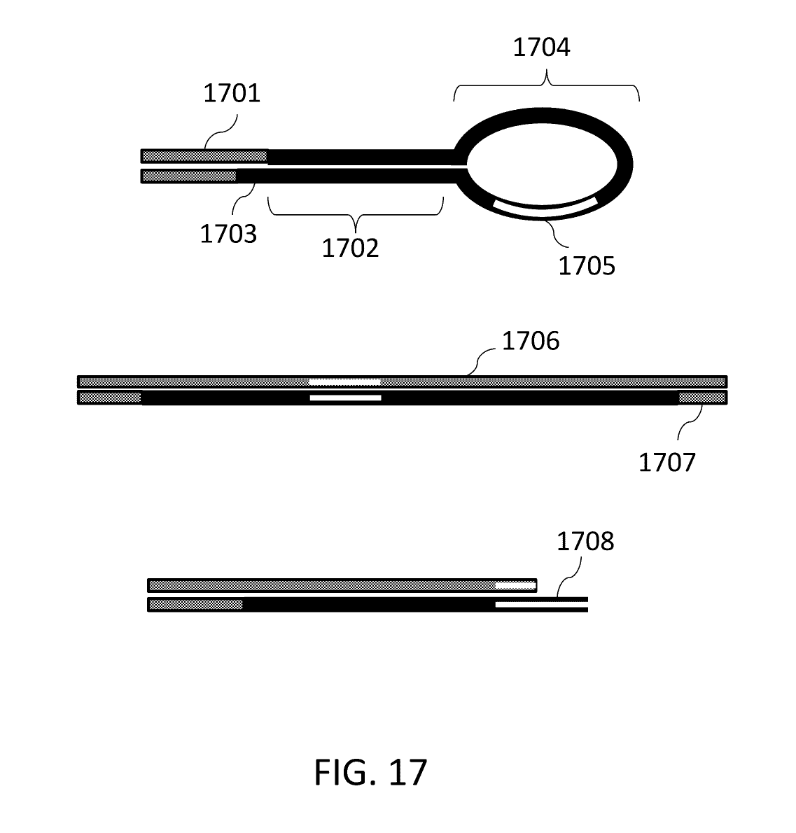

During step (f), 1604 and its surroundings are exposed to conditions to cause ligation, and to a ligation reaction solution comprising hairpin tail tags 1611, having T overhangs suitable for TA ligation to 1609 (its overhang 1610) and the template strand of 1604. Each 1611 tag also comprises at least one restriction site within its loop, which becomes functional in the event that a strand is constructed that is complementary to the loop (shown in FIG. 17 described later herein). 1611 has specific sequence that represents the predetermined base type comprised in 1608. It is worth noting that the nucleic acid molecule carrying a terminal blocking nucleotide tail formed in step (d) may also be ligated to 1611, but said nucleic acid molecule is not capable of participating in future tail tag attachments.

During step (g) in FIG. 16B, 1604 and its surroundings are exposed to conditions and reagents to cause selective cleavage of the backbone bond between the deoxyribonucleotide at the 5′ end side of 1608, and 1608, forming nick 1612. For example, in the event that 1608 is a ribonucleotide, the bond at its 5′ end can be cleaved by using RNase HII, as described elsewhere herein.

During step (h), 1604 and its surroundings are exposed to conditions and reagents to cleave the backbone bond at the 3′ end of the cleavable nucleotide 1608, forming gap 1613. The conditions and reagents used in step (h) are suitable for the type of cleavable nucleotides used (1608), and are described in detail in Examples 7 and 10, and elsewhere herein. For example, in the event that the cleavable nucleotide is a ribonucleotide, treatment with NaOH or lanthanides can cause hydrolysis, removing 1608. In the event that hydrolysis is conducted in denaturing conditions (such as NaOH treatment in high temperature), re-annealing is performed as described in Example 7. Attaching hairpin tail tag 1611 is advantageous under denaturing conditions, because the hairpin keeps strands linked to one another, thereby allowing re-annealing.

During step (i) (comprising (ii) and (i2)), gap 1613 is filled with a non-cleavable nucleotide comprising the predetermined base type in step (b). Nucleic acid molecules that comprise terminal blocking nucleotide tails remain unaffected. In one embodiment, step (i) comprises exposing 1604 and its surroundings to polymerization conditions, and to a template-dependent polymerization reaction solution comprising deoxyribonucleotides to complement 1604, and gap-filling polymerase molecules with 3′-to-5′ exonuclease activity, but without 5′-to-3′ exonuclease activity, such as T7 and T4 DNA polymerases (Huang and Lehman, 1972)(Kumar et al., 2004) (Tabor and Richardson, 1987). The modulation of conditions such as temperature and nucleotide concentration can alter the strength of 3′-to-5′ exonuclease activity of such polymerases, that can widen the gap 1613, forming a larger gap 1614, which can be filled by the polymerase action of said polymerases. In another embodiment, step (i) comprises (i2) filling the gap 1613 and incorporating deoxyribonucleotide 1615, using polymerase molecules (such as Sulfolobus DNA polymerase IV; (Choi et al., 2011)) that do not possess any exonuclease activity (no 3′-to-5′, and no 5′-to-3′ exonuclease activities) and do not possess any strand-displacing activity. After 1615 incorporation, an extendable 3′ end (the 3′ end of 1615) remains.

During step (j) in FIG. 16C, 1604 and its surroundings are exposed to polymerization conditions, and to template-dependent polymerization reaction solution comprising cleavable nucleotides comprising a predetermined base type other than the predetermined base type in step (b). In the event that a homopolymer segment is present in the template strand of the nucleic acid molecule with bases complementary to the predetermined base type, step (j) produces segment 1616 comprising cleavable nucleotides. In the event that strand-displacing polymerase molecules are used, segment 1616 generation causes displacement of segment 1617.

During step (k), 1604 and its surroundings are exposed to conditions and reagents to release the cleavable nucleotides of 1616 leaving a single cleavable nucleotide 1618 bound with its 5′ end to 1615. Said conditions and reagents are suitable for the type of cleavable nucleotides used, and are described in detail in Examples 7 and 10, and elsewhere herein. For example, in the event that the cleavable nucleotides are ribonucleotides, treatment with NaOH or lanthanides can cause hydrolysis leaving a single ribonucleotide still bound to DNA with its 5′ end. In the event that cleavage renders the 3′ end of the remaining cleavable nucleotide 1618 non-extendable, step (k) also comprises treatment with appropriate reagents (phosphatases, such as rSAP, for example, in the event that 3′ ends are phosphorylated).

In a step not shown following step (k), 1604 and its surroundings are exposed to conditions to cause ligation, and to a ligation reaction solution comprising ligase molecules. In the event that there is no incorporation of cleavable nucleotides in step (j), ligation seals the nick following 1615, forming a terminal blocking nucleotide tail. The absence of cleavable nucleotides and an extendable 3′ end in the nucleic acid molecule prevents the nucleic acid molecule from participating in future processes of constructing ligatable removable nucleotide tails, in the event that the nucleic acid molecule does not incorporate cleavable nucleotides comprising the predetermined base type in step (j).

During step (l), 1604 and its surroundings are exposed to polymerization conditions, and to a template-dependent polymerization reaction solution comprising deoxyribonucleotides, and polymerase molecules capable of initiating polymerization from the remaining cleavable nucleotide 1618. Such polymerases are described elsewhere herein. Polymerases may possess 5′-to-3′ exonuclease activity. In this case, the polymerase molecules produce segment 1619 and simultaneously cleave the previous strand (strand comprising 1617). Since the 5′-to-3′ exonuclease action of polymerases usually cleaves nucleotides from strand segments being at least partially complementary to the polymerases' template strand, polymerases in this embodiment may cleave the strand comprising 1617. Cleavage may include part of the hairpin tail tag 1611, up to the point where there is no more complementarity between strands, thus leaving hairpin loop 1620 intact. 1619 is shown to be complementary to the template strand and to the remaining part of hairpin tail tag 1611, including its loop 1620.

In another embodiment, polymerases with strand-displacing activity are used in step (l). In this case, polymerase molecules produce strand 1621. Since strand-displacing polymerases do not destroy the previous strand, the newly produced strand segment 1621 is complementary to the template strand, including the entire hairpin tail tag in open conformation (1622), and the previous strand 1623 comprising segment 1617 (not shown in proportion).

As mentioned previously, hairpin 1611 comprises the sequence of a restriction site within its loop. The restriction site is inactive (i.e. cannot be recognized by corresponding restriction enzymes), because the loop is single-stranded (non-complementary to another strand segment). When the loop becomes double-stranded during step (1), the restriction site becomes recognizable by its corresponding restriction enzyme molecules. This is shown in more detail in FIG. 17. FIG. 17 shows a hairpin tail tag comprising double-strand (self-complementarity) segment 1702 (termed “stem”), and loop 1704 that does not exhibit self-complementarity. 1704 comprises 1705, which is the single-strand segment of a double-stranded recognition site of a restriction endonuclease. 1702 comprises overhang 1703, which facilitates ligation of the hairpin tag to nucleic acid molecule 1701. In the event that there is an extension occurring, starting from an extendable 3′ end in 1701 (for example, the construction of a ligatable removable nucleotide tail) strand segment 1706 may be produced. In the event that strand-displacing polymerase molecules are performing said extension, 1706 is complementary to the entire hairpin and the strand part 1707 of 1701. 1707 is located downstream of said extendable 3′ end prior to said extension. Upon formation of 1706, 1705 becomes a double-stranded functional restriction site that can be recognized by its corresponding restriction endonuclease. 1708 is a 5′ end overhang formed by the action of a restriction enzyme recognizing the double-stranded 1705. In this case, the restriction enzyme cuts within its recognition site.

During step (m) in FIG. 16C, 1604 and its surroundings are exposed to conditions to cause restriction enzyme-mediated digestion, and to a digestion reaction solution comprising restriction enzyme molecules capable of cleaving the restriction site within the hairpin loop. FIG. 16C shows the generated cleavage site 1624 comprising an overhang which is complementary to the overhang 1625 of a tail tag 1626. 1626 has specific sequence that represents the predetermined base type comprised in the incorporated cleavable nucleotide 1618.

During step (n), 1604 and its surroundings are exposed to conditions to cause ligation, and to a ligation reaction solution comprising ligase molecules and hairpin tail tags 1626.

In order to attach more tail tags to 1604, the process can be continued by applying step (g) and subsequent steps, and choosing another predetermined base type.

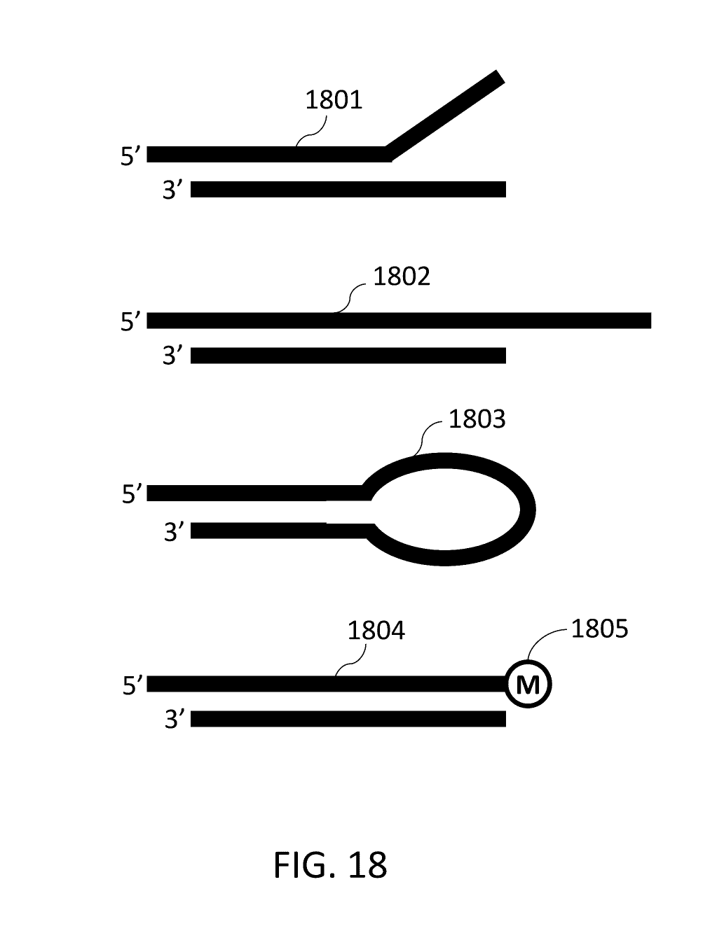

Tail tag designs such as the hairpin design used in the example of FIG. 16 are preferred in some embodiments, where denaturing conditions or exonucleases are used. The hairpin design may limit undesirable self-ligation, allow rehybridization of denaturing strands, or protect from exonuclease degradation. FIG. 18 shows examples of tail tag designs that protect from undesirable degradation by 3′-exonucleases acting on double-stranded nucleic acids. An example of such an enzyme is exonuclease III, which acts on blunt or recessed 3″-ends, or at nicks in duplex DNA. Tail tag 1801 has a ligatable end at the left side, and its end at the right side comprises two non-complementary segments. Tail tag 1802 has a ligatable end at the left side and a protruding 3′ end at its right side. Tail tag 1803 is a hairpin, explained in detail in FIG. 17. Tail tag 1804 has a ligatable end at the left side, and a blunt end at its right side, comprising modification 1805. Examples of modifications include, but are not limited to, inverted T, spacer, etc., that may block exonuclease activity and prevent self-ligation.

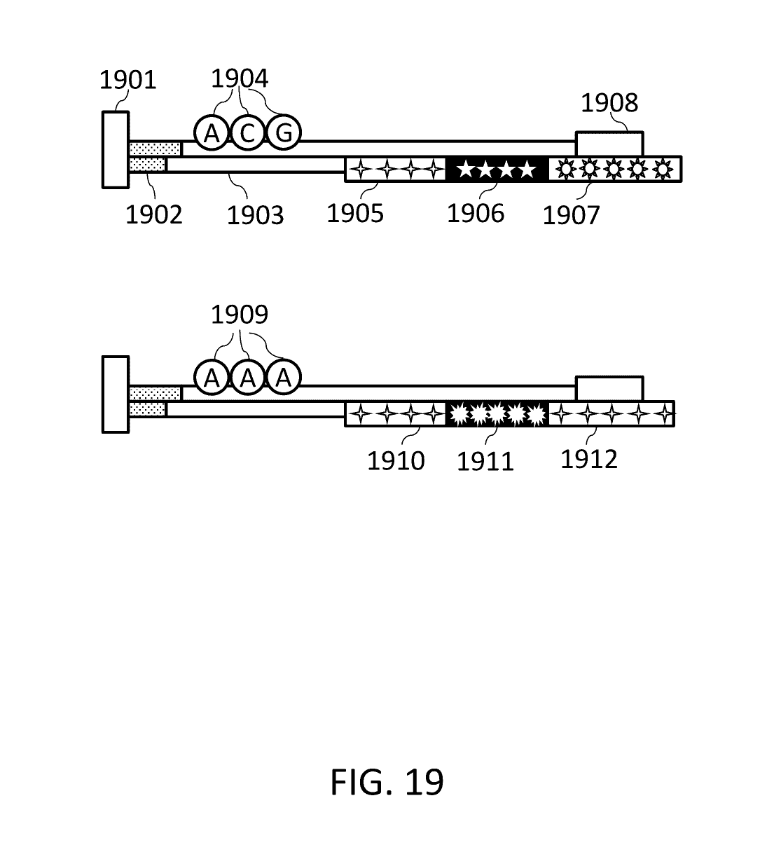

Each tail tag can comprise label types specific for the presence of a specific base type in each incorporated nucleotide. In one embodiment, the removable parts of tail tags are labeled and detected after each tail tag attachment, and removed during construction of a new ligatable removable nucleotide tail. In certain embodiments, tail tags can comprise labels within their remaining part, as explained in FIG. 11. Repetitive attachment of labeled tail tags and detection of their labels enables sequencing. FIG. 19 shows two nucleic acid molecules with attached labeled tail tags. Nucleic acid molecule 1903 is a double-stranded DNA attached to adaptor 1902, said adaptor being anchored to a solid support 1901. Nucleic acid molecule 1903 has three previously incorporated nucleotides (1904) comprising adenine (A), cytosine (C) and guanine (G). Each incorporation event of each of the said three previously incorporated nucleotides is matched by attachment of the corresponding labeled tail tag. The labeled remaining part of the tail tag 1905 corresponds to A, 1906 corresponds to C and 1907 corresponds to G. Each tail tag is labeled differently, because each tail tag is specific for a different base type. In order to adequately sequence a nucleic acid molecule, at least four differently labeled tail tag types are used: one for adenine, one for thymine or uracil, one for guanine, and one for cytosine. In a certain embodiment, at least eight differently labeled tail tag types are used, two for each base type, used in an alternating manner. This is demonstrated in the second nucleic acid molecule in FIG. 19. Said nucleic acid molecule has three previously incorporated nucleotides (1909), all of them comprising adenine (A). After the first incorporation event, tail tag 1910 was attached, after the second incorporation event, tail tag 1911 was attached, and after the third incorporation event, tail tag 1912 was attached. As shown, tail tag 1911 (the remaining part) comprises a different type of labels from tail tags 1910 and 1912. This alternating use of labels enables to distinguish individual bases within a homopolymer sequence.

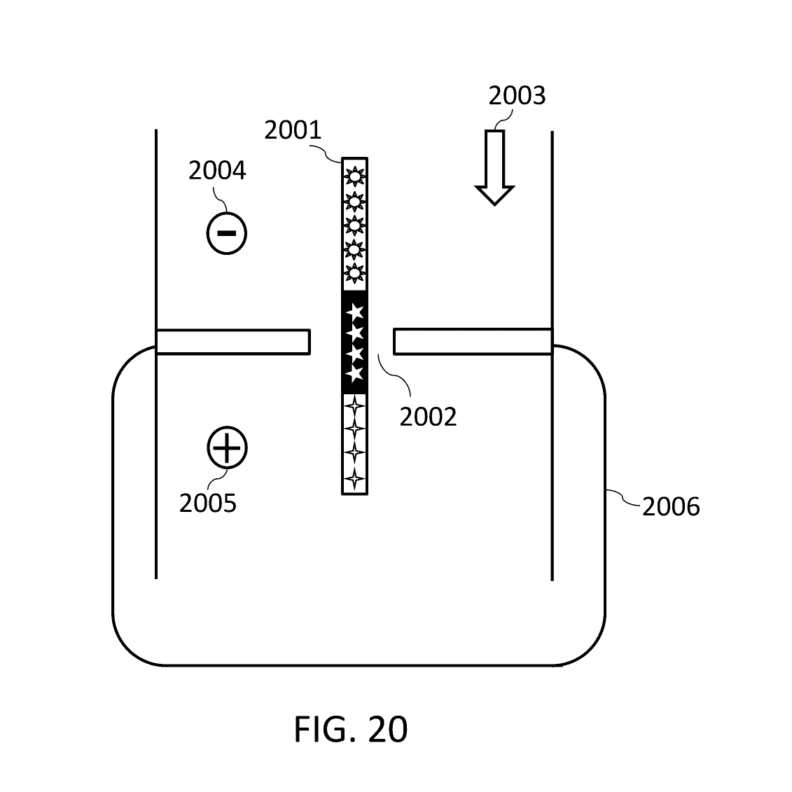

In a certain embodiment, tail tags comprising labels that alter conductivity when passed through a suitable nanopore device are attached to nucleic acid molecules based on the molecules' sequence. Nanopore devices and suitable labels are described elsewhere herein. In brief, nucleic acid molecules attached to tail tags such as those shown in FIG. 19 can be subjected to conditions that specifically cleave and release the part with the tail tags. This can be achieved for example by including a specific restriction endonuclease recognition site in the initial tail tag, and treating with the corresponding restriction endonuclease. Then, denaturing conditions can generate single strands that are capable of passing through nanopores. An example is shown in FIG. 20, wherein the remaining parts of connected tail tags previously attached to a nucleic acid molecule pass through a nanopore device as a single strand 2001. FIG. 20 schematically shows a nanopore device. A cathode 2004 and anode 2005 (e.g., platinum terminals connected to an appropriate power supply) are positioned to create an electrophoretic field in a buffer solution. The solution is divided into two chambers by a nanopore 2002. As the strand 2001 comprising tail tags is electrophoretically driven through the nanopore 2002 by the electrophoretic field (arrow 2003 shows the direction of the strand's motion), a detection circuit 2006 detects and records changes in conductivity. In a related embodiment, a plurality of strands comprising tail tags pass through one nanopore device. In another related embodiment, a plurality of strands comprising tail tags pass through multiple nanopore devices working in parallel (nanopore array). In another embodiment, strands comprise tail tags that have distinct sequence patterns causing distinct changes in conductivity when passing through a nanopore.

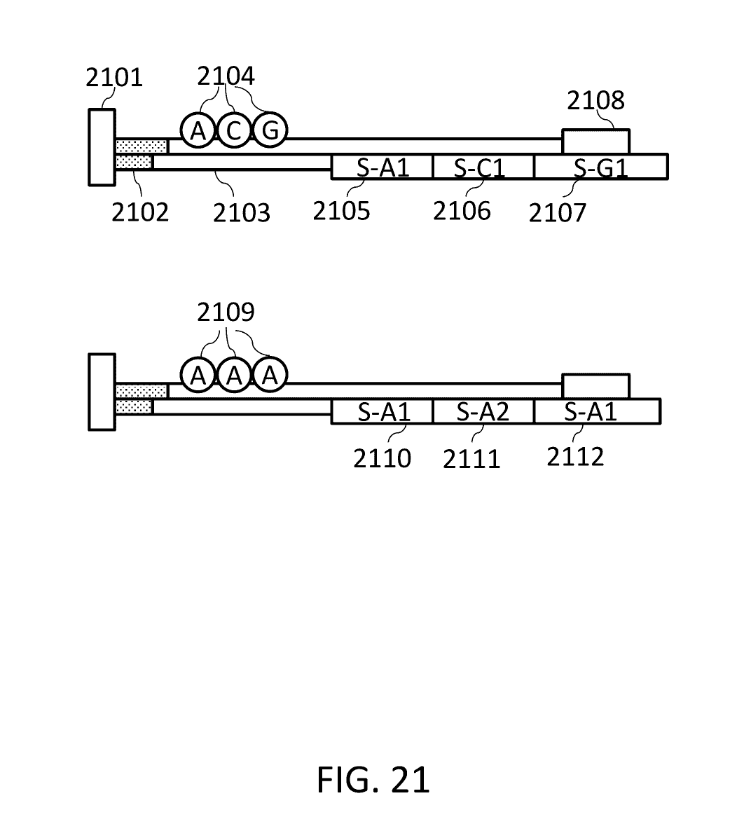

Each tail tag can comprise sequences specific for the presence of a specific base type in each incorporated nucleotide. Repetitive attachment of labeled tail tags and detection of their labels enables sequencing. FIG. 21 shows two nucleic acid molecules with attached tail tags. Nucleic acid molecule 2103 is a double-stranded DNA attached to adaptor 2102, said adaptor being anchored to a solid support 2101. Nucleic acid molecule 2103 has three previously incorporated nucleotides (2104) comprising adenine (A), cytosine (C) and guanine (G). Each incorporation event of each of the said three previously incorporated nucleotides is matched by attachment of the corresponding tail tag. The remaining part of the tail tag 2105 with sequence S-A1 corresponds to A, 2106 with sequence S-C1 corresponds to C and 2107 with sequence S-G1 corresponds to G. 2108 is the removable part of the tail tag with remaining part S-G1. In one embodiment, at least eight different tail tag types with a distinct sequence each are used, two for each base type, used in an alternating manner. This is demonstrated in the second nucleic acid molecule in FIG. 21. Said nucleic acid molecule has three previously incorporated nucleotides (2109), all of them comprising adenine (A). After the first incorporation event, tail tag 2110 was attached, after the second incorporation event, tail tag 2111 was attached, and after the third incorporation event, tail tag 2112 was attached. As shown, tail tag 2111 (the remaining part) comprises a different type of sequence (S-A2) from tail tags 2110 and 2112. This alternating use of distinct sequences enables to distinguish individual bases within a homopolymer sequence, by using methods that can detect different sequences. One such method comprises stretching the tail-tagged nucleic acid molecules onto an appropriate surface, denaturing them, and hybridizing them to labeled probes that can be detected. The method is described in more detail in another section herein, named “Sequencing of nucleic acid molecules and detection of tail tags using probes”.

In certain embodiments, a premade removable tail is attached to a nucleotide comprising a predetermined base type after said nucleotide is incorporated into a nucleic acid molecule. In one embodiment, the premade tail is an oligonucleotide that can hybridize to the nucleic acid molecule after incorporation of said nucleotide. Said oligonucleotide ligates to the 3′ end of the incorporated nucleotide. A nucleic acid molecule of interest is exposed to conditions to cause polymerization, and to a template-dependent polymerization reaction solution comprising reversibly blocked nucleotides comprising a predetermined base type. Then, the nucleic acid molecule is exposed to ligation reaction conditions, and a ligation reaction solution comprising random-sequence oligomers that serve as blocking tails. The blocking tails ligate to the nucleic acid molecule in the event that there is no nucleotide incorporation in the previous step. Random-sequence oligomers are single-stranded oligonucleotides generated to represent a plurality of sequences. Examples include random octamers that are commonly used, and are readily and commercially available from various sources (e.g., Roche, US Biological, Jena Bioscience, IDT, etc.). Random octamers can be readily produced to comprise cleavable nucleotides such as phosphorothioate-modified nucleotides in one or more positions at the 5′ end. In addition, random octamers can be readily modified at their 3′ end (for example, phosphorylated) to prevent off-site ligation of a removable tail in subsequent steps. Conditions suitable to perform hybridization and ligation of random octamers are known in the art (for example, see (Voelkerding et al., 2009); and (McKernan et al., 2009)).

The next step is to expose the nucleic acid molecule to conditions that unblock any incorporated nucleotide from the first step. Then, the nucleic acid molecule is exposed to conditions favoring ligation, and to a ligation reaction solution comprising random octamers that serve as removable tails. These octamers comprise one or more cleavable nucleotides at the 5′ end and also comprise one or more modified nucleotides carrying labels. Such octamers can be readily produced and hybridized to nucleic acid molecules using methods known to people skilled in the art.

### Example 1

### Extraction of Genomic DNA Molecules

Extraction of high quality genomic DNA from human blood can be achieved by using the Gentra Puregene reagents (Qiagen), per manufacturer's protocol. Briefly, add 3 ml of whole blood to a 15 ml tube containing 9 ml RBC Lysis Solution, invert to mix, then incubate for 5 min at room temperature. Invert again at least once during the incubation. Centrifuge for 2 min. Carefully discard the supernatant by pipetting, leaving approximately 200 μl of the residual liquid and the pellet. Vortex the tube vigorously to resuspend the pellet in the residual liquid. Add 3 ml of Cell Lysis Solution with 15 μl of RNase A Solution, and pipet up and down or vortex vigorously to lyse the cells. Add 1 ml Protein Precipitation Solution, and vortex vigorously for 20 sec at high speed. Centrifuge for 5 min at 3172 rpm. Add the supernatant from the previous step by pouring carefully into a 15 ml tube containing 3 ml isopropanol. Mix by inverting gently 50 times until the DNA is visible as threads or a clump. Centrifuge for 3 min. Carefully discard the supernatant. Add 3 ml of 70% ethanol and invert several times to wash the DNA pellet. Centrifuge for 1 min. Carefully discard the supernatant. Allow to air dry for 5-10 min. Add 100 μl TE buffer (10 mM Tris-HCl containing 1 mM EDTA). Vortex for 5 sec at medium speed to mix. Incubate at 65° C. for 2 h to dissolve the DNA. Incubate at room temperature overnight with gentle shaking. Centrifuge briefly and transfer to a 1.7 ml properly labeled sterile vial. Store at 4° C. overnight. Measure DNA concentration with NanoDrop (Thermo Scientific).

### Example 2

### Shearing of the Extracted DNA Molecules

The following protocol is adapted from Thompson J F, Steinmann K E. Single molecule sequencing with a HeliScope genetic analysis system. Curr Protoc Mol Biol. 2010 October; Chapter 7:Unit7.10. The materials used are: S2 instrument (Covaris, Inc., Woburn, Mass.), Preparation Station (Covaris, Inc., Woburn, Mass.), MicroTube holder (single tube) (Covaris, Inc., Woburn, Mass.), Snap-Cap microTube with AFA fiber and Pre-split, Teflon/silicone/Teflon septa (Covaris, Inc., Woburn, Mass.), Distilled Water (Invitrogen, Carlsbad, Calif.), 10×TE, pH 8.0 (Invitrogen, Carlsbad, Calif.), 1.5 mL MAXYMum Recovery tubes (Axygen Scientific, Union City, Calif.), Agencourt® AMPure® XP Kit (Agencourt Bioscience Corp., Beverly, Mass.), 100% Ethanol (Sigma, St Louis, Mo.), Dynal® Magnet: DynaMag®-2 Magnet (Invitrogen, Carlsbad, Calif.), Heatblock equipped with block milled for 1.5 mL tubes (VWR, Batavia, Ill.). Extracted genomic DNA can be sheared using the Covaris S2 instrument per manufacturer's instructions. Briefly, prepare 500 ng to 3 μg of DNA in 120 μl of TE, pH 8.0 and place the sample in a Covaris microTube. Slide the tube into the microTube holder, and insert the holder into the machine. On the Method Configuration Screen, set the Mode to Frequency Sweeping and the Bath Temperature Limit to 20° C. In the Treatment 1 box, set the Duty Cycle to 10%, the Intensity to 4 and the Cycles/Burst to 200. Set the time to 60 sec and start the treatment. The settings can produce 400-500 bases-long fragments. After shearing is complete, remove the tube from the holder. Transfer the sheared DNA to a new 1.5 mL tube. Samples may be stored at 20° C. after this step.

After shearing, size selection to remove very small fragments (<50 bp) can be done. This is accomplished by using the AMPure® XP beads per manufacturer's protocol. Briefly, add 360 μL of the AMPure® XP bead slurry to the tube of sheared DNA and mix. Incubate the sample for 5 to 10 minutes at room temperature. Capture the AMPure® XP beads by placing the tube on the Dynal™ magnet until the beads are separated from the solution (approximately 5 minutes). Carefully aspirate the supernatant keeping the tube on the magnet. Add 700 ml of 70% EtOH to each tube on the Dynal™ magnet. Wait 30 seconds. Keeping the tubes on the magnet, carefully aspirate the supernatant. Repeat ethanol washing. Briefly centrifuge the tubes to collect any remaining 70% EtOH to the bottom of the tube. Place the tubes back on the magnet and remove the last drops of 70% EtOH with a pipette. Dry the pellet at 37° C. Elute the sheared DNA sample from the AMPure beads by adding 20 μL of distilled water to each tube. A brief (1-2 sec) centrifugation may be performed to collect the beads at the bottom of the tube. Pipette the entire volume of each tube up and down 20 times so that the beads are completely resuspended. Place the tube back on the magnet. After the beads are separated from the solution, collect the 20 μL of solution and place it into a new 1.5 mL tube. This supernatant contains the sheared, size-selected DNA. Repeat elution with another 20 μL of water. The final sheared, size-selected DNA volume is 40 μL. The DNA can be stored at 20° C. after this step.

### Example 3

### Poly-A Tailing of Genomic DNA Molecules and Linking to Beads

The sheared genomic DNA from Example 2 can be subjected to poly-A tailing in order to be suitable for hybridization to magnetic beads covered with oligo-dT. Terminal transferase from New England BioLabs can be used. First, measure concentration of the DNA to be used in the reaction (NanoDrop). Then, mix: 5.0 μl 10×TdT Buffer, 5.0 μl 2.5 mM CoCl2 solution, 5.0 pmols DNA (˜0.7 μg for 400 bp; to determine approximate amount of DNA (ng/pmol), multiply the number of base pairs by 0.66), 1 μl 10 mM dATP, 0.5 μl Terminal Transferase (20 units/μl), and deionized water to a final volume of 50 μl. Incubate at 37° C. for 30 minutes. Stop the reaction by heating to 70° C. for 10 minutes or by adding 10 μl of 0.2 M EDTA (pH 8.0).

To process more DNA, three reactions can be done as described above, 50 μl each. After finishing, combine the three completed reactions into a single tube and purify the tailed DNA using the QIAquick PCR purification kit per manufacturer's protocol. The silica membranes in the columns provided with the kit bind the DNA, which is then eluted in distilled water (30 μl). Then, the eluted DNA is denatured to produce single strands and captured by oligo-dT magnetic beads (Dynabeads® Oligo (dT)25, Life Technologies). First, add the 30 μl of eluted DNA to 70 μl distilled DEPC-treated water. Then, add 100 μl of Binding Buffer (20 mM Tris-HCl, pH 7.5, 1.0 M LiCl, 2 mM EDTA). Heat to 65° C. for 2 min and immediately place on ice. Add the 200 μl to 100 μl of 1 mg pre-washed beads (beads need to be washed and resuspended in 100 μl of Binding Buffer prior to use). Mix thoroughly and anneal by rotating continuously on a mixer for 5 min at room temperature. Place the tube on the magnet for 1-2 min and carefully remove the supernatant. Remove the tube from the magnet and add 500 μl Washing Buffer (10 mM Tris-HCl, pH 7.5, 0.15 M LiCl, 1 mM EDTA). Mix by pipetting carefully a couple of times. Again use the magnet to pull the beads to the side of the tube. Carefully remove the supernatant. Repeat the washing step twice. The beads with the bound DNA are ready to use.

### Example 4

### Preparation of Tail Tags

What is shown in this example is the preparation of tail tags that are suitable for detection by a nanopore device comprising the protein nanopore α-hemolysin described in (Meller et al., 2000). Single-stranded DNA passes very fast through this nanopore, so the nanopore device cannot detect at a single-base or near-single-base resolution. Instead, it can discriminate changes in conductivity caused by specific sequence patterns such as “AC” or “TC” repeated 50 times, 50 A-nucleotides followed by 50 C-nucleotides, etc.

The following oligonucleotides can be prepared by commercial manufacturers. Oligonucleotides are phosphorylated at the 5′ end as shown (“5′-P-”) in order to be suitable for ligation.

[SEQ. ID. NO. 1]
Oligo A1: 5′-P- TCTACG (AC)50 GTCAAGCT -3′
[SEQ. ID. NO. 2]
Oligo A2: 5′-P-GCTTGAC(GT)50 -3′
[SEQ. ID. NO. 3]
Oligo C1: 5′-P- TCTACG (A)50 (C)50 GTCAAGCT -3′
[SEQ. ID. NO. 4]
Oligo C2: 5′- P-GCTTGAC(G)50(T)50 -3′
[SEQ. ID. NO. 5]
Oligo T1: 5′-P- TCTACG (TC)50 GTCAAGCT -3′
[SEQ. ID. NO. 6]
Oligo T2: 5′- P-GCTTGAC(GA)50-3′
[SEQ. ID. NO. 7]
Oligo G1: 5′-P- TCTACG (T)50 (C)50 GTCAAGCT -3′
[SEQ. ID. NO. 8]
Oligo G2: 5′- P-GCTTGAC(G)50(A)50 -3′

Oligo A2 is shorter than oligo A1 and complementary to oligo A1. Due to the shorter size, annealing of oligo A2 to oligo A1 leaves an overhang containing a single T at the 3′ of oligo A1, and a six nucleotide-long overhand at the 5′ end of oligo A1, which prevents self-ligation. The same applies to the pairs of oligos C1 and C2, oligos T1 and T2, and oligos G1 and G2.

In order to anneal the paired oligonucleotides, the following protocol is used:

Step 1: Resuspend complementary oligonucleotides at the same molar concentration, using 500 μl Annealing Buffer (10 mM Tris, pH 7.5-8.0, 50 mM NaCl, 1 mM EDTA), for each oligonucleotide.
Step 2: Annealing the Oligonucleotides: A) mix equal volumes of both complementary oligos in a 1.5 ml microfuge tube; b) place tube at 90-95° C. for 3-5 minutes; c) cool to room temperature; d) store on ice or at 4° C. until ready to use.

### Example 5

### Construction of Ligatable Removable Nucleotide Tails and Attachment of Tail Tags

The beads with the mixed population of genomic DNA molecules from Example 3 (referred to as “DNA beads”) are subjected to processes to construct ligatable removable nucleotide tails and attach tail tags. There are four different types of tail tags used, each specific for one of the DNA base types. The tail tags are attached to each DNA molecule in order according to the order that their corresponding base types are arranged in said DNA molecule.

Step 1: The DNA beads are re-suspended in 300 μl of 1× ThermoPol buffer [20 mM Tris-HCl, pH 8.8; 10 mM (NH4) 2SO4; 10 mM KCl; 2 mM MgSO4; 0.1% Triton X-100; New England BioLabs] comprising 6 units of Therminator (New England BioLabs), and 200 μM of 3′-O-amino-dATP (Firebird Biomolecular Sciences, LLC, Gainesville, Fla., USA). 10 μM of 3′-O-amino-dATP may be preferred, as it is suggested by studies that higher concentrations may lead to preferential incorporation of impurities (unmodified nucleotides) within the reversibly terminated nucleotide preparation (Gardner et al., 2012). The oligo-dTs that link the beads to the DNA molecules may act as primers to support extension. For reducing the chances of primer melting, oligo-dT primer extension at low temperature may be employed first, as described in Example 9 (extension using Klenow Fragment). The mixture described above is incubated in 72° C. for 1 min to allow extension. After the reaction is complete, the DNA beads are washed twice at room temperature using 0.5 ml of buffer comprising 10 mM Tris-HCl, pH 7.5, or 0.5 ml of 1× ThermoPol buffer.
Step 2: The DNA beads are re-suspended in 300 μl of 1× ThermoPol buffer with 6 units of Therminator and 200 μM each of ATP, UTP, GTP and CTP, and incubated in 72° C. for 1 min. After the reaction is complete, the DNA beads are washed twice as described in step 1. The DNA beads are re-suspended in 300 μl of 1× ThermoPol buffer with 6 units of Therminator and 1 M each of ddATP, ddTTP, ddGTP and ddCTP, and incubated in 72° C. for 1 min. After the reaction is complete, the DNA beads are washed twice as described. The reactions in Step 2 enable construction of a blocking nucleotide tail consisting of ribonucleotides and terminated with ddNTPs, said construction occurring in the event that 3′-O-amino-dATP is not incorporated in Step 1.
Step 3: The DNA beads are treated with 0.7 M NaNO2 and 1 M NaOAc, pH 5.5, at room temperature for 2 minutes, to cleave the terminator from the 3′-O-amino-dATP of Step 1. The DNA beads are then washed twice, as described before.
Step 4: The DNA beads are re-suspended in 300 μl of 1× ThermoPol buffer with 6 units of Therminator and 200 μM each of ATP, UTP, GTP and CTP, and incubated in 72° C. for 1 min. After the reaction is complete, the DNA beads are washed twice, as described. Then, the DNA beads are re-suspended in 300 μl of 1× ThermoPol buffer with 6 units of Therminator and 200 μM each of dATP, dTTP, dGTP and dCTP, and incubated in 72° C. for 1 min. After the reaction is complete, the DNA beads are washed twice, as described. To enable complete elongation and the addition of an adenine-comprising single-nucleotide overhang, the DNA beads are re-suspended in 300 μl of 1× LongAmp™ Taq Reaction Buffer (60 mM Tris-504, 20 mM (NH4) 2SO4, 2 mM MgSO4, 3% Glycerol, 0.06% IGEPAL® CA-630, 0.05% Tween® 20, pH 9 at 25° C.) comprising 30 units of LongAmp Taq DNA Polymerase (New England BioLabs) and 200 μM each of dATP, dTTP, dGTP and dCTP, and incubated at 65° C. for 3 min. After the reaction is complete, the DNA beads are washed twice, as described. The reactions in Step 4 enable construction of a ligatable removable nucleotide tail consisting of ribonucleotides, deoxyribonucleotides and a dATP overhang, said construction occurring in the event that 3′-O-amino-dATP is incorporated in Step 1.
Step 5: The DNA beads are re-suspended in 50 μl of sterile deionized water comprising 3 μg of tail tags made of the annealed oligos A1 and A2, shown in Example 4. Add 50 μl of Blunt/TA Ligase Master Mix (already comprising T4 DNA Ligase; New England BioLabs) and mix thoroughly by pipetting up and down 7-10 times or by finger-flicking. Incubate at room temperature (25° C.) for 15 min, place on ice. After the reaction is complete, the DNA beads are washed twice, as described.
Step 6: The DNA beads are re-suspended in 100 μl of 1× ThermoPol Buffer. Add 5 μl (25 units) of RNase HII (New England BioLabs) and mix thoroughly. Incubate at 37° C. for 5 minutes. After the reaction is complete, the DNA beads are washed twice, as described. This step removes the ribonucleotide parts of any blocking or removable nucleotide tails constructed in steps 2 and 4. According to the manufacturer, RNase HII preferentially nicks 5′ to a ribonucleotide within the context of a DNA duplex. The enzyme leaves 5′ phosphate and 3′ hydroxyl ends. RNase HIT also nicks at multiple sites along the RNA portion of RNA:DNA hybrids. Other RNase HIT preparations suitable for the application can be derived from T. kodakaraensis or B. subtilis, as described in studies referenced elsewhere herein.
Step 7: Repeat steps 1 through 6, using 3′-O-amino-dCTP (instead of 3′-O-amino-dATP) in step 1, and using tail tags made of the annealed oligos C1 and C2 (as shown in Example 4).
Step 8: Repeat steps 1 through 6, using 3′-O-amino-dTTP and tail tags made of oligos T1 and T2.
Step 9: Repeat steps 1 through 6, using 3′-O-amino-dGTP and tail tags made of oligos G1 and G2.
Step 10: Repeat steps 1 through 9 multiple times (for example, 30).

### Example 6

### Sequencing Using a Nanopore Device

For sequencing, the protein nanopore a-hemolysin is used as described in (Meller et al., 2000).

In brief, single channels are formed in a horizontal bilayer of diphytanoyl phosphatidylcholine by using the protein α-hemolysin from Staphylococcus aureus.

Prior to loading to the nanopore device, the DNA molecules attached to tail tags from Example 5 are incubated at 95° C. for 3 min to denature, and are cooled down in ice.

The experiment is performed in 1 M KCl/10 mM Tris.Cl, pH 8.5, and DNA is applied to the apparatus. 120 mV is applied across an α-hemolysin channel. The resultant ionic current flow through the a-hemolysin channel is amplified and measured by using a patch-clamp amplifier and head-stage (Axopatch 200B and CV203BU, Axon Instruments, Foster City, Calif.). The amplified signals are low-pass filtered at 100 KHz (3302 filter, Krohn-Hite, Avon, M A), and digitized at 333 KHz with a 12-bit analog/digital board (Axon). As DNA molecules translocate through the channel, the current drops according to the DNA sequence content. Currents are recorded using special acquisition software (CLAMPEX 7, Axon).

### Example 7

### Attachment of Tail Tags to Lambda Genome Fragments

Fragmentation, cleanup and size selection of genomic DNA

Lambda phage DNA was fragmented and the fragments were end-repaired and ligated to hairpin adaptors bound to streptavidin-coated magnetic beads.

5 μg of lambda phage DNA (New England BioLabs, Inc., Ipswich, Mass.) were fragmented using the NEBNext® dsDNA Fragmentase® kit (New England BioLabs, Inc., Ipswich, Mass.). Specifically, a 20 μl solution comprising 2 μl dsDNA fragmentase, 2 μl 10× Fragmentase Reaction Buffer v2, 1 μl of 200 mM MgCl₂, lambda phage DNA and sterile deionized water was incubated at 37° C. for 45 min. Fragmentation was stopped by adding 5 μl 0.5 M EDTA pH 8.0.

The fragmented DNA was cleaned and size-selected using Agencourt® AMPure® XP beads (Beckman Coulter, Brea, Calif.). 75 μl of sterile deionized water were added to the stopped fragmentation reaction (25 μl), followed by the addition of 150 μl AMPure® XP beads. The mixture was incubated at room temperature for 5 min, and then placed on magnet for bead separation. The beads were discarded and the supernatant, which contained DNA fragments of the desired size (approximately less than 200 bp) was kept and mixed with 300 μl AMPure® XP beads to capture DNA fragments. The mixture was incubated at room temperature for 5 min, and then placed on magnet. The supernatant was discarded and the beads were washed twice with 500 μl fresh 80% ethanol. The beads were left to dry. Bound DNA fragments were eluted by adding 40 μl sterile deionized water and incubating for 5 min at room temperature, before placing on magnet. The supernatant was carefully removed to prevent bead carry-over.

DNA fragment end-repair and A-tailing

The next step was to end-repair the eluted DNA fragments and add A tails suitable for TA ligation. 35 μl of the supernatant from the previous step were added to a solution comprising 5 μl 10× NEBuffer 2 (1×: 50 mM NaCl, 10 mM Tris-HCl, 10 mM MgCl₂, 1 mM DTT, pH 7.9) (New England BioLabs, Inc., Ipswich, Mass.), 1 μl ATP (100 mM), 0.4 μl dNTP (100 mM), 2 μl T4 DNA polymerase (3 units/μl), 2 μl T4 polynucleotide kinase (10 units/μl), 2 μl Taq DNA polymerase (5 units/μl), and sterile deionized water up to total reaction volume of 50 μl. The solution was first incubated at 25° C. for 20 min, and then at 72° C. for 20 min.

Anchoring of DNA fragments to streptavidin-coated beads

The repaired DNA fragments carrying 3′-end A-tails were TA-ligated to hairpin adaptors that were bound to streptavidin beads. The hairpin adaptors had the following sequence:

[SEQ. ID. NO. 9]
GACTCGGAAGAGCGTCGTGTAGGGAAAGAGTGTAGTTTTTTTCTACACTC
TTTCCCTACACGACGCTCTTCCGAGTCT

The hairpins had phosphorylated 5′ ends, T overhangs at the 3′ ends suitable for TA ligation, a stem of 35 base pairs and a loop of 7 Ts. The fourth Tin the loop was biotinylated to cause binding of hairpins to streptavidin through biotin-streptavidin interactions.

For proper hairpin formation, 50 pmoles of hairpin adaptors (1 μl of 50 μM stock) were diluted in 100 μl of Annealing Buffer (10 mM Tris-HCl pH 7.5, 100 mM NaCl), incubated at 95° C. for 5 min, and left in room temperature to gradually cool down.

In order to bind hairpin adaptors to streptavidin beads, 100 μl streptavidin-coated magnetic beads (Dynabeads® MyOne™ Streptavidin C1, Life Technologies, Carlsbad, Calif.) were first washed 3 times with 1 ml 1× Binding Buffer (5 mM Tris-HCl (pH 7.5), 0.5 mM EDTA, 1 M NaCl). Unless otherwise specified, washing of magnetic beads mentioned herein comprises adding appropriate buffer, placing on magnet (Ambion® 6 tube magnetic stand, Life Technologies, Carlsbad, Calif.) to collect the beads, and discarding the supernatant. After washing, the beads were re-suspended in 200 μl 2× Binding Buffer (10 mM Tris-HCl (pH 7.5), 1 mM EDTA, 2 M NaCl), 100 μl of annealed hairpin adaptors, and 100 μl sterile deionized water, and incubated in room temperature with gentle rotation for 15 min. After incubation, the beads were washed twice with 1 ml 1× Binding Buffer, and twice with 1 ml 1×T4 DNA ligase reaction buffer (50 mM Tris-HCl, 10 mM MgCl₂, 1 mM ATP, 10 mM DTT, pH 7.5)(New England BioLabs, Inc., Ipswich, Mass.).

After washing, the collected beads bound to hairpin adaptors were re-suspended in the 50 μl DNA repair and tailing reaction solution from the previous step, and 50 μl of Blunt/TA Ligase Master Mix (360 units T4 DNA ligase/μl)(New England BioLabs, Inc., Ipswich, Mass.). The ligation reaction was incubated at 25° C. for 1 hour. After incubation, the beads were placed on magnet, the supernatant was discarded, and the beads were washed three times with 600 μl 1× NEBuffer 3.1 (1×: 100 mM NaCl, 50 mM Tris-HCl, 10 mM MgCl₂, 100 μg/ml BSA, pH 7.9) (New England BioLabs, Inc., Ipswich, Mass.).

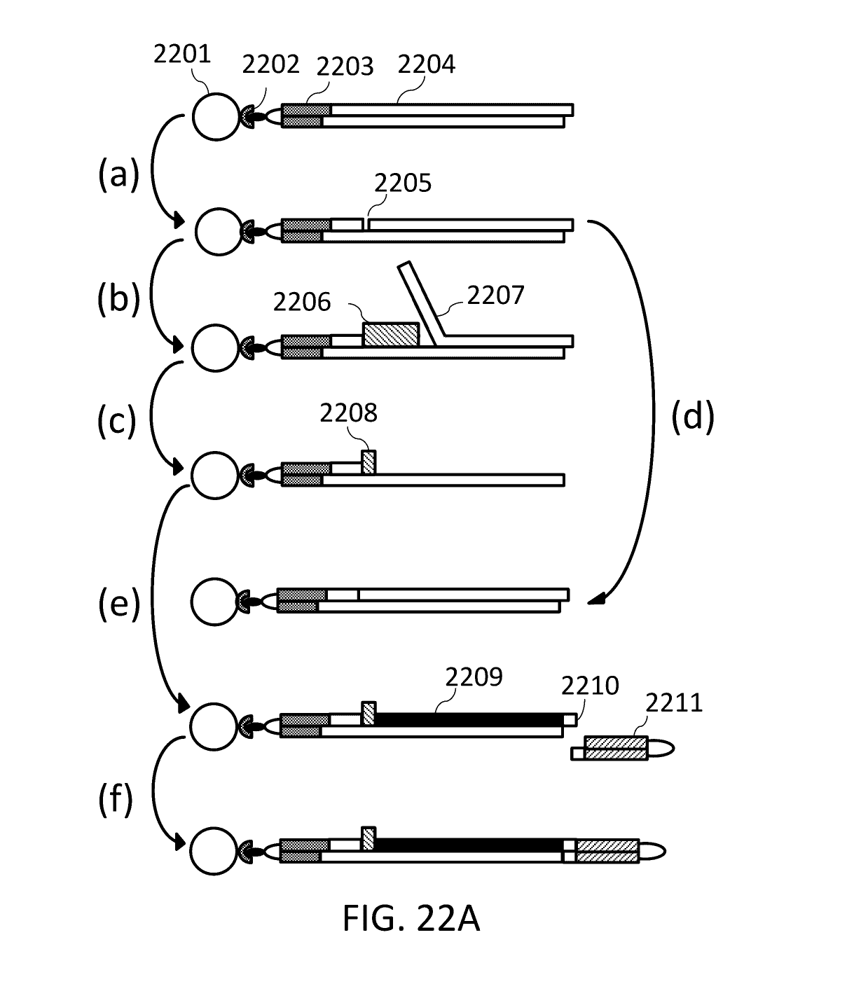

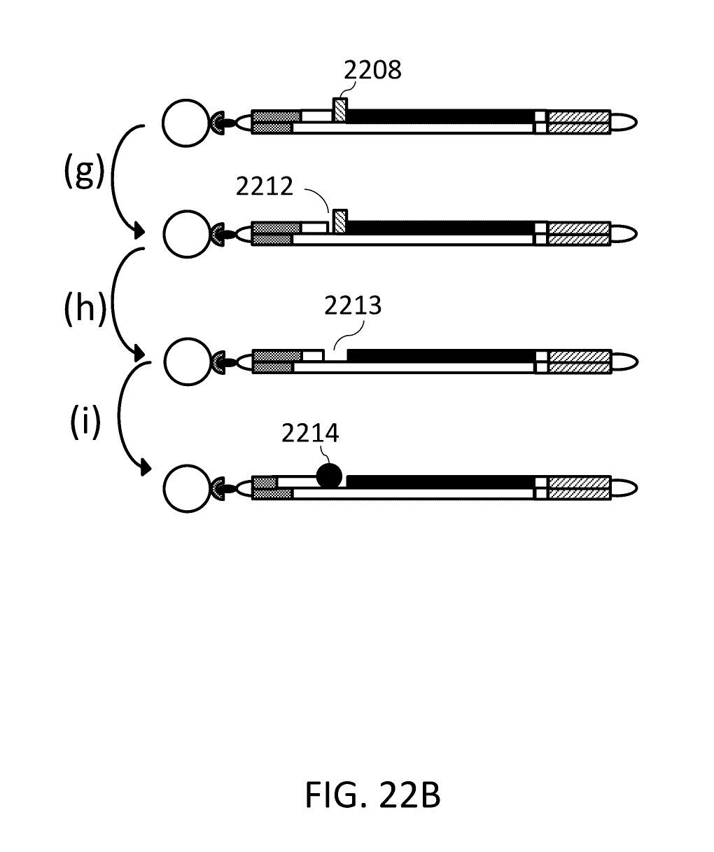

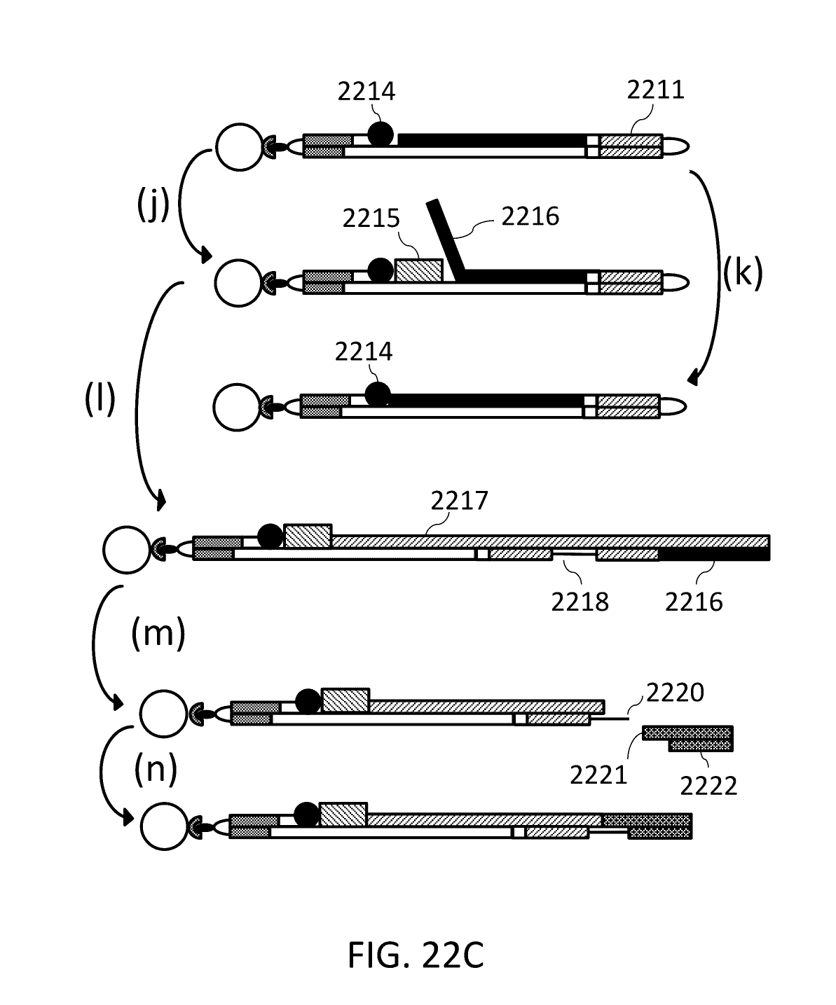

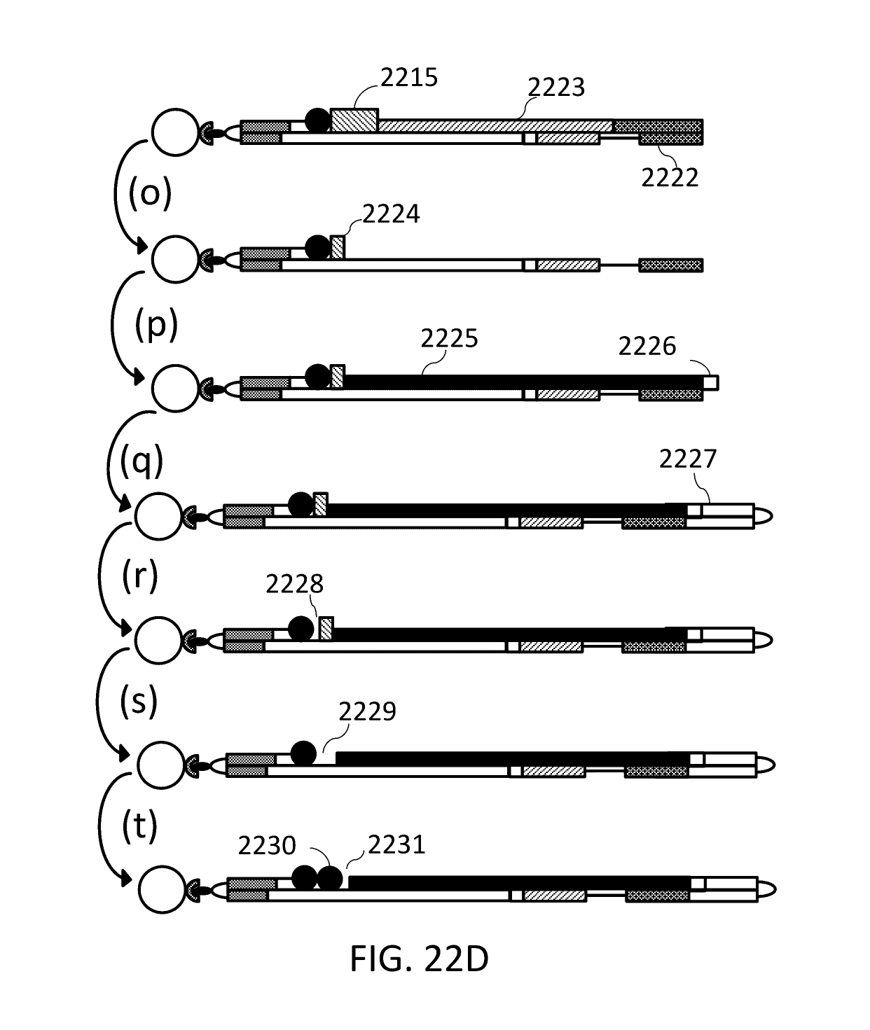

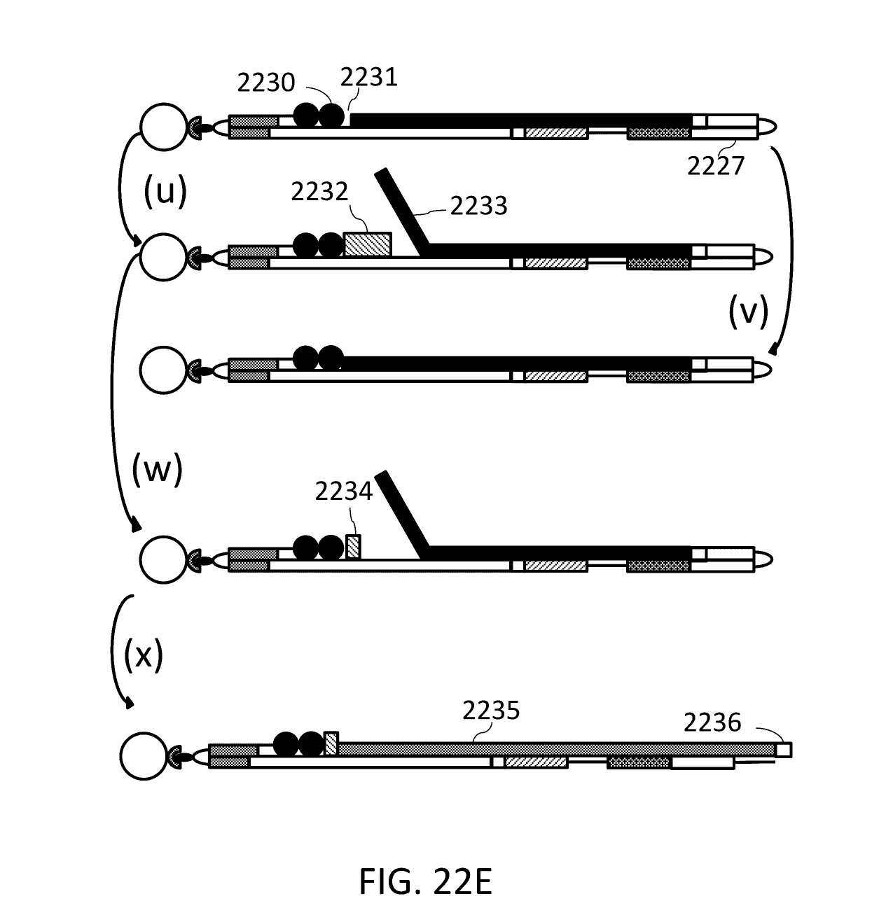

FIG. 22A shows a diagram of a construct produced by the experiment described above. A DNA fragment 2204 is shown ligated to a hairpin adaptor 2203, which is anchored to a streptavidin-coated magnetic bead 2201 by binding to streptavidin 2202.

### DNA Nicking

In order to generate nicks in the ligated DNA fragments, that introduce extendable 3′ ends, the beads were re-suspended in a solution comprising 172 μl sterile deionized water, 20 μl 10× NEBuffer 3.1, and 8 μl Nt.BstNBI (10 units/μl). The beads were mixed by pipetting and incubated at 55° C. for 30 min.

After incubation, the solution was placed on a magnet to separate beads. The supernatant was discarded and the beads were washed 3 times with 600 μl 1× ThermoPol® buffer (20 mM Tris-HCl, 10 mM (NH₄) 2SO₄, 10 mM KCl, 2 mM MgSO₄, 0.1% Triton® X-100, pH 8.8)(New England BioLabs, Inc., Ipswich, Mass.).

The procedure of nicking is depicted as step (a) in FIG. 22A, which produces nick 2205.

Incorporation of ribonucleotides comprising predetermined base types

The beads from the previous experiment were re-suspended in 400 μl 1× ThermoPol® buffer and divided in 4 samples, 100 μl each. The 4 samples were placed on magnet to collect the beads. The beads from the 4 samples were re-suspended in solutions comprising ribonucleotides comprising predetermined base types. Specifically, the solution in each sample had a total volume of 100 μl, comprising sterile deionized water, 10 μl 10× ThermoPol® buffer and 2.5 μl Therminator DNA polymerase (2 units/μl) (New England BioLabs, Inc., Ipswich, Mass.). The solution of one sample also comprised 0.2 μl ATP (100 mM), the solution of another sample comprised 0.2 μl UTP (100 mM), the solution of the third sample comprised 0.2 μl GTP (100 mM), and the solution of the fourth sample comprised 0.2 μl CTP (100 mM). The samples were incubated at 72° C. for 10 min. The samples placed on a magnet after incubation, the supernatants were discarded and the beads from each sample were washed 3 times with 200 μl 1× ThermoPol® buffer.

Ribonucleotide incorporation is represented by step (b) in FIG. 22A. During step (b), RNA segment 2206 is produced by the polymerizing action of Therminator, with simultaneous displacement of the strand 2207. 2206 comprises one ribonucleotide, or more ribonucleotides comprising the same base type in the event that there is a homopolymer segment in the nucleic acid template strand complementary to said base type.

### Ribonucleotide Cleavage and 3′ End Dephosphorylation

Incorporated ribonucleotides from the previous step were cleaved using NaOH. NaOH cleaves the RNA part of a DNA:RNA hybrid, leaving a single ribonucleotide bound to the 3′ end of the DNA strand. The remaining ribonucleotide has a phosphate at the 3′ end. The mechanism of alkaline hydrolysis and associated experiments are described in Example 10 and elsewhere herein. In the event that 2206 comprised only one ribonucleotide, there is no cleavage, and said ribonucleotide remains unaltered.

The beads from each sample were re-suspended in 100 μl 0.2N NaOH and incubated at 90° C. for 15 min. The solutions were put on a magnet and the separated beads were washed 3 times with 1× NEBuffer 2. NaOH causes denaturation of nucleic acid strands. The DNA molecules bound to the beads were re-annealed by adding 200 μl 1× NEBuffer 2 in each sample, incubating for 5 min at 95° C., and leaving in room temperature for gradual cooling down.

As mentioned above, the ribonucleotides remaining after NaOH treatment have phosphorylated 3′ ends. The phosphates can be removed by T4 polynucleotide kinase treatment. For this purpose, the beads from each sample were washed once with 200 μl 1×T4 polynucleotide kinase reaction buffer (70 mM Tris-HCl, 10 mM MgCl₂, 5 mM DTT, pH 7.6)(New England BioLabs, Inc., Ipswich, Mass.), and were placed in a solution comprising 10 μl 10×T4 polynucleotide kinase reaction buffer, 2 μl ATP (100 mM), 4 μl T4 polynucleotide kinase (10 units/μl) and sterile deionized water up to 100 μl of final volume. The solutions were incubated at 37° C. for 30 min, and then placed on magnet. The supernatants were discarded and the beads from each sample were washed twice with 200 μl 1×T4 DNA ligase reaction buffer.

The ribonucleotide cleavage and dephosphorylation step is shown in FIG. 22A as step (c), which cleaves 2206 (in the event that 2206 comprises more than one nucleotides) and leaves 2208 behind.

### Formation of Terminal Blocking Nucleotide Tails

The beads from each sample were re-suspended in a 40 μl solution comprising 15 μl of Blunt/TA Ligase Master Mix (360 units T4 DNA ligase/μl)(New England BioLabs, Inc., Ipswich, Mass.) and sterile deionized water. The reactions were incubated at 25° C. for 120 min. The purpose of this step was to seal nicks, leading to the formation of terminal blocking nucleotide tails; the extendable 3′ ends of nicked nucleic acid molecules that did not incorporate ribonucleotides in the previous step were sealed and rendered non-extendable. After completion of the incubation, the samples were placed on magnet, the supernatants were discarded and the beads from each sample were washed twice with 200 μl 1× ThermoPol® buffer.

Formation of a terminal blocking nucleotide tail is shown as step (d) in FIG. 16A, which leads to sealing of the nick 2205.

### Deoxyribonucleotide Incorporation and A-Tailing

The beads from each sample were re-suspended in a solution comprising 10 μl 10× ThermoPol® buffer, 0.8 μl dNTP (100 mM (25 mM of each nucleotide type)), 2 μl Therminator DNA polymerase (2 units/μl), 0.5 μl Taq DNA polymerase (5 units/μl), and sterile deionized water up to 100 μl of total reaction volume. The solutions were incubated at 72° C. for 5 min. The beads were separated using a magnet, the supernatants were discarded, and the beads were washed twice with 200 μl 1× ThermoPol® buffer.

This step is shown as step (e) in FIG. 22A. 2209 is the newly formed strand segment, and 2210 is the A overhang.

### Ligation of First Tail Tags

The beads from each sample from the previous experiment were re-suspended in 100 μl 1× ThermoPol® buffer. The DNA molecules in each sample were ligated to hairpin tail tags corresponding to a single nucleotide base type (A, T, C or G), according to the predetermined base type said samples were exposed to during the ribonucleotide incorporation step. Specifically, the sample that was subjected to ribonucleotide polymerization with ATP, was subjected to ligation with hairpin tail tags corresponding to adenine (A). The sample that was subjected to ribonucleotide polymerization with UTP, was subjected to ligation with hairpin tail tags corresponding to thymine (T). The sample that was subjected to ribonucleotide polymerization with GTP, was subjected to ligation with hairpin tail tags corresponding to guanine (G). The sample that was subjected to ribonucleotide polymerization with CTP, was subjected to ligation with hairpin tail tags corresponding to cytosine (C).

The hairpin tail tags used were:
Hairpin T corresponding to T:

[SEQ. ID. NO. 10]
CTTCTCTCTCTCTTCTCTCTTTTTGAGCTCGGTAACCTTGGTTTAAGAGA
GAAGAGAGAGAGAAGT

Hairpin A corresponding to A:

[SEQ. ID. NO. 11]
GAGAAGAAGGAGAAGAGAGGATTTGAGCTCGGTAACCTTGGTTTTCCTCT
CTTCTCCTTCTTCTCT

Hairpin G corresponding to G:

[SEQ. ID. NO. 12]
GTGTGGTTGTGTGTTGTGGTTTTTGAGCTCGGTAACCTTGGTTTAACCAC
AACACACAACCACACT

Hairpin C corresponding to C:

[SEQ. ID. NO. 13]
CCACACCACACACACCACACTTTGAGCTCGGTAACCTTGGTTTGTGTGGT
GTGTGTGGTGTGGT

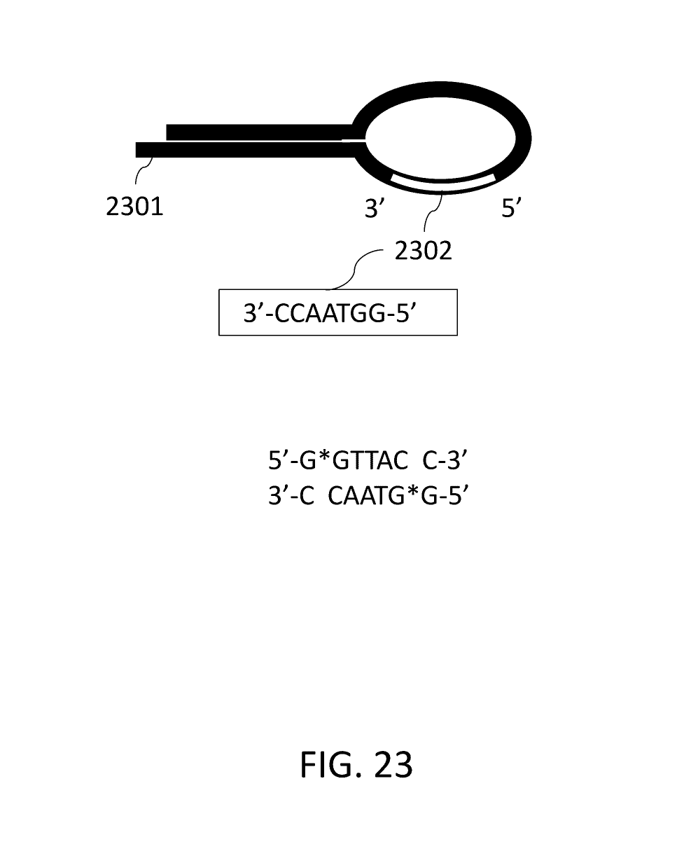

The hairpins had phosphorylated 5′ ends, T overhangs at the 3′ ends suitable for TA ligation, and recognition sites within their loops, specific for the restriction endonuclease BstEII. FIG. 23 shows the general structure of the hairpins; 2301 is the T overhang; 2302 is the segment of the hairpin loop comprising the BstEII restriction site. As 2302 is comprised in the hairpin loop, it is single-stranded (not complementary to another strand segment), and not yet recognized by restriction enzymes. The double-stranded sequence is shown, demonstrating the BstEII site. Stars mark the cleavage sites.

For proper hairpin formation, hairpin tail tags were diluted in 25 μl 1× NEBuffer 2 to a final concentration of 10 μM, incubated at 95° C. for 5 min, and left in room temperature to gradually cool down.

Subsequently, the samples were placed on magnet, and the supernatants were discarded. The beads were washed 3 times with 200 μl 1×T4 DNA ligase reaction buffer. Then, the beads were re-suspended in solutions comprising 25 μl of annealed hairpin tail tags (Hairpin A, T, C, or G; 10 μM) and 15 μl of Blunt/TA Ligase Master Mix. The samples were incubated for 30 min at 25° C.

The samples were placed on magnet, the supernatants were discarded, and the beads were washed twice with 400 μl 1× CutSmart buffer (50 mM Potassium Acetate, 20 mM Tris-acetate, 10 mM Magnesium Acetate, 100 μm/ml BSA, pH 7.9) (New England BioLabs, Inc., Ipswich, Mass.).

In order to prevent unligated DNA molecules from participating in future ligation reactions, the samples were treated with rSAP (recombinant shrimp alkaline phosphatase; New England BioLabs, Inc., Ipswich, Mass.), an enzyme that dephosphorylates 5′ ends. Specifically, the beads from each sample were re-suspended in solutions comprising 10 μl 10× CutSmart buffer, 15 μl rSAP (1 unit/μl), and sterile deionized water up to a final reaction volume of 100 μl. The reactions were incubated at 37° C. for 30 min, and then at 65° C. for 5 min (enzyme inactivation step). The reactions were placed on magnet, the supernatants were discarded and the beads were washed 3 times with 200 μl 1× ThermoPol® buffer.

Ligation of first tail tags is shown as step (f) in FIG. 22A. 2211 represents a hairpin tail tag.

### RNase HII-Mediated Nick Formation

Nicks were produced at the 5′ ends of the single ribonucleotides remaining after the NaOH treatment, using RNase HII. The beads of each sample were re-suspended in a solution comprising 10 μl 10× ThermoPol® buffer, 5 μl RNase HII (5 units/μl)(New England BioLabs, Inc., Ipswich, Mass.), and sterile deionized water up to 100 μl of total reaction volume. The solutions were incubated at 37° C. for 30 min. Then, the solutions were placed on a magnet to separate the beads, the supernatants were discarded, and the beads were washed twice with 200 μl 1× ThermoPol buffer.

This step is shown as step (g) in FIG. 22B, which produces nick 2212 at the 5′ end side of the ribonucleotide 2208.

### Single-Base Gap Formation

The beads from each sample were re-suspended in 100 μl of 0.2N NaOH and incubated at 90° C. for 15 min, in order to release the ribonucleotides still attached by their 3′ end to the DNA molecules that previously incorporated ribonucleotides at their nicked sites (shown as step (h) in FIG. 22B, which generates gap 2213). Since NaOH denatures nucleic acid molecules, the DNA molecules bound to the beads were first washed 3 times in 200 μl 1× NEBuffer 2, and re-annealed by adding 100 μl 1× NEBuffer 2, incubating for 5 min at 95° C., and leaving in room temperature for gradual cooling-down. The samples were placed on magnet to collect beads.

### Single-Base Gap Filling

The beads from each sample were re-suspended in a reaction volume of 100 μl comprising 10 μl 10× NEBuffer 2, 1 μl BSA (10 mg/ml), 0.8 μl dNTP (100 mM), 6 μl T4 DNA polymerase (3 units/μl) and sterile deionized water. The samples were incubated at 20° C. for 5 min. The samples were placed on magnet on ice (to suppress enzymatic activity), the supernatants were discarded and the beads were washed twice with cold 200 μl 1× ThermoPol buffer. To ensure gap filling, the beads of each sample were re-suspended in a reaction volume of 100 μl comprising 10 μl 10× ThermoPol buffer, 0.8 μl dNTP (100 mM), 2.5 μl Sulfolobus DNA polymerase IV (2 units/μl) (New England BioLabs, Inc., Ipswich, Mass.), and sterile deionized water. The samples were incubated at 55° C. for 5 min. After incubation, the beads from each sample were washed 3 times with 200 μl 1× ThermoPol® buffer. The process of gap filling is shown as step (i) in FIG. 22B. During step (i), deoxyribonucleotide 2214 is incorporated.

### Incorporation of Ribonucleotides Comprising a Predetermined Base Type

The beads of each sample were re-suspended in 100 μl 1× ThermoPol® buffer, then mixed together with the re-suspended beads from the other samples, and divided in 4 new samples. The DNA molecules in each sample were exposed to a solution comprising ribonucleotides comprising a single predetermined base type. Specifically, the solution in each sample had a total volume of 100 μl, comprising 10 μl 10× ThermoPol® buffer and 2.5 μl Therminator DNA polymerase. The solution of one sample also comprised 0.2 μl ATP (100 mM), the solution of another sample comprised 0.2 μl UTP (100 mM), the solution of the third sample comprised 0.2 μl GTP (100 mM), and the solution of the fourth sample comprised 0.2 μl CTP (100 mM). The reactions were incubated at 72° C. for 10 min. The beads were separated using a magnet, the supernatants were discarded, and the beads were washed 3 times with 200 μl 1×T4 DNA ligase reaction buffer.

Ribonucleotide incorporation is represented by step (j) in FIG. 22C. During step (j), RNA segment 2215 is produced by the polymerizing action of Therminator, with simultaneous displacement of the strand 2216. 2215 comprises one ribonucleotide, or more ribonucleotides comprising the same base type in the event that there is a homopolymer segment in the nucleic acid template strand complementary to said base type.

### Terminal Blocking Nucleotide Tail Formation by Ligation

The beads from each sample were re-suspended in a 40 μl solution comprising 15 μl of Blunt/TA Ligase Master Mix (360 units T4 DNA ligase/μl) (New England BioLabs, Inc., Ipswich, Mass.) and sterile deionized water. The reactions were incubated at 25° C. for 120 min. The purpose of this step was to seal nicks, leading to the formation of terminal blocking nucleotide tails; the extendable 3′ ends of nicked nucleic acid molecules that did not incorporate ribonucleotides in the previous step were sealed and rendered non-extendable. After completion of the incubation, the samples were placed on magnet, the supernatants were discarded and the beads were washed twice with 200 μl 1× ThermoPol® buffer. Terminal blocking nucleotide tail formation is shown as step (k) in FIG. 22C, during which the nick following nucleotide 2214 is sealed.

### Deoxyribonucleotide Extension

The beads of each sample were re-suspended in a solution comprising 10 μl 10× ThermoPol® buffer, 0.8 μl dNTP (100 mM (25 mM of each nucleotide type)), 2 μl Therminator DNA polymerase (2 units/μl), and sterile deionized water up to 100 μl of total reaction volume. The solutions were incubated at 72° C. for 10 min. The beads were separated using a magnet, the supernatants were discarded, and the beads were washed 3 times with 200 μl 1× CutSmart buffer.

The process of deoxyribonucleotide extension is represented by step (l) in FIG. 22C. 2217 is the newly formed strand extending from the 3′ end of 2215. Therminator is a strand displacing polymerase. For that reason, 2217 is complementary to the entire hairpin (loop 2218 of hairpin 2211 is marked for clarity) and to the displaced strand 2216 (not shown in proportion, to fit the page).

### Tail Tag Ligation

The extension of the previous step produced strands complementary to the single-stranded loops of Hairpin A, T, C, and G that were previously ligated to the DNA molecules. By becoming double-stranded, the loops could be recognized and cleaved by BstEII as shown in FIG. 23.

The beads from each sample were re-suspended in a solution comprising 5 μl 10× CutSmart, 1 μl BstEII-HF® (high fidelity; 20 units/μ1; New England BioLabs, Inc., Ipswich, Mass.), and sterile deionized water to a final reaction volume of 50 μl. The reactions were incubated at 37° C. for 15 min. Then, the samples were placed on a magnet, the supernatants were discarded, and the beads were washed 3 times with 1×T4 DNA ligase reaction buffer.

Digestion with BstEII is shown as step (m) in FIG. 22C. During step (m), the protruding 5′ end 2220 is formed comprising part of the hairpin loop 2218, which is complementary to the overhang 2221 of tail tag 2222.

After washing, each sample was subjected to ligation with tail tags that corresponded to the predetermined base type matching the specific sample (i.e. the predetermined base type comprised in the ribonucleotides that the sample was exposed to during the ribonucleotide incorporation step represented by step (j) in FIG. 22C). The tail tags were double-stranded oligonucleotides with one end blunted and unphosphorylated, and the other being phosphorylated at the 5′ end and carrying a 5′ overhang complementary to the excised BstEII sites generated during the immediately preceding step. The tail tags used were the following:

Tail tag T corresponding to the T base, and formed by annealing the oligonucleotide with sequence

[SEQ. ID. NO. 14]
/5Phos/GTTACCCTTCTCTCTCTCTTCTCTCTTCAACTCCAGTCACATC
AGGATCTCAGATGGCGTCTT

(where/5Phos/marks 5′ end phosphorylation) to the oligonucleotide with sequence

[SEQ. ID. NO. 15]
AAGACGCCATCTGAGATCCTGATGTGACTGGAGTTGAAGAGAGAAGAGAG
AGAGAAGG;

Tail tag A corresponding to the A base, and formed by annealing the oligonucleotide with sequence

[SEQ. ID. NO. 16]
/5Phos/GTTACCGAGAAGAAGGAGAAGAGAGGACAACTCCAGTCACATC
AGGATCTCAGATGGCGTCTT

to the oligonucleotide with sequence

[SEQ. ID. NO. 17]
AAGACGCCATCTGAGATCCTGATGTGACTGGAGTTGTCCTCTCTTCTCCT
TCTTCTCG;

Tail tag G corresponding to the G base, and formed by annealing the oligonucleotide with sequence

[SEQ. ID. NO. 18]
/5Phos/GTTACCGTGTGGTTGTGTGTTGTGGTTCAACTCCAGTCACATC
AGGATCTCAGATGGCGTCTT

to the oligonucleotide with sequence

[SEQ. ID. NO. 19]
AAGACGCCATCTGAGATCCTGATGTGACTGGAGTTGAACCACAACACACA
ACCACACG;

Tail tag C corresponding to the C base, and formed by annealing the oligonucleotide with sequence

[SEQ. ID. NO. 20]
/5Phos/GTTACCACCACACCACACACACCACACCAACTCCAGTCACATC
AGGATCTCAGATGGCGTCTT

to the oligonucleotide with sequence

[SEQ. ID. NO. 21]
AAGACGCCATCTGAGATCCTGATGTGACTGGAGTTGGTGTGGTGTGTGTG
GTGTGGTG.

To anneal the tail tag oligonucleotides, 10 μl of one oligonucleotide type (50 μM) and 10 μl of its complementary oligonucleotide type (50 μM) were mixed with 2.5 μl 10× NEBuffer 2 and 2.5 μl sterile deionized water (total volume: 25 μl), incubated at 95° C. for 5 min, and left in room temperature to gradually cool down.

The beads of each sample were re-suspended in 25 μl of annealed tail tags corresponding to the base type matching the specific sample, and 15 μl Blunt/TA Ligase Master Mix. The reactions were incubated at 25° C. for 30 min. Tail tag ligation is shown as step (n) in FIG. 22C.

### Amplification of DNA Fragments Ligated to Tail Tags

The beads from the previous step were pooled and washed 3 times with 600 μl 1× ThermoPol® buffer. After washing, the beads were re-suspended in 200 μl 1× ThermoPol® buffer. 35 μl of the re-suspended beads were used in 7 PCR reactions using Q5® Hot Start High-Fidelity DNA polymerase and associated reagents (New England BioLabs, Inc., Ipswich, Mass.), to amplify the DNA fragments ligated to tail tags. Each PCR had a total volume of 50 μl, comprising 5 μl of re-suspended beads, 10 μl of 5× Q5® High GC Enhancer, 10 μl of 5×Q5® Reaction Buffer, 0.4 μl dNTP (100 mM), 2.5 μl Forward Primer (10 μM) with sequence:

[SEQ. ID. NO. 22]
/5Phos/CTACACTCTTTCCCTACACGACGCTCTTCCGAGTCT

and 2.5 μl Reverse Primer (10 μM) with sequence:

[SEQ. ID. NO. 23]
/5Phos/AAGACGCCATCTGAGATCCTGATGTGACTGGAGTTG

(where/5Phos/marks 5′ end phosphorylation), 0.5 μl Q5® Hot Start High-Fidelity DNA polymerase (2 units/μl) and sterile deionized water. The reactions were placed on a thermocycler (Applied Biosystems® 2720 Thermal Cycler; Life Technologies, Carlsbad, Calif.) for an initial denaturation step for 30 sec at 98° C., 25 cycles comprising 3 steps each (98° C. for 10 sec; 63° C. for 20 sec; 72° C. for 20 sec), and a final extension step for 2 min at 72° C.

The amplified DNA products were cleaned and size-selected using Agencourt® AMPure® XP beads (Beckman Coulter, Brea, Calif.). The PCR reactions were pooled (total of 350 μl) and mixed with 280 μl AMPure® XP beads (0.8 ratio). The mixture was incubated at room temperature for 5 min, in order to bind undesirable amplified products (longer than approximately 400 bp) to the beads. After incubation, the sample was placed on a magnet to separate the beads. 575 μl of supernatant were recovered, and the beads were discarded. The supernatant was then incubated with 517.5 μl of AMPure® XP beads (0.9 ratio) for 5 min at room temperature, and then placed on magnet. The incubation served the purpose of binding amplified products of the desirable size (approximately between 50 and 400 bp) to the beads. The supernatant was discarded and the beads were washed twice with 500 μl fresh 80% ethanol. The beads were left to dry. Bound DNA fragments were eluted by adding 35 μl TE buffer (10 mM Tris-C1, pH 8.0, 1 mM EDTA) and incubating for 15 min at room temperature, before placing on magnet. 28 μl of supernatant was carefully removed to prevent bead carry-over.

### Sequencing of the Amplified Products

In order to establish the identity of ligated tail tags, the purified amplified products were sequenced. Sequencing was performed using Ion 314™ Chip v2 for 400 bp read length, in the Ion PGM™ platform, per manufacturer's protocols (Ion Torrent™, Life Technologies, Carlsbad, Calif.). The generated fastq file was analyzed using Excel software (Microsoft Corporation, Redmond, Wash.). The analysis was performed using Excel functions known to those skilled in the art. For example, tail tag sequences were located within a sequence by using the “find” function (e.g., FIND (“CTTCTCTCTCTCTTCTCTCTT”,B1) [SEQ. ID. NO. 32]; B1 is the cell in the spreadsheet holding the sequence), Nt.BstNBI restriction sites were located using FIND (“GAGTC”,B1), and the identities of the two bases immediately following the Nt.BstNBI-generated nick were retrieved using the “mid” function, as in MID(B1,D1+9,2), wherein B1 is the cell holding the sequence, and D1 is the cell holding the location of the start of the restriction site.

There were 44.8 M total bases sequenced, corresponding to 549,805 total reads. The mean length of the reads was 81 bp. The total number of reads comprising lambda phage DNA attached to two tail tags was 15,532 reads, of which 573 had at least one tail tag of the wrong type attached. The percentage of correct tail tag attachments was 96.31%.

### Example 8

### Attachment of More Tail Tags

Additional tail tags can be attached to the nucleic acid molecules of Example 7. The process is described in FIGS. 22D and 22E. The nucleic acid molecules that were attached to two tail tags in Example 7 have the general structure shown in FIG. 22D, top, comprising ribonucleotide segment 2215 (one or more ribonucleotides comprising the predetermined base type represented by tail tag 2222), extension 2223, and last-attached tail tag 2222.

During step (o), segment 2215 is cleaved to leave a single ribonucleotide 2224, using methods described in Example 7. T4 polynucleotide kinase treatment follows cleavage, to dephosphorylate any phosphate that may be present at the 3′ end of 2224, and phosphorylate the 5′ end of the remaining part of 2222, thereby allowing ligation to additional tail tags.

During step (p), 2224 is extended to produce segment 2225, and treated with Taq polymerase to add A-overhang 2226. Methods are described in detail in Example 7.

During step (q), a new tail tag 2227 comprising a T-overhang is attached by performing TA ligation as described in Example 7. 2227 represents a different base type from the base type comprised in 2224.

During step (r), a nick 2228 is generated by using RNase HII as described in Example 7.

During step (s), single-base gap 2229 is formed by using methods described in Example 7.

During step (t), the single-base gap 2229 is filled with deoxyribonucleotide 2231, as described in Example 7. The base type comprised in 2231 is represented by the previously attached tail tag 2222. Nick 2231 remains after nucleotide incorporation.

During step (u) in FIG. 22E, the nucleic acid molecule is exposed to conditions to cause incorporation of ribonucleotides comprising the predetermined base type represented by tail tag 2227, using methods as described in Example 7. The produced segment 2232 comprises one or more ribonucleotides. Strand segment 2233 is displaced during production of 2232.

During step (v), a terminal blocking nucleotide tail is formed in nucleic acid molecules that do not incorporate any ribonucleotides during step (u). Nick 2231 is sealed by ligation, as described in Example 7.

During step (w), 2232 is cleaved and treated as described in Example 7, to leave a single extendable ribonucleotide 2234.

During step (x), a ligatable removable nucleotide tail is formed, comprising strand segment 2235 and A-overhang 2236. Polymerases with 5′-to-3′ exonuclease activity are used to form 2235, resulting in the template strand (i.e., the strand complementary to 2235) ending at a position within the hairpin loop of 2227.

During subsequent steps, another tail tag can be attached, and the process from step (q) to step (x) can be repeated one or more times to attach more tail tags, based on the sequence of the nucleic acid molecule.

### Example 9

### Ribonucleotide Incorporation by Therminator DNA Polymerase

In order to test the ability of Therminator DNA polymerase to perform ribonucleotide incorporation, an experiment was performed involving primer extension. First, oligo(dT)₂₅ (oligonucleotide homopolymers comprising 25 deoxythymidine nucleotides) covalently bound with their 5′ ends to magnetic beads (Oligo d(T)₂₅ Magnetic Beads; 500 μg/100 μl; New England BioLabs, Inc., Ipswich, Mass.) were annealed to single-stranded oligonucleotides carrying poly-A 3′-end tails:

[SEQ. ID. NO. 24]
polyA-oligo; CGT TGC TGT TCT CTG TTC CCT CGT TGT
CGT TTG TCG TTC GTT CGT GAT CGA CTC TGT CGC CGC
GTG TGT TGC TGC TCC CGC GTGTGT TGC TGC TCC AAA
AAA AAA AAA AAA AAA AA

The annealing was done as follows: (i) The beads were first washed 3 times with Washing Buffer (10 mM Tris-HCl, pH 7.5, 0.15 M LiCl, 1 mM EDTA) (600 0 Washing Buffer per wash for every 500 μg of beads). Unless otherwise specified, washing of magnetic beads mentioned herein comprises adding appropriate buffer, placing on magnet (Ambion® 6 tube magnetic stand, Life Technologies, Carlsbad, Calif.) to collect the beads, and discarding the supernatant. (ii) The washed beads were re-suspended in Binding Buffer (20 mM Tris-HCl, pH 7.5, 1.0 M LiCl, 2 mM EDTA) and an equal volume of sterile deionized water comprising polyA-oligo molecules (200 μl Binding Buffer, 200 μl sterile deionized water and 2 μs polyA-oligos for every 500 μg beads), incubated at 95° C. for 5 min and at 53° C. for 15 min, in order to anneal poly-A tails to oligo(dT)s on the beads.

The beads were then washed twice with cold 1× NEBuffer 2 (50 mM NaCl, 10 mM Tris-HCl, 10 mM MgCl₂, 1 mM DTT, pH 7.9) (New England BioLabs, Inc., Ipswich, Mass.) (800 μl buffer per wash for every 500 μg of beads).

In order to extend the annealed oligo(dT)s and produce strands complementary to polyA-oligo molecules, the beads were re-suspended in polymerization solution comprising Klenow Fragment (3′→5′ exo minus) (200 μl solution for every 250 μg beads, comprising 20 μl 10× NEBuffer 2, 1.6 μl dNTP (100 mM), 3 μl Klenow Fragment (3′→5′ exo minus) (5 units/μl) and sterile deionized water). The re-suspended beads were incubated at 37° C. for 2 min, placed on magnet immediately after, and the supernatant was discarded. The beads were washed once with cold 1× ThermoPol® buffer (20 mM Tris-HCl, 10 mM (NH₄)₂SO₄, 10 mM KCl, 2 mM MgSO₄, 0.1% Triton® X-100, pH 8.8) (New England BioLabs, Inc., Ipswich, Mass.) (800 μl buffer for every 500 μg of beads).

The washed beads were re-suspended in polymerization solution comprising Taq DNA polymerase (200 μl solution for every 250 μg of beads, comprising 20 μl 10× ThermoPol® buffer, 1.6 μl dNTP (100 mM), 1 μl Taq DNA polymerase (5 units/μl) and sterile deionized water).

The beads were incubated at 68° C. for 2 min, placed on magnet immediately after, and the supernatant was discarded. The beads were washed twice with 1× ThermoPol® buffer (1 ml per wash for every 500 μg of beads).

In order to remove the polyA-oligos bound to the newly formed extensions of the oligo(dT)s, the beads were re-suspended in denaturing solution (10 mM Tris-HCl, pH 7.5, 20 mM EDTA; 40 μl buffer per 500 μg beads) and incubated at 95° C. for 15 min. The beads were placed on magnet, the supernatant was separated from the beads, and the beads were washed with 0.1N NaOH for 15 min at 65° C. (800 μl for every 500 μg beads), and then 4 times with TWB buffer (10 mM Tris-HCl, pH 7.5; 1 ml buffer per wash for every 500 μg of beads) at room temperature.

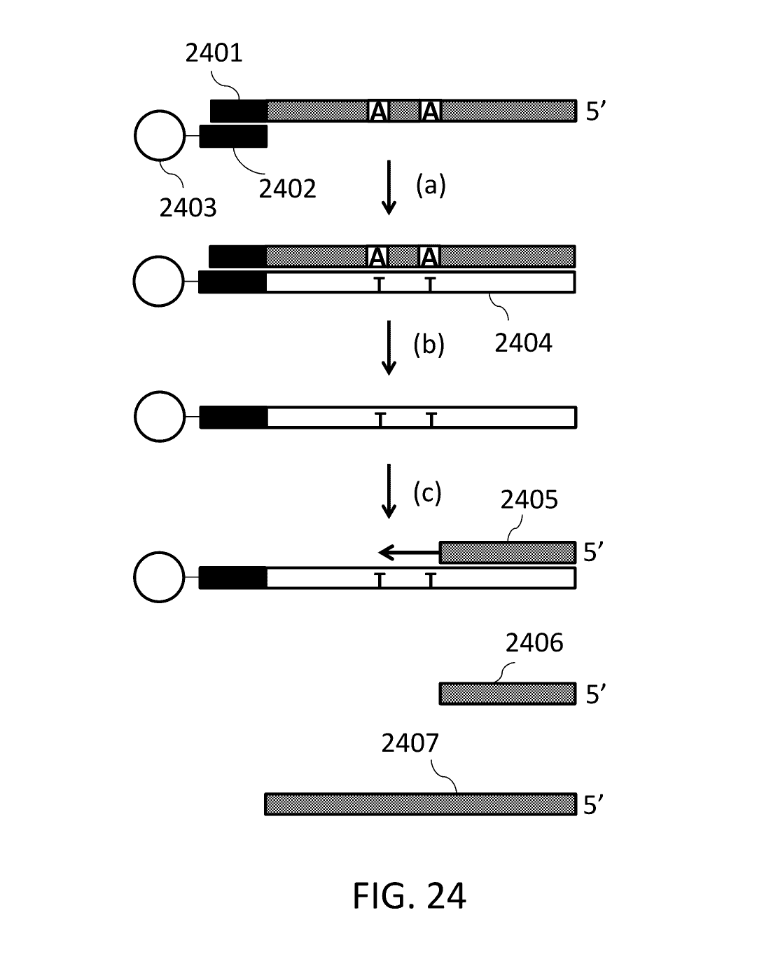

The beads carrying oligo(dT) extensions complementary to polyA-oligo were used as templates for primer extension, to test the ability of Therminator to perform ribonucleotide incorporation. FIG. 24 summarizes the experiments: first, polyA-oligo anneals with its polyA tail 2401 to oligo(dT) 2402 anchored to the magnetic bead 2403. As shown in FIG. 24, polyA-oligo was designed to have a polyA tail (2401) at the side of the 3′ end, and 3 segments comprising only nucleotides with bases T, C and G, separated by two nucleotides with the base A (segments sizes are not shown in proportion in FIG. 24; bases A are marked as “A”s in FIG. 24). Step (a) represents the oligo(dT) extension step described above, which produces complementary strand 2404. Step (b) represents the denaturation step that removes polyA-oligo. Step (c) represents annealing of primer 2405 to perform extension (with an arrow showing the direction of extension) described as follows.

Primer Molecules with Sequence

[SEQ. ID. NO. 25]
CGT TGC TGT TCT CTG TTC CCT CGT TGT CGT TTG TCG
TTC GTT CGT G

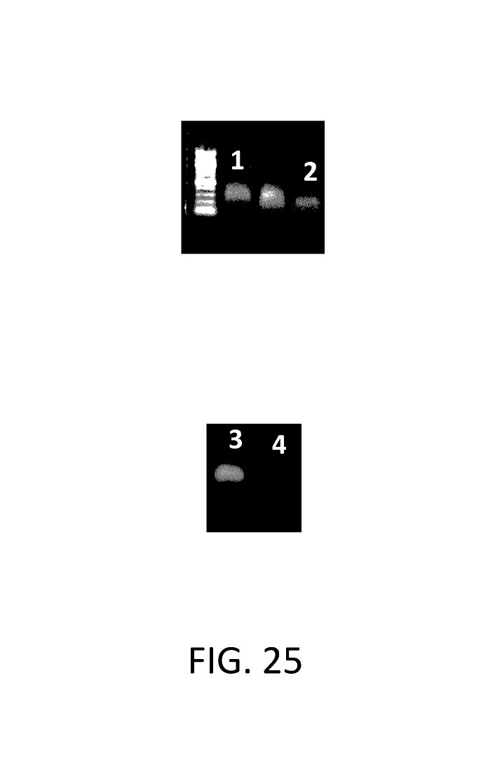

were annealed to the extensions bound to beads, and extended using either dNTP (sample 1 in FIG. 25) or NTP (sample 2 in FIG. 25). This was accomplished by re-suspending 250 μg of washed beads in a 200 μl solution comprising 20 μl 10× ThermoPol® buffer, 1 μg primer, 1.6 μl dNTP (sample 1 in FIG. 25) or NTP (sample 2 in FIG. 25)(from 100 mM dNTP or NTP stock), 5 μl Therminator DNA polymerase (2 units/μl) and dH₂O. The samples were incubated at 95° C. for 2 min and at 72° C. for 2 min, placed on magnet immediately after, and the supernatants were discarded. The beads were washed twice with 1× ThermoPol® buffer (600 μl per wash). The beads were re-suspended in 20 μl denaturing solution (per sample) and incubated at 95° C. for 15 min. The beads were placed on a magnet, the supernatants were separated, mixed with an equal volume of 50% glycerol, loaded on a 2% agarose gel and visualized after undergoing electrophoresis separation. The results are shown in FIG. 25.

As shown in FIG. 25, sample 2 had lower molecular weight than sample 1. This was either the result of incorporation of a limited number of ribonucleotides (consistent with previous published results), or the result of absence of incorporation. In order to further test ribonucleotide incorporation by Therminator, the following experiment was conducted: One sample (sample 3 in FIG. 25) comprised 250 μg beads carrying oligo(dT) extensions complementary to polyA-oligos and was subjected to primer annealing and extension using NTP (200 μl polymerization solution comprising 20 μl 10× ThermoPol® buffer, 1 μg primer, 1.6 μl NTP (100 mM), 5 μl Therminator DNA polymerase and dH₂O). The sample was incubated at 95° C. for 2 min and at 72° C. for 2 min, placed on magnet immediately after, and the supernatant was discarded. The beads were washed twice with 1× ThermoPol® buffer (600 μl per wash). The beads were then re-suspended in another 200 μl solution comprising 20 μl 10× ThermoPol® buffer, 0.4 μl dTTP, 0.4 μl dCTP, 0.4 μl dGTP, 5 μl Therminator DNA polymerase and dH₂O. As shown in FIG. 24, extension without dATP would be successful only in the event that the ribonucleotides incorporated during the previous step were enough to form a segment complementary to the template, long enough to cover both T sites. Failure of Therminator to perform NTP incorporation would lead to failure to extend without dATP, and would result to a short product 2406. Ability of Therminator to perform NTP incorporation past the T sites leads to a long extension product 2407. Another sample was run as control (sample 4 in FIG. 25), comprising 250 μg beads carrying oligo(dT) extensions complementary to polyA-oligos subjected to a single step of primer annealing and extension using dNTP without dATP. Samples 3 and 4 were treated with denaturing buffer, subjected to agarose gel electrophoresis and visualized as described above. As expected, sample 4 shown in FIG. 25 was a low molecular weight product, whereas sample 3 shown in FIG. 25 was a higher molecular weight product, consistent with the notion that at least 5 ribonucleotides (following the 3′ end of the primer and including the position complementary to the second T position on the template) were incorporated successfully.

### Example 10

### Alkaline Hydrolysis of RNA Segments

Alkaline hydrolysis is a well-known method for degrading ribonucleic acid molecules (Lipkin et al., 1954), and is widely used in a variety of applications where removal of RNA is desirable. The mechanism of alkaline hydrolysis involves the cleavage of the backbone bond at the 3′ end of a ribonucleotide, by forming a 2′, 3′-cyclic phosphate, which may open to generate either a 3′-phosphate or a 2′-phosphate remaining at the ribonucleotide. This mechanism suggests that the bond between the 3′ end of a deoxyribonucleotide and the 5′ end of a ribonucleotide is not cleaved by alkaline hydrolysis. Also, alkaline hydrolysis leads to cleaved 3′ ends that are not extendable by polymerization, because they contain phosphates (at the 2′- or 3′-end, or a cyclic phosphate at both ends). Such cleaved 3′ ends may be subjected to dephosphorylation and rendered extendable. Examples include using phosphatases such as rSAP (recombinant shrimp alkaline phosphatase) or 5′ end kinases with 3′ end phosphatase activity such as T4 polynucleotide kinase (PNK). A preferred method suggested by a previous study proposes using T4 polynucleotide kinase for 3′-end dephosphorylation of hydrolyzed RNA molecules (Huppertz et al., 2014).

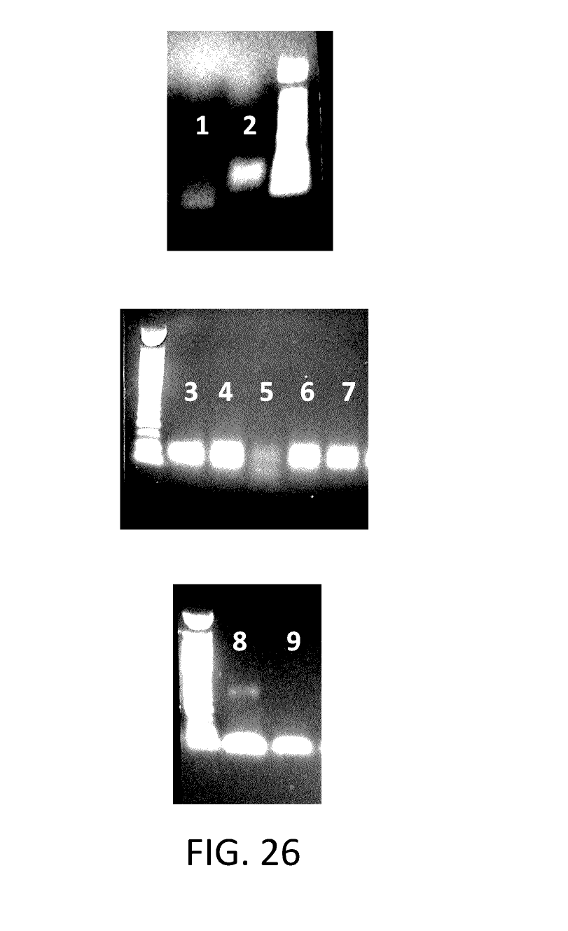

NaOH is a common reagent used to perform alkaline hydrolysis and can be used in a variety of conditions. For example, it has been shown that a 10-min incubation with 0.25N NaOH at 90° C. readily cleaves the backbone bond between the 3′ end of a ribonucleotide and the 5′ end of a deoxyribonucleotide (Wang et al., 2002). An experiment was conducted, shown in FIG. 26, which involved the incubation of oligonucleotides (comprising or not comprising ribonucleotides) in a NaOH solution. 100 pmoles of DNA-RNA hybrid oligonucleotides named “oligo-R” with sequence:

[SEQ. ID. NO. 26]
CGT TTG TCG TTC GTT CGT GAT CGrA rCrUrC rUrGrU
CACTGA CTC AGCTAC AGT CAT GGT

(rA, rU, rC, rG denote ribonucleotides), or 100 pmoles of DNA oligonucleotides named “oligo-D” with sequence:

[SEQ. ID. NO. 27]
CGT TTG TCG TTC GTT CGT GAT CGA CTC TGT CAC TGA
CTC AGC TAC AGT CAT GGT

were diluted in 20 μl 0.1N NaOH and incubated at 65° C. for 15 min. 20 μl of 50% glycerol was added and the samples were subjected to agarose gel electrophoresis and visualized. Oligo-D has the same length and sequence with oligo-R but with deoxyribonucleotides instead of ribonucleotides. Sample 1 in FIG. 26 is the NaOH-treated oligo-R, and sample 2 is the NaOH-treated oligo-D. As expected, oligo-D was not affected, whereas oligo-R which comprised ribonucleotides appeared as a lower molecular weight band, suggesting that oligo-R molecules were cleaved by NaOH treatment.

An experiment was conducted to test whether: (i) ribonucleotides bound with their 5′ ends to deoxyribonucleotides are not affected by alkaline hydrolysis, (ii) alkaline hydrolysis generates 3′ ends that are not extendable by polymerization, and (iii) T4 polynucleotide kinase treatment can render alkaline hydrolysis-generated 3′ ends extendable. Two oligonucleotide populations were tested for their ability to be extended by polymerization in polymerase chain reactions (PCR) after treatment with NaOH. One population was named “oligo-rG” with sequence

[SEQ. ID. NO. 28]
ACC ATG ACT GTA GCT GAGTCA GTG CGT TTG TCG TTC GTT
CGT GAT CrG

where rG is a ribonucleotide at the 3′ end of a DNA oligonucleotide. The other population was named “oligo-rA” with sequence

[SEQ. ID. NO. 29]
ACC ATG ACT GTA GCT GAGTCA GTG CGT TTG TCG TTC GTT
CGT GrAT CG

where rA is a ribonucleotide embedded within a DNA oligonucleotide.
32 μl oligo-rG or oligo-rA (100 μM) were mixed with 8 μl 1 N NaOH (final concentration: 0.2N) and incubated at 90° C. for 15 min. In order to precipitate the treated oligonucleotides, a solution comprising 200 μl isopropanol was added, and the mixture was incubated at room temperature for 35 min. After incubation, the mixtures were centrifuged at 6000 rpm for 25 min, and visible pellets were formed. The supernatants were carefully discarded and the pellets were washed with 300 μl freshly prepared cold 70% ethanol. After a brief centrifugation (1 min at 6000 rpm), residual ethanol was removed and the pellets were left to dry. The pellets were re-suspended in 25 μl sterile deionized water. Some of the treated oligo-rA was treated with T4 polynucleotide kinase, in a reaction comprising 20 μl sterile deionized water, 2.5 μl 10×T4 polynucleotide kinase reaction buffer (1×: 70 mM Tris-HCl, 10 mM MgCl₂, 5 mM DTT, pH 7.6), 1 μl treated oligo-rA, 0.5 μl ATP (100 mM), and 1 μl T4 polynucleotide kinase (PNK). The solution was incubated at 37° C. for 30 min.

To test the ability of untreated oligo-rA and -rG, NaOH-treated oligo-rA and -rG, and PNK-treated oligo-rA to be extended by polymerization when hybridized to a template, PCRs were conducted. Specifically, each PCR had a total volume of 50 μl and comprised 5 μl 10× ThermoPol® buffer (1×: 20 mM Tris-HCl, 10 mM (NH₄)₂SO₄, 10 mM KCl, 2 mM MgSO₄, 0.1% Triton® X-100, pH 8.8)(New England BioLabs, Inc., Ipswich, Mass.), 0.4 μl dNTP (100 mM), 0.25 μl Taq DNA polymerase (5 units/μl), oligo-rA or oligo-rG (untreated, or NaOH-treated, or PNK-treated) acting as forward primer to a final concentration of 0.2 μM, reverse primer to a final concentration of 0.2 μM, and template to a final concentration of 2 nM. The thermocycling conditions comprised an initial denaturation step at 94° C. for 30 sec, 25 cycles with 3 steps each (94° C. for 30 sec; 58° C. for 30 sec; 72° C. for 30 sec), and a final extension step at 72° C. for 5 min. Thermocycling was conducted using an Applied Biosystems® 2720 Thermal Cycler (Life Technologies, Carlsbad, Calif.). The sequence of the template was:

[SEQ. ID. NO. 30]
GAC CTA CGATGA GAC CTA GACTCA CCT CGATCA CGA ACG
AAC GAC AAA CGA CAA CGA;

the sequence of the reverse primer was:

[SEQ. ID. NO. 31]
GAC CTA CGATGA GAC CTA GACTCA CC.

After completion of thermocycling, 20 μl PCR solutions were added 4 μl loading buffer (Gel Loading Dye, Purple (6×); New England BioLabs, Inc., Ipswich, Mass.) and were subjected to agarose gel electrophoresis and visualization. FIG. 26 shows: sample 3 (PCR with NaOH-treated oligo-rG); sample 4 (PCR with untreated oligo-rG); sample 5 (PCR with NaOH-treated oligo-rA); sample 6 (PCR with untreated oligo-rA); sample 7 (PCR with PNK-treated oligo-rA). All samples show amplified products with the exception of sample 5, suggesting that NaOH treatment cleaved oligo-rA at the 3′ end side of the ribonucleotide rA and generated a non-extendable 3′ end at the cleavage site. Treated oligo-rG generated PCR product (sample 1), consistent with the notion that NaOH does not cleave ribonucleotides at their 5′ end side when bound to deoxyribonucleotides. PNK-treated oligo-rA generated PCR product (sample 7), suggesting that treatment with T4 polynucleotide kinase removes any phosphates present at NaOH-cleaved 3′ ends, and restores their ability to be extended by polymerization.

Alkaline hydrolysis conditions may lead to denaturation of DNA strands or disruption of other bonds. As shown in examples described herein, it may be desirable to use hairpins or covalently linked strands or other arrangements that can mediate re-annealing of strands that are denatured by alkaline treatments.

A system of nucleic acid anchoring, that was used in examples and experiments described herein, is the binding of biotin-labeled oligonucleotides or other molecules or constructs to streptavidin-coated beads. An experiment was conducted to test whether NaOH treatment disrupts the biotin-streptavidin bond. In brief, 250 μg streptavidin-coated beads (Dynabeads® MyOne™ Streptavidin C1; 10 mg/ml; New England BioLabs, Inc., Ipswich, Mass.) with bound biotin-labeled oligonucleotides were treated with 100 μl 0.2N NaOH solution at 90° C. for 15 min. The beads were placed on magnet, the supernatant was discarded, and the beads were washed 3 times with 500 μl of 20 mM Tris-HCl, pH 7.5. NaOH-treated beads and untreated beads of equal amount were added 20 μl of 0.5M EDTA and incubated at 100° C. for 10 min to elute the bound oligonucleotides. The bead samples were placed on magnet, the supernatants were collected, 20 μl of 50% glycerol were added to the supernatants, and the samples were subjected to agarose electrophoresis and visualized. As shown in FIG. 26, the NaOH-treated beads (sample 9) did not sustain significant loss of bound oligonucleotides compared to the untreated beads (sample 8).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are both chemically related can be substituted for the agents described herein while the same or similar results would be achieved. All such similar substitutes and modifications apparent to those skilled in the art are deemed to be within the spirit, scope and concept of the disclosure as defined by the appended claims.

### REFERENCES

Adessi, C., Kawashima, E., Mayer, P., Mermod, J.-J., Turcatti, G., 2014. Methods of nucleic acid amplification and sequencing. U.S. Pat. No. 8,652,810 B2.
Agrawal, S., 1994. Protocols for Oligonucleotide Conjugates: Synthesis and Analytical Techniques. Humana Press.
Akeson, M. A., Deamer, D. W., Branton, D., 2004. Monitoring ionic current through nanopore by measuring applied electrical field between chambers; for characterization of naturally occurring ion channels and polymers. U.S. Pat. No. 6,746,594 B2.
Akeson, M. A., Deamer, D. W., Branton, D., 2001. Miniature support for thin films containing single channels or nanopores and methods for using same. U.S. Pat. No. 6,267,872 B1.
Anderson, J. P., Angerer, B., Loeb, L. A., 2005. Incorporation of reporter-labeled nucleotides by DNA polymerases. BioTechniques 38, 257-264.
Ashkin, A., Dziedzic, J. M., 1987. Optical trapping and manipulation of viruses and bacteria. Science 235, 1517-1520.
Ashkin, A., Dziedzic, J. M., Bjorkholm, J. E., Chu, S., 1986. Observation of a single-beam gradient force optical trap for dielectric particles. Opt. Lett. 11, 288.
Ashkin, A., Dziedzic, J. M., Yamane, T., 1987. Optical trapping and manipulation of single cells using infrared laser beams. Nature 330, 769-771. doi:10.1038/330769a0
Atas, E., Singer, A., Meller, A., 2012. DNA sequencing and bar-coding using solid-state nanopores. Electrophoresis 33, 3437-3447. doi:10.1002/elps.201200266
Baldarelli, R., Branton, D., Church, G., Deamer, D. W., Akeson, M., Kasianowicz, J., 2000. Efficient sequencing of nucleic acids by using two separate but adjacent pools of medium with an interface having channel allowing sequential nucleotide passage, taking measurements as each nucleotide passes through the channel. U.S. Pat. No. 6,015,714 A.
Barnes, W. M., 1987. Sequencing DNA with dideoxyribonucleotides as chain terminators: hints and strategies for big projects. Methods Enzymol. 152, 538-556.
Beaucage, S. L., Iyer, R. P., 1993. The Functionalization of Oligonucleotides Via Phosphoramidite Derivatives. Tetrahedron 49, 1925-1963. doi:10.1016/50040-4020(01)86295-5
Benner, S. A., 1993. Oligonucleotide analogs containing sulfur linkages. U.S. Pat. No. 5,216,141 A.
Benner, S. A., CH, 1995. U.S. Pat. No. 5,432,272—Method for incorporating into a DNA or RNA oligonucleotide using nucleotides bearing heterocyclic bases. U.S. Pat. No. 5,432,272.
Bensimon, A., Bensimon, D., 2001. Apparatus for the parallel alignment of macromolecules, and use thereof. U.S. Pat. No. 6,225,055 B1.
Bensimon, A., Bensimon, D., Michalet, X., 2002. Determining copy numbers of genomic dna that has been aligned on a surface using molecular combing techniques, reacting the aligned dna with specific probes for controlled genomic sequences and detect hybridization and calculate copy numbers. U.S. Pat. No. 6,344,319 B1.
Bensimon, A., Simon, A., Chiffaudel, A., Croquette, V., Heslot, F., Bensimon, D., 1994. Alignment and sensitive detection of DNA by a moving interface. Science 265, 2096-2098.
Bensimon, D., Bensimon, A., Heslot, F., 2003. Immobilization. U.S. Pat. No. 6,548,255 B2.
Bensimon, D., Bensimon, A., Heslot, F., 2001a. Process for aligning macromolecules by passage of a meniscus and applications. U.S. Pat. No. 6,303,296 B1.
Bensimon, D., Bensimon, A., Heslot, F., 2001b. Adhering nucleotide sequences to a support; incubate nucleotide sequences with solid support, move slowly through curved surface of fluid, analyze the surface of support for fixed nucleotide sequences. U.S. Pat. No. 6,265,153 B1.
Bensimon, D., Bensimon, A., Heslot, F., 2000. Process for aligning macromolecules on a surface by passage through a meniscus. U.S. Pat. No. 6,054,327 A.
Bensimon, D., Bensimon, A., Heslot, F., 1998. Process for aligning, adhering and stretching nucleic acid strands on a support surface by passage through a meniscus. U.S. Pat. No. 5,840,862 A.
Bentley, D. R., Balasubramanian, S., Swerdlow, H. P., Smith, G. P., Milton, J., Brown, C. G., Hall, K. P., Evers, D. J., Barnes, C. L., Bignell, H. R., Boutell, J. M., Bryant, J., Carter, R. J., Cheetham, R. K., Cox, A. J., Ellis, D. J., Flatbush, M. R., Gormley, N. A., Humphray, S. J., Irving, L. J., Karbelashvili, M. S., Kirk, S. M., Li, H., Liu, X., Maisinger, K S., Murray, L. J., Obradovic, B., Ost, T., Parkinson, M. L., Pratt, M. R., Rasolonjatovo, I. M. J., Reed, M. T., Rigatti, R., Rodighiero, C., Ross, M. T., Sabot, A., Sankar, S. V., Scally, A., Schroth, G. P., Smith, M. E., Smith, V. P., Spiridou, A., Torrance, P. E., Tzonev, S. S., Vermaas, E. H., Walter, K., Wu, X., Zhang, L., Alam, M. D., Anastasi, C., Aniebo, I. C., Bailey, D. M. D., Bancarz, I. R., Banerjee, S., Barbour, S. G., Baybayan, P. A., Benoit, V. A., Benson, K. F., Bevis, C., Black, P. J., Boodhun, A., Brennan, J. S., Bridgham, J. A., Brown, R. C., Brown, A. A., Buermann, D. H., Bundu, A. A., Burrows, J. C., Carter, N. P., Castillo, N., Catenazzi, M. C. E., Chang, S., Cooley, R. N., Crake, N. R., Dada, O. O., Diakoumakos, K. D., Dominguez-Fernandez, B., Earnshaw, D. J., Egbuj or, U. C., Elmore, D. W., Etchin, S. S., Ewan, M. R., Fedurco, M., Fraser, L. J., Fajardo, K. V. F., Furey, W. S., George, D., Gietzen, K. J., Goddard, C. P., Golda, G. S., Granieri, P. A., Green, D. E., Gustafson, D. L., Hansen, N. F., Harnish, K., Haudenschild, C. D., Heyer, N. I., Hims, M. M., Ho, J. T., Horgan, A. M., Hoschler, K., Hurwitz, S., Ivanov, D. V., Johnson, M. Q., James, T., Jones, T. A. H., Kang, G.-D., Kerelska, T. H., Kersey, A. D., Khrebtukova, I., Kindwall, A. P., Kingsbury, Z., Kokko-Gonzales, P. I., Kumar, A., Laurent, M. A., Lawley, C. T., Lee, S. E., Lee, X., Liao, A. K., Loch, J. A., Lok, M., Luo, S., Mammen, R. M., Martin, J. W., McCauley, P. G., McNitt, P., Mehta, P., Moon, K. W., Mullens, J. W., Newington, T., Ning, Z., Ng, B. L., Novo, S. M., O'Neill, M. J., Osborne, M. A., Osnowski, A., Ostadan, O., Paraschos, L. L., Pickering, L., Pike, A. C., Pike, A. C., Pinkard, D. C., Pliskin, D. P., Podhasky, J., Quijano, V. J., Raczy, C., Rae, V. H., Rawlings, S. R., Rodriguez, A. C., Roe, P. M., Rogers, J., Rogert Bacigalupo, M. C., Romanov, N., Romieu, A., Roth, R. K., Rourke, N. J., Ruediger, S. T., Rusman, E., Sanches-Kuiper, R. M., Schenker, M. R., Seoane, J. M., Shaw, R. J., Shiver, M. K., Short, S. W., Sizto, N. L., Sluis, J. P., Smith, M. A., Sohna, J. E. S., Spence, E. J., Stevens, K., Sutton, N., Szajkowski, L., Tregidgo, C. L., Turcatti, G., vandeVondele, S., Verhovsky, Y., Virk, S. M., Wakelin, S., Walcott, G. C., Wang, J., Worsley, G. J., Yan, J., Yau, L., Zuerlein, M., Rogers, J., Mullikin, J. C., Hurles, M. E., McCooke, N. J., West, J. S., Oaks, F. L., Lundberg, P. L., Klenerman, D., Durbin, R., Smith, A. J., 2008. Accurate Whole Human Genome Sequencing using Reversible Terminator Chemistry. Nature 456, 53-59. doi:10.1038/nature07517
Ben Yehezkel, T., Linshiz, G., Buaron, H., Kaplan, S., Shabi, U., Shapiro, E., 2008. De novo DNA synthesis using single molecule PCR. Nucleic Acids Res. 36, e107. doi:10.1093/nar/gkn457
Berg, J. L. T. L. S. J., 2006. Biochemistry 6th Edition (Sixth Ed.) 6e By Jeremy Berg, John Tymoczko & Lubert Stryer 2006. Example Product Manufacturer.
Blanusa, M., Schenk, A., Sadeghi, H., Marienhagen, J., Schwaneberg, U., 2010. Phosphorothioate-based ligase-independent gene cloning (PLICing): An enzyme-free and sequence-independent cloning method. Anal. Biochem. 406, 141-146. doi:10.1016/j.ab.2010.07.011
Block, S. M., Goldstein, L. S., Schnapp, B. J., 1990. Bead movement by single kinesin molecules studied with optical tweezers. Nature 348, 348-352. doi:10.1038/348348a0
Branton, D., Golovchenko, J. A., Denison, T. J., 2003a. Rapid, reliable and inexpensive characterization of such as nucleic acids; increased temperature, bias voltage operating range and chemical environment accommodation; reduced noise levels; durable. U.S. Pat. No. 6,627,067 B1.
Branton, D., Laderman, S., Yakhini, Z., Sampson, J., Wang, H., 2003b. Method for characterization of nucleic acid molecules. US20030104428 A1.
Brian, F., Mark, M., 1995. Enhanced triple-helix and double-helix formation with oligomers containing modified purines. WO1994024144 A3.
Bridgham, J., Corcoran, K., Golda, G., Pallas, M. C., Brenner, S., 2013. System and apparatus for sequential processing of analytes. US20130184162 A1.
Brill, W. K. D., Tang, J. Y., Ma, Y. X., Caruthers, M. H., 1989. Synthesis of oligodeoxynucleoside phosphorodithioates via thioamidites. J. Am. Chem. Soc. 111, 2321-2322. doi:10.1021/ja00188a066
Brown, D. M., 1993. A brief history of oligonucleotide synthesis. Methods Mol. Biol. Clifton N. J. 20, 1-17. doi:10.1385/0-89603-281-7:1
Buermann, D., Moon, J. A., Crane, B., Wang, M., Hong, S. S., Harris, J., Hage, M., Nibbe, M. J., 2013. Integrated optoelectronic read head and fluidic cartridge useful for nucleic acid sequencing. US20130260372 A1.
Burgess, K., Jacutin, S. E., Lim, D., shiangkoon, A., 1997. An Approach to Photolabile, Fluorescent Protecting Groups. J. Org. Chem. 62, 5165-5168. doi:10.1021/jo9702608
Buzby, P. R., Meller, A., MCNALLY, B., Fan, A., OLEJNIK-KRZYNMANSKA, E., 2012. Sequence preserved dna conversion for optical nanopore sequencing. US20120316075 A1
Cariello, N. F., Swenberg, J. A., Skopek, T. R., 1991. Fidelity of Thermococcus litoralis DNA polymerase (Vent) in PCR determined by denaturing gradient gel electrophoresis. Nucleic Acids Res. 19, 4193-4198.
Carlsson, C., Jonsson, M., Nordén, B., Dulay, M. T., Zare, R. N., Noolandi, J., Nielsen, P. E., Tsui, L.-C., Zielenski, J., 1996. Screening for genetic mutations. Nature 380, 207-207. doi:10.1038/380207a0
Cerritelli, S. M., Crouch, R. J., 1998. Cloning, expression, and mapping of ribonucleases H of human and mouse related to bacterial RNase HI. Genomics 53, 300-307. doi:10.1006/geno.1998.5497
Chen, G. J., Qiu, N., Page, M. P. G., 2002. Universal restriction site-free cloning method using chimeric primers. BioTechniques 32, 516, 518-520.
Chidgeavadze, Z. G., Beabealashvilli, R. S., Atrazhev, A. M., Kukhanova, M. K., Azhayev, A. V., Krayevsky, A. A., 1984. 2′,3′-Dideoxy-3′ aminonucleoside 5′-triphosphates are the terminators of DNA synthesis catalyzed by DNA polymerases. Nucleic Acids Res. 12, 1671-1686.
Chidgeavadze, Z. G., Scamrov, A. V., Beabealashvilli, R. S., Kvasyuk, E. I., Zaitseva, G. V., Mikhailopulo, I. A., Kowollik, G., Langen, P., 1985. 3′-Fluoro-2′,3′-dideoxyribonucleoside 5′-triphosphates: terminators of DNA synthesis. FEBS Lett. 183, 275-278.
Chien, A., Edgar, D. B., Trela, J. M., 1976. Deoxyribonucleic acid polymerase from the extreme thermophile Thermus aquaticus. J. Bacteriol. 127, 1550-1557.
Chiu, R. W. K., Cantor, C. R., Lo, Y. M. D., 2009. Non-invasive prenatal diagnosis by single molecule counting technologies. Trends Genet. TIG 25, 324-331. doi:10.1016/j.tig.2009.05. 004
Choi, J.-Y., Eoff, R. L., Pence, M. G., Wang, J., Martin, M. V., Kim, E.-J., Folkmann, L. M., Guengerich, F. P., 2011. Roles of the Four DNA Polymerases of the Crenarchaeon Sulfolobus solfataricus and Accessory Proteins in DNA Replication. J. Biol. Chem. 286, 31180-31193. doi:10.1074/jbc.M111.258038
Chopra, N., Basile, D., Golovchenko, J., 2004. System with nano-scale conductor and nano-opening. US20040121525 A1.
Church, G., Deamer, D. W., Branton, D., Baldarelli, R., Kasianowicz, J., 1998. Measuring physical properties. U.S. Pat. No. 5,795,782 A.
Close, D. M., Anderson, R. S., 1973. Ultraviolet photobleaching of free radicals created in-irradiated amino acids. Radiat. Res. 53, 349-357.
Cook, P. D., Acevedo, O., Hebert, N., 1997. Phosphoramidate and phosphorothioamidate oligomeric compounds. U.S. Pat. No. 5,637,684 A.
Cook, P. D., Delecki, D. J., 1994. 7-deazapurine modified oligonucleotides. WO1994022892 A1.
Cook, P. D., Hoke, G., 1997. Oligonucleotides for modulating protein kinase C having phosphorothioate linkages of high chiral purity. U.S. Pat. No. 5,620,963 A.
Cook, P. D., Sanghvi, Y. S., 1992. Nuclease resistant, pyrimidine modified oligonucleotides that detect and modulate gene expression. WO1992002258 A1.
Deamer, D. W., 2003. Methods of determining the presence of double stranded nucleic acids in a sample. U.S. Pat. No. 6,617,113 B2.
De Mesmaeker, A., Waldner, A., S. Sanghvi, Y., Lebreton, J., 1994. Comparison of rigid and flexible backbones in antisense oligonucleotides. Bioorg. Med. Chem. Lett. 4, 395-398. doi:10.1016/0960-894X(94)80003-0
Dempcy, R. O., Browne, K. A., Bruice, T. C., 1995. Synthesis of a thymidyl pentamer of deoxyribonucleic guanidine and binding studies with DNA homopolynucleotides. Proc. Natl. Acad. Sci. 92, 6097-6101.
Denison, T. J., Sauer, A., Golovchenko, J., Meller, A., Brandin, E., Branton, D., 2004. Characterization of individual polymer molecules based on monomer-interface interactions. U.S. Pat. No. 6,673,615 B2.
Denison, T. J., Sauer, A., Golovchenko, J., Meller, A., Brandin, E., Branton, D., 2002. Characterization of individual polymer molecules based on monomer-interface interactions. U.S. Pat. No. 6,362,002 B1.
de Pablo, P. J., Moreno-Herrero, F., Colchero, J., Gómez Herrero, J., Herrero, P., Baró, A. M., Ordejón, P., Soler, J. M., Artacho, E., 2000. Absence of dc-conductivity in lambda-DNA. Phys. Rev. Lett. 85, 4992-4995.
Diaz, R. S., Sabino, E. C., 1998. Accuracy of replication in the polymerase chain reaction. Comparison between Thermotoga maritima DNA polymerase and Thermus aquaticus DNA polymerase. Braz. J. Med. Biol. Res. Rev. Bras. Pesqui. Médicas E Biológicas Soc. Bras. Biofisica Al 31, 1239-1242.
Dieffenbach, C. W., Dveksler, G. S., 2003. PCR Primer: A Laboratory Manual, 2 Lab edition. ed. Cold Spring Harbor Laboratory Press, Cold Spring Harbor, N.Y.
Drmanac, R., Callow, M., 2013. Nucleic acid sequencing and process. U.S. Pat. No. 8,518,640 B2.
Eckstein, F. (Ed.), 1992. Oligonucleotides and Analogues: A Practical Approach. Oxford University Press, Oxford; New York.
Eder, P. S., Walder, J. A., 1991. Ribonuclease H from K562 human erythroleukemia cells. Purification, characterization, and substrate specificity. J. Biol. Chem. 266, 6472-6479.
Eder, P. S., Walder, R. Y., Walder, J. A., 1993. Substrate specificity of human RNase H1 and its role in excision repair of ribose residues misincorporated in DNA. Biochimie 75, 123-126.
Edge, M. D., Green, A. R., Heathcliffe, G. R., Meacock, P. A., Schuch, W., Scanlon, D. B., Atkinson, T. C., Newton, C. R., Markham, A. F., 1981. Total synthesis of a human leukocyte interferon gene. Nature 292, 756-762.
Edwards, J., 2012. Polony sequencing methods. US20120270740 A1.
Efcavitch, J. W., Thompson, J. F., 2010. Single-molecule DNA analysis. Annu. Rev. Anal. Chem. Palo Alto Calif 3, 109-128. doi:10.1146/annurev.anchem.111808.073558
Efimov, V. A., Buryakova, A. A., Chakhmakhcheva, O. G., 1999. Synthesis of polyacrylamides N-substituted with PNA-like oligonucleotide mimics for molecular diagnostic applications. Nucleic Acids Res. 27, 4416-4426. doi:10.1093/nar/27.22.4416
Egholm, M., Buchardt, O., Christensen, L., Behrens, C., Freier, S. M., Driver, D. A., Berg, R. H., Kim, S. K., Norden, B., Nielsen, P. E., 1993. PNA hybridizes to complementary oligonucleotides obeying the Watson-Crick hydrogen-bonding rules. Nature 365, 566-568. doi:10.1038/365566a0
Egholm, M., Buchardt, O., Nielsen, P. E., Berg, R. H., 1992. Peptide nucleic acids (PNA). Oligonucleotide analogs with an achiral peptide backbone. J. Am. Chem. Soc. 114, 1895-1897. doi:10.1021/ja00031a062
Etienne, C. L., Opperman, K. K., Kaboord, B. J., Meier, S., Schultz, J.-S., 2012. Modified nucleotides methods and kits. US20120252691 A1.
Fasman, G. D., 1989. Practical Handbook of Biochemistry and Molecular Biology, 1 edition. ed. CRC Press, Boca Raton, Fla.
Ferree, S., Blanch, H. W., 2004. The hydrodynamics of DNA electrophoretic stretch and relaxation in a polymer solution. Biophys. J. 87, 468-475. doi:10.1529/biophysj.104.040014
Ferree, S., Blanch, H. W., 2003. Electrokinetic stretching of tethered DNA. Biophys. J. 85, 2539-2546. doi:10.1016/S0006-3495(03)74676-1
Fischbein, M. D., Drndić, M., 2008. Electron beam nanosculpting of suspended graphene sheets. Appl. Phys. Lett. 93, 113107. doi:10.1063/1.2980518
Froehler, B., Gutierrez, A. J., Jones, R. J., Matteucci, M., Pudlo, J., Wagner, R., 1993. Enhanced triple-helix and double-helix formation with oligomers containing modified pyrimidines. WO1993010820 A1.
Fuhrmann, M., Oertel, W., Hegemann, P., 1999. A synthetic gene coding for the green fluorescent protein (GFP) is a versatile reporter in Chlamydomonas reinhardtii. Plant J. Cell Mol. Biol. 19, 353-361.
Gait, M. J. (Ed.), 1984. Oligonucleotide Synthesis: A Practical Approach. Oxford University Press, Oxford Oxfordshire; Washington, D.C.
Gao, X., Jeffs, P. W., 1994. Unusual conformation of a 3′-thioformacetal linkage in a DNA duplex. J. Biomol. NMR 4, 17-34. doi:10.1007/BF00178333
Gao, X., Yo, P., Keith, A., Ragan, T. J., Harris, T. K., 2003. Thermodynamically balanced inside-out (TBIO) PCR-based gene synthesis: a novel method of primer design for high-fidelity assembly of longer gene sequences. Nucleic Acids Res. 31, e143.
Gardner, A. F., Jack, W. E., 2002. Acyclic and dideoxy terminator preferences denote divergent sugar recognition by archaeon and Taq DNA polymerases. Nucleic Acids Res. 30, 605-613. doi:10.1093/nar/30.2.605
Gardner, A. F., Jack, W. E., 1999. Determinants of nucleotide sugar recognition in an archaeon DNA polymerase. Nucleic Acids Res. 27, 2545-2553.
Gardner, A. F., Wang, J., Wu, W., Karouby, J., Li, H., Stupi, B. P., Jack, W. E., Hersh, M. N., Metzker, M. L., 2012. Rapid incorporation kinetics and improved fidelity of a novel class of 3′-OH unblocked reversible terminators. Nucleic Acids Res. 40, 7404-7415. doi:10.1093/nar/gks330
Geim, A. K., 2009. Graphene: status and prospects. Science 324, 1530-1534. doi:10.1126/science.1158877
Geiss, G. K., DAHL, T., Davidson, E., 2013. Methods and computer systems for identifying target-specific sequences for use in nanoreporters. U.S. Pat. No. 8,415,102 B2.
Gelfand, D. H., Gupta, A. P., 2012. Sequencing and genotyping using reversibly 2′-modified nucleotides. EP1974057 B1.
Gish, G., Eckstein, F., 1988. DNA and RNA sequence determination based on phosphorothioate chemistry. Science 240, 1520-1522.
Goksenin, A. Y., Zahurancik, W., LeCompte, K. G., Taggart, D. J., Suo, Z., Pursell, Z. F., 2012. Human DNA Polymerase? Is Able to Efficiently Extend from Multiple Consecutive Ribonucleotides. J. Biol. Chem. 287, 42675-42684. doi:10.1074/jbc.M112.422733
Golovchenko, J. A., Branton, D., Stein, D. M., McMullan, C. J., Li, J., 2002. Mechanical or electromechanical systems, real time feedback control operating on a time scale commensurate with the formation of nanoscale solid state features. U.S. Pat. No. 6,464,842 B1.
Golovchenko, J. A., Li, J., Stein, D., Gershow, M. H., 2010. determining the conformational distribution of a plurality of biological polymers and to detect binding or denaturation events; employ a nanopore which the polymer, e.g. a nucleic acid, traverses; as the polymer traverses the nanopore, measurements of transport properties of the nanopore yield data. U.S. Pat. No. 7,846,738 B2.
Goodman, M. F., Reha-Krantz, L. J., 1999. Using mutant family b polymerase to incorporate fluorophore labeled nucleotides into complementary dna; mutant retains 3′ to 5′ exonuclease activity which increases accuracy; for sequence determinations and production of genetic probes. U.S. Pat. No. 5,945,312 A.
Goodwin, S., Gurtowski, J., Ethe-Sayers, S., Deshpande, P., Schatz, M., McCombie, W. R., 2015. Oxford Nanopore Sequencing and de novo Assembly of a Eukaryotic Genome. bioRxiv 013490. doi:10.1101/013490
Gormley, N. A., Smith, G. P., Bentley, D., Rigatti, R., Luo, S., 2010. Used in solid-phase nucleic acid amplification; producing template polynucleotides that have common sequences at their 5′ ends and at their 3′ ends. U.S. Pat. No. 7,741,463 B2.
Green, E. D., 1997. Genome Analysis: A Laboratory Manual. Cold Spring Harbor Laboratory Press.
Green, M. R., Sambrook, J., 2012. Molecular Cloning: A Laboratory Manual (Fourth Edition): Three-volume set, 4th edition. ed. Cold Spring Harbor Laboratory Press, Avon, Mass.
Grier, D. G., 2003. A revolution in optical manipulation. Nature 424, 810-816. doi:10.1038/nature01935
Guo, J., Xu, N., Li, Z., Zhang, S., Wu, J., Kim, D. H., Marma, M. S., Meng, Q., Cao, H., Li, X., Shi, S., Yu, L., Kalachikov, S., Russo, J. J., Turro, N. J., Ju, J., 2008. Four-color DNA sequencing with 3′-O-modified nucleotide reversible terminators and chemically cleavable fluorescent dideoxynucleotides. Proc. Natl. Acad. Sci. 105, 9145-9150. doi:10.1073/pnas.0804023105
Hall, A. R., Scott, A., Rotem, D., Mehta, K. K., Bayley, H., Dekker, C., 2010. Hybrid pore formation by directed insertion of α-haemolysin into solid-state nanopores. Nat. Nanotechnol. 5, 874-877. doi:10.1038/nnano.2010.237
Hansen, C. J., Hogrefe, H. H., Sorge, J. A., 2001. Compositions and methods utilizing dna polymerases. WO2001032887 A1.
Hague, F., Geng, J., Montemagno, C., Guo, P., 2013a. Incorporation of a viral DNA-packaging motor channel in lipid bilayers for real-time, single-molecule sensing of chemicals and double-stranded DNA. Nat. Protoc. 8, 373-392. doi:10.1038/nprot.2013.001
Hague, F., Li, J., Wu, H.-C., Liang, X.-J., Guo, P., 2013b. Solid-State and Biological Nanopore for Real-Time Sensing of Single Chemical and Sequencing of DNA. Nano Today 8, 56-74. doi:10.1016/j.nantod.2012.12.008
Harris, T. D., 2010. Enhancing resolution of sequence analysis of short DNA stretches via defined length spacers; genetic mapping and genomics. U.S. Pat. No. 7,767,400 B2.
Hart, C., Lipson, D., Ozsolak, F., Raz, T., Steinmann, K., Thompson, J., Milos, P. M., 2010. Single-molecule sequencing: sequence methods to enable accurate quantitation. Methods Enzymol. 472, 407-430. doi:10.1016/S0076-6879(10)72002-4
Haruki, M., Hayashi, K., Kochi, T., Muroya, A., Koga, Y., Morikawa, M., Imanaka, T., Kanaya, S., 1998. Gene Cloning and Characterization of Recombinant RNase HII from a Hyperthermophilic Archaeon. J. Bacteriol. 180, 6207-6214.
Haruki, M., Tsunaka, Y., Morikawa, M., Kanaya, S., 2002. Cleavage of a DNA-RNA-DNA/DNA chimeric substrate containing a single ribonucleotide at the DNA-RNA junction with prokaryotic RNases HIT. FEBS Lett. 531, 204-208.
Healy, K., Schiedt, B., Morrison, A. P., 2007. Solid-state nanopore technologies for nanopore-based DNA analysis. Nanomed. 2, 875-897. doi:10.2217/17435889.2.6.875
Heiter, D. F., Lunnen, K. D., Wilson, G. G., 2005. Site-specific DNA-nicking mutants of the heterodimeric restriction endonuclease R.BbvCI. J. Mol. Biol. 348, 631-640. doi:10.1016/j.jmb.2005.02.034
Henegariu, O., Grober, L., Haskins, W., Bowers, P. N., State, M. W., Ohmido, N., Bray-Ward, P., Ward, D. C., 2001. Rapid DNA fiber technique for size measurements of linear and circular DNA probes. BioTechniques 31, 246-250.
Higgins, L. S., Besnier, C., Kong, H., 2001. The nicking endonuclease N.BstNBI is closely related to Type IIs restriction endonucleases MlyI and PleI. Nucleic Acids Res. 29, 2492-2501.
Hinnisdaels, S., Del-Favero, J., Vauterin, M., 1996. Direct cloning of PCR products amplified with Pwo DNA polymerase. BioTechniques 20, 186-188.
Hogrefe, H. H., Hogrefe, R. I., Walder, R. Y., Walder, J. A., 1990. Kinetic analysis of Escherichia coli RNase H using DNA-RNA-DNA/DNA substrates. J. Biol. Chem. 265, 5561-5566.
Horn, T., Chaturvedi, S., Balasubramaniam, T. N., Letsinger, R. L., 1996. Oligonucleotides with alternating anionic and cationic phosphoramidate linkages: Synthesis and hybridization of stereo-uniform isomers. Tetrahedron Lett. 37, 743-746. doi:10.1016/0040-4039(95)02309-7
Huang, L., Rumbaugh, J. A., Murante, R. S., Lin, R. J. R., Rust, L., Bambara, R. A., 1996. Role of Calf RTH-1 Nuclease in Removal of 5′-Ribonucleotides during Okazaki Fragment Processing†. Biochemistry (Mosc.) 35, 9266-9277. doi:10.1021/bi9603074
Huang, W. M., Lehman, I. R., 1972. On the exonuclease activity of phage T4 deoxyribonucleic acid polymerase. J. Biol. Chem. 247, 3139-3146.
Huppertz, I., Attig, J., D'Ambrogio, A., Easton, L. E., Sibley, C. R., Sugimoto, Y., Tajnik, M., Konig, J., Ule, J., 2014. iCLIP: Protein-RNA interactions at nucleotide resolution. Methods San Diego Calif. 65, 274-287. doi:10.1016/j.ymeth.2013.10.011
Hutter, D., Kim, M.-J., Karalkar, N., Leal, N. A., Chen, F., Guggenheim, E., Visalakshi, V., Olejnik, J., Gordon, S., Benner, S. A., 2010. Labeled nucleoside triphosphates with reversibly terminating aminoalkoxyl groups. Nucleosides Nucleotides Nucleic Acids 29, 879-895. doi:10.1080/15257770.2010.536191
Iqbal, S. M., Akin, D., Bashir, R., 2007. Solid-state nanopore channels with DNA selectivity. Nat. Nanotechnol. 2, 243-248. doi:10.1038/nnano.2007.78
Ireland, R. E., Varney, M. D., 1986. Approach to the total synthesis of chlorothricolide: synthesis of (.+−.)-19,20-dihydro-24-O-methylchlorothricolide methyl ester ethyl carbonate. J. Org. Chem. 51, 635-648. doi:10.1021/jo00355a013
Itaya, M., 1990. Isolation and characterization of a second RNase H (RNase HII) of Escherichia coli K-12 encoded by the rnhB gene. Proc. Natl. Acad. Sci. U.S.A 87, 8587-8591.
Itaya, M., Kondo, K., 1991. Molecular cloning of a ribonuclease H (RNase HI) gene from an extreme thermophile Thermus thermophilus HB8: a thermostable RNase H can functionally replace the Escherichia coli enzyme in vivo. Nucleic Acids Res. 19, 4443-4449.
Jacobson, K., Elson, E., Koppel, D., Webb, W., 1983. International workshop on the application of fluorescence photobleaching techniques to problems in cell biology. Fed. Proc. 42, 72-79.
Jenkins, G. N., Turner, N. J., 1995. The biosynthesis of carbocyclic nucleosides. Chem. Soc. Rev. 24, 169-176. doi:10.1039/C59952400169
Ju, J., Bi, L., Kim, D. H., Meng, Q., 2009a. Chemically Cleavable 3′-O-Allyl-DNTP-Allyl-Fluorophore Fluorescent Nucleotide Analogues and Related Methods. US20090263791 A1
Ju, J., Kim, D. H., Bi, L., Meng, Q., Bai, X., Li, Z., Li, X., Marma, M. S., Shi, S., Wu, J., Edwards, J. R., Romu, A., Turro, N. J., 2006. Four-color DNA sequencing by synthesis using cleavable fluorescent nucleotide reversible terminators. Proc. Natl. Acad. Sci. 103, 19635-19640. doi:10.1073/pnas.0609513103
Ju, J., Kumar, S., Li, Z., Tao, C., Chien, M., Russo, J. J., Kalachikov, S., Shepard, K., Rosenstein, J. K., 2013. Dna sequencing by synthesis using modified nucleotides and nanopore detection. US20130264207 A1.
Ju, J., Li, Z., Edwards, J. R., Itagaki, Y., 2009b. Sequencing; solid phase synthesis; primer hybridization. U.S. Pat. No. 7,635,578 B2.
Juncosa-Ginestà, M., Pons, J., Planas, A., Querol, E., 1994. Improved efficiency in site-directed mutagenesis by PCR using a Pyrococcus sp. GB-D polymerase. BioTechniques 16, 820-823.
Jung, P. M., Histand, G., Letsinger, R. L., 1994. Hybridization of Alternating Cationic/Anionic Oligonucleotides to RNA Segments. Nucleosides Nucleotides 13, 1597-1605. doi:10.1080/15257779408012174
Kamal, A., Laxman, E., Rao, N. V., 1999. A mild and rapid regeneration of alcohols from their allylic ethers by chlorotrimethylsilane/sodium iodide. Tetrahedron Lett. 40, 371-372. doi:10.1016/S0040-4039(98)02314-4
Kamitani, J., Sumaoka, J., Asanuma, H., Komiyama, M., 1998. Efficient RNA hydrolysis by lanthanide(III)-hydrogen peroxide combinations. Novel aggregates as the catalytic speciesl. J. Chem. Soc. Perkin Trans. 2 523-528. doi:10.1039/A708036K
Kawate, T., Jr, V. P. S., Verdine, G., Wolfe, J. L., 2009. A method for analyzing polynucleotides. EP1117838 B1.
Khorana, H. G., Agarwal, K. L., Bilchi, H., Caruthers, M. H., Gupta, N. K., Kleppe, K., Kumar, A., Otsuka, E., RajBhandary, U. L., Van de Sande, J. H., Sgaramella, V., Terao, T., Weber, H., Yamada, T., 1972. Studies on polynucleotides. 103. Total synthesis of the structural gene for an alanine transfer ribonucleic acid from yeast. J. Mol. Biol. 72, 209-217.
Kibbe, W. A., 2007. OligoCalc: an online oligonucleotide properties calculator. Nucleic Acids Res. 35, W43-46. doi:10.1093/nar/gkm234
Kim, M. J., Wanunu, M., Bell, D. C., Meller, A., 2006. Rapid Fabrication of Uniformly Sized Nanopores and Nanopore Arrays for Parallel DNA Analysis. Adv. Mater. 18, 3149-3153. doi:10.1002/adma.200601191
Kokoris, M. S., McRuer, R. N., 2013. U.S. Pat. No. 8,349,565—High throughput nucleic acid sequencing by expansion. U.S. Pat. No. 8,349,565.
Komiyama, M., Takeda, N., Shigekawa, H., 1999. Hydrolysis of DNA and RNA by lanthanide ions: mechanistic studies leading to new applications. Chem. Commun. 1443-1451. doi:10.1039/A901621J
Kornberg, A., Baker, T. A., 2005. DNA Replication. University Science Books.
Koshkin, A. A., Nielsen, P., Meldgaard, M., Rajwanshi, V. K., Singh, S. K., Wengel, J., 1998. LNA (Locked Nucleic Acid): An RNA Mimic Forming Exceedingly Stable LNA:LNA Duplexes. J. Am. Chem. Soc. 120, 13252-13253. doi:10.1021/ja9822862
Kowalczyk, S. W., Kapinos, L., Blosser, T. R., Magalhães, T., van Nies, P., Lim, R. Y. H., Dekker, C., 2011. Single-molecule transport across an individual biomimetic nuclear pore complex. Nat. Nanotechnol. 6, 433-438. doi:10.1038/nnano.2011.88
Kraus, J., Weber, R. G., Cremer, M., Seebacher, T., Fischer, C., Schurra, C., Jauch, A., Lichter, P., Bensimon, A., Cremer, T., 1997. High-resolution comparative hybridization to combed DNA fibers. Hum. Genet. 99, 374-380.
Kronick, M. N., Little, W. A., 1976. Fluorescent immunoassay employing total reflection for activation. U.S. Pat. No. 3,939,350 A.
Kumar, J. K., Chiu, E. T., Tabor, S., Richardson, C. C., 2004. A Unique Region in Bacteriophage T7 DNA Polymerase Important for Exonucleolytic Hydrolysis of DNA. J. Biol. Chem. 279, 42018-42025. doi:10.1074/jbc.M406103200
Kumar, S., Tao, C., Chien, M., Hellner, B., Balijepalli, A., Robertson, J. W. F., Li, Z., Russo, J. J., Reiner, J. E., Kasianowicz, J. J., Ju, J., 2012. PEG-labeled nucleotides and nanopore detection for single molecule DNA sequencing by synthesis. Sci. Rep. 2, 684. doi:10.1038/srep00684
Lecomte, P. J., Doubleday, O. P., 1983. Selective inactivation of the 3′ to 5′ exonuclease activity of Escherichia coli DNA polymerase I by heat. Nucleic Acids Res. 11, 7505-7515.
Letsinger, R. L., Bach, S. A., Eadie, J. S., 1986. Effects of pendant groups at phosphorus on binding properties of d-ApA analogues. Nucleic Acids Res. 14, 3487-3499. doi:10.1093/nar/14.8.3487
Letsinger, R. L., Mungall, W. S., 1970. Nucleotide chemistry. XVI. Phosporamidate analogs of oligonucleotides. J. Org. Chem. 35, 3800-3803. doi:10.1021/jo00836a048
Letsinger, R. L., Singman, C. N., Histand, G., Salunkhe, M., 1988. Cationic oligonucleotides. J. Am. Chem. Soc. 110, 4470-4471. doi:10.1021/ja00221a089
Levin, H. L., 1997. It's prime time for reverse transcriptase. Cell 88, 5-8.
Lexow, P., 2008. Method for sequencing a polynucleotide. WO2008032058 A2.
Li, J., Stein, D. M., Schurmann, G. M., King, G. M., Golovchenko, J., Branton, D., Aziz, M., 2007. Solid state molecular probe device. U.S. Pat. No. 7,258,838 B2.
Lipkin, D., Talbert, P. T., Cohn, M., 1954. The Mechanism of the Alkaline Hydrolysis of Ribonucleic Acids. J. Am. Chem. Soc. 76, 2871-2872. doi:10.1021/ja01640a004
Litman, D. J., Harel, Z., Ullman, E. F., 1981. Immunoassay. U.S. Pat. No. 4,275,149 A.
Liu, Q., Sommer, S. S., 2004. Pyrophosphorolysis by Type II DNA polymerases: implications for pyrophosphorolysis-activated polymerization. Anal. Biochem. 324, 22-28.
Loeb, L. A., Hood, L., Suzuki, M., 2002. Thermostable polymerases having altered fidelity and method of identifying and using same. U.S. Pat. No. 6,395,524 B2.
Lundberg, K. S., Shoemaker, D. D., Adams, M. W., Short, J. M., Sorge, J. A., Mathur, E. J., 1991. High-fidelity amplification using a thermostable DNA polymerase isolated from Pyrococcus furiosus. Gene 108, 1-6.
Mager, M. D., Melosh, N. A., 2008. Nanopore-Spanning Lipid Bilayers for Controlled Chemical Release. Adv. Mater. 20, 4423-4427. doi:10.1002/adma.200800969
Maggio, E. T., 1981. Chemiluminescent source, quencher molecule. U.S. Pat. No. 4,277,437 A.
Mag, M., Engels, J. W., 1989. Synthesis and selective cleavage of oligodeoxyribonucleotides containing non-chiral internucleotide phosphoramidate linkages. Nucleic Acids Res. 17, 5973-5988.
Mag, M., Silke, L., Engels, J. W., 1991. Synthesis and selective cleavage of an oligodeoxynucleotide containing a bridged intemucleotide 5′-phosphorothioate linkage. Nucleic Acids Res. 19, 1437-1441. doi:10.1093/nar/19.7.1437
Mandecki, W., Bolling, T. J., 1988. FokI method of gene synthesis. Gene 68, 101-107.
Manrao, E. A., Derrington, I. M., Laszlo, A. H., Langford, K. W., Hopper, M. K., Gillgren, N., Pavlenok, M., Niederweis, M., Gundlach, J. H., 2012. Reading DNA at single-nucleotide resolution with a mutant MspA nanopore and phi29 DNA polymerase. Nat. Biotechnol. 30, 349-353. doi:10.1038/nbt.2171
Ma, P. N.-T., 2013. Methods, compositions, and kits for amplifying and sequencing polynucleotides. U.S. Pat. No. 8,486,627 B2.
Mathies, R. A., Peck, K., Stryer, L., 1990. High sensitivity fluorescent single particle and single molecule detection apparatus and method. U.S. Pat. No. 4,979,824 A.
Matsumura, K., Komiyama, M., 1997. Enormously fast RNA hydrolysis by lanthanide(III) ions under physiological conditions: eminent candidates for novel tools of biotechnology. J. Biochem. (Tokyo) 122, 387-394.
Matsuura, S., Komatsu, J., Hirano, K., Yasuda, H., Takashima, K., Katsura, S., Mizuno, A., 2001. Real-time observation of a single DNA digestion by lambda exonuclease under a fluorescence microscope field. Nucleic Acids Res. 29, E79.
Matsuura, S., Kurita, H., Nakano, M., Komatsu, J., Takashima, K., Katsura, S., Mizuno, A., 2002. One-end immobilization of individual DNA molecules on a functional hydrophobic glass surface. J. Biomol. Struct. Dyn. 20, 429-436. doi:10.1080/07391102.2002.10506861
Mayer, P., Farinelli, L., Kawashima, E. H., 2013. Method of nucleic acid amplification. U.S. Pat. No. 8,476,044 B2.
McKernan, K. J., Peckham, H. E., Costa, G. L., McLaughlin, S. F., Fu, Y., Tsung, E. F., Clouser, C. R., Duncan, C., Ichikawa, J. K., Lee, C. C., Zhang, Z., Ranade, S. S., Dimalanta, E. T., Hyland, F. C., Sokolsky, T. D., Zhang, L., Sheridan, A., Fu, H., Hendrickson, C. L., Li, B., Kotler, L., Stuart, J. R., Malek, J. A., Manning, J. M., Antipova, A. A., Perez, D. S., Moore, M. P., Hayashibara, K. C., Lyons, M. R., Beaudoin, R. E., Coleman, B. E., Laptewicz, M. W., Sannicandro, A. E., Rhodes, M. D., Gottimukkala, R. K., Yang, S., Bafna, V., Bashir, A., MacBride, A., Alkan, C., Kidd, J. M., Eichler, E. E., Reese, M. G., De La Vega, F. M., Blanchard, A. P., 2009. Sequence and structural variation in a human genome uncovered by short-read, massively parallel ligation sequencing using two-base encoding. Genome Res. 19, 1527-1541. doi:10.1101/gr.091868.109
McNally, B., Wanunu, M., Meller, A., 2008. Electromechanical unzipping of individual DNA molecules using synthetic sub-2 nm pores. Nano Lett. 8, 3418-3422. doi:10.1021/n1802218f
Meier, C., Engels, J. W., 1992. Peptide Nucleic Acids(PNAs)—Unusual Properties of Nonionic Oligonucleotide Analogues. Angew. Chem. Int. Ed. Engl. 31, 1008-1010. doi:10.1002/anie.199210081
Meller, A., Nivon, L., Brandin, E., Golovchenko, J., Branton, D., 2000. Rapid nanopore discrimination between single polynucleotide molecules. Proc. Natl. Acad. Sci. U.S.A 97, 1079-1084.
Meller, A., Weng, Z., 2012. Sequence preserved dna conversion. US20120040869 A1.
Mesmaeker, A. D., Lebreton, J., Waldner, A., Cook, P. D., 1997. Backbone modified oligonucleotide analogs. U.S. Pat. No. 5,602,240 A.
Metzker, M. L., 2010. Sequencing technologies—the next generation. Nat. Rev. Genet. 11, 31-46. doi:10.1038/nrg2626
Metzker, M. L., 2005. Emerging technologies in DNA sequencing. Genome Res. 15, 1767-1776. doi:10.1101/gr.3770505
Metzker, M. L., Raghavachari, R., Richards, S., Jacutin, S. E., Civitello, A., Burgess, K., Gibbs, R. A., 1994. Termination of DNA synthesis by novel 3′-modified-deoxyribonucleoside 5′-triphosphates. Nucleic Acids Res. 22, 4259-4267.
Michalet, X., Ekong, R., Fougerousse, F., Rousseaux, S., Schurra, C., Hornigold, N., van Slegtenhorst, M., Wolfe, J., Povey, S., Beckmann, J. S., Bensimon, A., 1997. Dynamic molecular combing: stretching the whole human genome for high-resolution studies. Science 277, 1518-1523.
Morgan, R. D., Calvet, C., Demeter, M., Agra, R., Kong, H., 2000. Characterization of the specific DNA nicking activity of restriction endonuclease N.BstNBI. Biol. Chem. 381, 1123-1125. doi:10.1515/BC.2000.137
Mukaiyama, A., Takano, K., Haruki, M., Morikawa, M., Kanaya, S., 2004. Kinetically robust monomeric protein from a hyperthermophile. Biochemistry (Mosc.) 43, 13859-13866. doi:10.1021/bi0487645
Myers, T. W., Gelfand, D. H., 1991. Reverse transcription and DNA amplification by a Thermus thermophilus DNA polymerase. Biochemistry (Mosc.) 30, 7661-7666.
Nam, S.-W., Rooks, M. J., Kim, K.-B., Rossnagel, S. M., 2009. Ionic field effect transistors with sub-10 nm multiple nanopores. Nano Lett. 9, 2044-2048. doi:10.1021/nl900309s
Nelson, D. L., Cox, M. M., 2012. Lehninger Principles of Biochemistry, Sixth Edition edition. ed. W.H. Freeman, New York.
Nikiforov, T. T., Rendle, R. B., Kotewicz, M. L., Rogers, Y. H., 1994. The use of phosphorothioate primers and exonuclease hydrolysis for the preparation of single-stranded PCR products and their detection by solid-phase hybridization. Genome Res. 3, 285-291.
Obika, S., Tomizu, M., Negoro, Y., Osaki, T., Orita, A., Ueyama, Y., Nakagawa, O., Imanishi, T., 2007. Acid-mediated cleavage of oligonucleotide P3′->N5′ phosphoramidates triggered by sequence-specific triplex formation. Nucleosides Nucleotides Nucleic Acids 26, 893-896. doi:10.1080/15257770701505741
Ohtani, N., Haruki, M., Morikawa, M., Crouch, R. J., Itaya, M., Kanaya, S., 1999. Identification of the genes encoding Mn2+-dependent RNase HII and Mg2+-dependent RNase HIII from Bacillus subtilis: classification of RNases H into three families. Biochemistry (Mosc.) 38, 605-618. doi:10.1021/bi982207z
Ohtani, N., Haruki, M., Muroya, A., Morikawa, M., Kanaya, S., 2000. Characterization of ribonuclease HII from Escherichia coli overproduced in a soluble form. J. Biochem. (Tokyo) 127, 895-899.
Ohtani, N., Tomita, M., Itaya, M., 2008. Junction ribonuclease activity specified in RNases HII/2. FEBS J. 275, 5444-5455. doi:10.1111/j.1742-4658.2008.06673.x
Ohtani, N., Yanagawa, H., Tomita, M., Itaya, M., 2004. Identification of the first archaeal Type 1 RNase H gene from Halobacterium sp. NRC-1: archaeal RNase HI can cleave an RNA-DNA junction. Biochem. J. 381, 795-802. doi:10.1042/BJ20040153
Okabe, S., Hirokawa, N., 1993. Do photobleached fluorescent microtubules move?: re-evaluation of fluorescence laser photobleaching both in vitro and in growing Xenopus axon. J. Cell Biol. 120, 1177-1186.
Olejnik, J., Ludemann, H. C., Krzymanska-Olejnik, E., Berkenkamp, S., Hillenkamp, F., Rothschild, K J., 1999. Photocleavable peptide-DNA conjugates: synthesis and applications to DNA analysis using MALDI-MS. Nucleic Acids Res. 27, 4626-4631.
Olejnik, J., Sonar, S., Krzymariska-Olejnik, E., Rothschild, K. J., 1995. Photocleavable biotin derivatives: a versatile approach for the isolation of biomolecules. Proc. Natl. Acad. Sci. U.S.A 92, 7590-7594.
Otobe, K., Ohtani, T., 2001. Behavior of DNA fibers stretched by precise meniscus motion control. Nucleic Acids Res. 29, E109.
Patel, P. H., Loeb, L. A., 2003. Mutant enzymatic protein for use as tool in human therapeutics and diagnostics. U.S. Pat. No. 6,602,695 B2.
Patel, P. H., Loeb, L. A., 2001. A mutant polymerase having asptyrserglnilegluleuarg amino acid sequence in the active site and possesses altered fidelity or altered catalytic activity. U.S. Pat. No. 6,329,178 B1.
Perkins, T. T., Quake, S. R., Smith, D. E., Chu, S., 1994. Relaxation of a single DNA molecule observed by optical microscopy. Science 264, 822-826.
Piepenburg, O., Stemple, D. L., Armes, N. A., 2012. 2-nitrobenzyl-modified ribonucleotides. U.S. Pat. No. 8,299,226 B2.
Pierceall, W., Steinmann, K., Causey, M., Raz, T., Jarosz, M., Buzby, P., Thompson, J., 2010. Methods of sample preparation for nucleic acid analysis for nucleic acids available in limited amounts. WO2010048386 A1.
Pratt, M., Bryant, J., 2013. Fluorescence excitation and detection system and method. U.S. Pat. No. 8,436,999 B2.
Quake, S., 2011. Methods and kits for analyzing polynucleotide sequences. U.S. Pat. No. 7,981,604 B2.
RAWLS, R. L., 1997. OPTIMISTIC ABOUT ANTISENSE. Chem. Eng. News Arch. 75, 35-39. doi:10.1021/cen-v075n022.p035
Riehn, R., Lu, M., Wang, Y.-M., Lim, S. F., Cox, E. C., Austin, R. H., 2005. Restriction mapping in nanofluidic devices. Proc. Natl. Acad. Sci. U.S.A 102, 10012-10016. doi:10.1073/pnas.0503809102
Rigatti, R., Ost, T. W. B., 2010. Method for pair-wise sequencing a plurality of target polynucleotides. U.S. Pat. No. 7,754,429 B2.
Romaniuk, P. J., Eckstein, F., 1982. A study of the mechanism of T4 DNA polymerase with diastereomeric phosphorothioate analogues of deoxyadenosine triphosphate. J. Biol. Chem. 257, 7684-7688.
Roychoudhury, R., Wu, R., 1977. Novel properties of Escherichia coli exonuclease III. J. Biol. Chem. 252, 4786-4789.
R S Brody, S. A., 1982. Stereochemical course of nucleotidyl transfer catalyzed by bacteriophage T7 induced DNA polymerase. Biochemistry (Mosc.) 21, 2570-2. doi:10.1021/bi00539a042
Rubenstein, K., Ullman, E., 1974. Enzyme amplification assay. U.S. Pat. No. 3,817,837 A.
Ruth, J. L., Cheng, Y. C., 1981. Nucleoside analogues with clinical potential in antivirus chemotherapy. The effect of several thymidine and 2′-deoxycytidine analogue 5′-triphosphates on purified human (alpha, beta) and herpes simplex virus (types 1, 2) DNA polymerases. Mol. Pharmacol. 20, 415-422.
Rydberg, B., Game, J., 2002. Excision of misincorporated ribonucleotides in DNA by RNase H (type 2) and FEN-1 in cell-free extracts. Proc. Natl. Acad. Sci. U.S.A 99, 16654-16659. doi:10.1073/pnas.262591699
Samuelson, J. C., Zhu, Z., Xu, S., 2004. The isolation of strand-specific nicking endonucleases from a randomized SapI expression library. Nucleic Acids Res. 32, 3661-3671. doi:10.1093/nar/gkh674
Sanghvi, Y. S., Cook, P. D. (Eds.), 1994. Carbohydrate Modifications in Antisense Research. American Chemical Society, Washington, D.C.
Sato, A., Kanai, A., Itaya, M., Tomita, M., 2003. Cooperative regulation for Okazaki fragment processing by RNase HII and FEN-1 purified from a hyperthermophilic archaeon, Pyrococcus furiosus. Biochem. Biophys. Res. Commun. 309, 247-252.
Sauer, S., Lechner, D., Berlin, K., Lehrach, H., Escary, J.-L., Fox, N., Gut, I. G., 2000. A novel procedure for efficient genotyping of single nucleotide polymorphisms. Nucleic Acids Res. 28, e13-e13. doi:10.1093/nar/28.5.e13
Sawai, H., 1984. SYNTHESIS AND PROPERTIES OF OLIGOADENYLIC ACIDS CONTAINING 2&prime;-5&prime; PHOSPHORAMIDE LINKAGE. Chem. Lett. 13, 805-808. doi:10.1246/cl.1984.805
Schmidt, V. K., Sorensen, B. S., Sorensen, H. V., Alsner, J., Westergaard, O., 1994. Intramolecular and intermolecular DNA ligation mediated by topoisomerase II. J. Mol. Biol. 241, 18-25. doi:10.1006/jmbi.1994.1469
Schuurs, A., Van, W. B., 1974. Process for the demonstration and determination of low molecular compounds and of proteins capable of binding these compounds specifically. U.S. Pat. No. 3,850,752 A.
Seela, F., Thomas, H., 2000. Modified oligonucleotides, their preparation and their use. U.S. Pat. No. 6,150,510 A.
Shera, E. B., 1988. Single molecule tracking. U.S. Pat. No. 4,793,705 A.
Shinozaki, K., Okazaki, T., 1978. T7 gene 6 exonuclease has an RNase H activity. Nucleic Acids Res. 5, 4245-4261.
Shuga, J., Zeng, Y., Novak, R., Lan, Q., Tang, X., Rothman, N., Vermeulen, R., Li, L., Hubbard, A., Zhang, L., Mathies, R. A., Smith, M. T., 2013. Single molecule quantitation and sequencing of rare translocations using microfluidic nested digital PCR. Nucleic Acids Res. 41, e159. doi:10.1093/nar/gkt613
Simmons, R. M., Finer, J. T., Chu, S., Spudich, J. A., 1996. Quantitative measurements of force and displacement using an optical trap. Biophys. J. 70, 1813-1822. doi:10.1016/50006-3495(96)79746-1
Siwy, Z. S., Howorka, S., 2010. Engineered voltage-responsive nanopores. Chem. Soc. Rev. 39, 1115-1132. doi:10.1039/b909105j
Sorenson, J., Tang, S., Marks, P., CHIN, C.-S., 2013. Algorithms for sequence determination. U.S. Pat. No. 8,370,079 B2.
Sparks, J. L., Chon, H., Cerritelli, S. M., Kunkel, T. A., Johansson, E., Crouch, R. J., Burgers, P. M., 2012. RNase H2-Initiated Ribonucleotide Excision Repair. Mol. Cell 47, 980-986. doi:10.1016/j.molcel.2012.06.035
Sprinzl, M., Sternbach, H., Von Der Haar, F., Cramer, F., 1977. Enzymatic Incorporation of ATP and CTP Analogues into the 3′ End of tRNA. Eur. J. Biochem. 81, 579-589. doi:10.1111/j.1432-1033.1977.tb11985.x
Stemmer, W. P., Crameri, A., Ha, K. D., Brennan, T. M., Heyneker, H. L., 1995. Single-step assembly of a gene and entire plasmid from large numbers of oligodeoxyribonucleotides. Gene 164, 49-53.
Stigter, D., Bustamante, C., 1998. Theory for the hydrodynamic and electrophoretic stretch of tethered B-DNA. Biophys. J. 75, 1197-1210. doi:10.1016/S0006-3495(98)74039-1
Storm, A. J., Chen, J. H., Ling, X. S., Zandbergen, H. W., Dekker, C., 2003. Fabrication of solid-state nanopores with single-nanometre precision. Nat. Mater. 2, 537-540. doi:10.1038/nmat941
Summerton, J. E., Weller, D. D., 1991. Uncharged morpholino-based polymers having achiral intersubunit linkages. U.S. Pat. No. 5,034,506 A.
Summerton, J. E., Weller, D. D., Stirchak, E. P., 1993. Alpha-morpholino ribonucleoside derivatives and polymers thereof. U.S. Pat. No. 5,235,033 A.
Su, X., Berlin, A. A., 2006. Method and apparatus for nucleic acid sequencing and identification. U.S. Pat. No. 7,005,264 B2.
Tabor, S., Richardson, C. C., 1987. DNA sequence analysis with a modified bacteriophage T7 DNA polymerase. Proc. Natl. Acad. Sci. U.S.A 84, 4767-4771.
Takagi, M., Nishioka, M., Kakihara, H., Kitabayashi, M., Inoue, H., Kawakami, B., Oka, M., Imanaka, T., 1997. Characterization of DNA polymerase from Pyrococcus sp. strain KOD1 and its application to PCR. Appl. Environ. Microbiol. 63, 4504-4510.
Telser, J., Cruickshank, K. A., Morrison, L. E., Netzel, T. L., 1989. Synthesis and characterization of DNA oligomers and duplexes containing covalently attached molecular labels: comparison of biotin, fluorescein, and pyrene labels by thermodynamic and optical spectroscopic measurements. J. Am. Chem. Soc. 111, 6966-6976. doi:10.1021/ja00200a011
Thompson, J. F., Steinmann, K. E., 2010. Single molecule sequencing with a HeliScope genetic analysis system. Curr. Protoc. Mol. Biol. Ed. Frederick M Ausubel A1 Chapter 7, Unit7.10. doi:10.1002/0471142727.mb0710s92
Till, B. J., Burtner, C., Comai, L., Henikoff, S., 2004. Mismatch cleavage by single-strand specific nucleases. Nucleic Acids Res. 32, 2632-2641. doi:10.1093/nar/gkh599
Tomaney, A. B., Maxham, K. M., Holden, D., Hester, K., Murphy, D., Marks, P., 2009. Methods and processes for calling bases in sequence by incorporation methods. US20090024331 A1
Tom, H. K., Rowley, G. L., 1982. Concentrating zone method in heterogeneous immunoassays. U.S. Pat. No. 4,366,241 A.
Tseung, K. K., Takayama, G., Rhett, N. K., Corl, M. V., 2004. Method for automated staining of specimen slides. U.S. Pat. No. 6,746,851 B1.
Ts'o, POP., Miller, P. S., 1984. Nonionic nucleic acid alkyl and aryl phosphonates and processes for manufacture and use thereof. U.S. Pat. No. 4,469,863 A.
Turchi, J. J., Huang, L., Murante, R. S., Kim, Y., Bambara, R. A., 1994. Enzymatic completion of mammalian lagging-strand DNA replication. Proc. Natl. Acad. Sci. 91, 9803-9807.
Turner, S., Flusberg, B., Foquet, M., Callebaut, H., Sebra, R., Chaudhuri, B., Sorenson, J., Bjornson, K., Fehr, A., Korlach, J., Emig, R., 2010. Nanopore sequencing devices and methods. US20100331194 A1.
Turner, S., Korlach, J., 2013. Modified base detection with nanopore sequencing. US20130327644 A1.
Ullman, E. F., Schwarzberg, M., 1976. Fluorescence quenching with immunological pairs in immunoassays. U.S. Pat. No. 3,996,345 A.
Urdea, M. S., 1995. Polynucleotide reagents containing selectable cleavage sites. U.S. Pat. No. 5,380,833 A.
Vaghefi, M. (Ed.), 2005. Nucleoside Triphosphates and their Analogs: Chemistry, Biotechnology, and Biological Applications, 1 edition. ed. CRC Press.
Venkatesan, B. M., Dorvel, B., Yemenicioglu, S., Watkins, N., Petrov, I., Bashir, R., 2009. Highly Sensitive, Mechanically Stable Nanopore Sensors for DNA Analysis. Adv. Mater. Deerfield Beach Fla. 21, 2771.
Venkatesan, B. M., Polans, J., Comer, J., Sridhar, S., Wendell, D., Aksimentiev, A., Bashir, R., 2011. Lipid bilayer coated Al(2)O(3) nanopore sensors: towards a hybrid biological solid-state nanopore. Biomed. Microdevices 13, 671-682. doi:10.1007/s10544-011-9537-3
Venta, K., Shemer, G., Puster, M., Rodriguez-Manzo, J. A., Balan, A., Rosenstein, J. K., Shepard, K., Drndić, M., 2013. Differentiation of short, single-stranded DNA homopolymers in solid-state nanopores. ACS Nano 7, 4629-4636. doi:10.1021/nn4014388
Verma, I. M., 1977. The reverse transcriptase. Biochim. Biophys. Acta 473, 1-38.
Voelkerding, K. V., Dames, S. A., Durtschi, J. D., 2009. Next-generation sequencing: from basic research to diagnostics. Clin. Chem. 55, 641-658. doi:10.1373/clinchem.2008.112789
von Kiedrowski, G., Wlotzka, B., Helbing, J., Matzen, M., Jordan, S., 1991. Parabolic Growth of a Self-Replicating Hexadeoxynucleotide Bearing a 3′-5′-Phosphoamidate Linkage. Angew. Chem. Int. Ed. Engl. 30, 423-426. doi:10.1002/anie.199104231
Vyle, J. S., Connolly, B. A., Kemp, D., Cosstick, R., 1992. Sequence- and strand-specific cleavage in oligodeoxyribonucleotides and DNA containing 3′-thiothymidine. Biochemistry (Mosc.) 31, 3012-3018.
Walker, G. T., Little, M. C., Nadeau, J. G., Shank, D. D., 1992. Isothermal in vitro amplification of DNA by a restriction enzyme/DNA polymerase system. Proc. Natl. Acad. Sci. 89, 392-396.
Wang, H., Hays, J. B., 2000. Preparation of DNA substrates for in vitro mismatch repair. Mol. Biotechnol. 15, 97-104. doi:10.1385/MB:15:2:97
Wang, W., Billen, L. P., Li, Y., 2002. Sequence Diversity, Metal Specificity, and Catalytic Proficiency of Metal-Dependent Phosphorylating DNA Enzymes. Chem. Biol. 9, 507-517. doi:10.1016/S1074-5521(02)00127-8
Wanunu, M., Meller, A., 2007. Chemically modified solid-state nanopores. Nano Lett. 7, 1580-1585. doi:10.1021/n1070462b
Wedekind, P., Kubitscheck, U., Peters, R., 1994. Scanning microphotolysis: a new photobleaching technique based on fast intensity modulation of a scanned laser beam and confocal imaging. J. Microsc. 176, 23-33.
White, R. J., Zhang, B., Daniel, S., Tang, J. M., Ervin, E. N., Cremer, P. S., White, H. S., 2006. Ionic conductivity of the aqueous layer separating a lipid bilayer membrane and a glass support. Langmuir ACS J. Surf. Colloids 22, 10777-10783. doi:10.1021/1a061457a
Williams, J., Anderson, J., Urlacher, T., Steffens, D., 2007. Mutant polymerases for sequencing and genotyping. US20070048748 A1.
Williams, J. G. K., 2013. System and method for nucleic acid sequencing by polymerase synthesis. U.S. Pat. No. 8,530,154 B2.
Williams, P., Hayes, M. A., Rose, S. D., Bloom, L. B., Reha-Krantz, L. J., Pizziconi, V. B., 2006. Sequencing DNA using polymerase, fluorescence, chemiluminescence, thermopile, thermistor and refractive index measurements; microcalorimetric detection. U.S. Pat. No. 7,037,687 B2.
Wu, A. M., Gallo, R. C., 1975. Reverse transcriptase. CRC Crit. Rev. Biochem. 3, 289-347.
Wu, H., Lima, W. F., Crooke, S T., 1999. Properties of cloned and expressed human RNase H1. J. Biol. Chem. 274, 28270-28278.
Wu, H., Lima, W. F., Crooke, S T., 1998. Molecular cloning and expression of cDNA for human RNase H. Antisense Nucleic Acid Drug Dev. 8, 53-61.
Wu, J., Zhang, S., Meng, Q., Cao, H., Li, Z., Li, X., Shi, S., Kim, D. H., Bi, L., Turro, N. J., Ju, J., 2007. 3?-O-modified nucleotides as reversible terminators for pyrosequencing. Proc. Natl. Acad. Sci. U.S.A 104, 16462-16467. doi:10.1073/pnas.0707495104
Xie, X. S., Sims, P. A., Greenleaf, W. J., DUAN, H., 2013. Nucleic acid amplification and sequencing by synthesis with fluorogenic nucleotides. US20130053252 A1.
Xu, Y., Lunnen, K. D., Kong, H., 2001. Engineering a nicking endonuclease N.AlwI by domain swapping. Proc. Natl. Acad. Sci. 98, 12990-12995. doi:10.1073/pnas.241215698
Yang, Z., Sismour, A. M., Benner, S. A., 2007. Nucleoside alpha-thiotriphosphates, polymerases and the exonuclease III analysis of oligonucleotides containing phosphorothioate linkages. Nucleic Acids Res. 35, 3118-3127. doi:10.1093/nar/gkm168
Yau, E. K., 1997. Process for preparing phosphorothioate oligonucleotides. U.S. Pat. No. 5,644,048 A.
Yokota, H., Johnson, F., Lu, H., Robinson, R. M., Belu, A. M., Garrison, M. D., Ratner, B. D., Trask, B. J., Miller, D. L., 1997. A new method for straightening DNA molecules for optical restriction mapping. Nucleic Acids Res. 25, 1064-1070.
Yusko, E. C., Johnson, J. M., Majd, S., Prangkio, P., Rollings, R. C., Li, J., Yang, J., Mayer, M., 2011. Controlling protein translocation through nanopores with bio-inspired fluid walls. Nat. Nanotechnol. 6, 253-260. doi:10.1038/nnano.2011.12
Zavgorodny, S. G., Pechenov, A. E., Shvets, V. I., Miroshnikov, A. I., 2000. S,X-acetals in nucleoside chemistry. III. Synthesis of 2′- and 3′-O-azidomethyl derivatives of ribonucleosides. Nucleosides Nucleotides Nucleic Acids 19, 1977-1991. doi:10.1080/15257770008045472
Zhu, Z., Samuelson, J. C., Zhou, J., Dore, A., Xu, S.-Y., 2004. Engineering strand-specific DNA nicking enzymes from the type ITS restriction endonucleases BsaI, BsmBI, and BsmAI. J. Mol. Biol. 337, 573-583. doi:10.1016/j.jmb.2004.02.003
Zimmermann, R. M., Cox, E. C., 1994. DNA stretching on functionalized gold surfaces. Nucleic Acids Res. 22, 492-497.

What is claimed is:

1. A method of associating a removable tail with a nucleotide comprising a predetermined base type, said removable tail not being associated with said nucleotide prior to its incorporation into a nucleic acid molecule, said method applied to one or more nucleic acid molecules, and said method comprising the steps of:
(i) exposing a nucleic acid molecule comprising an extendable 3′ end to a solution and conditions to cause incorporation of a nucleotide comprising said predetermined base type into said nucleic acid molecule;
(ii) subjecting said nucleic acid molecule to a process to cause association of a blocking tail with said nucleic acid molecule, said association occurring in the event that no incorporation occurs in step (i); and
(iii)subjecting said nucleic acid molecule to a process to cause association of a removable tail with a nucleotide incorporated in step (i), said association occurring in the event that incorporation occurs in step (i).

2. The method according to claim 1, wherein a nucleic acid molecule comprises an extendable 3′ end; wherein step (ii) precedes step (i); wherein step (iii) is replaced by a step following step (ii) and preceding step (i), said step comprising subjecting the nucleic acid molecule to a process to cause association of a removable tail with the nucleic acid molecule, said association occurring in the event that no blocking tail is associated with the nucleic acid molecule in step (ii); and wherein step (i) is conducted last and comprises subjecting the nucleic acid molecule to a process to cause removal of the removable tail that may be associated with the nucleic acid molecule, restoring the extendable 3′ end of the nucleic acid molecule, and exposing the nucleic acid molecule to a solution and conditions to cause incorporation of a nucleotide comprising a predetermined base type at said extendable 3′ end.

3. The method according to claim 1, wherein a removable nucleotide tail extending from the 3′ end of a nucleotide comprising a predetermined base type is constructed; and wherein construction of a removable nucleotide tail in step (iii) is preceded by or concurrently conducted with unblocking in the event that the solution in step (i) comprises blocked nucleotides.

4. The method according to claim 3, wherein steps (i) and (ii) are conducted simultaneously; and wherein the blocking nucleotide tail is constructed to comprise a single nucleotide that is blocked and cleavable.

5. The method according to claim 4, wherein the removable nucleotide tail is a ligatable removable nucleotide tail, and further comprising step (iv) comprising a process to cause attachment of a tail tag to the nucleic acid molecule, said attachment occurring in the event that a ligatable removable nucleotide tail is constructed in step (iii), and said tail tag comprising one or more specific sequences, or one or more labels, or one or more other detectable features, or a combination thereof, designated to represent the predetermined base type in step (i).

6. The method according to claim 3, further comprising the steps of:
(iv) detecting the presence of the removable nucleotide tail constructed in step (iii), and removing the blocking nucleotide tail that may be constructed in step (ii) and the removable nucleotide tail that may be constructed in step (iii); and
(v) repeating steps (i) through (iv) at least one time, thereby allowing sequencing of the nucleic acid molecule.

7. The method according to claim 3, wherein the removable nucleotide tail is a ligatable removable nucleotide tail, and further comprising step (iv) comprising a process to cause attachment of a tail tag to the nucleic acid molecule, said attachment occurring in the event that a ligatable removable nucleotide tail is constructed in step (iii), said step (iv) optionally conducted concurrently with step (iii), and said tail tag comprising one or more specific sequences, or one or more labels, or one or more other detectable features, or a combination thereof, designated to represent the predetermined base type in step (i).

8. The method according to claim 3, wherein step (ii) is omitted; and wherein step (i) comprises exposing the nucleic acid molecule to conditions to cause nucleotide incorporation into said nucleic acid molecule, and to a polymerization reaction solution comprising a population of blocked nucleotides to complement the nucleic acid molecule, said population comprising: (a) nucleotides comprising one base type, that are reversibly blocked with a terminator type that is different from the types of terminators comprised in the nucleotides comprising other base types, and (b) one base type being a predetermined base type of step (i).

9. The method according to claim 3, wherein steps (i) and (ii) are conducted simultaneously; wherein any constructed blocking nucleotide tail comprises a single nucleotide that is blocked and cleavable; and wherein the combined steps (i) and (ii) comprise exposing the nucleic acid molecule to conditions to cause nucleotide incorporation into said nucleic acid molecule, and to a polymerization reaction solution comprising reversibly blocked nucleotides comprising a predetermined base type, and blocked cleavable nucleotides not comprising the predetermined base type.

10. The method according to claim 3, wherein the nucleic acid molecule comprises more than one extendable 3′ ends.

11. The method according to claim 7, wherein step (iv) is followed by steps (v) and (vi), said step (v) comprising subjecting the nucleic acid molecule to a process to cause removal of any nucleotide tails that may be constructed in previous steps, and said step (vi) comprising repeating steps (i) through (v) at least once.

12. The method according to claim 11, wherein tail tags comprise labels causing changes in conductivity or specific sequences causing changes in conductivity or both, and wherein at least part of the nucleic acid molecule comprising tail tags passes through a nanopore of a nanopore device, thereby allowing detection of labels or specific sequences or both.

13. The method according to claim 11, wherein tail tags comprise labels causing changes in conductivity or specific sequences causing changes in conductivity, wherein the predetermined base type in step (i) is represented by at least two different label types or at least two different tail tag sequences, and wherein at least part of the nucleic acid molecule comprising tail tags passes through a nanopore of a nanopore device, thereby allowing detection of labels or specific sequences.

14. The method according to claim 1, wherein step (ii) precedes step (i); wherein step (ii) is preceded by a step comprising forming a single-base gap beginning at the extendable 3′ end of the nucleic acid molecule; and wherein step (i) comprises exposing the nucleic acid molecule to conditions to cause nucleotide incorporation into said single-base gap.

15. The method according to claim 1, wherein step (ii) precedes step (i); and wherein step (ii) is followed by a step comprising subjecting the nucleic acid molecule to a process to cause formation of a single-base gap beginning at the extendable 3′ end of the nucleic acid molecule, said formation occurring in the event that there is no blocking nucleotide tail constructed in step (ii).

16. A method of incorporating a nucleotide into a nucleic acid molecule comprising an extendable 3′ end, said nucleotide comprising a predetermined base type and a 3′ end suitable for constructing a removable nucleotide tail, said method applied to one or more nucleic acid molecules, and said method comprising the steps of:
(i) exposing the nucleic acid molecule to conditions to cause nucleotide incorporation, and to a polymerization reaction solution comprising blocked nucleotides comprising a predetermined base type;
(ii) subjecting the nucleic acid molecule to a process to cause construction of a blocking nucleotide tail extending from the extendable 3′ end of the nucleic acid molecule, said construction occurring in the event that no nucleotide incorporation occurs in step (i); and
(iii) subjecting the nucleic acid molecule to a process to cause replacement of a blocked nucleotide by an unblocked nucleotide comprising the predetermined type of step (i), said replacement occurring in the event that nucleotide incorporation occurs in step (i), and said unblocked nucleotide maintaining an extendable 3′-end.

17. A method of constructing a removable nucleotide tail extending from the 3′ end of a nucleotide incorporated into a nucleic acid molecule, said nucleotide comprising a predetermined base type, said nucleic acid molecule comprising an extendable 3′ end, said method applied to one or more nucleic acid molecules, and said method comprising the steps of:
(i) exposing the nucleic acid molecule to conditions to cause nucleotide incorporation, and to a polymerization reaction solution comprising cleavable nucleotides comprising a predetermined base type;
(ii) subjecting the nucleic acid molecule to a process to cause a single cleavable nucleotide with extendable 3′ end to remain incorporated into the nucleic acid molecule, said nucleotide being incorporated during step (i);
(iii) subjecting the nucleic acid molecule to a process to cause construction of a terminal blocking nucleotide tail, said construction occurring in the event that no nucleotide incorporation occurs in step (i);
(iv) subjecting the nucleic acid molecule to a process to cause construction of a removable nucleotide tail extending from the 3′ end of the cleavable nucleotide in step (ii), said construction occurring in the event that nucleotide incorporation occurs in step (i); and
(v) subjecting the nucleic acid molecule to a process to cause replacement of the cleavable nucleotide in step (ii) with a non-cleavable nucleotide, said replacement occurring in the event that nucleotide incorporation occurs in step (i).

18. The method according to claim 17, wherein the removable nucleotide tail is ligatable, wherein step (iv) is followed by a step comprising a process to cause tail tag ligation, said ligation occurring in the event that a ligatable removable nucleotide tail is constructed in step (iv), and wherein the process of replacement in step (v) comprises gap formation and subsequent filling, and said tail tag comprising one or more specific sequences, or one or more labels, or one or more other detectable features, or a combination thereof, designated to represent the predetermined base type in step (i).


SEQUENCE LISTING (32 sequences)

SEQ ID NO: 1 - DNA, 114 bp, Artificial Sequence
  Oligo
tctacgacacacacacacacacacacacacacacacacacacacacacacacacacacacacacacacacacacacacacacacacacacacacacacacacacacgtcaagct

SEQ ID NO: 2 - DNA, 107 bp, Artificial Sequence
  Oligo
gcttgacgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgtgt

SEQ ID NO: 3 - DNA, 114 bp, Artificial Sequence
  Oligo
tctacgaaaaaaaaaaaaaaaaaaaaaaaaaaaaaaaaaaaaaaaaaaaaaaaaaaccccccccccccccccccccccccccccccccccccccccccccccccccgtcaagct

SEQ ID NO: 4 - DNA, 107 bp, Artificial Sequence
  Oligo
gcttgacggggggggggggggggggggggggggggggggggggggggggggggggggtttttttttttttttttttttttttttttttttttttttttttttttttt

SEQ ID NO: 5 - DNA, 114 bp, Artificial Sequence
  Oligo
tctacgtctctctctctctctctctctctctctctctctctctctctctctctctctctctctctctctctctctctctctctctctctctctctctctctctctcgtcaagct

SEQ ID NO: 6 - DNA, 107 bp, Artificial Sequence
  Oligo
gcttgacgagagagagagagagagagagagagagagagagagagagagagagagagagagagagagagagagagagagagagagagagagagagagagagagagaga

SEQ ID NO: 7 - DNA, 114 bp, Artificial Sequence
  Oligo
tctacgttttttttttttttttttttttttttttttttttttttttttttttttttccccccccccccccccccccccccccccccccccccccccccccccccccgtcaagct

SEQ ID NO: 8 - DNA, 107 bp, Artificial Sequence
  Oligo
gcttgacggggggggggggggggggggggggggggggggggggggggggggggggggaaaaaaaaaaaaaaaaaaaaaaaaaaaaaaaaaaaaaaaaaaaaaaaaaa

SEQ ID NO: 9 - DNA, 78 bp, Artificial Sequence
  Hairpin Adaptor
gactcggaagagcgtcgtgtagggaaagagtgtagtttttttctacactctttccctacacgacgctcttccgagtct

SEQ ID NO: 10 - DNA, 66 bp, Artificial Sequence
  Hairpin Tail Tag
cttctctctctcttctctctttttgagctcggtaaccttggtttaagagagaagagagagagaagt

SEQ ID NO: 11 - DNA, 66 bp, Artificial Sequence
  Hairpin Tail Tag
gagaagaaggagaagagaggatttgagctcggtaaccttggttttcctctcttctccttcttctct

SEQ ID NO: 12 - DNA, 66 bp, Artificial Sequence
  Hairpin Tail Tag
gtgtggttgtgtgttgtggtttttgagctcggtaaccttggtttaaccacaacacacaaccacact

SEQ ID NO: 13 - DNA, 64 bp, Artificial Sequence
  Hairpin Tail Tag
ccacaccacacacaccacactttgagctcggtaaccttggtttgtgtggtgtgtgtggtgtggt

SEQ ID NO: 14 - DNA, 63 bp, Artificial Sequence
  Tail Tag
gttacccttctctctctcttctctcttcaactccagtcacatcaggatctcagatggcgtctt

SEQ ID NO: 15 - DNA, 58 bp, Artificial Sequence
  Oligo
aagacgccatctgagatcctgatgtgactggagttgaagagagaagagagagagaagg

SEQ ID NO: 16 - DNA, 63 bp, Artificial Sequence
  Tail Tag
gttaccgagaagaaggagaagagaggacaactccagtcacatcaggatctcagatggcgtctt

SEQ ID NO: 17 - DNA, 58 bp, Artificial Sequence
  Oligo
aagacgccatctgagatcctgatgtgactggagttgtcctctcttctccttcttctcg

SEQ ID NO: 18 - DNA, 63 bp, Artificial Sequence
  Tail Tag
gttaccgtgtggttgtgtgttgtggttcaactccagtcacatcaggatctcagatggcgtctt

SEQ ID NO: 19 - DNA, 58 bp, Artificial Sequence
  Oligo
aagacgccatctgagatcctgatgtgactggagttgaaccacaacacacaaccacacg

SEQ ID NO: 20 - DNA, 63 bp, Artificial Sequence
  Tail Tag
gttaccaccacaccacacacaccacaccaactccagtcacatcaggatctcagatggcgtctt

SEQ ID NO: 21 - DNA, 58 bp, Artificial Sequence
  Oligo
aagacgccatctgagatcctgatgtgactggagttggtgtggtgtgtgtggtgtggtg

SEQ ID NO: 22 - DNA, 36 bp, Artificial Sequence
  Primer
ctacactctttccctacacgacgctcttccgagtct

SEQ ID NO: 23 - DNA, 36 bp, Artificial Sequence
  Primer
aagacgccatctgagatcctgatgtgactggagttg

SEQ ID NO: 24 - DNA, 116 bp, Artificial Sequence
  Oligo
cgttgctgttctctgttccctcgttgtcgtttgtcgttcgttcgtgatcgactctgtcgccgcgtgtgttgctgctcccgcgtgtgttgctgctccaaaaaaaaaaaaaaaaaaaa

SEQ ID NO: 25 - DNA, 46 bp, Artificial Sequence
  Primer
cgttgctgttctctgttccctcgttgtcgtttgtcgttcgttcgtg

SEQ ID NO: 26 - DNA, 54 bp, Artificial Sequence
  Oligo
cgtttgtcgttcgttcgtgatcgacucugucactgactcagctacagtcatggt

SEQ ID NO: 27 - DNA, 54 bp, Artificial Sequence
  Oligo
cgtttgtcgttcgttcgtgatcgactctgtcactgactcagctacagtcatggt

SEQ ID NO: 28 - DNA, 47 bp, Artificial Sequence
  Oligo
accatgactgtagctgagtcagtgcgtttgtcgttcgttcgtgatcg

SEQ ID NO: 29 - DNA, 47 bp, Artificial Sequence; identical to SEQ ID NO: 28
  Oligo

SEQ ID NO: 30 - DNA, 57 bp, Artificial Sequence
  Template
gacctacgatgagacctagactcacctcgatcacgaacgaacgacaaacgacaacga

SEQ ID NO: 31 - DNA, 26 bp, Artificial Sequence
  Primer
gacctacgatgagacctagactcacc

SEQ ID NO: 32 - DNA, 21 bp, Artificial Sequence
  Oligo
cttctctctctcttctctctt